(19) United States
(12) Patent Application Publication
(10) Pub. No.: US 2025/0049929 A1
(43) Pub. Date: Feb. 13, 2025

(54) NOVEL TETRAHYDROQUINOLINES AND PROTEOLYSIS TARGETING CHIMERA (PROTACS) COMPRISING THEM AS DEGRADERS OF SMARCA

(71) Applicant: Boehringer Ingelheim International GmbH, Ingelheim am Rhein (DE)

(72) Inventors: Alessio CIULLI, Dundee, Scotland (GB); Emelyne DIERS, Mölndal (SE); William FARNABY, St. Andrews, Fife, Scotland (GB); Peter GREB, Vienna (AT); Christiane KOFINK, Perchtoldsdorf (AT); Steffen STEURER, Vienna (AT); Nicole TRAINOR, Melbourne (AU); Harald WEINSTABL, Vienna (AT)

(21) Appl. No.: 18/695,941

(22) PCT Filed: Sep. 27, 2022
(86) PCT No.: PCT/EP2022/076850

§ 371 Date: Mar. 27, 2024

(30) Foreign Application Priority Data
Sep. 29, 2021 (EP) 21199982.6

(51) Int. Cl. A61K 47/55; C07D 487/04; C07D 487/14; C07D 519/00

(52) U.S. Cl. CPC A61K 47/55; C07D 487/04; C07D 487/14; C07D 519/00

(57) ABSTRACT

The present invention encompasses compounds of formula (I), wherein the groups A, R¹, R³ and R⁴ have the meanings given in the claims and specification, proteolysis targeting chimera (PROTACs) comprising such compounds of formula (I), their use as degraders of SMARCA, pharmaceutical compositions which contain PROTACs of this kind and their medical uses, especially as agents for treatment and/or prevention of oncological diseases.

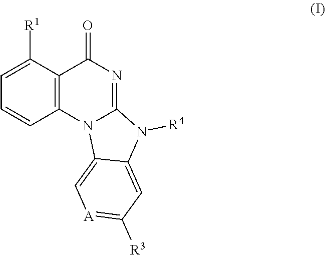

### FIELD OF THE INVENTION

The present invention relates to new tetrahydroquinolines and derivatives of formula (I):

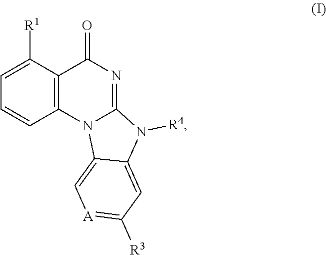

wherein the groups A, R¹, R and R have the meanings given in the claims and specification, which can be used as SMARCA binders and/or to prepare proteolysis targeting chimera (PROTACs). The present invention further relates to such PROTACs and derivatives, their use as degraders of SMARCA, pharmaceutical compositions which contain PROTACs of this kind and their medical uses, especially as agents for treatment and/or prevention of oncological diseases.

### BACKGROUND OF THE INVENTION

Classical small molecule drugs bind to their target proteins to modulate their activities, in most cases inhibiting them. In contrast, proteolysis targeting chimeras (PROTACs) bind to their target proteins to cause their degradation. PROTACs are tripartite molecules consisting of a part binding to the protein that is to be degraded, a second part that binds to an E3 ubiquitin ligase, and a linker. Whenever a trimeric complex consisting of the drug target, the PROTAC, and the ligase is formed, the close proximity of the ligase to the target results in target protein ubiquitylation. The multi-ubiquitin chain on the target protein is then recognized by the proteasome and the target protein is degraded (Collins et al., 2017; Hughes and Ciulli, 2017; Toure and Crews, 2016).

In contrast to classical small molecule drugs, PROTAC driven degradation functions in a sub-stoichiometric nature thus requiring lower systemic exposures to achieve efficacy (Bondeson et al., 2015; Winter et al., 2015). PROTACs have been shown to display higher degrees of selectivity for protein degradation than the target ligand itself due to complementarity differences in the protein-protein-interaction interfaces of the formed ternary complexes (Bondeson et al., 2018; Gadd et al., 2017; Nowak et al., 2018; Zengerle et al., 2015). In addition, PROTACs promise to expand the druggable proteome as degradation is not limited to the protein domain functionally responsible for the disease. In the case of challenging multidomain proteins, traditionally viewed as undruggable targets, the most ligandable domain can be targeted for degradation independent of its functionality or vulnerability to small molecule blockade (Gechijian et al., 2018).

The ATP-dependent activities of the BAF (SWI/SNF) chromatin remodeling complexes affect the positioning of nucleosomes on DNA and thereby many cellular processes related to chromatin structure, including transcription, DNA repair and decatenation of chromosomes during mitosis (Kadoch and Crabtree, 2015; St Pierre and Kadoch, 2017).

Several subunits of the BAF complex are recurrently mutated in human cancers, adding up to roughly 20% of human tumors in which at least one BAF complex subunit is mutated. The complex contains two mutually exclusive ATPases, SMARCA2 and SMARCA4.

SMARCA4 is amongst the recurrently mutated subunits in several tumor indications including lung, liver and colon. Mutations are not clustered in a particular part of the protein and therefore presumed to be mostly loss of function events (Hodges et al., 2016; Kadoch et al., 2013; Shain and Pollack, 2013; St Pierre and Kadoch, 2017). While SMARCA4 acts as a tumor suppressor in solid tumors, the role of SMARCA4 in acute myeloid leukemia (AML) is markedly different, such that it is required to maintain the oncogenic transcription program and drive proliferation (Shi et al., 2013). Selective suppression of SMARCA2 activity has been proposed as a therapeutic concept for SMARCA4 mutated cancers (Hoffman et al., 2014; Oike et al., 2013; Wilson et al., 2014).

Small molecule ligands targeting the bromodomains of SMARCA2 and SMARCA4 (SMARCA2/SMARCA4^{BD}) have been reported (Gerstenberger et al., 2016; Hoffman et al., 2014; Sutherell et al., 2016, Lu et al., 2018; WO 2016/138114).

PROTACs that degrade SMARCA2 and/or SMARCA4 have also been reported (Farnaby et al., 2019 and WO 2020/078933). These PROTACs are not selective for one ATPase over the other.

### REFERENCES

Bondeson, D. P. et al. (2015). Catalytic in vivo protein knockdown by small-molecule PROTACs. Nature Chemical Biology 11, 611.
Bondeson, D. P. et al. (2018). Lessons in PROTAC Design from Selective Degradation with a Promiscuous Warhead. Cell Chemical Biology 25, 78-87.e75.
Collins, I. et al. (2017). Chemical approaches to targeted protein degradation through modulation of the ubiquitin-proteasome pathway. Biochem J 474, 1127-1147.
Farnaby, W. et al. (2019). BAF complex vulnerabilities in cancer demonstrated via structure-based PROTAC design. Nature Chemical Biology 15,_672-680.
Gadd, M. S. et al. (2017). Structural basis of PROTAC cooperative recognition for selective protein degradation. Nature Chemical Biology 13, 514-521.
Gechijian, L. N. et al. (2018). Functional TRIM24 degrader via conjugation of ineffectual bromodomain and VHL ligands. Nature Chemical Biology 14, 405-412.
Gerstenberger, B. S. et al. (2016). Identification of a Chemical Probe for Family VIII Bromodomains through Optimization of a Fragment Hit. Journal of Medicinal Chemistry 59, 4800-4811.
Hodges, C. et al. (2016). The Many Roles of BAF (mSWI/SNF) and PBAF Complexes in Cancer. Cold Spring Harb Perspect Med 6.
Hoffman, G. R. et al. (2014). Functional epigenetics approach identifies BRM/SMARCA2 as a critical synthetic lethal target in BRG1-deficient cancers. Proceedings of the National Academy of Sciences 111, 3128-3133.
Hughes, S. J. and Ciulli, A. (2017). Molecular recognition of ternary complexes: a new dimension in the structure-guided design of chemical degraders. Essays Biochem 61, 505-516.
Kadoch, C. and Crabtree, G. R. (2015). Mammalian SWI/SNF chromatin remodeling complexes and cancer: Mechanistic insights gained from human genomics. Science Advances 1, e1500447-e1500447.
Kadoch, C. et al. (2013). Proteomic and bioinformatic analysis of mammalian SWI/SNF complexes identifies extensive roles in human malignancy. Nature Genetics 45, 592-601.
Lu, T. et al. (2018). Identification of small molecule inhibitors targeting the SMARCA2 bromodomain from a high-throughput screening assay. Acta Pharmacologoca Sinica 39, 1-9.
Nowak, R. P. et al. (2018). Plasticity in binding confers selectivity in ligand-induced protein degradation. Nature Chemical Biology.
Oike, T. et al. (2013). A synthetic lethality-based strategy to treat cancers harboring a genetic deficiency in the chromatin remodeling factor BRG1. Cancer Res 73, 5508-5518.
Shain, A. H. and Pollack, J. R. (2013). The spectrum of SWI/SNF mutations, ubiquitous in human cancers. PLoS One 8, e55119.
Shi, J. et al. (2013). Role of SWI/SNF in acute leukemia maintenance and enhancer-mediated Myc regulation. Genes Dev 27, 2648-2662.
St Pierre, R. and Kadoch, C. (2017). Mammalian SWI/SNF complexes in cancer: emerging therapeutic opportunities. Curr Opin Genet Dev 42, 56-67.
Sutherell, C. L. et al. (2016). Identification and Development of 2,3-Dihydropyrrolo[1,2-a]quinazolin-5(1H)-one Inhibitors Targeting Bromodomains within the Switch/Sucrose Nonfermenting Complex. Journal of Medicinal Chemistry 59, 5095-5101.
Toure, M. and Crews, C. M. (2016). Small-Molecule PROTACS: New Approaches to Protein Degradation. Angew Chem Int Ed Engl 55, 1966-1973.
Wilson, B. G. et al. (2014). Residual complexes containing SMARCA2 (BRM) underlie the oncogenic drive of SMARCA4 (BRG1) mutation. Mol Cell Biol 34, 1136-1144.
Winter, G. E. et al. (2015). Phthalimide conjugation as a strategy for in vivo target protein degradation. Science 348, 1376-1381.
Zengerle, M. et al. (2015). Selective Small Molecule Induced Degradation of the BET Bromodomain Protein BRD4. ACS Chemical Biology 10, 1770-1777.

### DETAILED DESCRIPTION OF THE INVENTION

### Compounds

It has now been found that, surprisingly, compounds of the present invention have additional advantages. In particular, compounds of formula (I), wherein the groups A, R¹, R³ and R⁴ have the meanings given hereinafter act as binders of SMARCA and/or can be used to prepare PROTAC degraders of SMARCA. In addition, compounds of formula (III), wherein the groups A, R¹, R³, R⁴, L and E have the meanings given hereinafter, act as degraders of SMARCA and are selective for SMARCA2 over SMARCA4. Thus, the compounds according to the invention may be used for example for the treatment of diseases characterised by excessive or abnormal cell proliferation.

It is therefore an object of the present invention a compound of formula (I):

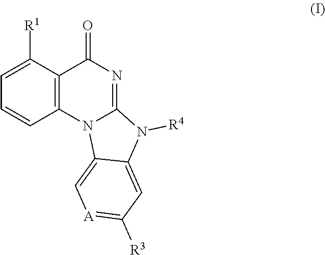

wherein:
A is —C(R²)— or —N—;
R¹ is halogen or —NH₂;
R² is selected from the group consisting of: hydrogen, halogen and —OX;
R³ is selected from the group consisting of: halogen, C₅₋₇-carbocyclyl and 4-12 membered heterocyclyl, wherein said C₅₋₇-carbocyclyl or 4-12 membered heterocyclyl is optionally substituted with at least one substituent selected from the group consisting of: C₁₋₃-alkyl, —NR^{a}R^{b}, —N(R^{a})COOR^{b} and —COOR^{a};
R⁴ is selected from the group consisting of: C₁₋₄-alkyl, C₃₋₆-carbocyclyl and 4-6 membered heterocyclyl, wherein said C₃₋₆-carbocyclyl or 4-6 membered heterocyclyl is optionally substituted with at least one substituent selected from the group consisting of: C₁₋₃-alkyl and —OH;
X is selected from the group consisting of: hydrogen, C₁₋₄-alkyl, —C(O)OC₁₋₄-alkyl, —(CH₂)ₙ-[O(CH₂)₂]ₘ—Y and 4-7 membered heterocyclyl, wherein said C₁₋₄-alkyl is optionally substituted with at least one substituent selected from the group consisting of: —COOR^{a} and —NR^{a}R^{b};
Y is selected from the group consisting of: —OR^{a}, —NR^{a}R^{b} and 4-7 membered heterocyclyl optionally substituted with at least one substituent selected from the group consisting of: C₁₋₃-alkyl, —COOR^{a} and —NR^{a}R^{b};
R^{a} and R^{b} are independently at each occurrence hydrogen or C₁₋₄-alkyl;
n is an integer selected from the group consisting of: 1, 2, 3 and 4;
m is an integer selected from the group consisting of: 0, 1, 2, 3, 4 and 5; or a salt thereof.

In one aspect of formula (I), n is 1, 2 or 3.

In another aspect of formula (I), m is 0 or 1.

In another aspect of formula (I), n is 1, 2 or 3 and m is 0 or 1.

In another aspect of formula (I), n is 1 and m is 0.

In another aspect of formula (I), n is 1 and m is 1.

In another aspect of formula (I), n is 2 and m is 0.

In another aspect of formula (I), n is 2 and m is 1.

In another aspect of formula (I), n is 3 and m is 0.

In another aspect of formula (I), n is 3 and m is 1.

In another aspect of formula (I), the sum of m+n does not exceed 8, preferably it does not exceed 7, preferably it does not exceed 6, preferably it does not exceed 5, preferably it does not exceed 4, preferably it does not exceed 3.

In another aspect of formula (I), A is —C(R²)—. Preferably, A is —C(H)—.

In another aspect of formula (I), R¹ is bromine, chlorine or —NH₂. Preferably, R¹ is bromine.

In another aspect of formula (I), R² is selected from the group consisting of: hydrogen, halogen, —O—C₁₋₄-alkyl, —O—(CH₂)ₙ-[O(CH₂)₂]ₘ—Y and —O-heterocyclyl, wherein said heterocyclyl is 4-7 membered, wherein said C₁₋₄-alkyl is optionally substituted with at least one —NR^{a}R^{b}.

In another aspect of formula (I), R² is selected from the group consisting of: hydrogen, halogen, —O—C₁₋₃-alkyl, —O—CH₂-heterocyclyl, —O—(CH₂)₂—O—(CH₂)₂heterocyclyl, —O—(CH₂)₂—O—(CH₂)₂OH, —O—(CH₂)₂—O—(CH₂)₂—O—C₁₋₃-alkyl and —O-heterocyclyl wherein said heterocyclyl is 4-7 membered, and wherein said heterocyclyl or C₁₋₃-alkyl is optionally substituted with at least one substituent selected from the group consisting of: C₁₋₃-alkyl and —NR^{a}R^{b}.

In another aspect of formula (I), R² is selected from the group consisting of: hydrogen, fluorine, —OCH₃,

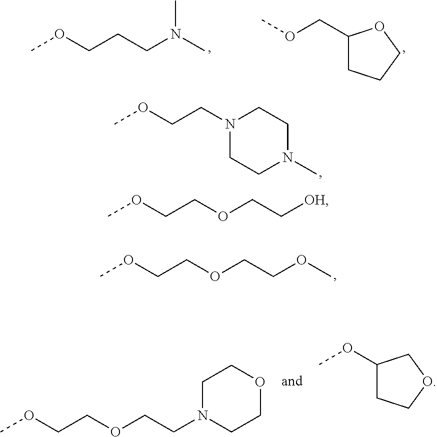

Preferably, R² is hydrogen.

In another aspect of formula (I), R³ is selected from the group consisting of: halogen, C₅₋₇-carbocyclyl and 5-8 membered heterocyclyl, wherein said C₅₋₇-carbocyclyl or 5-8 membered heterocyclyl is optionally substituted with at least one substituent selected from the group consisting of: C₁₋₃-alkyl, —NR^{a}R^{b}, —N(R^{a})COOR^{b} and —COOR^{a}.

In another aspect of formula (I), R³ is selected from the group consisting of:

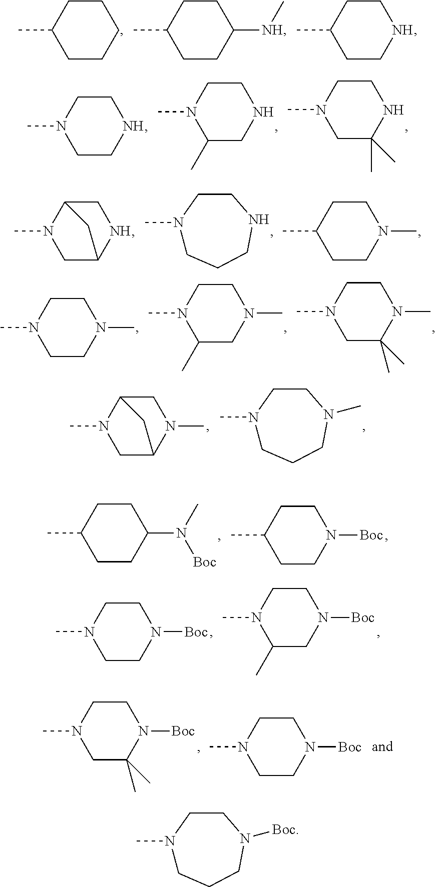

Preferably, R³ is

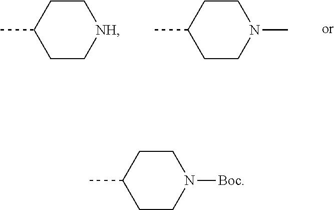

In another aspect, R⁴ is selected from the group consisting of: C₁₋₄-alkyl, C₄₋₆-carbocyclyl and 4-6 membered heterocyclyl, wherein said C₄₋₆ carbocyclyl is optionally substituted with at least one substituent selected from the group consisting of: C₁₋₃-alkyl and —OH.

In another aspect of formula (I), R⁴ is selected from the group consisting of: C₁₋₃-alkyl, cyclopentyl, oxiranyl and tetrahydrofuranyl, wherein said cyclopentyl is optionally substituted with at least one substituent selected from the group consisting of: C₁₋₃-alkyl and —OH.

In another aspect of formula (I), R⁴ is selected from the group consisting of: ethyl,

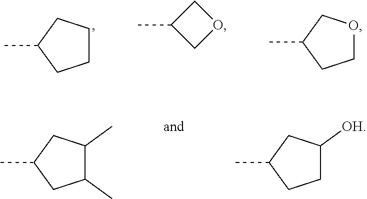

Preferably, R⁴ is cyclopentyl or ethyl.

In another aspect of formula (I), X is selected from the group consisting of: C₁₋₄-alkyl, —(CH₂)ₙ-[O(CH₂)₂]ₘ—Y and 4-7 membered heterocyclyl, wherein said C₁₋₄-alkyl is optionally substituted with at least one —NR^{a}R^{b}.

In another aspect of formula (I), X is selected from the group consisting of: C₁₋₃-alkyl, —CH₂— heterocyclyl, —(CH₂)₂—O—(CH₂)₂heterocyclyl, —(CH₂)₂—O—(CH₂)₂OH, —(CH₂)₂—O—(CH₂)₂—O—C₁₋₃-alkyl and heterocyclyl wherein any of said heterocyclyl is 4-7 membered, and wherein said C₁₋₃-alkyl is optionally substituted with at least one —NR^{a}R^{b}.

In another aspect of formula (I), Y is selected from the group consisting of: —OR^{a}, —NR^{a}R^{b} and 4-7 membered heterocyclyl optionally substituted with at least one substituent selected from the group consisting of: C₁₋₃-alkyl and —NR^{a}R^{b}.

In another aspect of formula (I), Y is selected from the group consisting of: —OR^{a}, —NR^{a}R^{b} and 4-7 membered heterocyclyl optionally substituted with at least one C₁₋₃-alkyl.

In another aspect, Y is selected from the group consisting of: —OH, —O—C₁₋₄-alkyl, —N(C₁₋₃-alkyl)₂, and 4-7 membered heterocyclyl optionally substituted with at least one substituent selected from the group consisting of: C₁₋₃-alkyl, —COOR^{a} and —NR^{a}R^{b}.

In another aspect of formula (I), Y is selected from the group consisting of: —OH, —O—C₁₋₄-alkyl, —N(C₁₋₃-alkyl)₂ and 4-7 membered heterocyclyl optionally substituted with at least one C₁₋₃-alkyl.

In another aspect of formula (I), Y is selected from the group consisting of: —OH, —OCH₃, —N(CH₃)₂, morpholinyl and piperazinyl, wherein said piperazinyl is optionally substituted with —CH₃.

In another aspect of formula (I), R^{a} and R^{b} are independently at each occurrence selected from the group consisting of: hydrogen, methyl, ethyl, propyl, butyl, iso-propyl, iso-butyl, sec-butyl and tert-butyl.

It is to be understood that any two or more aspects and/or preferred embodiments of formula (I) may be combined in any way to obtain further aspects and/or preferred embodiments of formula (I).

Preferred embodiments of compounds of formula (I) are represent by compounds of formulas 27 to 35 and relative subformulas as defined in the synthetic schemes hereinbelow. Preferably, the compound of formula (I) is selected among the group consisting of compounds 28a to 28aa and 32a as defined hereinbelow.

In a preferred aspect, the present invention provides a compound of formula (I) selected among the group consisting of compounds 28a to 28aa and 32a as defined hereinbelow or a pharmaceutically acceptable salt thereof.

It is a further object of the present invention a compound of formula (II):

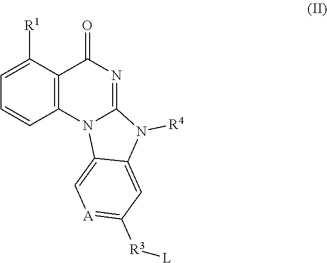

wherein:
A, R¹ and R⁴ are as defined hereinabove;
R³ is C₅₋₇-carbocyclyl or 4-12 membered heterocyclyl, wherein said C₅₋₇-carbocyclyl or 4-12 membered heterocyclyl is optionally substituted with at least one substituent selected from the group consisting of: C₁₋₃-alkyl and —NR^{a}R^{b}; wherein R^{a} and R^{b} are as defined hereinabove;
L is C₁₋₁₅-alkyl optionally substituted by one or more substituents each independently selected from the group consisting of: C₃₋₅-carbocyclyl and —OH, wherein any one or more carbon atom of said C₁₋₁₅-alkyl is optionally replaced by oxygen or nitrogen, or a salt thereof.

It is to be understood that the valency of R³ changes to accommodate bonding to L. For example, R³ is C₅₋₇-carbocyclylene or 4-12 membered heterocyclylene, wherein said C₅₋₇-carbocyclylene or 4-12 membered heterocyclylene is optionally substituted with at least one substituent selected from the group consisting of: C₁₋₃-alkyl and —NR^{a}R^{b}, in particular when R³ is directly (i.e. not via its optional C₁₋₃-alkyl or —NR^{a}R^{b} substituents) bound to L. Still for example, when R³ is substituted with —NR^{a}R^{b} and is bonded to L via —NR^{a}R^{b}, R^{a} or R^{b} is absent.

All aspects and preferred embodiments of A, R¹, R², R³, R⁴, n, m, X, Y, R^{a} and R^{b} described above for formula (I) equally apply to formula (11).

In one aspect of formula (II), R³ is C₅₋₇-carbocyclyl or 5-8 membered heterocyclyl, wherein said C₅₋₇-carbocyclyl or 5-8 membered heterocyclyl is optionally substituted with at least one substituent selected from the group consisting of: C₁₋₃-alkyl and —NR^{a}R^{b}. Preferably, in this embodiment, R^{a} and R^{b} are each independently hydrogen.

In another aspect of formula (II), R³ is selected from the group consisting of:

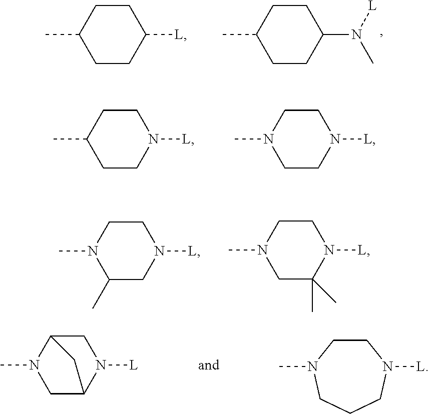

Preferably, R³ is or

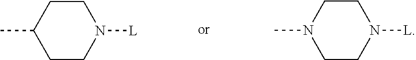

In another aspect of formula (II), L is linear C₁₋₃-alkyl optionally substituted by one or more substituents each independently selected from the group consisting of: C₁₋₃-alkyl, C₃₋₅-carbocyclyl and —OH, wherein any one or more carbon atom of said linear C₁₋₃-alkyl is optionally replaced by oxygen or nitrogen.

In another aspect of formula (II), L is C₁₋₃-alkyl optionally substituted by one or more substituents each independently selected from the group consisting of: methyl, cyclopropyl and —OH, wherein any one or more carbon atom of said C₁₋₃-alkyl is optionally replaced by oxygen or nitrogen.

In another aspect of formula (II), L is selected from the group consisting of:
linear or branched C₁₋₃-alkyl optionally substituted by —OH,
linear C₁₋₃-alkyl wherein one carbon atom is replaced by oxygen, said linear C₁₋₃-alkyl being optionally substituted with at least one substituent selected from the group consisting of: C₃₋₅-carbocyclyl, —OH and C₁₋₃-alkyl, and
linear C₁₋₃-alkyl wherein one carbon atom is replaced by nitrogen, said linear C₁₋₃-alkyl or nitrogen being optionally substituted with at least one C₁₋₃-alkyl.

It is to be understood that when any one or more carbon atom of the C₁₋₁₅-alkyl at position L is replaced by nitrogen, said nitrogen is optionally substituted by the one or more substituents.

In another aspect of formula (II), L has formula (IIa):

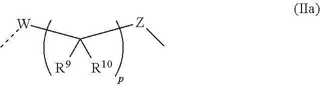

wherein:
W is —CH₂— or —N(R^{a})—;
R⁹ is selected from the group consisting of: hydrogen, C₃₋₅-carbocyclyl, —OH and C₁₋₃-alkyl;
R¹⁰ is hydrogen or C₁₋₃-alkyl;
p is an integer from 0 to 15;
Z is —O— or a bond.

In another aspect of formula (II), W is —CH₂— or —N(CH₃)—. Preferably, W is —CH₂—.

In another aspect of formula (II), R⁹ is selected from the group consisting of: hydrogen, methyl, cyclopropyl and —OH.

In another aspect of formula (II), R¹⁰ is hydrogen or methyl.

In another aspect of formula (II),
R⁹ is hydrogen and R¹⁰ is hydrogen; or
R⁹ is methyl and R¹⁰ is hydrogen; or
R⁹ is —OH and R¹⁰ is hydrogen; or
R⁹ is cyclopropyl and R¹⁰ is hydrogen; or
R⁹ is methyl and R¹⁰ is methyl.

Preferably, R⁹ and R¹⁰ are hydrogen.

In another aspect of formula (II), p is an integer from 1 to 8.

In another aspect of formula (II), p is an integer selected from the group consisting of: 1, 2, 3, 4 and 5.

It is to be understood that any two or more aspects and/or preferred embodiments of formula (II) may be combined in any way to obtain further aspects and/or preferred embodiments of formula (II).

It is a further object of the present invention a conjugate comprising:
a compound of formula (I) as defined above,
a linker, and
an E3 ubiquitin ligase binding moiety,
wherein said linker connects said compound of formula (I) to said E3 ubiquitin ligase binding moiety, or a salt thereof.

All aspects and preferred embodiments of A, R¹, R², R³, R⁴, R⁹, R¹⁰, n, m, p, W, X, Y, Z, R^{a} and R^{b} described above for formula (I) equally apply to the conjugate.

It is a further object of the present invention a compound of formula (III):

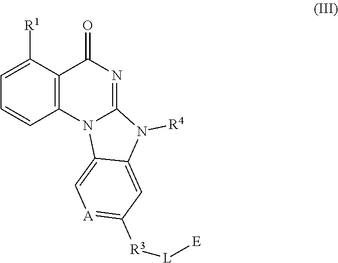

wherein:
A, R¹, R³, R⁴ and L are as defined hereinabove;
E is a group of formula (IIIa):

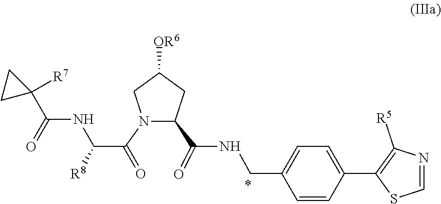

wherein:
R⁵ is selected from the group consisting of: hydrogen, C₁₋₃-alkyl and —COOR^{a}
R⁶ is hydrogen or —C(O)C₁₋₅alkyl;
R⁷ is selected from the group consisting of: halogen, —NR^{a}R^{b}, —CN, C₁₋₃-alkyl, C₁₋₃-haloalkyl, —C(O)OC₁₋₃-alkyl, C₃₋₇-carbocyclyl and 4-7 membered heterocyclyl; wherein R^{a} and R^{b} are as defined hereinabove;
or R⁷ is a C₃₋₅-alkyl forming a carbocyclyl together with the cyclopropyl to which R⁷ is bonded;
R⁸ is:
   a branched C₃₋₅-alkyl optionally substituted by C₃₋₄-carbocyclyl; or C₄₋₁₁-carbocyclyl optionally substituted with at least one substituent selected from the group consisting of: C₁₋₃-alkyl and halogen;
* represents the atom to which L is bonded;
or a salt thereof.

It is to be understood that the valency of L changes to accommodate bonding to E, e.g. L is C₁₋₁₅-alkylene optionally substituted by one or more substituents each independently selected from the group consisting of: C₃₋₅-carbocyclyl and —OH, wherein any one or more carbon atom of said C₁₋₁₅-alkylene is optionally replaced by oxygen or nitrogen.

All aspects and preferred embodiments of A, R¹, R², R³, R⁴, R⁹, R¹⁰, n, m, p, W, X, Y, Z, R^{a} and R^{b} described above for formula (I) and (II) equally apply to formula (III).

In one aspect of formula (III), R⁵ is selected from the group consisting of: hydrogen, C₁₋₃-alkyl and —COOC₁₋₃-alkyl.

In another aspect of formula (III), R⁵ is selected from the group consisting of: hydrogen, methyl and —C(O)OCH₂CH₃.

Preferably, R⁵ is methyl.

In another aspect of formula (III), R⁶ is selected from the group consisting of: hydrogen, —C(O)CH₃ and —C(O)(CH₂)₃CH₃.

Preferably, R⁶ is hydrogen.

In another aspect of formula (III), R⁷ is selected from the group consisting of: halogen, —N(C₁₋₃-alkyl)₂, —CN, C₁₋₃-alkyl, C₁₋₃-haloalkyl, —C(O)OC₁₋₃-alkyl, C₃₋₄-cycloalkyl and 4-7 membered heterocyclyl; or R⁷ is a C₃₋₅-alkyl forming a carbocyclyl together with the cyclopropyl to which R⁷ is bonded.

In another aspect of formula (III), R⁷ is selected from the group consisting of: fluorine,

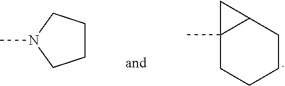

—N(CH₃)₂, —CN, methyl, —CF₃, —C(O)OCH₃, cyclopropyl, and.

Preferably, R⁷ is fluorine.

In another aspect of formula (III), R⁸ is selected from the group consisting of:

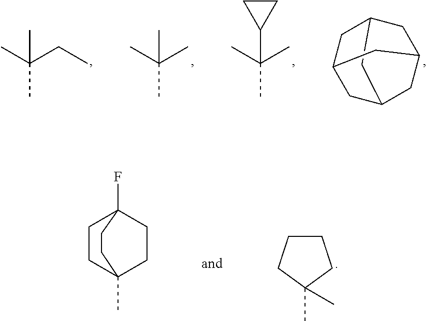

Preferably, R⁸ is

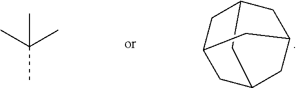

In another aspect of formula (III), R⁵ is selected from the group consisting of: hydrogen, methyl and —C(O)OCH₂CH₃;
R⁶ is selected from the group consisting of: hydrogen, —C(O)CH₃ and —C(O)(CH₂)₃CH₃;
R⁷ is selected from the group consisting of: fluorine, —N(CH₃)₂, —CN, methyl, —CF₃, —C(O)OCH₃, cyclopropyl,

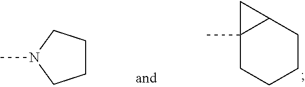

and
R⁸ is selected from the group consisting of:

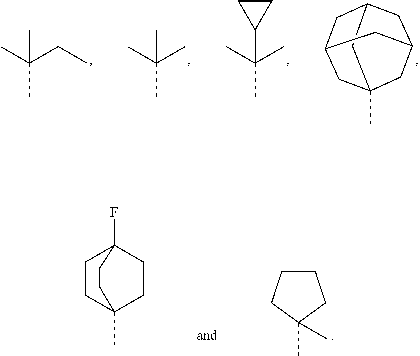

Preferably, R⁵ is methyl; R⁶ is hydrogen; R¹ is fluorine and R⁸ is or

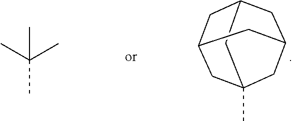

In another aspect of formula (III), L has formula (IIa):

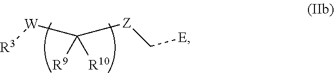

wherein W, Z, p, R⁹ and R¹⁰ are as described above for formula (II) or any of its aspects.

In another aspect of formula (III), L is selected from the group consisting of:

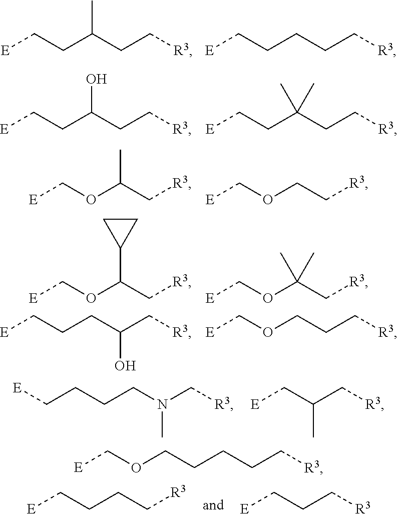

wherein [symbol] R³ denotes the bond between L and R³ and E [symbol] denotes the bond between E and L.

Preferred embodiments of compounds of formula (III) are represent by compounds of formulas 42 as defined in the synthetic schemes hereinbelow, and any subset thereof.

Preferably, the compound of formula (III) is selected among the group of compounds 42a to 42bk as defined hereinbelow.

In a preferred aspect, the present invention provides a compound of formula (III) selected among the group of compounds 42a to 42bk as defined hereinbelow or a pharmaceutically acceptable salt thereof.

It is to be understood that any two or more aspects and/or preferred embodiments of formula (III) may be combined in any way to obtain further aspects and/or preferred embodiments of formula (III).

All synthetic intermediates generically defined as well as specifically disclosed herein and their salts are also part of the invention.

All individual synthetic reaction steps as well as reaction sequences comprising these individual synthetic reaction steps, both generically defined or specifically disclosed herein, are also part of the invention.

The present invention further relates to hydrates, solvates, polymorphs, metabolites, derivatives, isomers, isotopes and prodrugs of a compound of formula (I), (II) and (III) (including all its embodiments).

The present invention further relates to a hydrate of a compound of formula (I), (II) and (III) (including all its embodiments).

The present invention further relates to a solvate of a compound of formula (I), (II) and (III) (including all its embodiments).

Compounds of formula (I), (II) and (III) (including all its embodiments) which bear e.g. ester groups are potential prodrugs the ester being cleaved under physiological conditions and are also part of the invention.

The present invention further relates to a pharmaceutically acceptable salt of a compound of formula (I), (II) and (III) (including all its embodiments), in particular with anorganic or organic acids or bases.

### Medical Uses—Methods of Treatment

The present invention is directed to SMARCA, in particular SMARCA2, binding compounds, in particular compounds of formula (I) (including all its embodiments), which can be useful in the synthesis of conjugates as defined above and/or of compounds of formula (III).

The present invention is directed to SMARCA, in particular SMARCA2, degrading compounds, in particular conjugates as defined above and/or compounds of formula (III) (including all its embodiments), which can be useful in the treatment and/or prevention of a disease and/or condition associated with or modulated by SMARCA, in particular SMARCA2, especially wherein the degradation of SMARCA, in particular SMARCA2, is of therapeutic benefit, including but not limited to the treatment and/or prevention of cancer.

In another aspect, the invention relates to a conjugate as defined above or a compound of formula (III)—or a pharmaceutically acceptable salt thereof—for use as a medicament.

In another aspect the invention relates to a conjugate as defined above or a compound of formula (III)—or a pharmaceutically acceptable salt thereof—for use in a method of treatment of the human or animal body.

In another aspect the invention relates to a SMARCA, in particular SMARCA2, degrading compound, in particular a conjugate as defined above or a compound of formula (III)—or a pharmaceutically acceptable salt thereof—for use in the treatment and/or prevention of a disease and/or condition wherein the degradation of SMARCA, in particular SMARCA2 is of therapeutic benefit, including but not limited to the treatment and/or prevention of cancer.

In another aspect the invention relates to a SMARCA2, degrading compound, in particular a conjugate as defined above or a compound of formula (III)—or a pharmaceutically acceptable salt thereof—for use in the treatment and/or prevention of a disease and/or condition wherein the degradation of SMARCA2 is of therapeutic benefit, including but not limited to the treatment and/or prevention of cancer.

In another aspect the invention relates to a conjugate as defined above or a compound of formula (III)—or a pharmaceutically acceptable salt thereof—for use in the treatment and/or prevention of cancer.

In another aspect the invention relates to a conjugate as defined above or a compound of formula (III)—or a pharmaceutically acceptable salt thereof—for use in a method of treatment and/or prevention of cancer in the human or animal body.

In another aspect the invention relates to the use of a conjugate as defined above or a compound of formula (III)—or a pharmaceutically acceptable salt thereof—for preparing a pharmaceutical composition for the treatment and/or prevention of cancer.

In another aspect the invention relates to a method for the treatment and/or prevention of a disease and/or condition wherein degradation of SMARCA, in particular SMARCA2, is of therapeutic benefit comprising administering a therapeutically effective amount of a conjugate as defined above or a compound of formula (III)—or a pharmaceutically acceptable salt thereof—to a human being.

In another aspect the invention relates to a method for the treatment and/or prevention of cancer comprising administering a therapeutically effective amount of a conjugate as defined above or a compound of formula (III)—or a pharmaceutically acceptable salt thereof
to a human being.

In another aspect the invention relates to a method for the treatment as hereinbefore and hereinafter defined.

For example, the following cancers, tumors and other proliferative diseases may be treated with compounds of the invention, without being restricted thereto:
Cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas, gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor; sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma; cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer; neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors; lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP);

All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:

Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;

Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

In another aspect the disease/condition/cancer to be treated/prevented with the compound of the invention is a disease/condition/cancer defined as exhibiting one or more of the following molecular features:
Impaired or loss of function of BAF complex subunits, including but not limited to SMARCB1, ARID1A, ARID1B, ARID2, PBRM1, SMARCA2 and SMARCA4 due either to inactivating mutations in these genes or loss of their expression through alternative mechanisms other than inactivating mutations;
Impaired or loss of SMARCA2 or SMARCA4 function due either to inactivating mutations in the SMARCA2 or SMARCA4 genes or loss of SMARCA2 or SMARCA4 expression through alternative mechanisms other than inactivating mutations; Inactivating mutations affecting SMARCA2 or SMARCA4 function include
nonsense or insertion/deletion (e.g. frameshift) mutations that result in loss of protein or activity; and/or
missense mutations that inactivate the function of the protein; and/or
changes in gene expression levels; and/or
changes in protein levels; and/or
changes in protein function.

In another aspect the cancer to be treated/prevented with the compound of the invention is a cancer found
to harbor mutations in either SMARCA2 or SMARCA4, preferably in SMARCA2, and/or
to show loss of either SMARCA2 or SMARCA4 expression, preferably in SMARCA2, and/or
to show loss or impairment of either SMARCA2 or SMARCA4 protein function, preferably in SMARCA2, and/or
to show changes in either SMARCA2 or SMARCA4 gene expression or either SMARCA2 or SMARCA4 protein levels, preferably in SMARCA2,
while at the same time, in each case, retaining a functional copy of either SMARCA2 or SMARCA4 protein and/or exhibiting proper expression of either SMARCA2 or SMARCA4.

Any disease/condition/cancer, medical use, use, method of treatment and/or prevention as disclosed or defined herein (including molecular/genetic features) may be treated/performed with any conjugate as defined above or any compound of formula (III) as disclosed or defined herein (including all individual embodiments or generic subsets defined above).

### Combination Therapy

In another aspect the invention relates to a conjugate as defined above or a compound of formula (III)—or a pharmaceutically acceptable salt thereof—for use as hereinbefore defined wherein said compound is administered before, after or together with at least one other pharmacologically active substance.

In another aspect the invention relates to a conjugate as defined above or a compound of formula (III)—or a pharmaceutically acceptable salt thereof—for use as hereinbefore defined, wherein said compound is administered in combination with at least one other pharmacologically active substance.

In another aspect the invention relates to a pharmacologically active substance prepared for being administered before, after or together with a conjugate as defined above or a compound of formula (III)—or a pharmaceutically acceptable salt thereof—for use as hereinbefore defined for the use of the conjugate as defined above or compound of formula (III).

In another aspect the invention relates to the use of a conjugate as defined above or a compound of formula (III)—or a pharmaceutically acceptable salt thereof—as hereinbefore defined wherein said compound is administered before, after or together with at least one other pharmacologically active substance.

In another aspect the invention relates to a method for the treatment and/or prevention as hereinbefore defined wherein e a conjugate as defined above or a compound of formula (III)—or a pharmaceutically acceptable salt thereof—is administered before, after or together with at least one other pharmacologically active substance.

In another aspect the invention relates to a method for the treatment and/or prevention as hereinbefore defined wherein a conjugate as defined above or a compound of formula (III)—or a pharmaceutically acceptable salt thereof—is administered in combination with a therapeutically effective amount of at least one other pharmacologically active substance.

In another aspect the pharmacologically active substance to be used together/in combination with the conjugate as defined above or with the compound of formula (III) (including all individual embodiments or generic subsets thereof), or in the medical uses, uses, methods of treatment and/or prevention as herein (above and below) defined can be selected from any one or more of the following (preferably there is only one additional pharmacologically active substance used in all these embodiments):
Hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib, bevacizumab and trastuzumab); antimetabolites (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumour antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors, ERK inhibitors, FLT3 inhibitors, BRD4 inhibitors, IGF-1R inhibitors, TRAILR2 agonists, Bcl-xL inhibitors, Bcl-2 inhibitors, Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors, immunotherapeutic agents such as immune checkpont inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), t-cell engagers (e.g. bi-specific T-cell engagers (BiTEs®) like e.g. CD3×BCMA, CD3×CD33, CD3×CD19), PSMA×CD3), tumor vaccines and various chemotherapeutic agents such as amifostin, anagrelid, clodronat, filgrastin, interferon, interferon alpha, leucovorin, procarbazine, levamisole, mesna, mitotane, pamidronate and porfimer.

When two or more substances or principles are to be used as part of a combined treatment regimen, they can be administered via the same route of administration or via different routes of administration, at essentially the same time (i.e. simultaneously, concurrently) or at different times (e.g. sequentially, successively, alternately, consecutively, or according to any other sort of alternating regime).

When the substances or principles are to be administered simultaneously via the same route of administration, they may be administered as different pharmaceutical formulations or compositions or as part of a combined pharmaceutical formulation or composition. Also, when two or more active substances or principles are to be used as part of a combined treatment regimen, each of the substances or principles may be administered in the same amount and according to the same regimen as used when the compound or principle is used on its own, and such combined use may or may not lead to a synergistic effect.

### Pharmaceutical Compositions—Kits

In another aspect the invention relates to a pharmaceutical composition comprising at least one (preferably one) conjugate as defined above—or a pharmaceutically acceptable salt thereof—and one or more pharmaceutically acceptable excipient(s).

In another aspect the invention relates to a pharmaceutical composition comprising at least one (preferably one) compound of formula (III)—or a pharmaceutically acceptable salt thereof—and one or more pharmaceutically acceptable excipient(s).

In another aspect the invention relates to a pharmaceutical preparation comprising a conjugate as defined above—or a pharmaceutically acceptable salt thereof—and at least one (preferably one) other pharmacologically active substance.

In another aspect the invention relates to a pharmaceutical preparation comprising a compound of formula (III)—or a pharmaceutically acceptable salt thereof—and at least one (preferably one) other pharmacologically active substance.

In another aspect the invention relates to a kit comprising
a first pharmaceutical composition or dosage form comprising a conjugate as defined above or a compound of formula (III) and, optionally, one or more pharmaceutically acceptable carriers, excipients and/or vehicles, and
at least a second pharmaceutical composition or dosage form comprising another pharmacologically active substance and, optionally, one or more pharmaceutically acceptable carriers, excipients and/or vehicles.

Suitable preparations for administering the compounds of the invention will be apparent to those with ordinary skill in the art and include for example tablets, pills, capsules, suppositories, lozenges, troches, solutions—particularly solutions for injection (s.c., i.v., i.m.) and infusion (injectables)—elixirs, syrups, sachets, emulsions, inhalatives or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) of the invention with known excipients, for example inert diluents, carriers, disintegrants, adjuvants, surfactants, binders and/or lubricants. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods:

For oral administration the tablets may of course contain, apart from the above-mentioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage range of the conjugate as defined above or compound of formula (III) applicable per day is usually from 1 mg to 2000 mg, preferably from 500 to 1500 mg.

The dosage for intravenous use is from 1 mg to 1000 mg with different infusion rates, preferably between 5 mg and 500 mg with different infusion rates.

However, it may sometimes be necessary to depart from the amounts specified, depending on the body weight, age, the route of administration, severity of the disease, the individual response to the drug, the nature of its formulation and the time or interval over which the drug is administered (continuous or intermittent treatment with one or multiple doses per day). Thus, in some cases it may be sufficient to use less than the minimum dose given above, whereas in other cases the upper limit may have to be exceeded. When administering large amounts it may be advisable to divide them up into a number of smaller doses spread over the day.

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The use of the prefix C_{x-y}, wherein x and y each represent a positive integer (x<y), indicates that the chain or ring structure specified and mentioned in direct association, may consist of a maximum of y and a minimum of x carbon atoms.

The indication of the number of members in groups that contain one or more heteroatom(s) (e.g. heterocyclyl) relates to the total number of atoms of all the ring members.

In general, for groups comprising two or more subgroups (e.g. heteroarylalkyl, heterocycylalkyl, cycloalkylalkyl, arylalkyl) the last named subgroup is the radical attachment point, for example, the substituent aryl-C₁₋₆alkyl means an aryl group which is bound to a C₁₋₆alkyl group, the latter of which is bound to the core or to the group to which the substituent is attached.

In groups like HO, H₂N, NC (cyano), HOOC, F₃C or the like, the skilled artisan can see the radical attachment point(s) to the molecule from the free valences of the group itself.

Alkyl denotes monovalent, saturated hydrocarbon chains, which may be present in both straight-chain (unbranched) and branched form. If an alkyl is substituted, the substitution may take place independently of one another, by mono- or polysubstitution in each case, on all the hydrogen-carrying carbon atoms.

The term “C₁₋₅alkyl” includes for example H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

Further examples of alkyl are methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂), 1-butyl (n-butyl; n-Bu; —CH₂CH₂CH₂CH₃), 2-methyl-1-propyl (iso-butyl; i-Bu; —CH₂CH(CH₃)₂), 2-butyl (sec-butyl; sec-Bu; —CH(CH₃)CH₂CH₃), 2-methyl-2-propyl (tert-butyl; t-Bu; —C(CH₃)₃), 1-pentyl (n-pentyl; —CH₂CH₂CH₂CH₂CH₃), 2-pentyl (—CH(CH₃)CH₂CH₂CH₃), 3-pentyl (—CH(CH₂CH₃)₂), 3-methyl-1-butyl (iso-pentyl; —CH₂CH₂CH(CH₃)₂), 2-methyl-2-butyl (—C(CH₃)₂CH₂CH₃), 3-methyl-2-butyl (—CH(CH₃)CH(CH₃)₂), 2,2-dimethyl-1-propyl (neo-pentyl; —CH₂C(CH₃)₃), 2-methyl-1-butyl (—CH₂CH(CH₃)CH₂CH₃), 1-hexyl (n-hexyl; —CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), 2,3-dimethyl-1-butyl (—CH₂CH(CH₃)CH(CH₃)CH₃), 2,2-dimethyl-1-butyl (—CH₂C(CH₃)₂CH₂CH₃), 3,3-dimethyl-1-butyl (—CH₂CH₂C(CH₃)₃), 2-methyl-1-pentyl (—CH₂CH(CH₃)CH₂CH₂CH₃), 3-methyl-1-pentyl (—CH₂CH₂CH(CH₃)CH₂CH₃), 1-heptyl (n-heptyl), 2-methyl-1-hexyl, 3-methyl-1-hexyl, 2,2-dimethyl-1-pentyl, 2,3-dimethyl-1-pentyl, 2,4-dimethyl-1-pentyl, 3,3-dimethyl-1-pentyl, 2,2,3-trimethyl-1-butyl, 3-ethyl-1-pentyl, 1-octyl (n-octyl), 1-nonyl (n-nonyl); 1-decyl (n-decyl) etc.

By the terms propyl, butyl, pentyl, hexyl, heptyl, octyl, nonyl, decyl etc. without any further definition are meant saturated hydrocarbon groups with the corresponding number of carbon atoms, wherein all isomeric forms are included.

The above definition for alkyl also applies if alkyl is a part of another (combined) group such as for example C_{x-y}-haloalkyl.

The term alkylene can also be derived from alkyl. Alkylene is bivalent, unlike alkyl, and requires two binding partners. Formally, the second valency is produced by removing a hydrogen atom in an alkyl. Corresponding groups are for example —CH₃ and —CH₂—, —CH₂CH₃ and —CH₂CH₂— or >CHCH₃ etc.

The term “C-alkylene” includes for example —(CH₂)—, —(CH₂—CH₂)—, —(CH(CH₃))—, —(CH₂—CH₂—CH₂)—, —(C(CH₃)₂)—, —(CH(CH₂CH₃))—, —(CH(CH₃)—CH₂)—, —(CH₂—CH(CH₃))—, —(CH₂—CH₂—CH₂—CH₂)—, —(CH₂—CH₂—CH(CH₃))—, —(CH(CH₃)—CH₂—CH₂)—, —(CH₂—CH(CH₃)—CH₂)—, —(CH₂—C(CH₃)₂)—, —(C(CH₃)₂—CH₂)—, —(CH(CH₃)—CH(CH₃))—, —(CH₂—CH(CH₂CH₃))—, —(CH(CH₂CH₃)—CH₂)—, —(CH(CH₂CH₂CH₃))—, —(CH(CH(CH₃))₂)—, and —C(CH₃)(CH₂CH₃)—.

Other examples of alkylene are methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene, hexylene etc.

By the generic terms propylene, butylene, pentylene, hexylene etc. without any further definition are meant all the conceivable isomeric forms with the corresponding number of carbon atoms, i.e. propylene includes 1-methylethylene and butylene includes 1-methylpropylene, 2-methylpropylene, 1,1-dimethylethylene and 1,2-dimethylethylene.

The above definition for alkylene also applies if alkylene is part of another (combined) group such as for example in HO—C_{x-y}alkyleneamino or H₂N—C_{x-y}alkyleneoxy.

Unlike alkyl, alkenyl consists of at least two carbon atoms, wherein at least two adjacent carbon atoms are joined together by a C═C double bond and a carbon atom can only be part of one C═C double bond. If in an alkyl as hereinbefore defined having at least two carbon atoms, two hydrogen atoms on adjacent carbon atoms are formally removed and the free valencies are saturated to form a second bond, the corresponding alkenyl is formed.

Examples of alkenyl are vinyl (ethenyl), prop-1-enyl, allyl (prop-2-enyl), isopropenyl, but-1-enyl, but-2-enyl, but-3-enyl, 2-methyl-prop-2-enyl, 2-methyl-prop-1-enyl, 1-methyl-prop-2-enyl, 1-methyl-prop-1-enyl, 1-methylidenepropyl, pent-1-enyl, pent-2-enyl, pent-3-enyl, pent-4-enyl, 3-methyl-but-3-enyl, 3-methyl-but-2-enyl, 3-methyl-but-1-enyl, hex-1-enyl, hex-2-enyl, hex-3-enyl, hex-4-enyl, hex-5-enyl, 2,3-dimethyl-but-3-enyl, 2,3-dimethyl-but-2-enyl, 2-methylidene-3-methylbutyl, 2,3-dimethyl-but-1-enyl, hexa-1,3-dienyl, hexa-1,4-dienyl, penta-1,4-dienyl, penta-1,3-dienyl, buta-1,3-dienyl, 2,3-dimethylbuta-1,3-diene etc.

By the generic terms propenyl, butenyl, pentenyl, hexenyl, butadienyl, pentadienyl, hexadienyl, heptadienyl, octadienyl, nonadienyl, decadienyl etc. without any further definition are meant all the conceivable isomeric forms with the corresponding number of carbon atoms, i.e. propenyl includes prop-1-enyl and prop-2-enyl, butenyl includes but-1-enyl, but-2-enyl, but-3-enyl, 1-methyl-prop-1-enyl, 1-methyl-prop-2-enyl etc.

Alkenyl may optionally be present in the cis or trans or E or Z orientation with regard to the double bond(s).

The above definition for alkenyl also applies when alkenyl is part of another (combined) group such as for example in C_{x-y}alkenylamino or C_{x-y}alkenyloxy.

Unlike alkylene, alkenylene consists of at least two carbon atoms, wherein at least two adjacent carbon atoms are joined together by a C═C double bond and a carbon atom can only be part of one C═C double bond. If in an alkylene as hereinbefore defined having at least two carbon atoms, two hydrogen atoms at adjacent carbon atoms are formally removed and the free valencies are saturated to form a second bond, the corresponding alkenylene is formed.

Examples of alkenylene are ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene, hexenylene etc.

By the generic terms propenylene, butenylene, pentenylene, hexenylene etc. without any further definition are meant all the conceivable isomeric forms with the corresponding number of carbon atoms, i.e. propenylene includes 1-methylethenylene and butenylene includes 1-methylpropenylene, 2-methylpropenylene, 1,1-dimethylethenylene and 1,2-dimethylethenylene.

Alkenylene may optionally be present in the cis or trans or E or Z orientation with regard to the double bond(s).

The above definition for alkenylene also applies when alkenylene is a part of another (combined) group as for example in HO—C_{x-y}alkenyleneamino or H₂N—C_{x-y}alkenyleneoxy.

By heteroatoms are meant oxygen, nitrogen and sulphur atoms.

Haloalkyl (haloalkenyl) is derived from the previously defined alkyl (alkenyl) by replacing one or more hydrogen atoms of the hydrocarbon chain independently of one another by halogen atoms, which may be identical or different. If a haloalkyl (haloalkenyl) is to be further substituted, the substitutions may take place independently of one another, in the form of mono- or polysubstitutions in each case, on all the hydrogen-carrying carbon atoms.

Examples of haloalkyl (haloalkenyl) are —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCI═CH₂, —CBr═CH₂, —CHFCH₂CH₃, —CHFCH₂CF₃ etc.

From the previously defined haloalkyl (haloalkenyl) are also derived the terms haloalkylene (haloalkenylene). Haloalkylene (haloalkenylene), unlike haloalkyl (haloalkenyl), is bivalent and requires two binding partners. Formally, the second valency is formed by removing a hydrogen atom from a haloalkyl (haloalkenyl).

Corresponding groups are for example —CH₂F and —CHF—, —CHFCH₂F and —CHFCHF— or >CFCH₂F etc.

The above definitions also apply if the corresponding halogen-containing groups are part of another (combined) group.

Halogen relates to fluorine, chlorine, bromine and/or iodine atoms.

The term “carbocyclyl”, either alone or in combination with another radical, means a mono-, bi- or tricyclic ring structure consisting of the specified number of carbon atoms. The term “carbocyclyl” refers to fully saturated, partially saturated and aromatic ring systems.

The term “carbocyclyl” encompasses fused, bridged and spirocyclic systems.

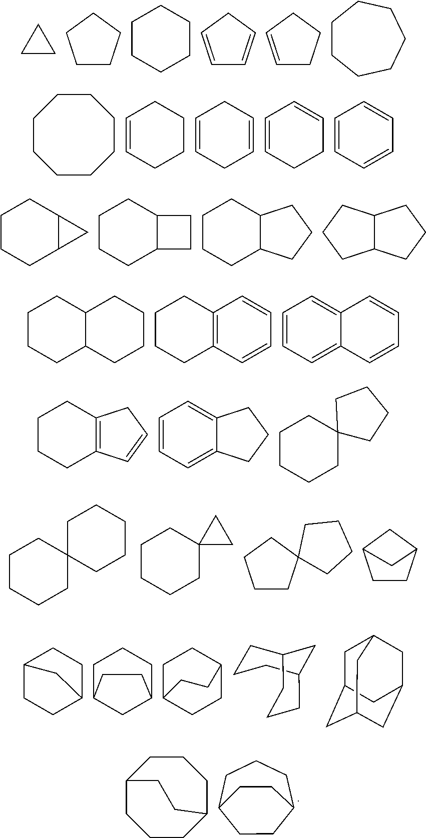

Preferably “carbocyclyl” as used herein refers to a cycloalkyl.

Carbocylylene, unlike carbocyclyl, is bivalent and requires two pinding partner. Formally, the second valency is obtained by removing a hydrogen atom from a carbocyclyl.

Cycloalkyl is made up of the subgroups monocyclic hydrocarbon rings, bicyclic hydrocarbon rings and spiro-hydrocarbon rings. The systems are saturated. In bicyclic hydrocarbon rings two rings are joined together so that they have at least two carbon atoms in common. In spiro-hydrocarbon rings one carbon atom (spiroatom) belongs to two rings together.

If a cycloalkyl is to be substituted, the substitutions may take place independently of one another, in the form of mono- or polysubstitutions in each case, on all the hydrogen-carrying carbon atoms. Cycloalkyl itself may be linked as a substituent to the molecule via every suitable position of the ring system.

Examples of cycloalkyl are cyclopropyl, cyclobutyl, cyclopentyl, cyclohexyl, cycloheptyl, bicyclo[2.2.0]hexyl, bicyclo[3.2.0]heptyl, bicyclo[3.2.1]octyl, bicyclo[2.2.2]octyl, bicyclo[4.3.0]nonyl (octahydroindenyl), bicyclo[4.4.0]decyl (decahydronaphthyl), bicyclo[2.2.1]heptyl (norbornyl), bicyclo[4.1.0]heptyl (norcaranyl), bicyclo[3.1.1]heptyl (pinanyl), spiro[2.5]octyl, spiro[3.3]heptyl etc.

The above definition for cycloalkyl also applies if cycloalkyl is part of another (combined) group as for example in C_{x-y}cycloalkylamino, C_{x-y}cycloalkyloxy or C_{x-y}cycloalkylalkyl.

If the free valency of a cycloalkyl is saturated, then an alicyclic group is obtained.

The term cycloalkylene can thus be derived from the previously defined cycloalkyl.

Cycloalkylene, unlike cycloalkyl, is bivalent and requires two binding partners. Formally, the second valency is obtained by removing a hydrogen atom from a cycloalkyl. Corresponding groups are for example:
cyclohexyl and

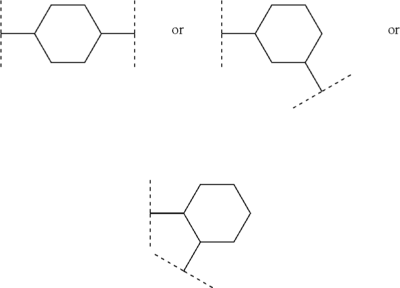

(cyclohexylene).

The above definition for cycloalkylene also applies if cycloalkylene is part of another (combined) group as for example in HO—C_{x-y}cycloalkyleneamino or H₂N—C_{x-y}cycloalkyleneoxy.

Heterocyclyl denotes ring systems, which are derived from the previously defined carbocyclyl and cycloalkyl by replacing one or more of the groups —CH₂— independently of one another in the hydrocarbon rings by the groups —O—, —S— or —NH— or by replacing one or more of the groups ═CH— by the group ═N—, wherein a total of not more than five heteroatoms may be present, at least one carbon atom must be present between two oxygen atoms and between two sulphur atoms or between an oxygen and a sulphur atom and the ring as a whole must have chemical stability. Heteroatoms may optionally be present in all the possible oxidation stages (sulphur→sulphoxide —SO—, sulphone —SO₂—; nitrogen→N-oxide). In a heterocyclyl there is no heteroaromatic ring, i.e. no heteroatom is part of an aromatic system.

Heterocyclyl is made up of the subgroups monocyclic heterorings, bicyclic heterorings, tricyclic heterorings and spiro-heterorings, which may be present in saturated or unsaturated form.

By unsaturated is meant that there is at least one double bond in the ring system in question, but no heteroaromatic system is formed. In bicyclic heterorings two rings are linked together so that they have at least two (hetero)atoms in common. In spiro-heterorings one carbon atom (spiroatom) belongs to two rings together.

If a heterocyclyl is substituted, the substitutions may take place independently of one another, in the form of mono- or polysubstitutions in each case, on all the hydrogen-carrying carbon and/or nitrogen atoms. Heterocyclyl itself may be linked as a substituent to the molecule via every suitable position of the ring system. Substituents on heterocyclyl do not count for the number of members of a heterocyclyl.

Examples of heterocyclyl are tetrahydrofuryl, pyrrolidinyl, pyrrolinyl, imidazolidinyl, thiazolidinyl, imidazolinyl, pyrazolidinyl, pyrazolinyl, piperidinyl, piperazinyl, oxiranyl, aziridinyl, azetidinyl, 1,4-dioxanyl, azepanyl, diazepanyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, 1,3-dioxolanyl, tetrahydropyranyl, tetrahydrothiopyranyl, [1,4]-oxazepanyl, tetrahydrothienyl, homothiomorpholinyl-S,S-dioxide, oxazolidinonyl, dihydropyrazolyl, dihydropyrrolyl, dihydropyrazinyl, dihydropyridyl, dihydro-pyrimidinyl, dihydrofuryl, dihydropyranyl, tetrahydrothienyl-S-oxide, tetrahydrothienyl-S,S-dioxide, homothiomorpholinyl-S-oxide, 2,3-dihydroazet, 2H-pyrrolyl, 4H-pyranyl, 1,4-dihydropyridinyl, 8-aza-bicyclo[3.2.1]octyl, 8-aza-bicyclo[5.1.0]octyl, 2-oxa-5-azabicyclo[2.2.1]heptyl, 8-oxa-3-aza-bicyclo[3.2.1]octyl, 3,8-diaza-bicyclo[3.2.1]octyl, 2,5-diaza-bicyclo[2.2.1]heptyl, 1-aza-bicyclo[2.2.2]octyl, 3,8-diaza-bicyclo[3.2.1]octyl, 3,9-diaza-bicyclo[4.2.1]nonyl, 2,6-diaza-bicyclo[3.2.2]nonyl, 1,4-dioxa-spiro[4.5]decyl, 1-oxa-3,8-diaza-spiro[4.5]decyl, 2,6-diaza-spiro[3.3]heptyl, 2,7-diaza-spiro[4.4]nonyl, 2,6-diaza-spiro[3.4]octyl, 3,9-diaza-spiro[5.5]undecyl, 2.8-diaza-spiro[4,5]decyl etc.

Further examples are the structures illustrated below, which may be attached via each hydrogen-carrying atom (exchanged for hydrogen):

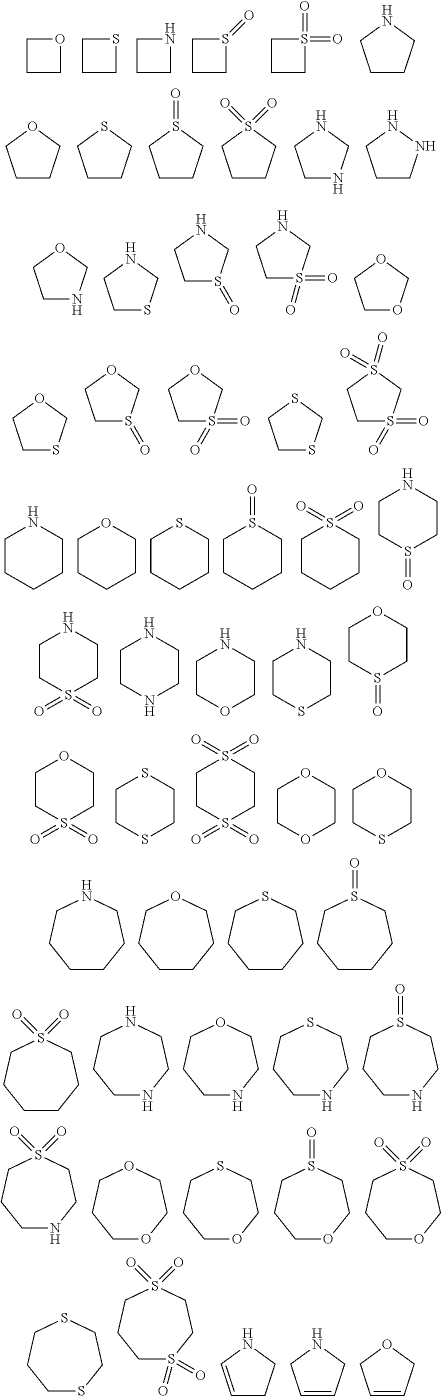

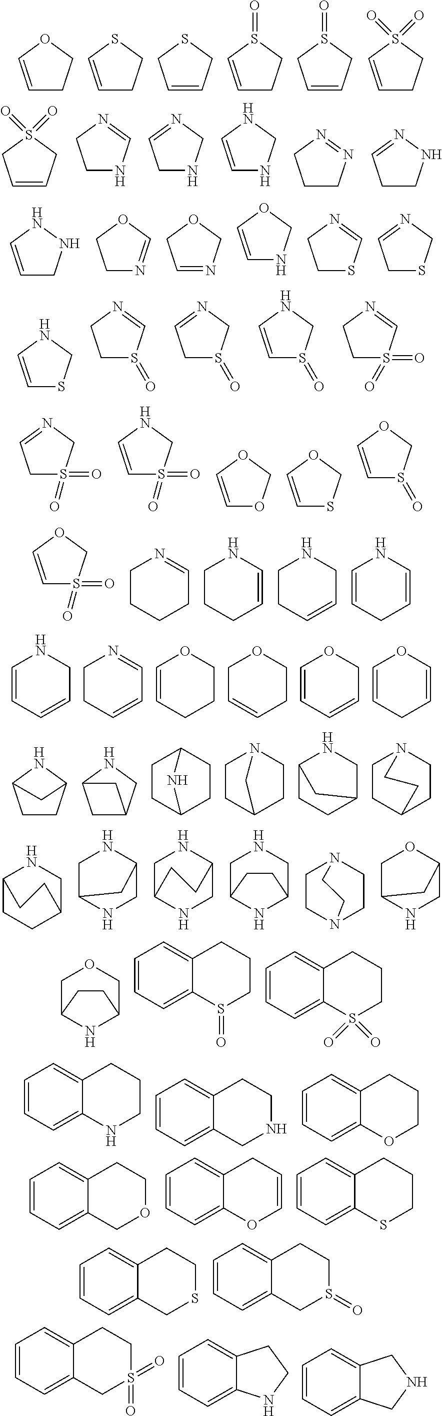

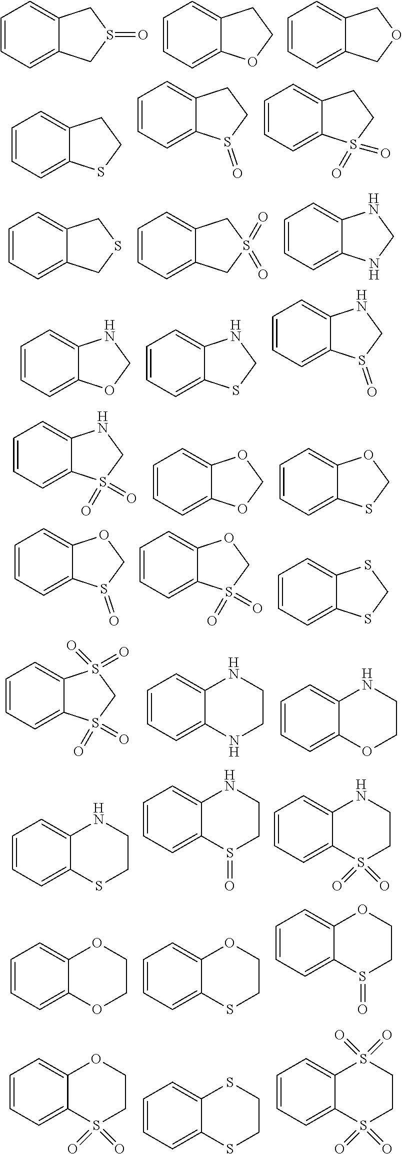

Preferably, heterocyclyls are 4 to 8 membered, monocyclic and have one or two heteroatoms independently selected from oxygen, nitrogen and sulfur.

Preferred heterocyclyls are: piperazinyl, piperidinyl, morpholinyl, pyrrolidinyl, azetidinyl, tetrahydropyranyl, tetrahydrofuranyl.

The above definition of heterocyclyl also applies if heterocyclyl is part of another (combined) group as for example in heterocyclylamino, heterocyclyloxy or heterocyclylalkyl.

If the free valency of a heterocyclyl is saturated, then a heterocyclic group is obtained.

The term heterocyclylene is also derived from the previously defined heterocyclyl.

Heterocyclylene, unlike heterocyclyl, is bivalent and requires two binding partners. Formally, the second valency is obtained by removing a hydrogen atom from a heterocyclyl.

Corresponding groups are for example:

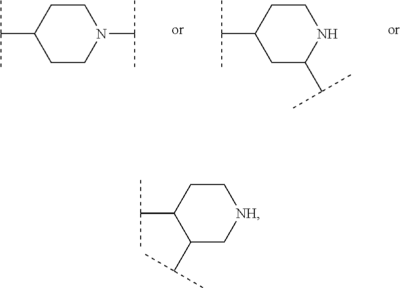

piperidinyl and
2,3-dihydro-1H-pyrrolyl and

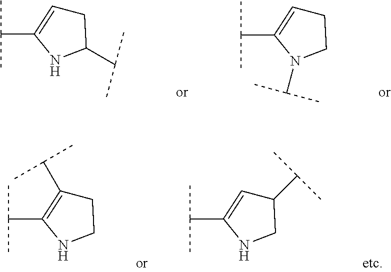

The above definition of heterocyclylene also applies if heterocyclylene is part of another (combined) group as for example in HO-heterocyclyleneamino or H₂N-heterocyclyleneoxy.

An asterisk or a dashed line [symbol] may be used to indicate the attachment point of one substitutent to another.

By substituted is meant that a hydrogen atom which is bound directly to the atom under consideration, is replaced by another atom or another group of atoms (substituent). Depending on the starting conditions (number of hydrogen atoms) mono- or polysubstitution may take place on one atom. Substitution with a particular substituent is only possible if the permitted valencies of the substituent and of the atom that is to be substituted correspond to one another and the substitution leads to a stable compound (i.e. to a compound which is not converted spontaneously, e.g. by rearrangement, cyclisation or elimination).

Bivalent substituents such as ═S, ═NR, ═NOR, ═NNRR, ═NN(R)C(O)NRR, ═N₂ or the like, may only be substituents on carbon atoms, whereas the bivalent substituents ═O and ═NR may also be a substituent on sulphur. Generally, substitution may be carried out by a bivalent substituent only at ring systems and requires replacement of two geminal hydrogen atoms, i.e. hydrogen atoms that are bound to the same carbon atom that is saturated prior to the substitution. Substitution by a bivalent substituent is therefore only possible at the group —CH₂— or sulphur atoms (═O group or ═NR group only, one or two ═O groups possible or, e.g., one ═O group and one ═NR group, each group replacing a free electron pair) of a ring system.

Stereochemistry/solvates/hydrates: Unless specifically indicated, throughout the specification and appended claims, a given chemical formula or name shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates and hydrates of the free compound or solvates and hydrates of a salt of the compound.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases, or by resolution of a racemic mixture using an appropriate resolving agent, e.g. by means of diastereomeric salt formation of the racemic compound with optically active acids or bases, subsequent resolution of the salts and release of the desired compound from the salt, or by derivatization of the corresponding racemic compounds with optically active chiral auxiliary reagents, subsequent diastereomer separation and removal of the chiral auxiliary group, or by kinetic resolution of a racemate (e.g. by enzymatic resolution); by enantioselective crystallization from a conglomerate of enantiomorphous crystals under suitable conditions, or by (fractional) crystallization from a suitable solvent in the presence of an optically active chiral auxiliary.

Salts: The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, and commensurate with a reasonable benefit/risk ratio.

As used herein “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts), also comprise a part of the invention.

Groups or substituents are frequently selected from among a number of alternative groups/substituents with a corresponding group designation (e.g. R^{a}, R^{b} etc). If such a group is used repeatedly to define a compound according to the invention in different parts of the molecule then the various uses are to be regarded as totally independent of one another.

By a “therapeutically effective amount” for the purposes of this invention is meant a quantity of substance that is capable of obviating symptoms of illness or of preventing or alleviating these symptoms, or which prolong the survival of a treated patient.

As used herein, “linker” refers to any chemical group capable of connecting a compound of formula (I) to a moiety of formula (IIIa). Examples of such linker include alkylene and poly-ethylene-glycol. Preferably, the linker is L as defined in any of the above aspects or preferred embodiments.

As used herein, “E3 ubiquitin ligase binding moiety” refers to any chemical group capable of binding any E3 ubiquitin ligase protein. For example, the E3 ubiquitin ligase binding moiety could be any VHL, cereblon, MDM2, DCAF15, DCAF16, IAPs and/or RNF114 binder. Preferably, the E3 ubiquitin ligase binding moiety is a VHL binder, such as the one of formula (IIIa). Binding of a chemical group to a E3 ubiquitin ligase protein may be measured by any method known in the art, included but not limited to Surface Plasmon Resonance (SPR) and Time-Resolve-Fluorescence Resonance Electron Transfer TR-FRET), e.g. as described hereinbelow.

By “E3 ubiquitin ligase”, it is meant a protein capable of recruiting an E2 ubiquitin-conjugating enzyme loaded with ubiquitin and/or assisting or catalyzes the transfer of ubiquitin from the E2 ubiquitin-conjugating enzyme to SMARCA 2 and/or 4. Examples of E3 ubiquitin ligase include VHL, cereblon, MDM2, DCAF15, DCAF16, IAPs and RNF114. A preferred example is VHL.

A SMARCA degrading compound in the context of this invention is a compound, which binds to SMARCA and simultaneously to a ubiquitin ligase protein, thereby inducing ubiquitylation of SMARCA and subsequent degradation of SMARCA by the proteasome. More specifically the SMARCA degrading compound preferably binds to the bromodomain of SMARCA. Suitable test systems to measure the binding of compounds according to the invention to SMARCA and their degradation are disclosed herein.

List of abbreviations

| | |
|---|---|
| Ac | acetyl |
| ACN | acetonitrile |
| aq. | aquatic, aqueous |
| ATP | adenosine triphosphate |
| Bn | Benzyl |
| Boc | tert-butyloxycarbonyl |
| Boc-Tle-OH | (S)-N-Boc-2-amino-3,3-dimethylbutyric acid |
| Boc-Hyp-OH | Boc-L-hydroxyproline |
| BRG1 | SMARCA4 |
| BRM | SMARCA2 |
| Bu | butyl |
| c | concentration |
| CDI | N,N′-carbonyldiimidazole |
| CH₂Cl₂ | dichloro methane |
| CM5 | SPR Sensor Chip CM5 |
| d | day(s) |
| dba | dibenzylideneacetone |
| TLC | thin layer chromatography |
| Davephos | 2-dimethylamino-2′-dicyclohexylaminophosphinobiphenyl |
| DBA | dibenzylidene acetone |
| DCE | dichloro ethane |
| DCM | dichloro methane |
| DEA | diethyl amine |
| DEAD | diethyl azodicarboxylate |
| DIPEA | N-ethyl-N,N-diisopropylamine (Hünig's base) |
| DMAc | N,N-dimethylacetamide |
| DMAP | 4-N,N-dimethylaminopyridine |
| DME | 1,2-dimethoxyethane |
| DMEM | Dulbecco's Modified Eagle Medium |
| DMF | N,N-dimethylformamide |
| DMSO | dimethylsulfoxide |
| DPPA | diphenylphosphorylazide |
| dppf | 1,1′-bis(diphenylphosphino)ferrocene |
| DTT | dithiothreitol |
| EDC | 1-Ethyl-3-(3-dimethyllaminopropyl)carbodiimide hydrochloride |
| EDTA | ethylenediaminetetraacetic acid |
| EGTA | ethyleneglycoltetraacetic acid |
| eq | equivalent(s) |
| equiv. | equivalent(s) |
| ESI | electron spray ionization |
| Et | ethyl |
| Et₂O | diethyl ether |
| EtOAc | ethyl acetate |
| EtOH | ethanol |
| FBS | Fetal Bovine Serum |
| FCS | Fetal Calf Serum |
| h or hr | hour |
| GAPDH | Glyceraldehyde 3-phosphate dehydrogenase |
| HATU | O-(7-azabenzotriazol-1-yl)-N,N,N′,N′-tetramethyl-uronium |
| | hexafluorophosphate |
| HOBt | 1-hydroxybenzotriazole |
| HPLC | high performance liquid chromatography |
| IBX | 2-iodoxy benzoic acid |
| i | iso |
| conc. | concentrated |
| LAH | lithium aluminium hydride |
| LC | liquid chromatography |
| LiHMDS | lithium bis(trimethylsilyl)amide |
| sln. | solution |
| Me | methyl |
| MeCN | acetonitrile |
| MeOH | methanol |
| min | minutes |
| MPLC | medium pressure liquid chromatography |
| MS | mass spectrometry |
| Ms | mesylate or methanesulfonyl |
| MsCl | methanesulfonyl chloride |
| MTBE | methyl tert-butyl ether |
| NBS | N-bromo-succinimide |
| NHS | N-hydroxy-succinimide |
| NIS | N-iodo-succinimide |
| NMM | N-methylmorpholine |
| NMP | N-methylpyrrolidone |
| NP | normal phase |
| n.a. | not available |
| PBS | phosphate-buffered saline |
| PG | protecting group |
| Ph | phenyl |
| Pr | propyl |
| Py | pyridine |
| rac | racemic |
| red. | reduction |
| Rf (R_{f}) | retention factor |
| RP | reversed phase |
| rt or RT | ambient temperature |
| sat. | saturated |
| SFC | supercritical fluid chromatography |
| S_{N} | nucleophilic substitution |
| SPR | surface plasmon resonance |
| T3P | propanephosphonic acid anhydride |
| TBAF | tetrabutylammonium fluoride |
| TBDMS | tert-butyldimethylsilyl |
| TBME | tert-butylmethylether |
| TBTU | O-(benzotriazol-1-yl)-N,N,N′,N′-tetramethyl-uronium tetrafluoroborate |
| tBu | tert-butyl |
| TCEP | 3-[bis(2-carboxyethyl)phosphanyl]propanoic acid; hydrochloride |
| TEA | triethyl amine |
| temp. | temperature |
| TEMPO | (2,2,6,6-Tetramethylpiperidin-1-yl)oxyl |
| tert | tertiary |
| Tf | triflate |
| TFA | trifluoroacetic acid |
| THF | tetrahydrofuran |
| TMS | trimethylsilyl |
| t_{Ret.} | retention time (HPLC) |
| TRIS | tris(hydroxymethyl)-aminomethane |
| TsOH | p-toluenesulfonic acid |
| UV | ultraviolet |
| VHL | Von-Hippel Lindau |
| Vol | Volumes (calculated based on mass or volume of starting material) |

Features and advantages of the present invention will become apparent from the following detailed examples which illustrate the principles of the invention by way of example without restricting its scope.

### Biological Methods

Binary SMARCA2 Binding Affinity Determination (A2 Binary [nM]):

### SMARCA2 SPR Binding Experiments

SPR experiments were performed on Biacore 8K or T200 instruments (GE Healthcare). Immobilization of target protein was carried out at 25° C. on a CM5 chip using amine coupling (EDC/NHS, GE Healthcare) in HBS-P+ running buffer, containing 2 mM TCEP, pH 7.4. Following activation of the surface with EDC/NHS (contact time 600 s, flow rate 10 μL/min), the SMARCA2BD prepared at 0.5-0.7 mg/mL in coupling buffer consisting of 10 mM Na-Acetate pH 6.5, 0.005% Tween-20 and 50 μM PFI-3 (Gerstenberger, B. S. et al. Identification of a chemical probe for family VIII bromodomains through optimization of a fragment hit. Journal of Medicinal Chemistry 59, 4800-4811 (2016)), were coupled to a density of 500-5000 Response Units (RU). The surface was deactivated using 1 M ethanolamine. For VHL target protein, streptavidin (Sigma Aldrich) (prepared at 1 mg/mL in 10 mM sodium acetate coupling buffer, pH 5.0) was first immobilized by amine coupling to a density of 500-5000 RU, after which biotinylated VCB complex (2.8 μM in running buffer) was streptavidin-coupled to a density of 1000-5000 RU. Biotinylated VCB was prepared as previously described (Gadd, M. S. et al. Structural basis of PROTAC cooperative recognition for selective protein degradation. Nature Chemical Biology 13, 514-521 (2017). The reference surface consisted of an EDC/NHS-treated surface deactivated with 1 M ethanolamine.

All interaction experiments were performed at 6° C. in running buffer containing 20 mM TRIS, 150 mM potassium chloride, 2 mM magnesium chloride, 2 mM TCEP, 0.005% TWEEN 20, 1% dimethyl sulfoxide; pH 8.3. Sensorgrams from reference surfaces and blank injections were subtracted from the raw data prior to data analysis using Biacore T200 or Biacore 8K evaluation software. Sensorgrams recorded at different compound concentrations in multi-cycle experiments were fitted using a 1:1 interaction model, with a term for mass-transport included.

### SMARCA2 ULight Time-Resolved Fluorescence Resonance Electron Transfer (TR-FRET) Assay

This assay was used to identify compounds which inhibit the binding of a biotinylated SMARCA2 binder to SMARCA2. His-tagged SMARCA2 protein corresponding to SMARCA2 pdb 4QY4 with N-terminal His-tag and TEV cleavage site was expressed in E. coli. A known SMARCA2 binder chemically fused to biotin was used as SMARCA2 binding partner in the assay. Test compounds dissolved in DMSO were dispensed onto assay plates (Proxiplate 384 PLUS, white, PerkinElmer; 6008289) using an Access Labcyte Workstation with the Labcyte Echo 55×. For the chosen highest assay compound concentration of 100 μM, 150 nL of compound solution was transferred from a 10 mM DMSO compound stock solution. A series of eleven fivefold dilutions per compound was transferred to the assay plate, compound dilutions were tested in duplicates. DMSO was added as backfill to a total volume of 150 nl.

The assay runs on a fully automated robotic system. 5 nL of the biotinylated probe (10 mM stock in 100% DMSO) was added to rows 1-23 using the Labcyte Echo 55× for transfer. 5 nL of 100% DMSO was added to row 24. 15 μL of reaction mix including SMARCA2 (40 nM final assay concentration), Lance-Eu—W1024 labeled Streptavidin (Perkin Elmer Cat No AD0062, 2.5 nM final assay concentration) and ULight-anti 6×His antibody (Perkin Elmer TRF0105-M, 50 nM final assay concentration) was added to rows 1-24. Plates are kept at room temperature. After 60 minutes incubation time the signal is measured in a PerkinElmer Envision HTS Multilabel Reader using the TR-FRET LANCE Ultra specs from PerkinElmer. Each plate contains 16 wells of a negative control (diluted DMSO instead of test compound; column 23 with biotinylated probe) and 16 wells of a positive control (diluted DMSO instead of test compound; column 24 without biotinylated probe). As internal control non-biotinylated SMARCA2 binders can be measured on each compound plate. DC50 values are calculated and analyzed using a 4 parametric logistic model.

Determination of SMARCA2 and SMARCA4 Degradation (DC50 A2 and A4 [nM])

### Protein Degradation Assays for SMARCA2 and SMARCA4

For capillary electrophoresis, 35000 A549 SMARCA4 revertant cells (ATCC) were seeded in 100 μL F12K medium (F12K Nut Mix, Gibco #21127-022) supplemented with 10% FBS (Hyclone) into a Greiner 96-well F-bottom plate (#655182) and incubated at 37° C. overnight. Compounds were added from DMSO stock solution using an Access Labcyte Workstation with a Labcyte Echo 550 or 555 acoustic dispenser and incubated at 37° C. for 18 h. Medium was removed, cells washed with PBS and lysed in 30 μL ice cold lysis buffer (1% Triton, 350 mM KCl, 10 mM TRIS pH 7.4, phosphatase-protease inhibitor cocktail (Thermo Scientific no. 1861281), 10 mM DTT, benzonase 0.5 μL mL⁻¹ (Novagen no. 70746 10KU, 25 U per μL) for 20 min at 4° C. on a bioshake at 800 rpm before insoluble debris was pelleted by centrifugation for 20 minutes at 4000 rpm at 4° C. The supernatant was transferred to a fresh twin-tec 96-well PCR plate (Fisher Scientific #0030 128.575). SMARCA2 and SMARCA4 levels were determined on a Sally Sue capillary-based immunoassay platform (ProteinSimple) using rabbit anti-SMARCA2 antibody (1:25, Sigma no. HPA029981), rabbit anti-SMARCA4 antibody (CellSignaling no. 49360, 1:25) and rabbit anti-GAPDH antibody (1:100, abcam no. ab9485) for normalization. Bands were quantified, normalized to GAPDH and DMSO control and DC50 values computed using a four-parametric logistic model.

For degradation analysis by imaging, 1250 RKO cells (CRL-2577, ATCC) per well were seeded into 60 μL DMEM (Sigma Aldrich) supplemented with 10% FBS (Hyclone) in 384-well flat bottom plates (CellCarrier Ultra, Perkin Elmer) and incubated at 37° C. and 5% CO₂ in a humidified atmosphere overnight. Compounds were added the next day using an Access Labcyte Workstation with a Labcyte Echo 550 or 555 acoustic dispenser and incubated with the cells for 4 or 24 h. Cells were fixed by adding 25 μL fixation buffer (7.4% formaldehyde (Sigma Aldrich F8775), 0.2% Triton TX100 (Sigma Aldrich F93443) in PBS) for 15 minutes at room temperature. After aspirating the fixing solution, the cells were washed once with 25 μL PBS and 25 μL of Blocking Buffer (10% Goat Serum in PBS) were added to each well and incubated for 30 minutes. Cells were washed with PBS and incubated with 20 μL mouse anti-Smarca4 (BRG1) antibody (Cell Signaling #52251) or rabbit anti-Smarca2 (BRM) antibody (Sigma Aldrich HPA029981) in blocking solution (PBS with 10% FCS) for ca. 2-4 h at RT. For detection of the nuclei, 25 μL of 5 μg/mL Hoechst 33342 (stock 10 mg/mL in H2O; Invitrogen H1399) were added together with Alexa Fluor 647 goat anti-mouse IgG (Invitrogen A-21235) or Alexa Fluor 488 goat anti-rabbit IgG (Invitrogen A-11034) in blocking solution and incubated for 60 min at RT. The cell layer was washed with 25 μL PBS, the wells were filled with 25 μL PBS and the plates sealed with an adhesive sheet. The mean intensity at 488 or 647 nm in the nucleus was measured using an Opera Phenix Plus High-Content Screening System (Perkin Elmer), values were normalized to the background and DMSO control and DC50 values were calculated and analyzed using a four-parametric logistic model.

### Preparation of the Compounds According to the Invention

### General

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

Compounds described herein are named in accordance with CAS rules using the software Marvin Sketch. If a compound is to be represented both by a structural formula and by its nomenclature, in the event of a conflict the structural formula is decisive.

Microwave reactions are carried out in an initiator/reactor made by Biotage or in an Explorer made by CEM or in Synthos 3000 or Monowave 3000 made by Anton Paar in sealed containers (preferably 2, 5 or 20 mL), preferably with stirring.

### Chromatography

The thin layer chromatography is carried out on ready-made silica gel 60 TLC plates on glass (with fluorescence indicator F-254) made by Merck.

The preparative high pressure chromatography (RP HPLC) of the intermediates and final example compounds is carried out on Agilent or Gilson systems with columns made by Waters (names: SunFire™ Prep C18, OBD™ 10 μm, 50×150 mm or SunFire™ Prep C18 OBD™ 5 μm, 30×50 mm or XBridge™ Prep C18, OBD™ 10 μm, 50×150 mm or XBridge™ Prep C18, OBD™ 5 μm, 30×150 mm or XBridge™ Prep C18, OBD™ 5 μm, 30×50 mm) and YMC (names: Actus-Triart Prep C18, 5 μm, 30×50 mm).

Different gradients of H₂O/acetonitrile are used to elute the compounds, while for Agilent systems 5% acidic modifier (20 mL HCOOH to 1 L H₂O/acetonitrile (1/1)) is added to the water (acidic conditions). For Gilson systems 0.1% HCOOH is added to water.

For the chromatography under basic conditions for Agilent systems H₂O/acetonitrile gradients are used as well, while the water is made alkaline by addition of 5% basic modifier (50 g NH₄HCO₃+50 mL NH₃ (25% in H₂O) to 1 L with H₂O). For Gilson systems the water is made alkaline as follows: 5 mL NH₄HCO₃ solution (158 g in 1 L H₂O) and 2 mL NH₃ (28% in H₂O) are replenished to 1 L with H₂O.

The supercritical fluid chromatography (SFC) of the intermediates and example compounds is carried out on a JASCO SFC-system with the following columns: Chiralcel OJ (250×20 mm, 5 μm), Chiralpak AD (250×20 mm, 5 μm), Chiralpak AS (250×20 mm, 5 μm), Chiralpak IC (250×20 mm, 5 μm), Chiralpak IA (250×20 mm, 5 μm), Chiralcel OJ (250×20 mm, 5 μm), Chiralcel OD (250×20 mm, 5 μm), Phenomenex Lux C2 (250×20 mm, 5 μm).

The analytical HPLC (reaction control) of intermediate and final compounds is carried out using columns made by Waters (names: XBridge™ C18, 2.5 μm, 2.1×20 mm or XBridge™ C18, 2.5 μm, 2.1×30 mm or Aquity UPLC BEH C18, 1.7 μm, 2.1×50 mm) and YMC (names: Triart C18, 3.0 μm, 2.0×30 mm) and Phenomenex (names: Luna C18, 5.0 μm, 2.0×30 mm). The analytical equipment is also equipped with a mass detector in each case.

### HPLC-Mass Spectroscopy/UV-Spectrometry

The retention times/MS-ESI⁺ for characterizing the intermediates and final example compounds are produced using an HPLC-MS apparatus (high performance liquid chromatography with mass detector). Compounds that elute at the injection peak are given the retention time t_{Ret.}=0.00. The exact methods are as follows.

### HPLC Methods

Method 1

| HPLC | Agilent 1100/1200 system | |
|---|---|---|
| MS | 1200 Series LC/MSD (API-ES +/− 3000/3500 V, | |
| | Quadrupol, G6140A) | |
| MSD signal | Scan pos/neg 150-750 | |
| settings | | |
| column | YMC; Part. No. TA12S03-0302WT; Triart C18, 3 μm, | |
| | 12 nm; 30 × 2.0 mm column | |
| eluant | A: H2O + 0.11% formic acid | |
| | B: MeCN + 0.1% formic acid (HPLC grade) | |
| detection | UV 254 nm, 230 nm, 214 nm (bandwidth 10, reference off) | |
| signal | | |
| spectrum | range: 190-400 nm; slit: 4 nm | |
| peak width | >0.0031 min (0.063 s response time, 80 Hz) | |
| injection | 0.5 μL standard injection | |
| flow | 1.4 mL/min | |
| column | 45° C. | |
| temperature | | |
| gradient | 0.0-1.0 min | 15% → 95% B |
| | 1.0-1.1 min | 95% B |

Stop time: 1.23 min

Method 2

| HPLC | Agilent 1100/1200 system | |
|---|---|---|
| MS | 1200 Series LC/MSD (API-ES +/− 3000/3500 V, | |
| | Quadrupol, G6140A) | |
| MSD signal | Scan pos 700-1350 | |
| settings | | |
| column | YMC; Part. No. TA12S03-0302WT; Triart C18, 3 μm, | |
| | 12 nm; 30 × 2.0 mm column | |
| eluant | A: H2O + 0.11% formic acid | |
| | B: MeCN + 0.1% formic acid (HPLC grade) | |
| detection | UV 254 nm, 230 nm, 214 nm (bandwidth 10, reference off) | |
| signal | | |
| spectrum | range: 190-400 nm; slit: 4 nm | |
| peak width | >0.0031 min (0.063 s response time, 80 Hz) | |
| injection | 0.5 μL standard injection | |
| flow | 1.4 mL/min | |
| column | 45° C. | |
| temperature | | |
| gradient | 0.0-1.0 min | 15% → 95% B |
| | 1.0-1.1 min | 95% B |

Stop time: 1.23 min

Method 3

| HPLC | Agilent 1100/1200 system | |
|---|---|---|
| MS | 1200 Series LC/MSD (MM-ES + APCl +/− 3000 V, | |
| | Quadrupol, G6130B) | |
| MSD signal | Scan pos/neg 150-750 | |
| settings | | |
| column | Waters, Part. No. 186003389, XBridge BEH C18, | |
| | 2.5 μm, 2.1 × 30 mm) column | |
| eluant | A: 5 mM NH4HCO3/18 mM NH3 (pH = 9.2) | |
| | B: acetonitrile (HPLC grade) | |
| detection | UV 254 nm, 230 nm, 214 nm (bandwidth 8, reference off) | |
| signal | | |
| spectrum | range: 190-400 nm; slit: 4 nm | |
| peak width | >0.0031 min (0.063 s response time, 80 Hz) | |
| injection | 0.5 μL standard injection | |
| flow | 1.4 mL/min | |
| column | 45° C. | |
| temperature | | |
| gradient | 0.0-1.0 min | 15% → 95% B |
| | 1.0-1.1 min | 95% B |

Stop time: 1.3 min

Method 4

| HPLC | Agilent 1260 Infinity II system | |
|---|---|---|
| MS | 1260 Series Infinity Lab LC/MSD (API-ES +/− 3000/ | |
| | 3500 V, Quadrupol, G6125B) | |
| MSD signal | Scan pos/neg 150-700 | |
| settings | | |
| column | Waters, Part. No. 186003389, XBridge BEH C18, | |
| | 2.5 μm, 2.1 × 30 mm) column | |
| eluant | A: 5 mM NH4HCO3/18 mM NH3 (pH = 9.2) | |
| | B: acetonitrile (HPLC grade) | |
| detection | UV 254 nm, 230 nm, 214 nm (bandwidth 8, reference off) | |
| signal | | |
| spectrum | range: 190-400 nm; slit: 4 nm | |
| peak width | >0.0031 min (0.063 s response time, 80 Hz) | |
| injection | 0.5 μL standard injection | |
| Flow | 1.4 mL/min | |
| column | 45° C. | |
| temperature | | |
| gradient | 0.0-1.0 min | 15% → 95% B |
| | 1.0-1.1 min | 95% B |

Stop time: 1.3 min

Method 5

| HPLC | Agilent 1100/1200 system | |
|---|---|---|
| MS | 1200 Series LC/MSD (MM-ES + APCl +/− 3000 V, | |
| | Quadrupol, G6130B) | |
| MSD signal | Scan pos 700-1350 | |
| settings | | |
| column | Waters, Part. No. 186003389, XBridge BEH C18, 2.5 μm, | |
| | 2.1 × 30 mm) column | |
| eluant | A: 5 mM NH4HCO3/18 mM NH3 (pH = 9.2) | |
| | B: acetonitrile (HPLC grade) | |
| detection | UV 254 nm, 230 nm, 214 nm (bandwidth 8, reference off) | |
| signal | | |
| spectrum | range: 190-400 nm; slit: 4 nm | |
| peak width | >0.0031 min (0.063 s response time, 80 Hz) | |
| injection | 0.5 μL standard injection | |
| flow | 1.4 mL/min | |
| column | 45° C. | |
| temperature | | |
| gradient | 0.0-1.0 min | 15% → 95% B |
| | 1.0-1.1 min | 95% B |
| | Stop time: 1.3 min | |

Method 6

| HPLC | Agilent 1100/1200 system | |
|---|---|---|
| MS | 1200 Series LC/MSD (API-ES +/− 3000/3500 V, | |
| | Quadrupol, G6140A) | |
| MSD signal | Scan pos 150-750 | |
| settings | | |
| column | YMC; Part. No. TA12S03-0302WT; Triart C18, 3 μm, | |
| | 12 nm; 30 × 2.0 mm column | |
| eluant | A: H2O + 0.11% formic acid | |
| | B: MeCN + 0.1% formic acid (HPLC grade) | |
| detection | UV 254 nm, 230 nm, 214 nm (bandwidth 10, reference off) | |
| signal | | |
| spectrum | range: 400-1350 nm; slit: 4 nm | |
| peak width | >0.0031 min (0.063 s response time, 80 Hz) | |
| injection | 0.5 μL standard injection | |
| flow | 1.4 mL/min | |
| column | 45° C. | |
| temperature | | |
| gradient | 0.0-1.0 min | 15% → 95% B |
| | 1.0-1.1 min | 95% B |

Stop time: 1.23 min

Method 7

| HPLC | Agilent 1100/1200 system | |
|---|---|---|
| MS | 1200 Series LC/MSD (MM-ES + APCl +/− 3000 V, | |
| | Quadrupol, G6130B) | |
| MSD signal | Scan pos/neg 150-750 | |
| settings | | |
| column | Waters, Part. No. 186003389, XBridge BEH C18, 2.5 μm, | |
| | 2.1 × 30 mm) column | |
| eluant | A: 5 mM NH4HCO3/18 mM NH3 (pH = 9.2) | |
| | B: acetonitrile (HPLC grade) | |
| detection | UV 254 nm, 230 nm, 214 nm (bandwidth 8, reference off) | |
| signal | | |
| spectrum | range: 400-1350 nm; slit: 4 nm | |
| peak width | >0.0031 min (0.063 s response time, 80 Hz) | |
| injection | 0.5 μL standard injection | |
| flow | 1.4 mL/min | |
| column | 45° C. | |
| temperature | | |
| gradient | 0.0-1.0 min | 15% → 95% B |
| | 1.0-1.1 min | 95% B |

Stop time: 1.3 min

### Method 8

HPLC: Agilent RRLC
Column: XBridge C18, 4.6×75 mm, 3.5 μm
Mobile Phase-A: 10 mM Ammonium Acetate
Mobile Phase-B: Acetonitrile
Gradient program: [Time in min/% of B]: 0/10, 0.2/10, 2.5/75, 3.0/100, 4.8/100, 5.0/10
Detection signal: UV 215/254 nm (Bandwidth 4, Reference off)
Spectrum: Range: 200-400 nm; step: 2 nm
Peak width: >0.1 min (2.0 s response time) (2.5 Hz)
Injection: 4.0 μL injection with needle wash. Wash vial location at 100
Flow rate: 2.0 mL/min
Column temperature: 35° C.
MS: Agilent Technologies-6130 Quadrupole LC/MS
MSD signal settings: Scan positive 70-1200, Scan negative 70-1200
MS Mode: Multi Mode
MS Parameters: Capillary Voltage Positive 3500, Negative 2500;
Corona current (μA): Positive 10, Negative 12; Charging Voltage: Positive 2000, Negative
2000.
Drying gas Flow (1/min): 11; Nebulizer gas (psig): 40, Drying gas Temp (° C.): 300;
Vaporizer Temp (° C.): 240

### Method 9

LCWaters Acquity UPLC
MS Waters SQD2 (ESI Pos/Neg)
Capillary (kV)—3.50
Source Temperature (° C.)—120
Desolvation Temperature (° C.)—350
Cone Gas Flow (L/Hr)—50
Desolvation Gas Flow (L/Hr)—750
MSD signal settings Scan Pos 100-1500, Scan Neg 100-1500
Column Acquity BEH C18, 2.1×50 mm, 1.7 μm
Eluent A: 0.07% Formic Acid in water
B: 0.07% Formic Acid in Acetonitrile
Detection signal PDA Detector
Spectrum UV Range: 200-400 nm;
Injection 0.5 μL
Flow rate 0.6 mL/min
Column temperature 35° C.

| Gradient0.0-0.3 min | | 3% B |
|---|---|---|
| 0.3-2.2 min | 3% | 97% B |
| 2.2-3.3 min | 98% B | |
| 3.3-4.5 min | 98% | 3% B |

### Method 10

UPLC-MS Waters UPLC-Xevo TQ-S Triple quad
MSD signal settings Scan Pos & Neg 100-1500,
   Source Voltage: Capillary Vol (kV)—3.00, Cone (V): 20
Source Temp: Desolvation Temp (° C.): 350
Source Gas Flow: Desolvation (L/Hr): 750
Column ACQUITY UPLC BEH C18 1.7 μm, 2.1×50 mm
Column temperature 35° C.
Eluent A: 0.07% formic acid in water
B: 0.07% formic acid in Acetonitrile
Detection signal PDA Detector
Spectrum UV Range: 200-400 nm
Sampling rate 10 point/sec
Injection 0.5 μL
Flow 0.6 mL/min

| Gradient0.0-0.30 min | | 3% B |
|---|---|---|
| 0.30-2.70 min | 3% | 98% B |
| 2.70-3.50 min | 98% B | |
| 3.50-3.51 min | 98% | 3% B |

### Method 11

LCWaters Acquity UPLC
MS Waters SQD2 (ESI Pos/Neg)
Capillary (kV)—3.50
Source Temperature (° C.)—120
Desolvation Temperature (° C.)—350
Cone Gas Flow (L/Hr)—50
Desolvation Gas Flow (L/Hr)—700
MSD signal settings Scan Pos 100-1500, Scan Neg 100-1500
Column Acquity BEH C18, 2.1×50 mm, 1.7 μm
Eluent A: 0.07% Formic Acid in water
B: 0.07% Formic Acid in Acetonitrile
Detection signal PDA Detector
Spectrum UV Range: 200-400 nm;
Injection 0.5 μL
Flow rate 0.6 mL/min
Column temperature 35° C.

| | | |
|---|---|---|
| Gradient0.0-0.4 min | | 3% B |
| 0.4-2.5 min | 3% | 98% B |
| 2.5-3.4 min | 98% B | |
| 3.4-3.5 min | 98% | 3% B |
| 3.5-4.0 min | 3% B | |

### Method 12

HPLC: Agilent 1260 Series
MS: Agilent LC/MSD Quadrupole
Column: Waters X-Bridge BEH C18, 2.5 μm, 2.1×30 mm XP
Solvent: A: 20 mM NH₄HCO₃/30 mM NH₃ in H₂O; B: acetonitrile (HPLC grade)
Detection: MS: positive and negative mode
Mass range: 200-1200 m/z
Flow: 1.40 mL/min
Column temperature: 45° C.
Gradient: 0.00-1.00 min: 15% B to 95% B
   1.0-1.30 min: 95% B

### Method 13

HPLC: Agilent 1260 Series
MS: Agilent LC/MSD Quadrupole
Column: Waters X-Bridge BEH C18, 2.5 μm, 2.1×30 mm XP
Solvent: A: 20 mM NH₄HCO₃/30 mM NH₃ in H₂O; B: acetonitrile (HPLC grade)
Detection: MS: positive and negative mode
Mass range: 500-1300 m/z
Flow: 1.40 mL/min
Column temperature: 45° C.
Gradient: 0.00-1.00 min: 15% B to 95% B
   1.00-1.30 min: 95% B

### Method 14

HPLC Agilent 1260 System
MS 1200 Series LC/MSD (API-ES+/−3000/3500 V, Quadrupole, G6130A)
MSD signal settings Scan pos and neg 150-900
column Waters; Part. No. 186003020; XBridge C18, 3 μm; 30×2.1 mm column
eluent A: H2O+10 mM NH4HCO3
B: MeCN (HPLC grade)
detection signal UV 315 nm (bandwidth 170 nm, reference off)
spectrum range: 190-400 nm; slit: 1 nm
peak width>0.0063 min (0.13 s response time, 40 Hz)
injection 5 μL standard injection
flow 1.0 mL/min
column temperature 60° C.

| | | |
|---|---|---|
| gradient 0.0-1.5 min | 5% A | 95% B |
| 1.5-2.0 min | 95% B | |

Stop time: 3.1 min

The compounds according to the present invention and their intermediates may be obtained using methods of synthesis which are known to the one skilled in the art and described in the literature of organic synthesis. Preferably, the compounds according to the invention are prepared by the methods of synthesis described hereinafter in which the substituents of the general formulae have the meanings given hereinbefore. These methods are intended as an illustration of the invention without restricting its subject matter and the scope of the compounds claimed to these examples. It is to be understood that, in certain cases, a specific substituent may be present in a synthetic scheme only for ease of representation, when, in fact, different substituents could be present at the same position, in accordance with the definitions of the substituents herein. For example, allyl may be depicted when, in fact, any alkene may be used. Where the preparation of starting compounds is not described, they are commercially obtainable or may be prepared analogously to known compounds or methods described herein. Substances described in the literature are prepared according to the published methods of synthesis. It is to be understood that compounds of a certain formula may be converted into different compounds of the same formula. In some cases, the order in carrying out the reaction steps may be varied. Variants of the reaction methods that are known to the one skilled in the art but not described in detail here may also be used. Any functional groups in the starting materials or intermediates may be protected using conventional protecting groups. These protecting groups may be cleaved again at a suitable stage within the reaction sequence using methods familiar to the one skilled in the art.

### Synthesis Towards Intermediate 10 and 11

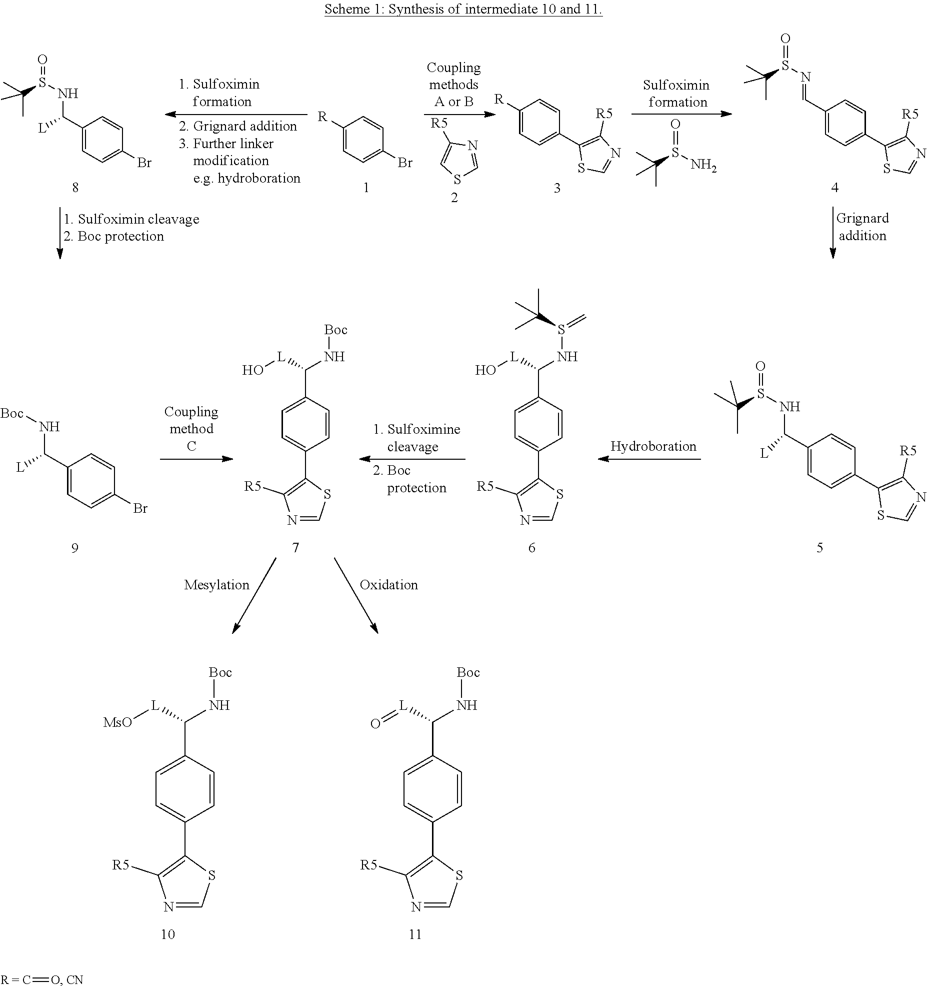

The first step to generate aldehyde 3 can be realized via various metal catalyzed cross-coupling or CH activating reactions (methods A or B) starting from an aldehyde or nitrile 1 and thiazole 2. Aldehyde 3 is transformed into the corresponding sulfoximine 4 using e.g. Ellman's auxiliary. The chiral auxiliary allows subsequent treatment with a broad variety of alkene Grignard reagents to install linkers with different chain length in the benzylic position leading to intermediate 5. At this stage the linker can be further modified e.g. using a hydroboration reaction to install the corresponding alcohol 6, which can then be used for further transformations. Cleavage of the chiral auxiliary and reprotection using e.g. (Boc)₂O is leading to amine 7. To enable the final coupling to the SMARCA binder, alcohol 7 can be modified in e.g. a mesylate 10 or e.g. an aldehyde 11. An alternative way to intermediate 7 is starting from aldehyde 1, which is transferred into the corresponding sulfoximine. Grignard addition and further modification of the alkene, e.g. hydroboration is leading to intermediate 8. Cleavage of the chiral auxiliary followed by reprotection e.g. using (Boc)₂O is giving intermediate 9. Intermediate 9 can be transformed e.g. using a Suzuki coupling into amine 7.

### Synthesis Towards Intermediates 3 Using Method a (Suzuki Coupling)

### Experimental Procedure for the Synthesis of 3a

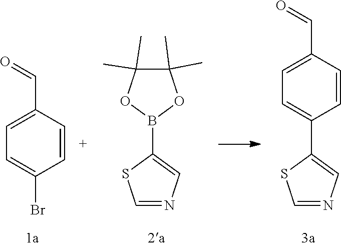

4-bromobenzaldehyde 1a (0.60 g, 3.24 mmol, 1.0 equiv.), thiazole-5-boronic acid pinacol ester 2′a (1.37 g, 6.48 mmol, 2.0 equiv.), sodium carbonate (0.86 g, 8.10 mmol, 2.5 equiv.) and tetrakis-(triphenylphoshine)palladium(0) (0.19 g, 0.16 mmol, 0.05 equiv.) are dissolved in dioxane (12.0 mL) and water (3.6 mL). The reaction mixture is degassed with argon for 5 min and stirred at 80° C. for 1.5 hours. Then the reaction mixture is cooled to rt, diluted with DCM (50 mL) and filtered over a pad of Celite. The mixture is washed with water (10 mL) and sat. NaCk-solution (10 mL). The organic layer is passed through a phase separator cartridge and concentrated. The crude product is purified via NP chromatography (10-50% EtOAc in cyclohexane) to afford 3a (0.60 g, 96%).

TABLE 1

| | | | | HPLC |
|---|---|---|---|---|
| # | structure | tᵣₑₜ [min] | [M + H]⁺ | method |
| 3a | 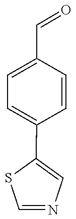 [SMILES: O=Cc1ccc(-c2cncs2)cc1] | 0.94 | 190 | 14 |

### Synthesis Towards Intermediates 3 Using Method B (C—H Activation)

### Experimental Procedure for the Synthesis of 3b

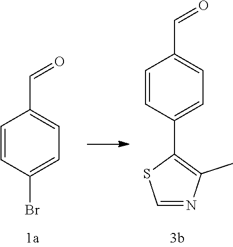

4-bromobenzaldehyde 1a (100 g, 0.54 mol, 1.0 equiv.), 4-methylthiazole (98.3 mL, 1.08 mol, 2.0 equiv.) and potassium acetate (106 g, 1.08 mol, 2.0 equiv.) are taken up in DMAc (100 mL) and purged with argon. Then palladium(II) acetate (1.21 g, 5.40 mmol, 0.01 equiv.) is added and the mixture is stirred at 120° C. for 60 min under nitrogen atmosphere. The reaction mixture is cooled to rt, quenched with water (1 L) and stirred for 60 min. The solids are collected by filtration, rinsed with water and dried under vacuum at 50° C. to afford 3b (98.2 g, 89%).

The following intermediates 3 (table 2) are available in an analogous manner using different starting materials 1. The crude product 3 is purified by chromatography if necessary.

TABLE 2

| | | | | HPLC |
|---|---|---|---|---|
| # | Structure | tᵣₑₜ [min] | [M + H]⁺ | method |
| 3b | 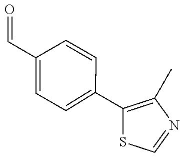 | 1.01 | 204 | 14 |
| 3c | 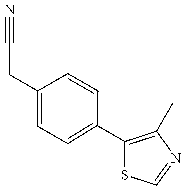 | 1.90 | 214 | 9 |

### Experimental Procedure for the Synthesis of 4a

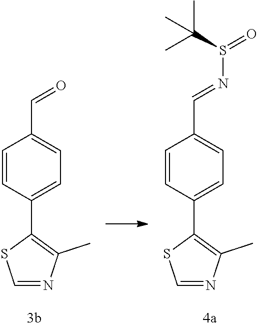 [SMILES: Cc1ncsc1-c1ccc(C=O)cc1 ; Cc1ncsc1-c1ccc(/C=N/[S@](=O)C(C)(C)C)cc1]

3b (100 g, 0.49 mol, 1.0 equiv.) and (R)-(+)-2-methyl-2-propanesulfinamide (89.4 g, 0.74 mol, 1.5 equiv.) are taken up in anhydrous THF (1.0 L). Then titanium(IV) isopropoxide (223 mL, 0.74 mol, 1.5 equiv.) is added dropwise at 0° C. under nitrogen atmosphere. After complete addition, the cooling is removed and the mixture is stirred at rt for 16 h. The reaction is quenched with ice-cold water. The precipitating solids are filtered through a pad of Celite and rinsed with EtOAc (3×1 L). The filtrate layers are separated. The organic layer is dried over Na₂SO₄, filtered and concentrated under reduced pressure. The obtained crude is purified by silica gel column chromatography (30-60% EtOAc in hexanes) to give 4a (110 g, 73%) The following intermediates 4 and 8′a (table 3) are available in an analogous manner using different starting materials of formula 3 or 1, respectively. The crude product 4 or 8′a is purified by chromatography if necessary.

TABLE 3

| | | tᵣₑₜ | [M + | HPLC |
|---|---|---|---|---|
| # | structure | [min] | H]⁺ | method |
| 4a | 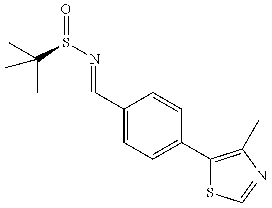 [SMILES: Cc1ncsc1-c1ccc(/C=N/[S@](=O)C(C)(C)C)cc1] | 1.29 | 307 | 14 |
| 4b | 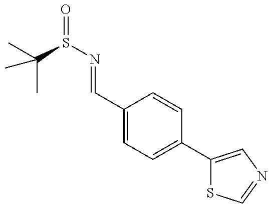 [SMILES: CC(C)(C)[S@@](=O)/N=C/c1ccc(-c2cncs2)cc1] | 1.27 | 293 | 14 |
| 8′a | 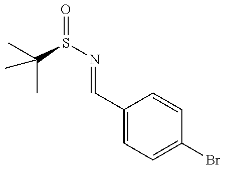 | 1.52 | 288 | 14 |

### Experimental Procedure for the Synthesis of 5a

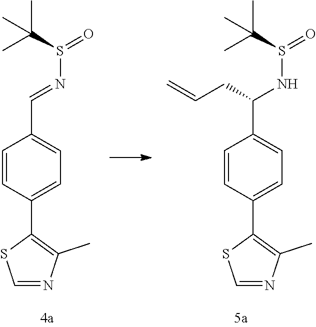 [SMILES: Cc1ncsc1-c1ccc(/C=N/[S@](=O)C(C)(C)C)cc1 ; C=CC[C@H](N[S@](=O)C(C)(C)C)c1ccc(-c2scnc2C)cc1]

4a (35.0 g, 0.11 mol, 1.0 equiv.) is dissolved in anhydrous THF (350 mL) under nitrogen atmosphere and cooled to 0° C. Then 1 M THF solution of allylmagnesium bromide (137 mL, 0.14 mol, 1.2 equiv.) is added slowly at 0° C. under nitrogen atmosphere. After complete addition, the cooling is removed and the mixture is stirred at rt for 2 h. The reaction is quenched with sat. ammonium chloride solution and diluted with EtOAc. The layers are separated. The organic layer is dried over Na₂SO₄, filtered and concentrated under reduced pressure. The obtained crude is purified by silica gel column chromatography (40-80% EtOAc in hexanes) to give 5a (25.0 g, 63%).

The following intermediates 5 and 8′b (table 4) are available in an analogous manner using different starting materials of formula 4 and 8′a, respectively, and different Grignard reagents. The crude product 5 is purified by chromatography if necessary.

TABLE 4

| | | | | HPLC |
|---|---|---|---|---|
| # | structure | tᵣₑₜ [min] | [M + H]⁺ | method |
| 5a | 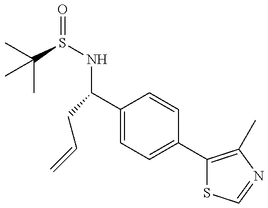 [SMILES: C=CC[C@H](N[S@](=O)C(C)(C)C)c1ccc(-c2scnc2C)cc1] | 2.12 | 349 | 9 |
| 5b | 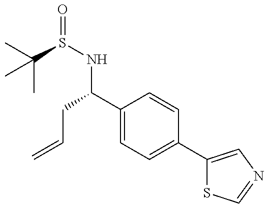 [SMILES: C=CC[C@H](N[S@](=O)C(C)(C)C)c1ccc(-c2cncs2)cc1] | 1.24 | 335 | 14 |
| 5c | 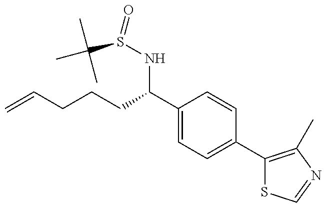 [SMILES: C=CCCC[C@H](N[S@](=O)C(C)(C)C)c1ccc(-c2scnc2C)cc1] | 2.99/3.14 | 377 | 8 |
| 5d | 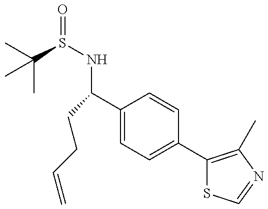 [SMILES: C=CCC[C@H](N[S@](=O)C(C)(C)C)c1ccc(-c2scnc2C)cc1] | 2.21 | 363 | 9 |
| 5e | 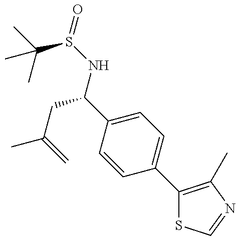 [SMILES: C=C(C)C[C@H](N[S@](=O)C(C)(C)C)c1ccc(-c2scnc2C)cc1] | 0.68 | 363 | 4 |
| 5f | 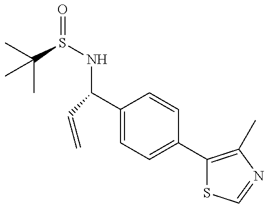 [SMILES: C=C[C@H](N[S@](=O)C(C)(C)C)c1ccc(-c2scnc2C)cc1] | 0.55 | 335 | 4 |
| 8′b | 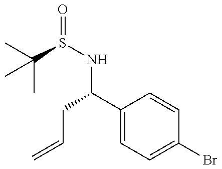 [SMILES: C=CC[C@H](N[S@](=O)C(C)(C)C)c1ccc(Br)cc1] | 1.41 | 330 | 14 |

### Experimental Procedure for the Synthesis of 6a

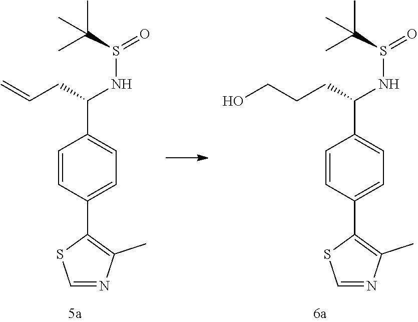 [SMILES: C=CC[C@H](N[S@](=O)C(C)(C)C)c1ccc(-c2scnc2C)cc1 ; Cc1ncsc1-c1ccc([C@H](CCCO)N[S@](=O)C(C)(C)C)cc1]

5a (220 g, 0.63 mol, 1.0 equiv.) is dissolved in THF (2.0 L) under nitrogen atmosphere and cooled to 0° C. Then 0.5 M THF solution of 9-borabicyclo[3.3.1]nonane (9-BBN, 3.79 L, 1.89 mol, 3.0 equiv.) is added slowly at 0° C. After complete addition, the cooling is removed and the mixture is stirred at rt for 2 h until complete conversion of 5a. The reaction mixture is cooled to 0° C. again and 30% aq. hydrogen peroxide solution (0.644 L, 6.31 mol, 10.0 equiv.) is added dropwise, followed by 4 N NaOH solution (1.58 L, 6.31 mol, 10.0 equiv.). The cooling is removed and the mixture is stirred at rt for 1 h. The reaction is slowly quenched and acidified to pH 4 by carefully adding 1 N aq. hydrochloric acid. It is extracted with DCM twice. The combined organic layers are dried over Na₂SO₄, filtered and concentrated under reduced pressure. The crude product is purified by combi flash column chromatography (5-8% MeOH in DCM) to give 6a (120 g, 52%) as pure diastereoisomer. The following intermediates 6 and 8 (table 5) are available in an analogous manner using different starting materials 5 and 8′b. The crude product 6 and 8 is purified by chromatography if necessary.

TABLE 5

| | | tᵣₑₜ | [M + | HPLC |
|---|---|---|---|---|
| # | structure | [min] | H]⁺ | method |
| 6a | 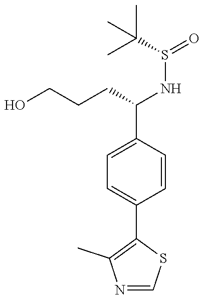 [SMILES: Cc1ncsc1-c1ccc([C@H](CCCO)N[S@@](=O)C(C)(C)C)cc1] | 1.82 | 367 | 9 |
| 6b | 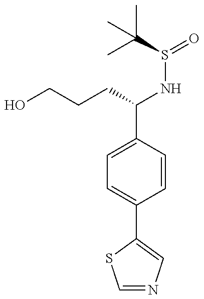 [SMILES: CC(C)(C)[S@@](=O)N[C@@H](CCCO)c1ccc(-c2cncs2)cc1] | 1.03 | 353 | 14 |
| 8a | 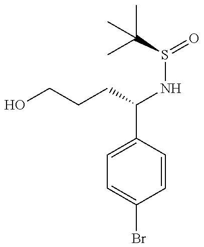 [SMILES: CC(C)(C)[S@@](=O)N[C@@H](CCCO)c1ccc(Br)cc1] | 1.17 | 348 | 14 |
| 6c | 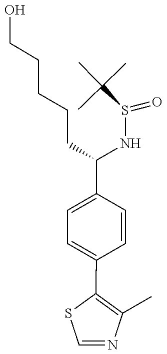 [SMILES: Cc1ncsc1-c1ccc([C@H](CCCCCO)N[S@](=O)C(C)(C)C)cc1] | 1.89 | 395 | 9 |
| 6d | 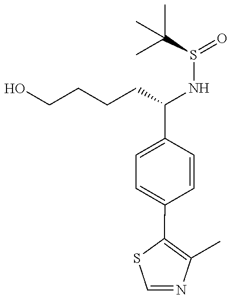 [SMILES: Cc1ncsc1-c1ccc([C@H](CCCCO)N[S@](=O)C(C)(C)C)cc1] | 1.86 | 381 | 9 |
| 6e | 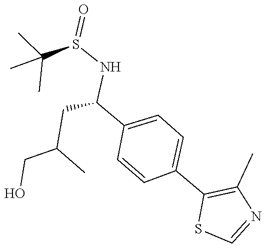 [SMILES: Cc1ncsc1-c1ccc([C@H](CC(C)CO)N[S@](=O)C(C)(C)C)cc1] | 1.10/ 1.13 | 381 | 14 |
| 6f | 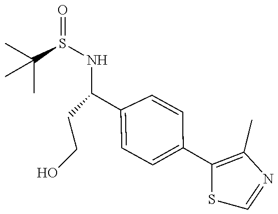 [SMILES: Cc1ncsc1-c1ccc([C@H](CCO)N[S@](=O)C(C)(C)C)cc1] | 1.75 | 353 | 9 |

### Experimental Procedure for the Synthesis of 7a

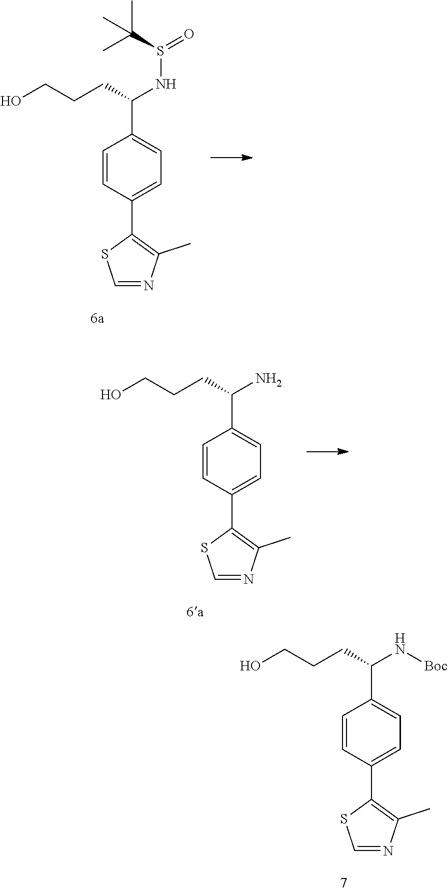 [SMILES: Cc1ncsc1-c1ccc([C@H](CCCO)N[S@](=O)C(C)(C)C)cc1 ; Cc1ncsc1-c1ccc([C@@H](N)CCCO)cc1 ; Cc1ncsc1-c1ccc([C@H](CCCO)NC(=O)OC(C)(C)C)cc1]

6a (60.0 g, 0.16 mol, 1.0 equiv.) is dissolved in DCM (600 mL) and cooled to 0° C. Then hydrochloric acid, 4 N in dioxane (409 mL, 1.64 mol, 10.0 equiv.) is added dropwise at 0° C.

The mixture is stirred at the same temperature for 2 h and then allowed to reach rt. The solvents are removed under reduced pressure. The residue is triturated with diethyl ether and the solids are dried to give 6′a (48.0 g, 98%) as a hydrochloride salt, which is used for the next step.

6′a (15.0 g, 50.0 mmol, 1.0 equiv.) is dissolved in 1,4-dioxane (75.0 mL) and water (75.0 mL) and cooled to 0° C. Then triethylamine (21.1 mL, 150.0 mmol, 3.0 equiv.) and di-tert-butyl dicarbonate (17.3 mL, 75.0 mmol, 1.5 equiv.) is added dropwise at 0° C. The mixture is stirred at the same temperature for 2 h and then allowed to reach rt. The reaction mixture is diluted with water and extracted with EtOAc (2×500 mL). The combined organics are washed with brine, dried over Na₂SO₄, filtered and concentrated under reduced pressure. The obtained crude is purified by silica gel column chromatography (50-80% EtOAc in hexanes) to give 7a (14.0 g, 77%).

The following intermediates 7 and 9 (table 6) are available in an analogous manner using different starting materials 6 and 8, respectively. The crude product 7 and 9 is purified by chromatography if necessary.

TABLE 6

| | | tᵣₑₜ | [M + | HPLC |
|---|---|---|---|---|
| # | structure | [min] | H]⁺ | method |
| 7a | 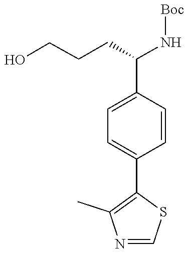 [SMILES: Cc1ncsc1-c1ccc([C@H](CCCO)NC(=O)OC(C)(C)C)cc1] | 1.36 | 363 | 9 |
| 7b | 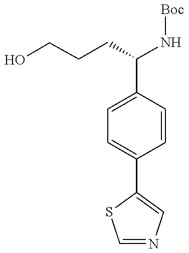 [SMILES: CC(C)(C)OC(=O)N[C@@H](CCCO)c1ccc(-c2cncs2)cc1] | 0.53 | 349 | 4 |
| 9a | 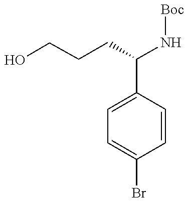 [SMILES: CC(C)(C)OC(=O)N[C@@H](CCCO)c1ccc(Br)cc1] | 0.62 | 344/346 | 4 |
| 7c | 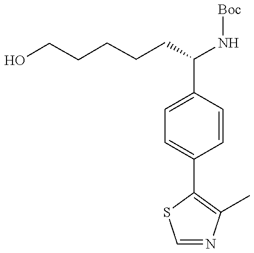 [SMILES: Cc1ncsc1-c1ccc([C@H](CCCCCO)NC(=O)OC(C)(C)C)cc1] | 0.61 | 391 | 3 |
| 7d | 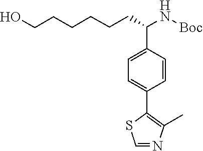 [SMILES: Cc1ncsc1-c1ccc([C@H](CCCCCCO)NC(=O)OC(C)(C)C)cc1] | 1.98 | 377 | 9 |
| 7e | 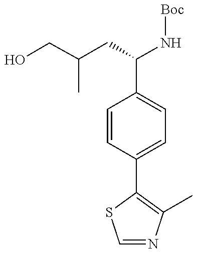 [SMILES: Cc1ncsc1-c1ccc([C@H](CC(C)CO)NC(=O)OC(C)(C)C)cc1] | 0.57 | 377 | 4 |
| 7f | 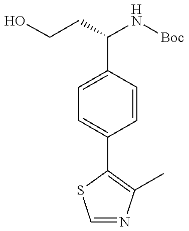 [SMILES: Cc1ncsc1-c1ccc([C@H](CCO)NC(=O)OC(C)(C)C)cc1] | 1.75 | 353 | 9 |

### Synthesis Towards Intermediate 7g Using Method C

### Experimental Procedure for the Synthesis of 9′a

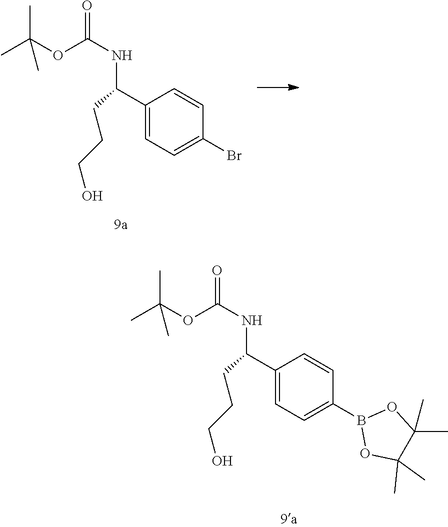 [SMILES: CC(C)(C)OC(=O)N[C@@H](CCCO)c1ccc(Br)cc1 ; CC(C)(C)OC(=O)N[C@@H](CCCO)c1ccc(B2OC(C)(C)C(C)(C)O2)cc1]

A stirred solution of 9a (5.0 g; 0.015 mol; 1.0 equiv.), 4,4,5,5,4′,4′,5′,5′-Octamethyl-[2,2′]bi[[1,3,2]dioxaborolanyl] (5.53 g; 0.022 mol; 1.5 equiv.), potassium acetate (2.86 g; 0.029 mol; 2.0 equiv.) and [1,1′-Bis(diphenylphosphino)ferrocene]dichloropalladium(II) complex with dichloro methane (1.06 g; 0.001 mol; 0.1 equiv.) in 1,4-Dioxane (50.0 mL) is purged with argon and stirred at 90° C. for 6 h. The reaction mixture is concentrated under reduced pressure and the remaining residue is purified by column chromatography to get the desired product 9′a (2.6 g, 46%).

TABLE 7

| | | | | HPLC |
|---|---|---|---|---|
| # | Structure | tᵣₑₜ [min] | [M + H]⁺ | method |
| 9′a | 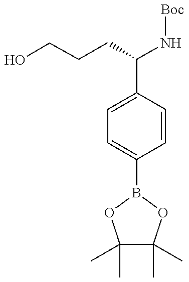 [SMILES: CC(C)(C)OC(=O)N[C@@H](CCCO)c1ccc(B2OC(C)(C)C(C)(C)O2)cc1] | 2.40 | 392 | 9 |

### Experimental Procedure for the Synthesis of 7g

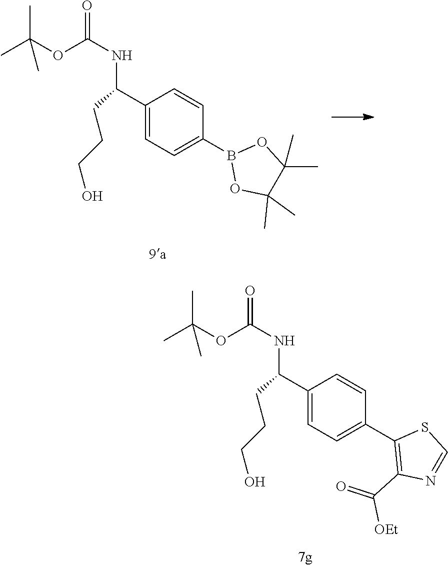

Dioxoborolan 9′a (150 mg; 0.37 mmol; 1.0 equiv.), ethyl 5-bromothiazole-4-carboxylate (118 mg; 0.49 mmol; 1.3 equiv.), sodium carbonate (159 mg; 1.50 mmol; 4.0 equiv.) and tetrakis(triphenylphosphine)palladium(0) (44 mg; 0.037 mmol; 0.1 equiv.) are dissolved in dimethoxy ethane (3.0 mL) and water (0.9 mL). The reaction mixture is purged with Argon for 5 min, then stirred at 90° C. for 2 h. The reaction mixture is diluted with DCM (10 mL) and water (6 mL). The layers are separated and the aqueous layer is extracted with DCM three times. The combined organic layers are concentrated and purified by reverse phase chromatography to get 7g (108 mg; 69%)

TABLE 8

| | | | | HPLC |
|---|---|---|---|---|
| # | structure | tᵣₑₜ [min] | [M + H]⁺ | method |
| 7g | 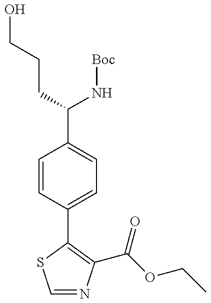 [SMILES: CCOC(=O)c1ncsc1-c1ccc([C@H](CCCO)NC(=O)OC(C)(C)C)cc1] | 0.524 | 421 | 4 |

### Synthesis Towards Intermediate 10

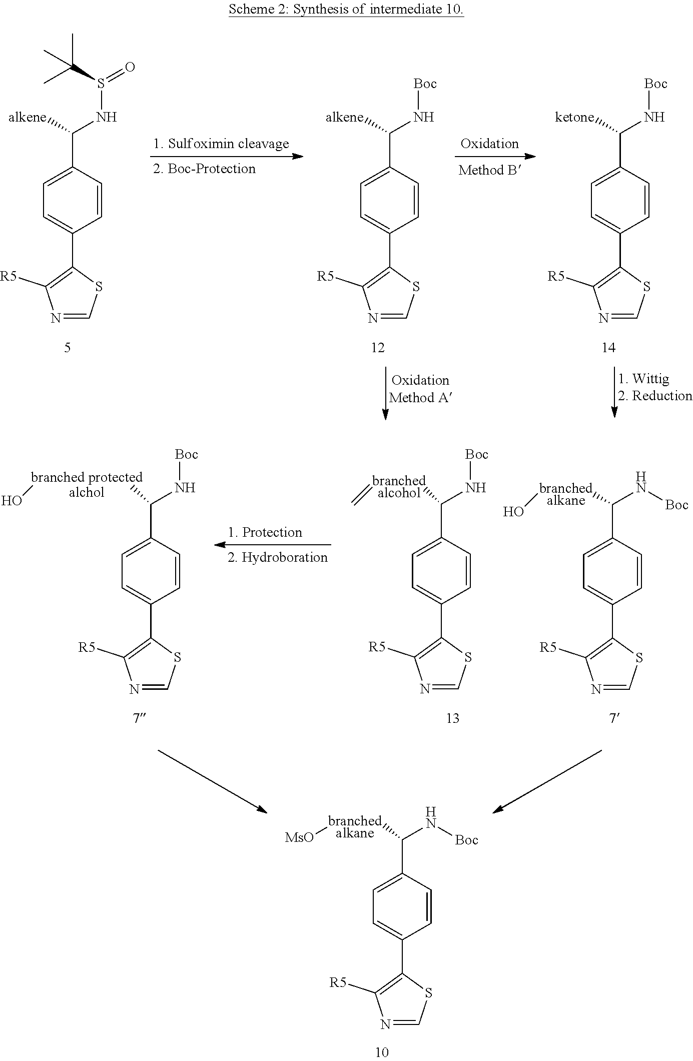

Cleavage of the chiral auxiliary on intermediate 5 using e.g. TFA followed by e.g. installation of a boc protecting group is leading to intermediate 12, which can be further functionalized e.g. by installation of an additional oxygen in the linker in various positions leading to intermediate 13 (method A′) or intermediate 14 (method B′). To further modify the linker via method A′, the newly installed oxygen has to be protected e.g. using TBDMSCI. This allows then further modification of the double bond e.g. using standard hydroboration conditions giving alcohol 7″. Installation of an additional methyl group in the linker can be achieved via oxidation using method B′ leading to intermediate 14. Subsequent chain elongation e.g. under Wittig type reaction conditions, using a reagent bearing an ester functionality, is leading after standard reduction of the ester to alcohol 7′. Alcohols 7 are then mesylated giving intermediate 10.

### Experimental Procedure for the Synthesis of 5′a

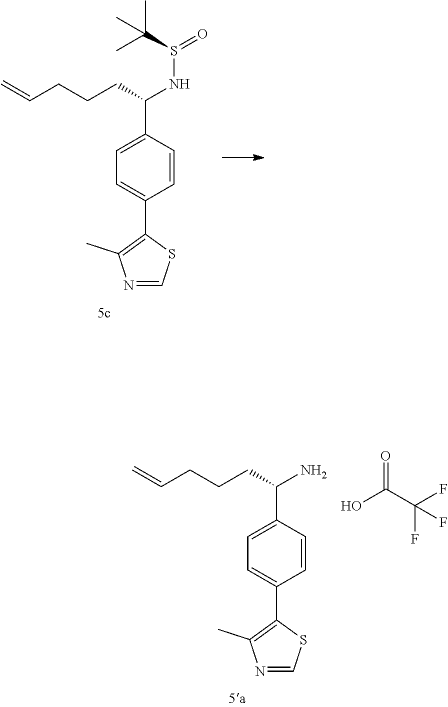 [SMILES: C=CCCC[C@H](N[S@](=O)C(C)(C)C)c1ccc(-c2scnc2C)cc1 ; C=CCCC[C@H](N)c1ccc(-c2scnc2C)cc1 ; O=C(O)C(F)(F)F]

Sulfinamide 5c (2.00 g, 5.31 mmol; 1.0 equiv.) is dissolved in dichloromethane (20 mL), cooled to 0° C. and trifluoroacetic acid (3.03 g, 26.6 mmol, 5.0 equiv.) is added dropwise. The mixture is stirred at 0° C. for 16 h. The solvents are removed under reduced pressure. The obtained crude is washed with diethyl ether and dried to give 5′a (1.50 g, 73.1%) as a trifluoroacetate salt.

The following intermediates 5′ (table 9) are available in an analogous manner using different starting materials 5. The crude product 5′ is purified by chromatography if necessary.

TABLE 9

| | | | | HPLC |
|---|---|---|---|---|
| # | Structure | tᵣₑₜ [min] | [M + H]⁺ | method |
| 5′a | 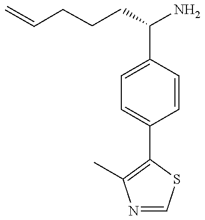 [SMILES: C=CCCC[C@H](N)c1ccc(-c2scnc2C)cc1] | 2.18 | 273 | 8 |
| 5′b | 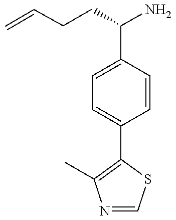 [SMILES: C=CCC[C@H](N)c1ccc(-c2scnc2C)cc1] | 2.05 | 259 | 8 |

### Experimental Procedure for the Synthesis of 12a

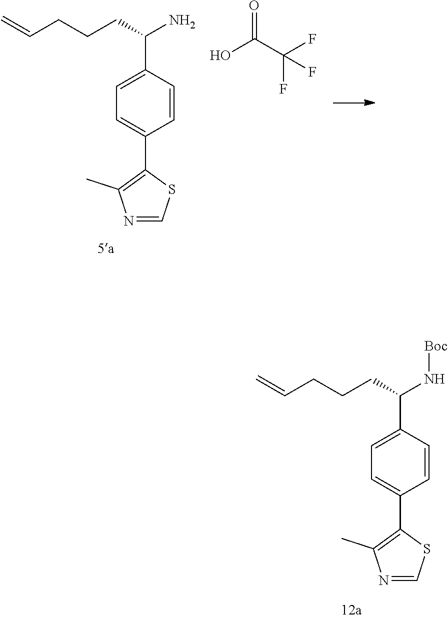 [SMILES: C=CCCC[C@H](N)c1ccc(-c2scnc2C)cc1 ; O=C(O)C(F)(F)F ; C=CCCC[C@H](NC(=O)OC(C)(C)C)c1ccc(-c2scnc2C)cc1]

Amine 5′a (1.50 g, 3.88 mmol; 1.0 equiv.) is dissolved in dioxane (10 mL) and water (10 mL) and cooled to 0° C. Then TEA (1.96 g, 19.4 mmol, 5.0 equiv.) and Boc anhydride (1.27 g, 5.82 mmol, 1.5 equiv.) are added dropwise. The mixture is stirred at rt for 2 h.

The reaction is diluted with water and extracted with EtOAc (2×250 mL). The combined organics are dried over Na₂SO₄, filtered and concentrated under reduced pressure. The obtained crude is purified by silica gel chromatography followed by RP-chromatography to give 12a (0.50 g, 34.6%).

The following intermediates 12 (table 10) are available in an analogous manner using different starting materials 5′. The crude product 12 is purified by chromatography if necessary.

TABLE 10

| | | | | HPLC |
|---|---|---|---|---|
| # | structure | tᵣₑₜ [min] | [M + H]⁺ | method |
| 12a | 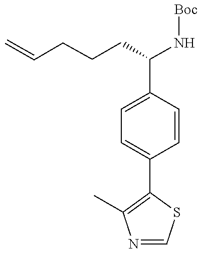 [SMILES: C=CCCC[C@H](NC(=O)OC(C)(C)C)c1ccc(-c2scnc2C)cc1] | 2.33 | 373 | 11 |
| 12b | 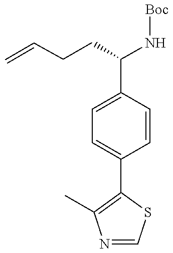 [SMILES: C=CCC[C@H](NC(=O)OC(C)(C)C)c1ccc(-c2scnc2C)cc1] | 2.43 | 359 | 9 |

### Experimental Procedure for the Synthesis of 13a

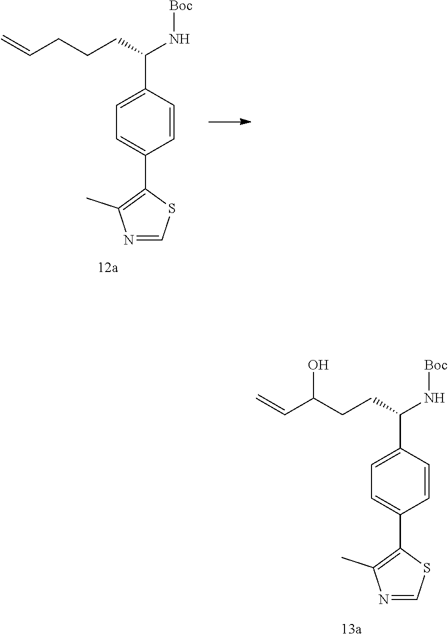 [SMILES: C=CCCC[C@H](NC(=O)OC(C)(C)C)c1ccc(-c2scnc2C)cc1 ; C=CC(O)CC[C@H](NC(=O)OC(C)(C)C)c1ccc(-c2scnc2C)cc1]

In a sealable tube selenium dioxide (305 mg, 2.75 mmol, 3.5 equiv.) is taken up in dichloromethane, dry (6.0 mL) and cooled to 0° C. before tert-butyl hydroperoxide (0.521 mL; 2.87 mmol; 3.7 equiv.) is added. The mixture is stirred at 0° C. for 30 minutes. Then alkene 12a (300 mg; 0.785 mmol, 1.0 equiv.), dissolved in 1.5 mL DCM, is added dropwise. The reaction mixture is allowed to reach RT and is stirred for 42 h. The reaction mixture is quenched with 10% aq. Na₂S₂O₃-solution and diluted with DCM. The layers are separated.

The organic layer is passed through a phase separator cartridge and concentrated under reduced pressure. The residue is dissolved in ACN/MeOH/H₂O, filtered through a syringe filter and purified by RP-chromatography (15-85% MeCN in H₂O) to give 13a (154 mg, 50.5%).

TABLE 11

| | | | | HPLC |
|---|---|---|---|---|
| # | structure | tᵣₑₜ [min] | [M + H]⁺ | method |
| 13a | 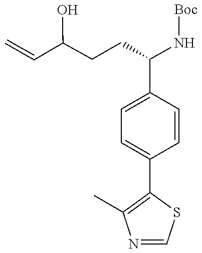 [SMILES: C=CC(O)CC[C@H](NC(=O)OC(C)(C)C)c1ccc(-c2scnc2C)cc1] | 0.64 | 389 | 4 |

### Experimental Procedure for the Synthesis of 13b

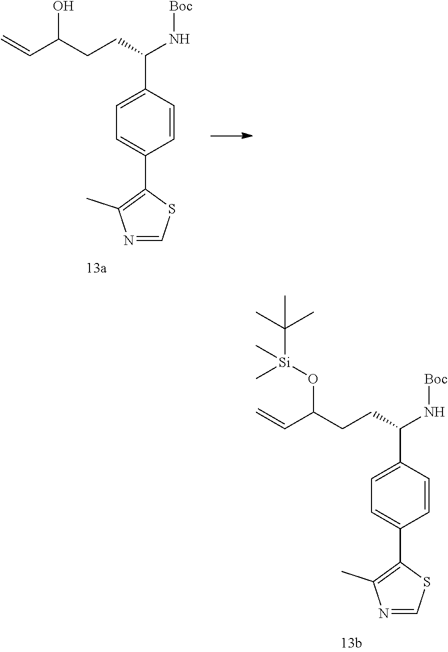 [SMILES: C=CC(O)CC[C@H](NC(=O)OC(C)(C)C)c1ccc(-c2scnc2C)cc1 ; C=CC(CC[C@H](NC(=O)OC(C)(C)C)c1ccc(-c2scnc2C)cc1)O[Si](C)(C)C(C)(C)C]

In a sealable tube alcohol 13a (154 mg, 0.396 mmol, 1.0 equiv.) and imidazole (82 mg, 1.19 mmol, 3.0 equiv.) are dissolved in dichloromethane, dry (3.0 mL) and cooled to 0° C. Then tert-butyldimethylchlorosilane (0.92 mg; 0.595 mmol; 1.5 equiv.), dissolved in 0.5 mL DCM, is added. The mixture is allowed to reach RT and is stirred overnight. The reaction mixture is diluted with half sat. sodium bicarbonate solution and EtOAc. The layers are separated. The aqueous layer is extracted with EtOAc. The combined organic layers are washed with brine, dried over MgSO₄ and concentrated under reduced pressure. The crude is dissolved in DCM and purified by silica gel column chromatography (0-20% EtOAc in cyclohexane) to give 13b (184 mg, 92.3%).

TABLE 12

| | | | | HPLC |
|---|---|---|---|---|
| # | structure | tᵣₑₜ [min] | [M + H]⁺ | method |
| 13b | 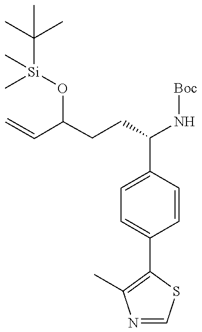 [SMILES: C=CC(CC[C@H](NC(=O)OC(C)(C)C)c1ccc(-c2scnc2C)cc1)O[Si](C)(C)C(C)(C)C] | 1.11 | 503 | 4 |

### Experimental Procedure for the Synthesis of 7j

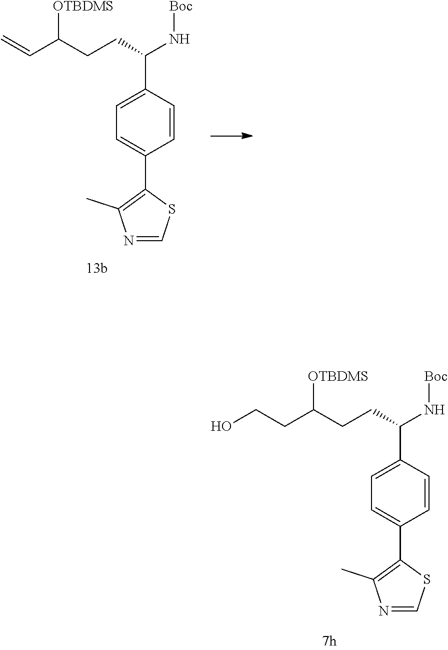 [SMILES: C=CC(CC[C@H](NC(=O)OC(C)(C)C)c1ccc(-c2scnc2C)cc1)O[Si](C)(C)C(C)(C)C ; Cc1ncsc1-c1ccc([C@H](CCC(CCO)O[Si](C)(C)C(C)(C)C)NC(=O)OC(C)(C)C)cc1]

In a 5 mL MW-tube 13b (45.0 mg; 0.090 mmol; 1.00 eq.) is dissolved in THF anh. (1.0 mL) and cooled to 0° C. 9-borabicyclo[3.3.1]nonane 0.5M in THF (0.36 mL; 0.179 mmol; 2.00 eq.) is added slowly. The ice-bath is removed after 5 min and the reaction mixture is stirred at RT for 1h. Additional 9-borabicyclo[3.3.1]nonane 0.5M in THF (0.36 mL; 0.179 mmol; 2.00 eq.) is added again at 0° C. and stirring is continued at RT for an additional hour. The reaction mixture is cooled to 0° C. and hydrogen peroxide (0.091 mL; 0.895 mmol; 10.00 eq.), followed by NaOH 4M (0.224 mL; 0.895 mmol; 10.0 eq.) are added at 0° C. and the ice bath is removed after 5 min. The reaction mixture is stirred at RT for 30 minutes. The mixture is diluted with DCM and sat. NH4Cl. The layers are separated and the aq. layer is extracted with DCM twice. The combined organic layers are passed through a phase separator cartridge and concentrated. The crude is dissolved in ACN/MeOH/H₂O, filtered through a syringe filter and purified by prep. HPLC giving 7h (27 mg, 58% yield).

TABLE 13

| | | tᵣₑₜ | | HPLC |
|---|---|---|---|---|
| # | structure | [min] | [M + H]⁺ | method |
| 7h | 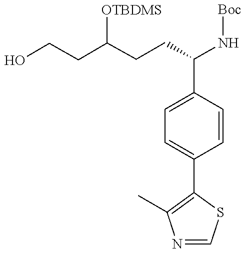 [SMILES: Cc1ncsc1-c1ccc([C@H](CCC(CCO)O[Si](C)(C)C(C)(C)C)NC(=O)OC(C)(C)C)cc1] | 1.64 | 521 | 4 |

### Experimental Procedure for the Synthesis of 14a

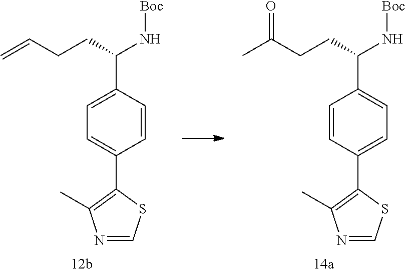 [SMILES: C=CCC[C@H](NC(=O)OC(C)(C)C)c1ccc(-c2scnc2C)cc1 ; CC(=O)CC[C@H](NC(=O)OC(C)(C)C)c1ccc(-c2scnc2C)cc1]

Alkene 12b (8.50 g, 23.7 mmol, 1.0 equiv.) is dissolved in MeCN (200 mL) and water (30 mL). Then PdCl₂ (629 mg, 3.5 mmol, 0.15 equiv.) and CrO₃ (4.70 g, 47.0 mmol, 2.0 equiv.) is added. The mixture is stirred at 60° C. for 6 h. The reaction mixture is cooled to rt, diluted with EtOAc, filtered through a Celite pad and rinsed with EtOAc. The layers are separated. The aqueous layer is extracted with EtOAc. The organic layer is dried over Na₂SO₄ and concentrated under reduced pressure. The crude is purified by silica gel column chromatography (30-60% EtOAc in petrol ether) to give 14a (4.00 g, 45.0%).

TABLE 14

| # | structure | tᵣₑₜ [min] | [M + H]⁺ | HPLC method |
|---|---|---|---|---|
| 14a | 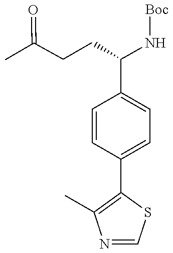 [SMILES: CC(=O)CC[C@H](NC(=O)OC(C)(C)C)c1ccc(-c2scnc2C)cc1] | 2.30 | 375 | 9 |

### Experimental Procedure for the Synthesis of 14b

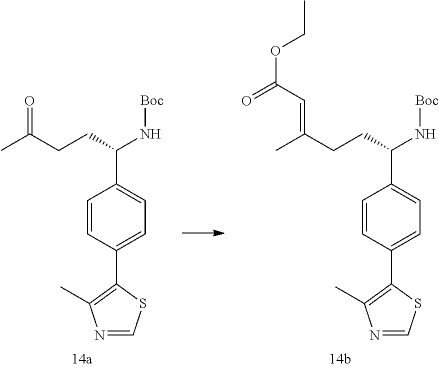 [SMILES: CC(=O)CC[C@H](NC(=O)OC(C)(C)C)c1ccc(-c2scnc2C)cc1 ; CCOC(=O)/C=C(\C)CC[C@H](NC(=O)OC(C)(C)C)c1ccc(-c2scnc2C)cc1]

Sodium hydride 60% (288 mg, 7.2 mmol, 2.7 equiv.) is dissolved in dry THF (10 mL) and cooled to 0° C. Triethyl-phosphonoacetate (1.50 g, 6.7 mmol, 2.5 equiv.) is added dropwise. After complete addition the mixture is stirred at 0° C. for 20 minutes and then cooled to −10° C. Ketone 14a (1.00 g, 2.7 mmol, 1.0 equiv.), dissolved in a minimal amount of THF, is added dropwise. The mixture is allowed to reach rt and stirred for 16 h. The reaction is quenched with ice-cold water and extracted with EtOAc. The combined organic layers are dried over Na₂SO₄ and concentrated under reduced pressure. The crude is purified by silica gel column chromatography (20-50% EtOAc in petrol ether) to give 14b (0.55 g, 46.3%) as a mixture of E/Z-isomers.

TABLE 15

| | | | | HPLC |
|---|---|---|---|---|
| # | structure | tᵣₑₜ [min] | [M + H]⁺ | method |
| 14b | 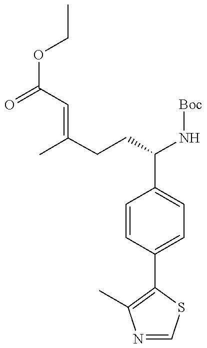 [SMILES: CCOC(=O)/C=C(\C)CC[C@H](NC(=O)OC(C)(C)C)c1ccc(-c2scnc2C)cc1] | 2.49/2.54 | 445 | 9 |

### Experimental Procedure for the Synthesis of 14c

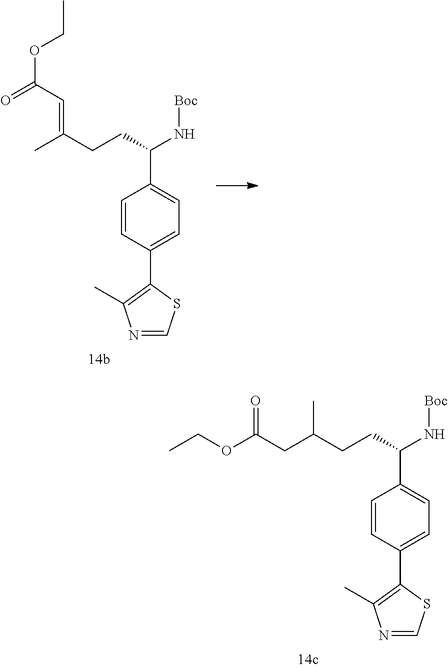 [SMILES: CCOC(=O)/C=C(\C)CC[C@H](NC(=O)OC(C)(C)C)c1ccc(-c2scnc2C)cc1 ; CCOC(=O)CC(C)CC[C@H](NC(=O)OC(C)(C)C)c1ccc(-c2scnc2C)cc1]

Alkene 14b (2.00 g, 4.5 mmol, 1.0 equiv.) is dissolved in MeOH (60 mL) and palladium (10% on carbon, 2.00 g) is added. The reaction mixture is stirred under a hydrogen pressure of 80 PSI at 50° C. for 40 h. The reaction is filtered through a Celite bed, rinsed with 10% MeOH in DCM and concentrated under reduced pressure to give 14c (1.70 g, 84.6%).

TABLE 16

| | | tᵣₑₜ | [M + | HPLC |
|---|---|---|---|---|
| # | structure | [min] | H]⁺ | method |
| 14c | 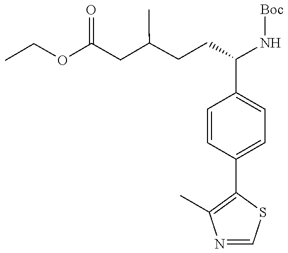 [SMILES: CCOC(=O)CC(C)CC[C@H](NC(=O)OC(C)(C)C)c1ccc(-c2scnc2C)cc1] | 2.50 | 447 | 9 |

### Experimental Procedure for the Synthesis of 7i and 7j

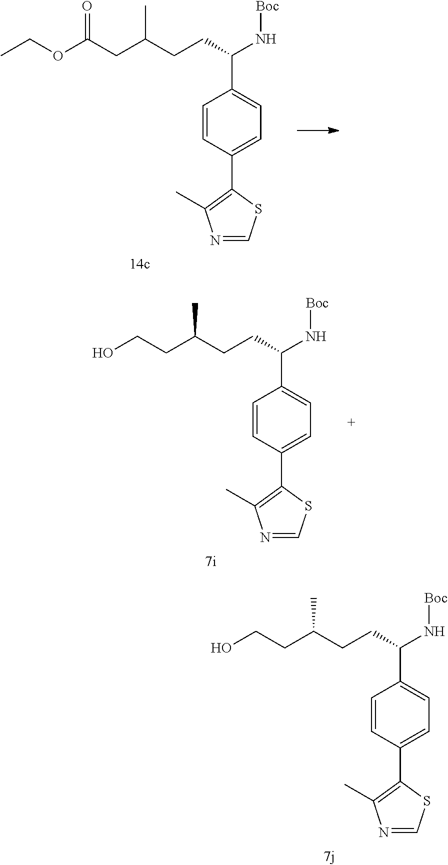 [SMILES: CCOC(=O)CC(C)CC[C@H](NC(=O)OC(C)(C)C)c1ccc(-c2scnc2C)cc1 ; Cc1ncsc1-c1ccc([C@H](CC[C@H](C)CCO)NC(=O)OC(C)(C)C)cc1 ; Cc1ncsc1-c1ccc([C@H](CC[C@@H](C)CCO)NC(=O)OC(C)(C)C)cc1]

Ester 14c (1.70 g, 3.8 mmol, 1.0 equiv.) is dissolved in dry THF (17 mL) and cooled to 0° C. in an icebath. Then LAH 2M in THF (3.80 mL, 7.6 mmol, 2.0 equiv.) is added dropwise. The reaction mixture is stirred at 0° C. for 1 h. The reaction mixture is cautiously quenched with sat. NH₄Cl-solution at 0° C. It is diluted with DCM and water. The salts are filtered off over a Celite pad. The layers are separated and the aqueous phase is extracted with DCM. The combined organic layers are dried over Na₂SO₄ and concentrated under reduced pressure. The crude is purified by silica gel column chromatography (50-70% EtOAc in petrol ether) to give 7i and 7j (1.10 g, 71.4%) as a mixture of diastereoisomers.

The diastereomeric mixture is further purified by SFC (25% MeOH, to obtain the desired products as pure diastereoisomers 7i (0.343 g, 31.2%) and 7j (0.359 g, 32.6%).

The following intermediates 7 (table 17) are available in an analogous manner using different starting materials 14. The crude product 7 is purified by chromatography if

TABLE 17

| | | tᵣₑₜ | [M + | HPLC |
|---|---|---|---|---|
| # | structure | [min] | H]⁺ | method |
| 7i | 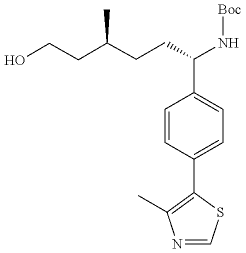 [SMILES: Cc1ncsc1-c1ccc([C@H](CC[C@H](C)CCO)NC(=O)OC(C)(C)C)cc1] | 2.19 | 405 | 9 |
| 7j | 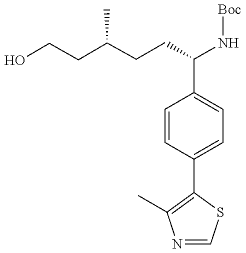 [SMILES: Cc1ncsc1-c1ccc([C@H](CC[C@@H](C)CCO)NC(=O)OC(C)(C)C)cc1] | 2.19 | 405 | 9 |

### Experimental Procedure for the Synthesis of 10b

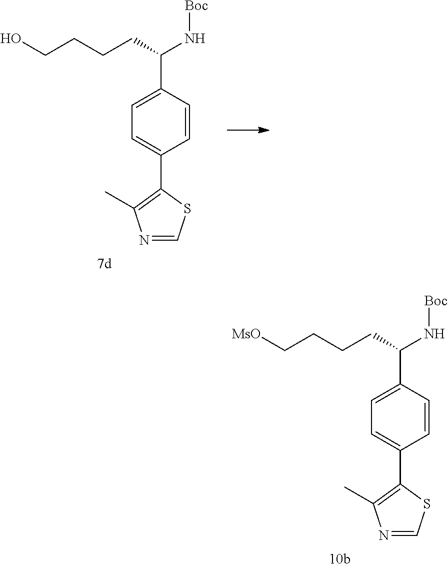 [SMILES: Cc1ncsc1-c1ccc([C@H](CCCCO)NC(=O)OC(C)(C)C)cc1 ; Cc1ncsc1-c1ccc([C@H](CCCCOS(C)(=O)=O)NC(=O)OC(C)(C)C)cc1]

In a glass vial compound 7d (2 g; 5.11 mmol; 1 equiv) and TEA (2.21 mL; 15.34 mmol; 3.0 equiv) are dissolved in DCE (20 mL) and cooled to 0° C. in an icebath. Then MsCl (0.992 mL; 12.8 mmol; 2.5 equiv) is added slowly and the reaction is stirred at 0° C. for 5 min. Complete conversion to the desired product is observed. The reaction is quenched with 20 mL of sat. sodium bicarbonate solution and stirred for 20 min at rt. The layers are separated and the aq. layer is washed with DCM (2×10 mL). The combined organic layers are dried and concentrated. The residue is load on silica and purified by NP-chromatography giving the desired product 10b (2.46 g, quantitative yield).

The following intermediates 10 (table 18) are available in an analogous manner using different starting materials 7. The crude product 10 is purified by chromatography if

TABLE 18

| # | structure | tᵣₑₜ [min] | [M + H]⁺ | HPLC method |
|---|---|---|---|---|
| 10a | 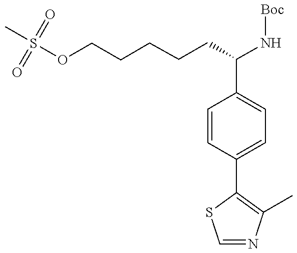 | 0.68 | 469 | 2 |
| 10b | 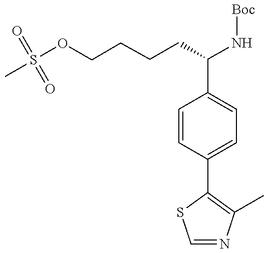 | 0.67 | 455 | 2 |
| 10c | 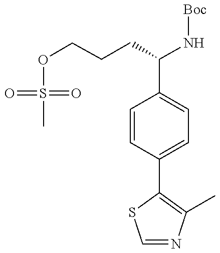 [SMILES: Cc1ncsc1-c1ccc([C@H](CCCOS(C)(=O)=O)NC(=O)OC(C)(C)C)cc1] | 0.63 | 441 | 2 |
| 10d | 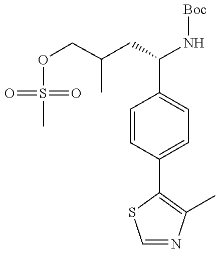 [SMILES: Cc1ncsc1-c1ccc([C@H](CC(C)COS(C)(=O)=O)NC(=O)OC(C)(C)C)cc1] | 0.69 | 455 | 1 |
| 10e | 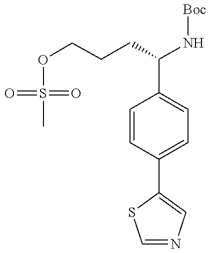 [SMILES: CC(C)(C)OC(=O)N[C@@H](CCCOS(C)(=O)=O)c1ccc(-c2cncs2)cc1] | 0.63 | 427 | 1 |
| 10f | 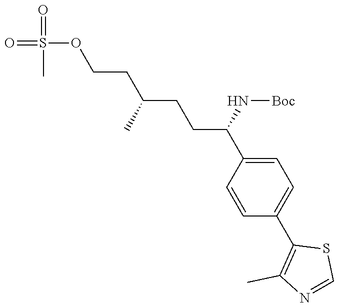 [SMILES: Cc1ncsc1-c1ccc([C@H](CC[C@H](C)CCOS(C)(=O)=O)NC(=O)OC(C)(C)C)cc1] | 0.76 | 483 | 2 |
| 10g | 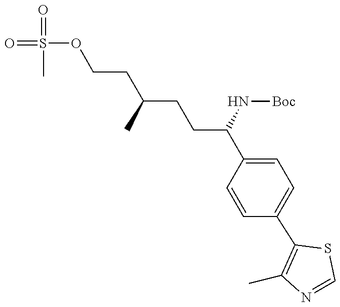 | 0.76 | 483 | 2 |
| 10h | 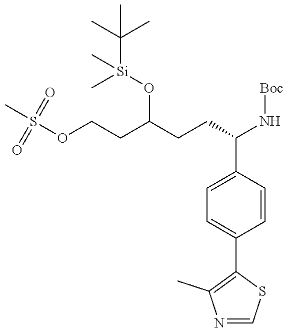 [SMILES: Cc1ncsc1-c1ccc([C@H](CCC(CCOS(C)(=O)=O)O[Si](C)(C)C(C)(C)C)NC(=O)OC(C)(C)C)cc1] | 0.97 | 599 | 4 |
| 10i | 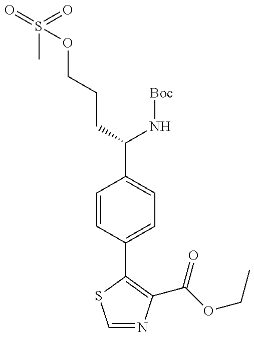 [SMILES: CCOC(=O)c1ncsc1-c1ccc([C@H](CCCOS(C)(=O)=O)NC(=O)OC(C)(C)C)cc1] | 0.64 | 499 | 1 |

### Synthesis Towards Intermediate 11a

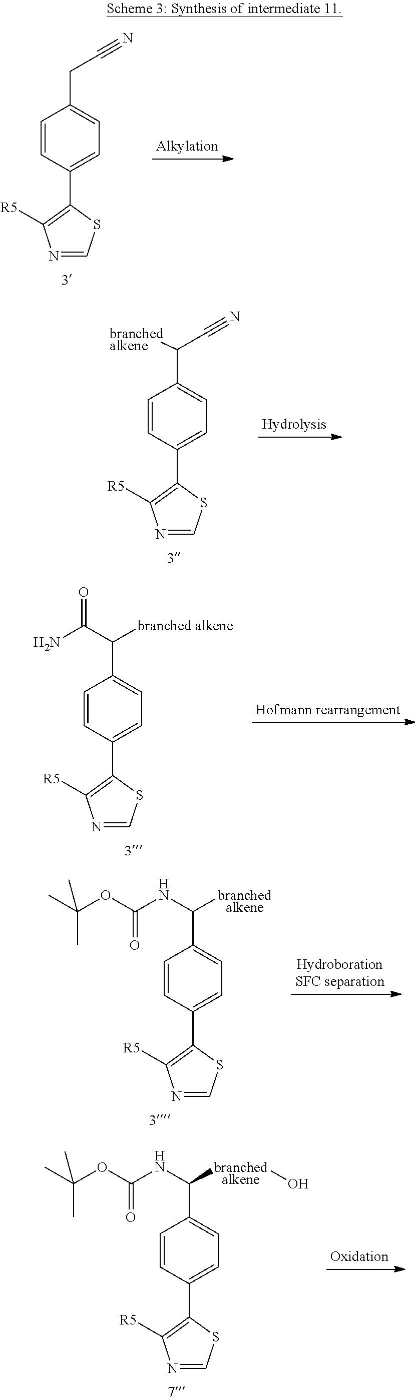

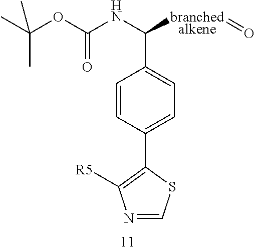

Starting from Nitrile 3′ a standard alkylation reaction under basic conditions allows the installation of a branched linker motive leading to Nitrile 3″, which is then hydrolyzed under basic conditions to the primary amide 3′″. A Hofmann rearrangement is giving the desired intermediate 3″″, which after hydroboration of the double bond under standard conditions is giving the desired intermediate 7′″. Intermediate 7′″ is oxidized to the corresponding aldehyde 11 using e.g. TEMPO.

### Experimental Procedure for the Synthesis of 3″a

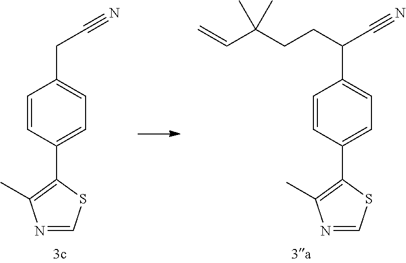 [SMILES: Cc1ncsc1-c1ccc(CC#N)cc1 ; C=CC(C)(C)CCC(C#N)c1ccc(-c2scnc2C)cc1]

Nitrile 3c (11.0 g; 51.0 mmol, 1.0 equiv.) is dissolved in THF (110 mL) and cooled to −78° C. 1.0M LiHMDS in THF (154 mL, 154 mmol, 3.0 equiv.) is added dropwise and stirred at same temperature for 20 min. Then 5-Iodo-3,3-dimethyl-pent-1-ene (15.0 g, 67.0 mmol; 1.3 equiv.) is added dropwise and the reaction mixture is slowly warmed to −20° C. and stirred for 60 min. It is cooled to −78° C. and slowly quenched with ammonium chloride solution (150 mL) and extracted with EtOAc (2×350 mL). The combined organics are dried over Na₂SO₄, filtered and concentrated under reduced pressure. The obtained crude is purified by silica gel column chromatography (20-40% EtOAc in petrol ether) to give 3″a (7.50 g, 47.1%).

TABLE 19

| | | | | HPLC |
|---|---|---|---|---|
| # | structure | tᵣₑₜ [min] | [M + H]⁺ | method |
| 3″a | 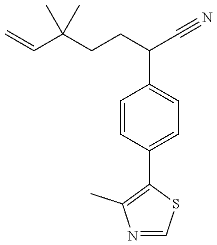 [SMILES: C=CC(C)(C)CCC(C#N)c1ccc(-c2scnc2C)cc1] | 2.53 | 311 | 9 |

### Experimental Procedure for the Synthesis of 3′″a

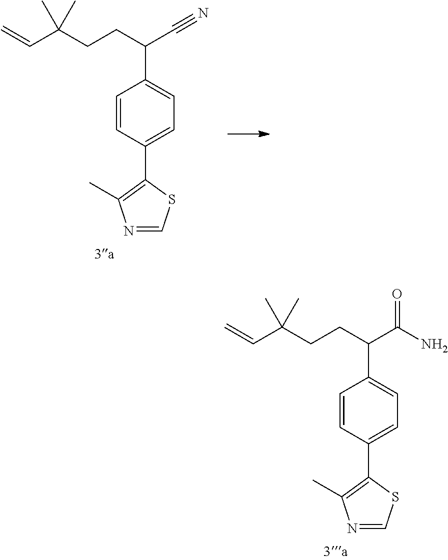 [SMILES: C=CC(C)(C)CCC(C#N)c1ccc(-c2scnc2C)cc1 ; C=CC(C)(C)CCC(C(N)=O)c1ccc(-c2scnc2C)cc1]

A mixture of nitrile 3″a (6.00 g, 19.3 mmol, 1.0 equiv.) and sodium hydroxide (7.73 g, 193 mmol; 10 equiv.) in MeOH (90 mL) and water (30 mL) is refluxed at 100° C. for 4 h. The reaction mixture is concentrated under reduced pressure. The obtained residue is dissolved in 100 mL water and extracted with EtOAc (2×150 mL). The combined organics are dried over Na₂SO₄, filtered and concentrated under reduced pressure. The obtained crude is washed with a mixture of n-pentane and diethyl ether to give 3′″a (4.00 g, 63.0%).

TABLE 20

| | | tᵣₑₜ | | HPLC |
|---|---|---|---|---|
| # | structure | [min] | [M + H]⁺ | method |
| 3″′a | 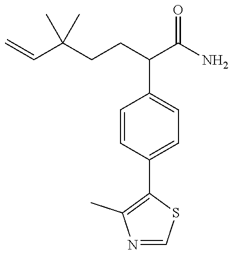 [SMILES: C=CC(C)(C)CCC(C(N)=O)c1ccc(-c2scnc2C)cc1] | 2.20 | 329 | 9 |

### Experimental Procedure for the Synthesis of 3″″a

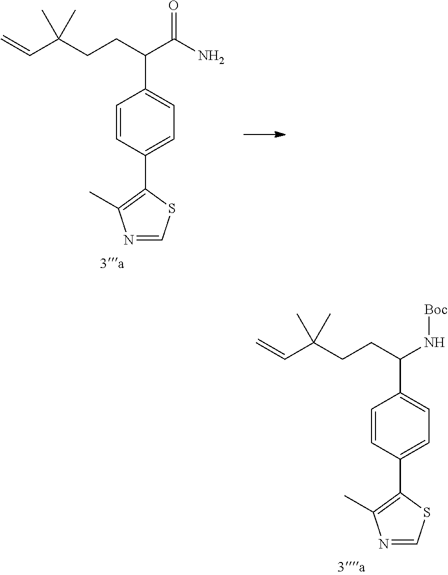 [SMILES: C=CC(C)(C)CCC(C(N)=O)c1ccc(-c2scnc2C)cc1 ; C=CC(C)(C)CCC(NC(=O)OC(C)(C)C)c1ccc(-c2scnc2C)cc1]

Amide 3′″a (2.20 g, 6.69 mmol, 1.0 equiv.) is dissolved in MeCN (50 mL) and water (17 mL). [Bis(trifluoroacetoxy)iodo]benzene (3.46 g, 8.05 mmol, 1.2 equiv.) is added and the reaction mixture is stirred at rt for 16 h. It is cooled to 0° C. and triethyl amine (1.88 mL, 13.3 mmol; 2.0 equiv.) and Boc anhydride (2.31 mL, 10.0 mmol, 1.5 equiv.) are added. Stirring is continued at rt for 4 h.

The reaction mixture is diluted with water (150 mL) and extracted with EtOAc (2×75 mL). The combined organics are dried over Na₂SO₄, filtered and concentrated under reduced pressure. The obtained crude is purified by silica gel column chromatography (20-40% EtOAc in petrol ether to give 3″″a (2.20 g, 82.0%).

TABLE 21

| # | structure | tᵣₑₜ [min] | [M + H]⁺ | HPLC method |
|---|---|---|---|---|
| 3′′′′a | 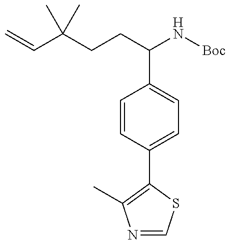 [SMILES: C=CC(C)(C)CCC(NC(=O)OC(C)(C)C)c1ccc(-c2scnc2C)cc1] | 2.67 | 401 | 9 |

### Experimental Procedure for the Synthesis of 7k and 7l

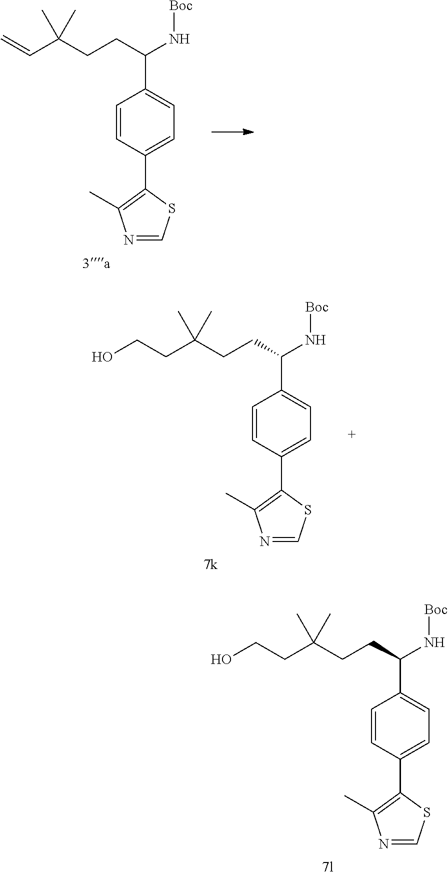 [SMILES: C=CC(C)(C)CCC(NC(=O)OC(C)(C)C)c1ccc(-c2scnc2C)cc1 ; Cc1ncsc1-c1ccc([C@H](CCC(C)(C)CCO)NC(=O)OC(C)(C)C)cc1 ; Cc1ncsc1-c1ccc([C@@H](CCC(C)(C)CCO)NC(=O)OC(C)(C)C)cc1]

Alkene 3″″a (3.00 g, 7.49 mmol, 1.0 equiv.) is dissolved in THF (30 mL) under nitrogen atmosphere and cooled to 0° C. Then 0.5 M THF solution of 9-borabicyclo[3.3.1]nonane (9-BBN, 44.9 mL, 22.4 mmol, 3.0 equiv.) is added slowly at 0° C. After complete addition, the cooling is removed and the mixture is stirred at rt for 2 h until complete conversion of 3′c. The reaction mixture is cooled to 0° C. again and 30% aq. hydrogen peroxide solution (2.55 g, 74.9 mmol, 10 equiv.) is added dropwise, followed by 4 N NaOH solution (18.7 mL, 74.9 mmol, 10 equiv.). The cooling is removed and the mixture is stirred at rt for 1 hr. The reaction is slowly quenched and acidified to pH 4 by carefully adding 1 N aq. hydrochloric acid. It is extracted with DCM twice. The combined organic layers are dried over Na₂SO₄, filtered and concentrated under reduced pressure. The obtained crude is purified by combi flash column chromatography (0-10% MeOH in DCM) to give 7k and 7l (1.40 g, 44.7%). The racemic mixture is then further purified by SFC (25% MeOH, to obtain the desired product as a pure enantiomer 7k (0.39 g, 28.5%).

TABLE 22

| # | structure | tᵣₑₜ [min] | [M + H]⁺ | HPLC method |
|---|---|---|---|---|
| 7k | 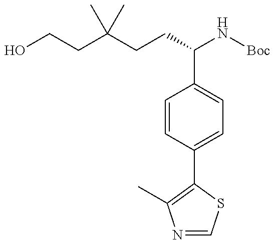 [SMILES: Cc1ncsc1-c1ccc([C@H](CCC(C)(C)CCO)NC(=O)OC(C)(C)C)cc1] | 2.05 | 419 | 11 |

### Experimental Procedure for the Synthesis of 11a

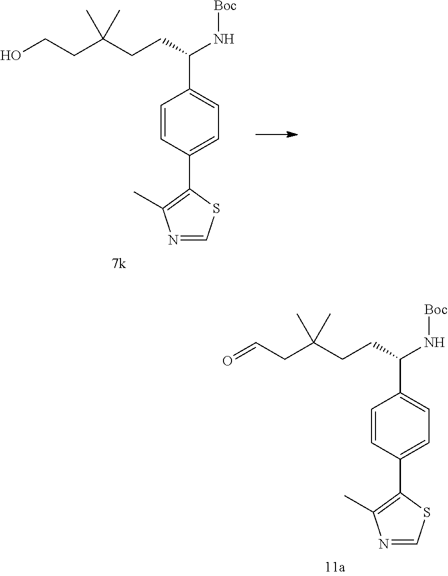 [SMILES: Cc1ncsc1-c1ccc([C@H](CCC(C)(C)CCO)NC(=O)OC(C)(C)C)cc1 ; Cc1ncsc1-c1ccc([C@H](CCC(C)(C)CC=O)NC(=O)OC(C)(C)C)cc1]

Alcohol 7k (50.0 mg, 0.118 mmol; 1.0 equiv.) is dissolved in dichloromethane (1.50 mL) and iodosobenzene diacetate (49.5 mg, 0.154 mmol, 1.3 equiv.) and TEMPO (4.71 mg, 0.030 mmol, 0.25 equiv.) are added. The mixture is stirred at rt overnight. The reaction mixture is diluted with DCM and purified by silica gel column chromatography (0-2% MeOH in DCM) to give 11a (38.0 mg, 77.2%).

The following intermediates 11 (table 23) are available in an analogous manner using different starting materials 7. The crude product 11 is purified by chromatography if necessary.

TABLE 23

| # | structure | tᵣₑₜ [min] | [M + H]⁺ | HPLC method |
|---|---|---|---|---|
| 11a | 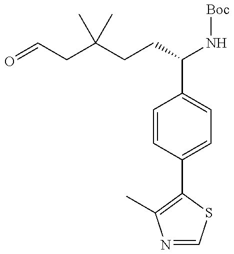 [SMILES: Cc1ncsc1-c1ccc([C@H](CCC(C)(C)CC=O)NC(=O)OC(C)(C)C)cc1] | 0.78 | 417 | 4 |
| 11b | 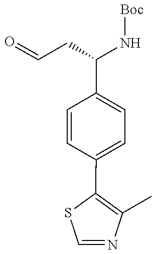 [SMILES: Cc1ncsc1-c1ccc([C@H](CC=O)NC(=O)OC(C)(C)C)cc1] | 1.21 | 347 | 14 |

### Synthesis Towards Intermediate 10 or 20

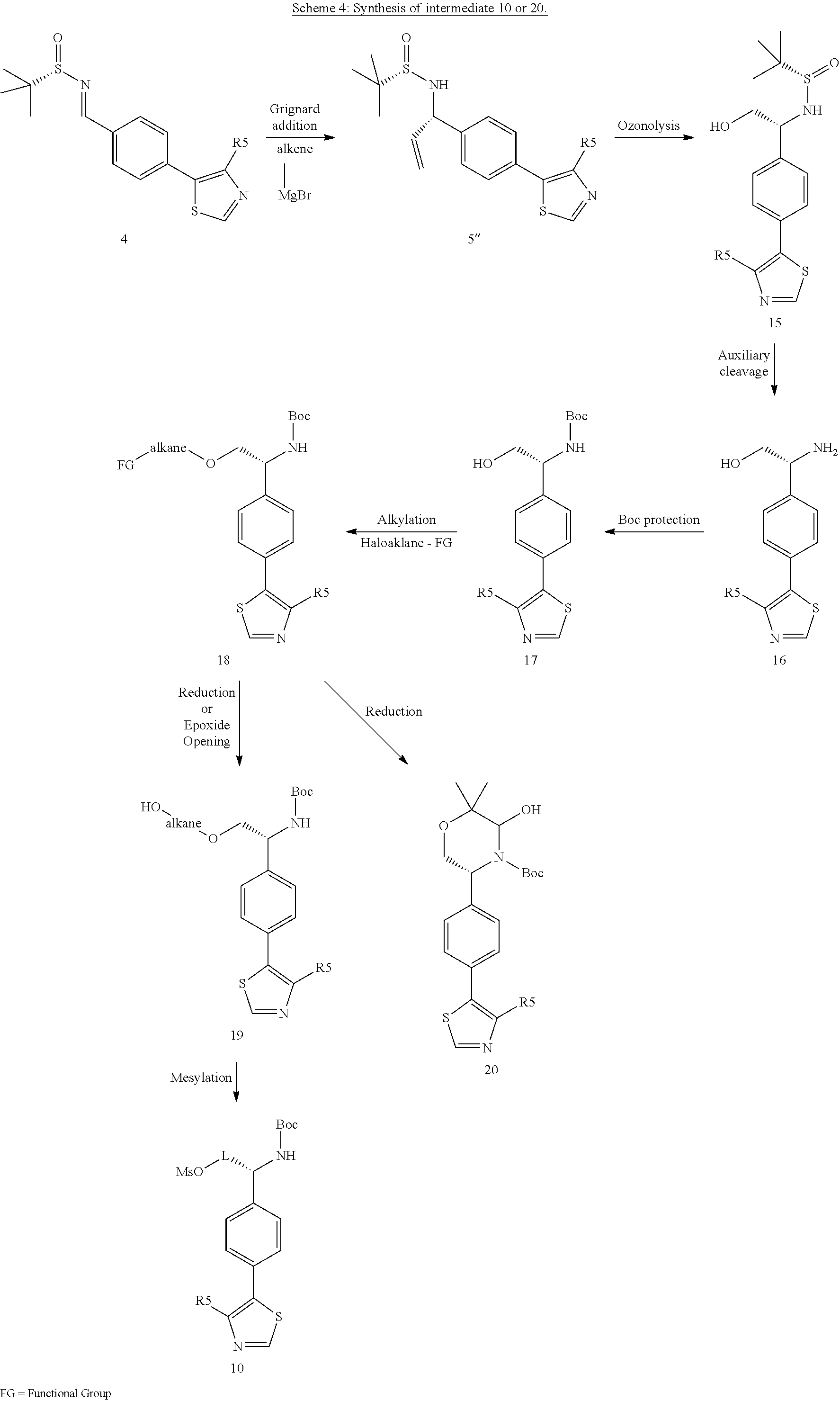

Alkene Grignard addition to sulfoximine 4 is leading to intermediate 5, which is transformed e.g. via ozonolysis into the alcohol 15. Cleavage of the chiral auxiliary under acidic conditions gives amino-alcohol 16, which is reprotected e.g. using (Boc)₂O to give the desired alcohol 17. Alkylation of alcohol 17 under basic conditions is leading to intermediate 18. Intermediate 18 can bear various functional groups such as esters, epoxides, etc. that can further be transformed into the corresponding alcohol e.g. via reduction or ring opening leading to intermediate 19 or reduction leading to aminal 20 The alcohol is transformed into mesylate 10 using e.g. mesyl chloride under basic conditions.

### Experimental Procedure for the Synthesis of 5f

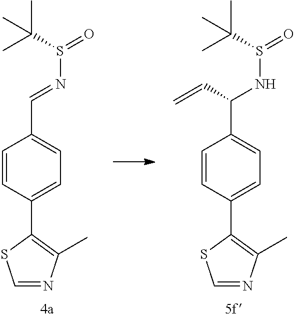 [SMILES: Cc1ncsc1-c1ccc(/C=N/[S@@](=O)C(C)(C)C)cc1 ; C=C[C@H](N[S@@](=O)C(C)(C)C)c1ccc(-c2scnc2C)cc1]

A 1 M THF solution of vinylmagnesium bromide (122 mL, 122 mmol, 1.5 equiv.) is added to a 1 M toluene solution of dimethyl zinc (139 mL, 139 mmol, 1.7 equiv.) at 0° C. and the resulting solution is stirred at rt for 15 min. The so prepared organozincate solution is then transferred dropwise to a solution of 4a (25.0 g, 81.6 mmol, 1.0 equiv.) in anhydrous THF (250 mL, 10 Vol) at −78° C. under argon atmosphere. The resulting mixture is stirred at −78° C. for 1 h. The reaction is quenched with ice-cold sat. ammonium chloride solution (250 mL) and diluted with EtOAc (250 mL). The mixture is filtered through a Celite pad. The filtrate layers are separated. The aqueous layer is extracted with EtOAc (250 mL). The combined organics are dried over Na₂SO₄, filtered and concentrated under reduced pressure. The obtained crude is purified by silica gel column chromatography (0-50% EtOAc in hexanes) to give 5f (21.0 g, 77.0%).

TABLE 24

| # | structure | tᵣₑₜ [min] | [M + H]⁺ | HPLC method |
|---|---|---|---|---|
| 5f′ | 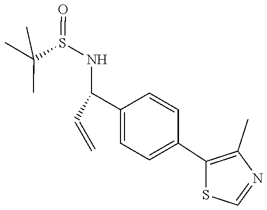 [SMILES: C=C[C@H](N[S@@](=O)C(C)(C)C)c1ccc(-c2scnc2C)cc1] | 2.08 | 335 | 9 |

### Experimental Procedure for the Synthesis of 15a

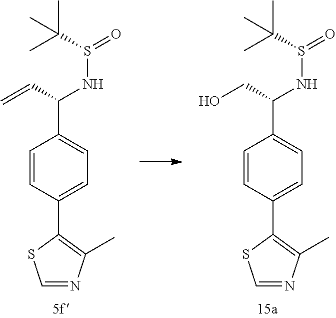 [SMILES: C=C[C@H](N[S@@](=O)C(C)(C)C)c1ccc(-c2scnc2C)cc1 ; Cc1ncsc1-c1ccc([C@H](CO)N[S@@](=O)C(C)(C)C)cc1]

5f′ (30.0 g, 89.7 mmol, 1.0 equiv.) is dissolved in methanol (300 mL, 10 Vol) and cooled to −78° C. The solution is purged with ozone gas for 1.5 hrs and then purged with air before sodium borohydride (10.2 g, 269 mmol, 3.0 equiv.) is added portionwise at −78° C. The mixture is allowed to slowly reach rt and is stirred for 16 hrs. The reaction mass is concentrated under reduced pressure and quenched with ice-cold water (600 mL). The obtained solids are collected by filtration, rinsed with water and diethyl ether and dried at 45° C. under vacuum to give 15a (23.0 g, 75.8%).

TABLE 25

| # | structure | tᵣₑₜ [min] | [M + H]⁺ | HPLC method |
|---|---|---|---|---|
| 15a | 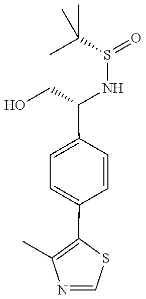 [SMILES: Cc1ncsc1-c1ccc([C@H](CO)N[S@@](=O)C(C)(C)C)cc1] | 1.45 | 339 | 11 |

### Experimental Procedure for the Synthesis of 17a

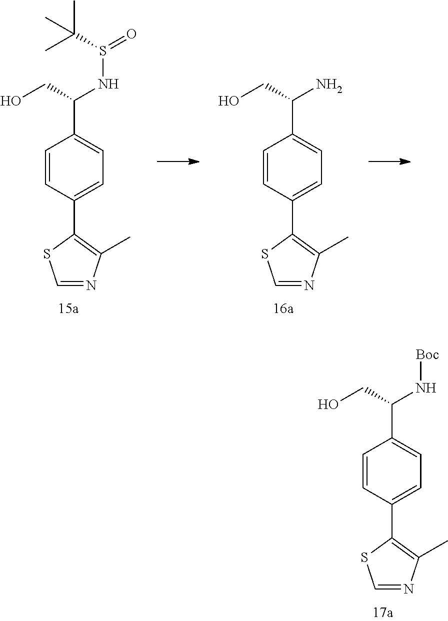 [SMILES: Cc1ncsc1-c1ccc([C@H](CO)N[S@@](=O)C(C)(C)C)cc1 ; Cc1ncsc1-c1ccc([C@@H](N)CO)cc1 ; Cc1ncsc1-c1ccc([C@H](CO)NC(=O)OC(C)(C)C)cc1]

Sulfoximine 15a (42.0 g, 0.124 mol, 1.0 equiv.) is dissolved in DCM (400 mL, 9.5 Vol) and cooled to 0° C. Then hydrochloric acid, 4 N in dioxane (155 mL, 0.620 mol, 5.0 equiv.) is added dropwise at 0° C. The mixture is allowed to reach rt and is stirred for 2 hrs. The solvents are removed under reduced pressure. The residue is triturated with diethyl ether and the solids are dried to give alcohol 16a (33.0 g, 98.2%) as a hydrochloride salt, which is used for the next step.

Alcohol 16a (23.0 g, 84.9 mmol, 1.0 equiv.) is dissolved in 1,4-dioxane (120.0 mL, 5.2 Vol) and water (120.0 mL, 5.2 Vol) and cooled to 0° C. Then triethylamine (38.8 mL, 255 mmol, 3.0 equiv.) and di-tert-butyl dicarbonate (22.2 mL, 102 mmol, 1.2 equiv.) are added dropwise at 0° C. The mixture is allowed to reach rt and is stirred for 4 hrs. The reaction mixture is concentrated to about half of the volume, diluted with water (200 mL) and extracted with EtOAc (2×500 mL). The combined organics are washed with brine, dried over Na₂SO₄, filtered and concentrated under reduced pressure. The obtained residue is triturated with a 1:1 mixture of diethyl ether and n-pentane. The obtained solid is dried to give 17a (24.0 g, 84.5%).

TABLE 26

| # | structure | tᵣₑₜ [min] | [M + H]⁺ | HPLC method |
|---|---|---|---|---|
| 17a | 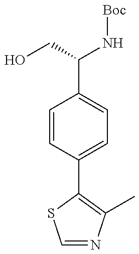 [SMILES: Cc1ncsc1-c1ccc([C@H](CO)NC(=O)OC(C)(C)C)cc1] | 1.87 | 335 | 9 |

### Experimental Procedure for the Synthesis of 18a

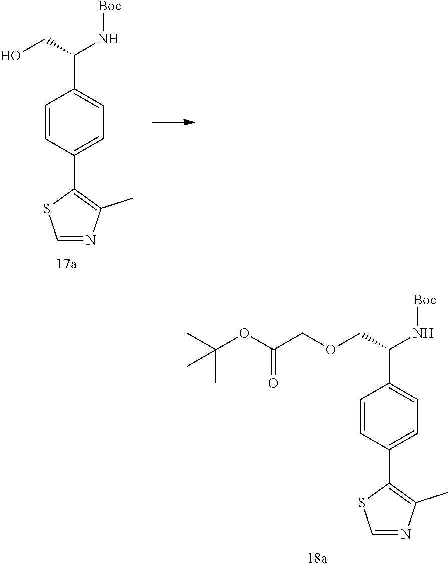 [SMILES: Cc1ncsc1-c1ccc([C@H](CO)NC(=O)OC(C)(C)C)cc1 ; Cc1ncsc1-c1ccc([C@H](COCC(=O)OC(C)(C)C)NC(=O)OC(C)(C)C)cc1]

Alcohol 17a (500 mg, 1.21 mmol, 1.0 equiv.) and tetrabutylammonium hydrogen sulfate (165 mg, 0.49 mmol, 0.4 equiv.) are suspended in dichloromethane (10.0 mL) and 4 M sodium hydroxide solution (7.50 mL, 30.0 mmol, 25 equiv.) is added. Then tert-butyl bromoacetate (0.270 mL, 1.82 mmol, 1.5 equiv.) is dissolved in 2.5 mL DCM and added dropwise. The reaction mixture is stirred at rt for 12 h. Additional tert-butyl bromoacetate (0.180 mL, 1.21 mmol, 1.0 equiv.) is added and the reaction is stirred at rt for additional 24 h. The reaction mixture is diluted with 5 mL water and acidified with 4 N HCl. It is extracted with DCM three times. The combined organics are dried over MgSO₄ and concentrated under reduced pressure. The crude residue is purified by silica gel column chromatography (0-5% MeOH in 0CM) to give 18a (584 mg, quantitative).

The following intermediates 18 (table 27) are available in an analogous manner using different functionalized haloalkanes. The crude product 18 is purified by chromatography if necessary.

TABLE 27

| # | structure | tᵣₑₜ [min] | [M + H]⁺ | HPLC method |
|---|---|---|---|---|
| 18a | 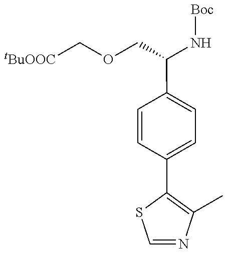 | 0.786 | 449 | 4 |
| 18b | 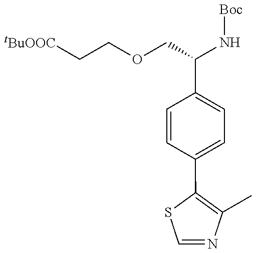 | 0.807 | 463 | 4 |
| 18c | 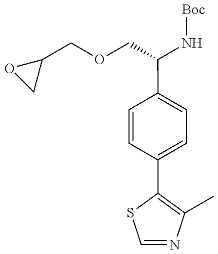 [SMILES: Cc1ncsc1-c1ccc([C@H](COCC2CO2)NC(=O)OC(C)(C)C)cc1] | 0.612 | 391 | 4 |
| 18d | 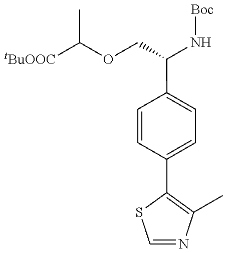 | 0.854 | 463 | 1 |
| 18e | 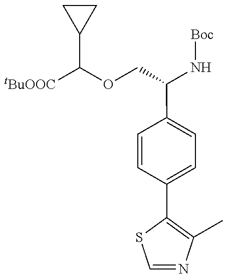 | 3.33 | 489 | 8 |
| 18f | 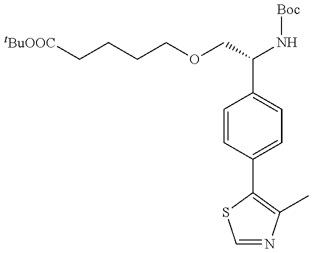 | 0.890 | 491 | 1 |
| 18g | 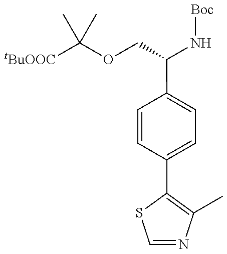 | 1.65 | 477 | 14 |

### Experimental Procedure for the Synthesis of 10j

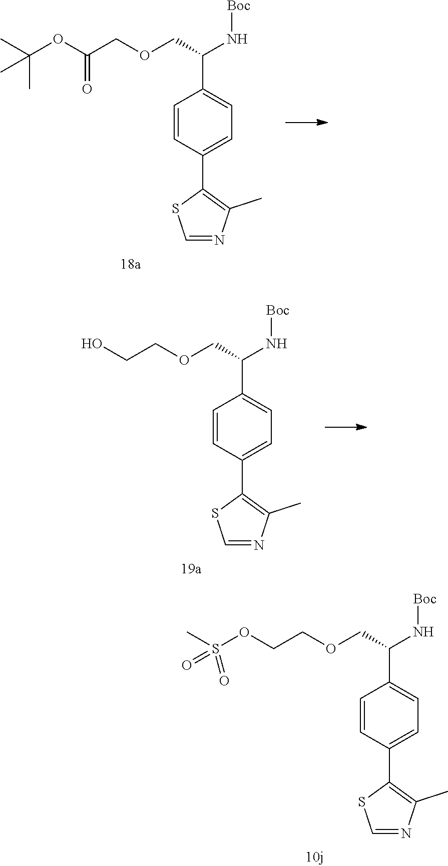 [SMILES: Cc1ncsc1-c1ccc([C@H](COCC(=O)OC(C)(C)C)NC(=O)OC(C)(C)C)cc1 ; Cc1ncsc1-c1ccc([C@H](COCCO)NC(=O)OC(C)(C)C)cc1 ; Cc1ncsc1-c1ccc([C@H](COCCOS(C)(=O)=O)NC(=O)OC(C)(C)C)cc1]

Ester 18a (50.0 mg, 0.11 mmol, 1.0 equiv.) is dissolved in THF, dry (1.00 mL) and cooled to 0° C. in an icebath. Then LAH 2M in THF (0.084 mL, 0.17 mmol, 1.5 equiv.) is added. The reaction mixture is stirred at 0° C. for 1 h. The reaction mixture is cautiously quenched with water at 0° C. It is diluted with DCM and water. The salts are filtered off over a Celite pad. The layers are separated and the aqueous phase is extracted with DCM. The combined organic layers are dried over MgSO₄ and concentrated under reduced pressure to give 19a (36 mg, 85%) as a crude product.

Alcohol 19a (520 mg; 1.37 mmol; 1 equiv) and TEA (0.572 mL; 4.1 mmol; 3.0 equiv.) are dissolved in DCM (15.0 mL) and cooled to 0° C. in an ice bath. Then MsCl (0.212 mL; 2.74 mmol; 2.0 equiv.) is added slowly and the reaction is stirred at 0° C. for 15 min. Complete conversion to the desired product. The reaction is quenched with 20 mL of sat. sodium bicarbonate solution and stirred for 20 min at rt. The layers are separated and the aq. layer is washed with DCM (2×10 mL). The combined organic layers are dried and concentrated. The residue is load on silica and purified by NP-chromatography giving the desired product 10j (590 mg, 94.5%).

The following intermediates 10 (table 28) are available in an analogous manner using different starting materials 18. The crude product 10 is purified by chromatography if

TABLE 28

| # | structure | tᵣₑₜ [min] | [M + H]⁺ | HPLC method |
|---|---|---|---|---|
| 10j | 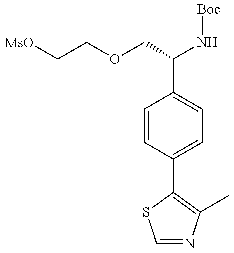 [SMILES: Cc1ncsc1-c1ccc([C@H](COCCOS(C)(=O)=O)NC(=O)OC(C)(C)C)cc1] | 0.62 | 457 | 4 |
| 10k | 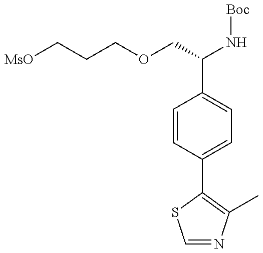 [SMILES: Cc1ncsc1-c1ccc([C@H](COCCCOS(C)(=O)=O)NC(=O)OC(C)(C)C)cc1] | 0.550 | 393 | 4 |
| 10l | 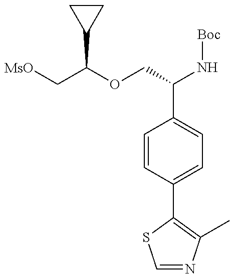 [SMILES: Cc1ncsc1-c1ccc([C@H](CO[C@@H](COS(C)(=O)=O)C2CC2)NC(=O)OC(C)(C)C)cc1] | 0.692 | 497 | 4 |
| 10m | 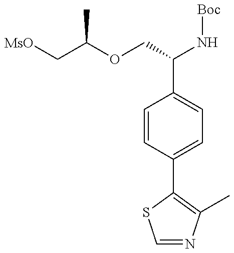 [SMILES: Cc1ncsc1-c1ccc([C@H](CO[C@H](C)COS(C)(=O)=O)NC(=O)OC(C)(C)C)cc1] | 0.670 | 471 | 1 |
| 10n | 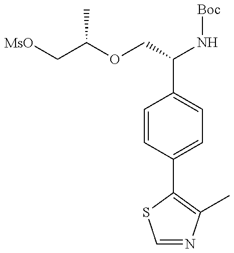 [SMILES: Cc1ncsc1-c1ccc([C@H](CO[C@@H](C)COS(C)(=O)=O)NC(=O)OC(C)(C)C)cc1] | 1.29 | 471 | 14 |
| 10o | 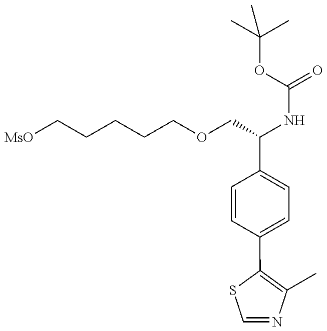 [SMILES: Cc1ncsc1-c1ccc([C@H](COCCCCCOS(C)(=O)=O)NC(=O)OC(C)(C)C)cc1] | 0.719 | 499 | 1 |

### Experimental Procedure for the Synthesis of 20a

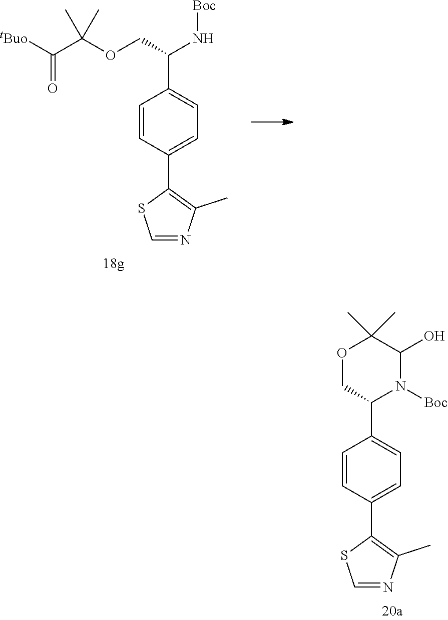

Ester 18g (1.74 g, 3.65 mmol, 1.0 equiv.) is dissolved in dry THF (30 mL) and cooled to 0° C. Then LAH 2M in THF (1.83 mL, 3.65 mmol, 1.0 equiv.) is added dropwise. The reaction mixture is stirred at 0° C. for 1 h.

The reaction mixture is cautiously quenched with water at 0° C. It is diluted with DCM and water. The salts are filtered off over a Celite pad. The layers are separated and the aqueous phase is extracted with DCM. The combined organic layers are dried over MgSO₄ and concentrated under reduced pressure. The obtained crude is purified by silica gel column chromatography (0-2.5% MeOH in DCM) to give 20a (1.09 g, 73.8%).

TABLE 29

| | | | | HPLC |
|---|---|---|---|---|
| # | Structure | tᵣₑₜ [min] | [M + H]⁺ | method |
| 20a | 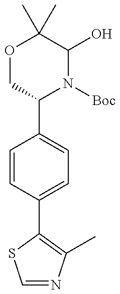 | 1.34 | 405 | 14 |

### Synthesis Towards Compound 28

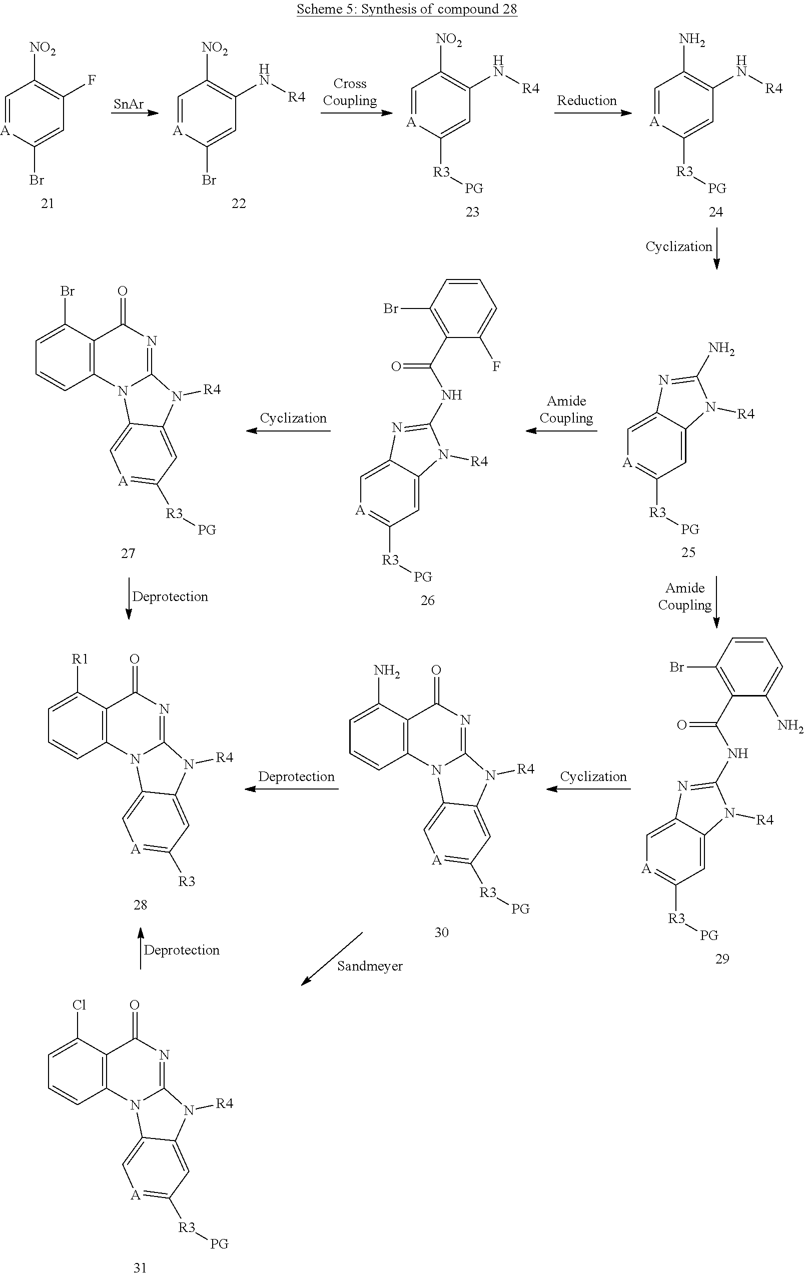

Nucleophilic aromatic substitution under standard conditions on an aromatic starting material 21 using various primary amines leads to intermediate 22. Further functionalizing using cross coupling reactions such as e.g. Suzuki or Buchwald Hartwig couplings is leading to intermediate 23. Reduction of the Nitro group on intermediate 23 can be realized e.g. using Pd/C under hydrogen atmosphere leading to intermediate 24. Subsequent ring closure using cyanogen bromide leads to benzimidazoles 25. Dependent on the coupling partner used in the following amide coupling intermediate 26 or 29 is obtained. The ring closure to the tetrahydroquinoline core can be realized under basic conditions using potassium phosphate leading to intermediate 27 or using copper catalyzed Ullmann-type coupling conditions leading to intermediate 30. Deprotection of intermediates 27 and 30 is leading to 28. To install other halogen atoms, intermediate 30 can be further modified using Sandmayer conditions leading e.g. to intermediate 31, which after final deprotection under acidic conditions is leading to 28.

### Experimental Procedure for the Synthesis of 21b

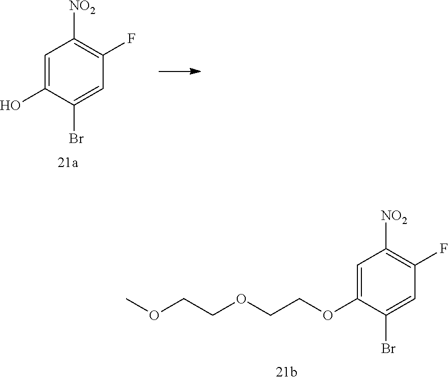 [SMILES: O=[N+]([O-])c1cc(O)c(Br)cc1F ; COCCOCCOc1cc([N+](=O)[O-])c(F)cc1Br]

To a stirred solution of 2-bromo-4-fluoro-5-nitrophenol 21a (2 g, 8.47 mmol, 1.00 equiv.) in dimethylformamide (40 ml), potassium carbonate (1.3 g, 9.32 mmol, 1.10 equiv.) is added at rt. The solution is stirred for 10 min at rt. 1-Brom-2-(2-methoxyethoxy)-ethane (1.41 ml, 9.32 mmol, 1.10 equiv.) are added and stirred for 1 h at 80° C. The reaction mixture was quenched with water and extracted with ethylacetate (2×30 ml). The combined organic layer is dried an evaporated to dryness. The crude product is purified by chromatography to give 21b (2.15 g, 75%).

TABLE 30

| | | | | HPLC |
|---|---|---|---|---|
| # | Structure | tᵣₑₜ [min] | [M + H]⁺ | method |
| 21b | 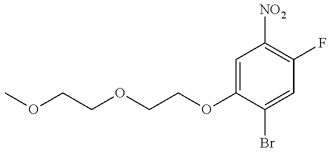 [SMILES: COCCOCCOc1cc([N+](=O)[O-])c(F)cc1Br] | 0.62 | / | 4 |

### Experimental Procedure for the Synthesis of 22a

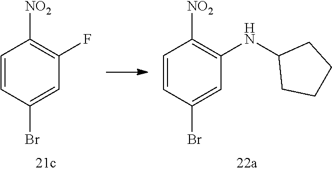 [SMILES: O=[N+]([O-])c1ccc(Br)cc1F ; O=[N+]([O-])c1ccc(Br)cc1NC1CCCC1]

To a stirred solution of 4-bromo-2-fluoro-1-nitro-benzene 21c (300 g, 1.36 mol, 1.00 equiv.) and cyclopentylamine (128 g, 1.50 mol, 1.10 equiv.) in dimethylformamide (2.51l), potassium carbonate (471 g, 3.41 mol, 2.50 equiv.) is added at rt. The solution is stirred for 16 h at rt. The reaction mixture is diluted with ice cold water. The obtained solid product is filtered and rinsed with water. The residue is dried under reduced pressure to give 22a (300 g, 77.2%).

The following intermediates 22 (table 31) are available in an analogous manner using different starting materials 21 and amines. The crude product 22 is purified by chromatography if necessary.

TABLE 31

| # | Structure | tᵣₑₜ [min] | [M + H]⁺ | HPLC method |
|---|---|---|---|---|
| 22a | 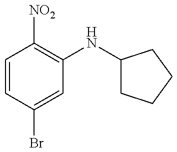 [SMILES: O=[N+]([O-])c1ccc(Br)cc1NC1CCCC1] | 2.71 | 285/287 | 9 |
| 22b | 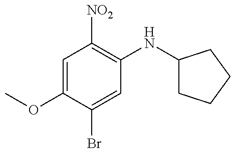 [SMILES: COc1cc([N+](=O)[O-])c(NC2CCCC2)cc1Br] | 2.61 | 315/317 | 9 |
| 22c | 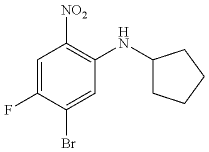 [SMILES: O=[N+]([O-])c1cc(F)c(Br)cc1NC1CCCC1] | 2.71 | 303/305 | 9 |
| 22d | 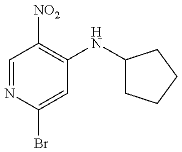 [SMILES: O=[N+]([O-])c1cnc(Br)cc1NC1CCCC1] | 0.742 | 286/288 | 4 |
| 22e | 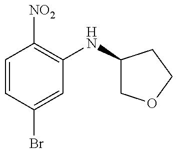 [SMILES: O=[N+]([O-])c1ccc(Br)cc1N[C@H]1CCOC1] | 2.25 | 287/289 | 9 |
| 22f | 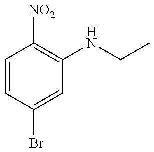 | 2.41 | 245/247 | 9 |
| 22g | 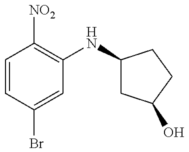 [SMILES: O=[N+]([O-])c1ccc(Br)cc1N[C@H]1CC[C@@H](O)C1] | 1.22 | 301 | 14 |
| 22h | 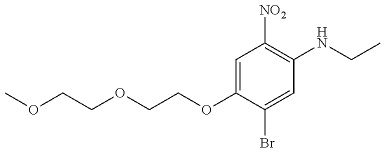 [SMILES: CCNc1cc(Br)c(OCCOCCOC)cc1[N+](=O)[O-]] | 2.53 | 364/366 | 9 |
| 22i | 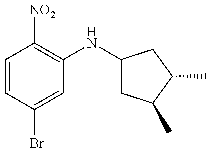 [SMILES: C[C@H]1CC(Nc2cc(Br)ccc2[N+](=O)[O-])C[C@@H]1C] | 3.21 | 313/315 | 9 |
| 22j | 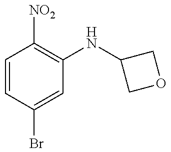 [SMILES: O=[N+]([O-])c1ccc(Br)cc1NC1COC1] | 2.07 | 273/275 | 9 |

### Experimental Procedure for the Synthesis of 23a

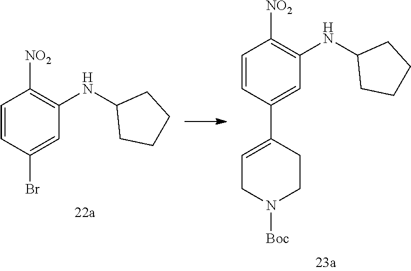 [SMILES: O=[N+]([O-])c1ccc(Br)cc1NC1CCCC1 ; CC(C)(C)OC(=O)N1CC=C(c2ccc([N+](=O)[O-])c(NC3CCCC3)c2)CC1]

To a stirred solution of 22a (200 g, 0.701 mol, 1.00 equiv.) in 1,4-dioxane (1.4 l) and water (600 ml), 4-(4,4,5,5-Tetramethyl-[1,3,2]dioxaborolan-2-yl)-3,6-dihydro-2H-pyridine-1-carboxylic acid tert-butyl ester (239 g, 0.77 mol, 1.10 equiv.) and cesium carbonate (570 g, 1.75 mol, 2.50 equiv.) is added at rt. The mixture is degassed for 15 min with argon and followed by the addition of palladium tetrakis (8.11 g, 7.01 mmol, 0.01 equiv.) at rt. The mixture is stirred over 16 h at 100° C. After complete conversion to desired product the 1,4-dioxane is removed under reduced pressure and the residue is diluted with ethyl acetate. This mixture is filtered through a Celite bed which is washed twice with ethyl acetate. The organic layer is washed with brine and dried over sodium sulfate, filtered off and concentrated under reduced pressure. The obtained crude product is purified by column chromatography to give 23a (190 g, 69.9%) which is used for the next step.

The following intermediates 23 (table 32) are available in an analogous manner using different starting materials 22.

TABLE 32

| # | structure | tᵣₑₜ [min] | [M + H]⁺ | HPLC method |
|---|---|---|---|---|
| 23a | 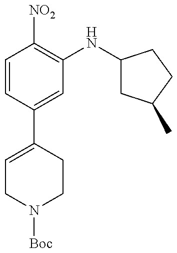 [SMILES: C[C@@H]1CCC(Nc2cc(C3=CCN(C(=O)OC(C)(C)C)CC3)ccc2[N+](=O)[O-])C1] | 2.96 | 388 | 9 |
| 23b | 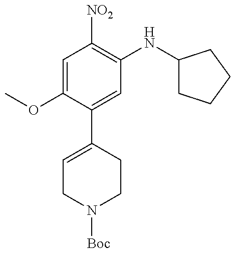 [SMILES: COc1cc([N+](=O)[O-])c(NC2CCCC2)cc1C1=CCN(C(=O)OC(C)(C)C)CC1] | 2.82 | 418 | 9 |
| 23c | 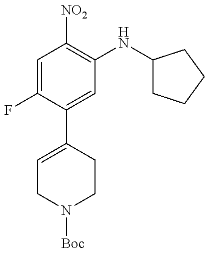 [SMILES: CC(C)(C)OC(=O)N1CC=C(c2cc(NC3CCCC3)c([N+](=O)[O-])cc2F)CC1] | 2.86 | 406 | 9 |
| 23d | 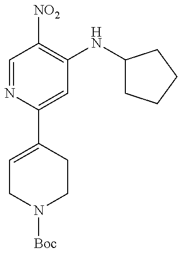 [SMILES: CC(C)(C)OC(=O)N1CC=C(c2cc(NC3CCCC3)c([N+](=O)[O-])cn2)CC1] | 2.84 | 389 | 9 |
| 23e | 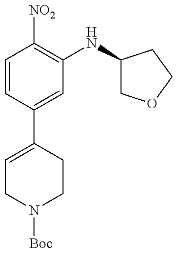 [SMILES: CC(C)(C)OC(=O)N1CC=C(c2ccc([N+](=O)[O-])c(N[C@H]3CCOC3)c2)CC1] | 2.49 | 390 | 9 |
| 23f | 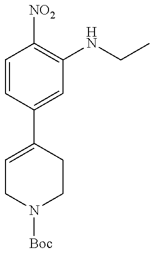 [SMILES: CCNc1cc(C2=CCN(C(=O)OC(C)(C)C)CC2)ccc1[N+](=O)[O-]] | 2.57 | 348 | 9 |
| 23g | 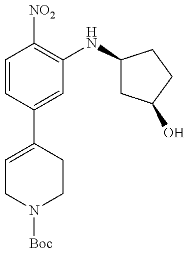 [SMILES: CC(C)(C)OC(=O)N1CC=C(c2ccc([N+](=O)[O-])c(N[C@H]3CC[C@@H](O)C3)c2)CC1] | 1.40 | 404 | 14 |
| 23h | 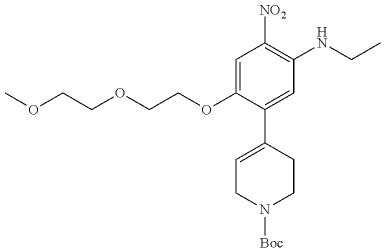 [SMILES: CCNc1cc(C2=CCN(C(=O)OC(C)(C)C)CC2)c(OCCOCCOC)cc1[N+](=O)[O-]] | 2.55 | 466 | 9 |
| 23i | 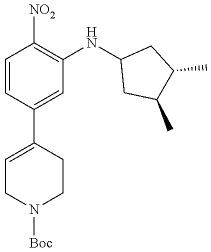 [SMILES: C[C@H]1CC(Nc2cc(C3=CCN(C(=O)OC(C)(C)C)CC3)ccc2[N+](=O)[O-])C[C@@H]1C] | 3.31 | 416 | 9 |
| 23j | 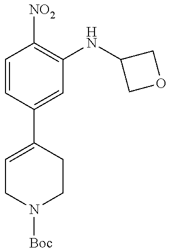 [SMILES: CC(C)(C)OC(=O)N1CC=C(c2ccc([N+](=O)[O-])c(NC3COC3)c2)CC1] | 2.33 | 376 | 9 |

### Experimental Procedure for the Synthesis of 23k

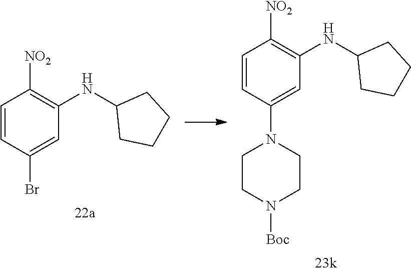 [SMILES: O=[N+]([O-])c1ccc(Br)cc1NC1CCCC1 ; CC(C)(C)OC(=O)N1CCN(c2ccc([N+](=O)[O-])c(NC3CCCC3)c2)CC1]

22a (120 g, 421 mmol, 1.00 equiv.) is dissolved in 1,4-dioxane (1.2 l). To this solution sodium tert-butoxide (80.9 g, 842 mmol, 2.00 equiv.) is added at rt followed by palladium(II) acetate (9.45 g, 42.1 mmol, 0.10 equiv.) and 4,5-bis(diphenylphosphino)-9,9-dimethylxanthene (24.4 g, 42.1 mmol, 0.10 equiv.). This mixture is stirred for 10 min. Then piperazine-1-carboxylic acid tert-butyl ester (78.4 g, 421 mmol, 1.00 equiv.) is added. The reaction mixture is heated to 120° C. for 16 h. After cooling to rt the mixture is filtered through a Celite bed followed by washing with dichloromethane and methanol. The filtrate is concentrated under reduced pressure to get the crude compound which is purified by column chromatography to get pure 23k (90.0 g, 54.8%).

The following intermediates 23 (table 33) are available in an analogous manner using different amines.

TABLE 33

| # | structure | tret [min] | [M+H]+ | HPLC method |
|---|---|---|---|---|
| 23k | 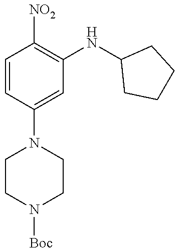 [SMILES: CC(C)(C)OC(=O)N1CCN(c2ccc([N+](=O)[O-])c(NC3CCCC3)c2)CC1] | 2.65 | 391 | 9 |
| 23l | 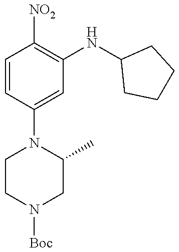 [SMILES: C[C@@H]1CN(C(=O)OC(C)(C)C)CCN1c1ccc([N+](=O)[O-])c(NC2CCCC2)c1] | 2.72 | 404 | 9 |
| 23m | 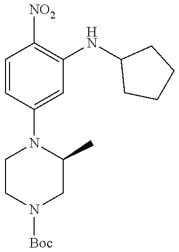 [SMILES: C[C@H]1CN(C(=O)OC(C)(C)C)CCN1c1ccc([N+](=O)[O-])c(NC2CCCC2)c1] | 2.73 | 405 | 9 |
| 23n | 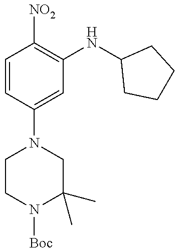 [SMILES: CC(C)(C)OC(=O)N1CCN(c2ccc([N+](=O)[O-])c(NC3CCCC3)c2)CC1(C)C] | 2.98 | 419 | 10 |

### Experimental Procedure for the Synthesis of 24a

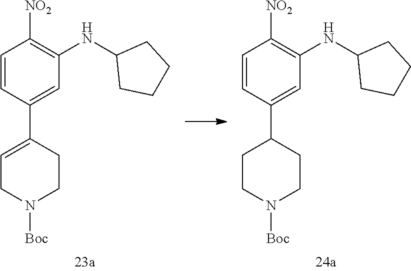 [SMILES: CC(C)(C)OC(=O)N1CC=C(c2ccc([N+](=O)[O-])c(NC3CCCC3)c2)CC1 ; CC(C)(C)OC(=O)N1CCC(c2ccc([N+](=O)[O-])c(NC3CCCC3)c2)CC1]

Palladium (10% on carbon, 14.0 g) is added to a solution of 23a (65.0 g, 0.17 mol, 1.00 equiv.) in THF (600 ml). The reaction mixture is stirred for 14 h with a hydrogen pressure of 80 PSI at rt. The reaction is filtered through a Celite bed followed by washing twice with ethyl acetate. The filtrate is concentrated under reduced pressure to give 24a (50.0 g, 82.9%).

The following intermediates 24 (table 34) are available in an analogous manner using different starting materials 23. The crude product 24 is purified by chromatography if

TABLE 34

| # | structure | tᵣₑₜ [min] | [M + H]⁺ | HPLC method |
|---|---|---|---|---|
| 24a | 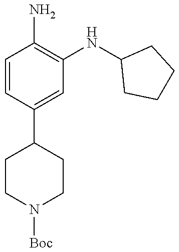 [SMILES: CC(C)(C)OC(=O)N1CCC(c2ccc(N)c(NC3CCCC3)c2)CC1] | 2.00 | 360 | 9 |
| 24b | 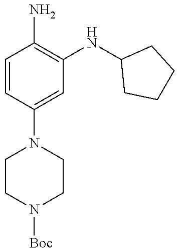 [SMILES: CC(C)(C)OC(=O)N1CCN(c2ccc(N)c(NC3CCCC3)c2)CC1] | 1.82 | 361 | 9 |
| 24c | 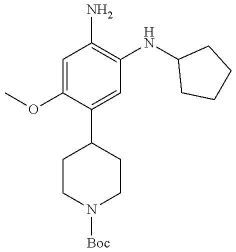 [SMILES: COc1cc(N)c(NC2CCCC2)cc1C1CCN(C(=O)OC(C)(C)C)CC1] | 2.16 | 390 | 9 |
| 24d | 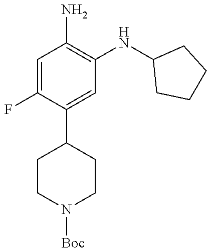 [SMILES: CC(C)(C)OC(=O)N1CCC(c2cc(NC3CCCC3)c(N)cc2F)CC1] | 2.12 | 378 | 9 |
| 24e | 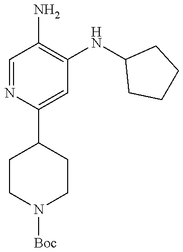 [SMILES: CC(C)(C)OC(=O)N1CCC(c2cc(NC3CCCC3)c(N)cn2)CC1] | 2.14 | 361 | 9 |
| 24f | 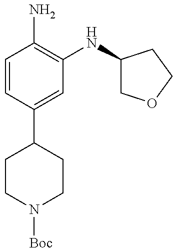 [SMILES: CC(C)(C)OC(=O)N1CCC(c2ccc(N)c(N[C@H]3CCOC3)c2)CC1] | 1.93 | 362 | 9 |
| 24g | 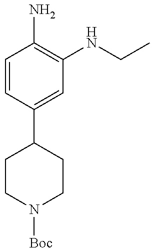 [SMILES: CCNc1cc(C2CCN(C(=O)OC(C)(C)C)CC2)ccc1N] | 2.85 | 320 | 8 |
| 24h | 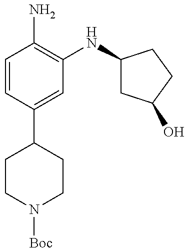 [SMILES: CC(C)(C)OC(=O)N1CCC(c2ccc(N)c(N[C@H]3CC[C@@H](O)C3)c2)CC1] | 0.63 | 376 | 4 |
| 24i | 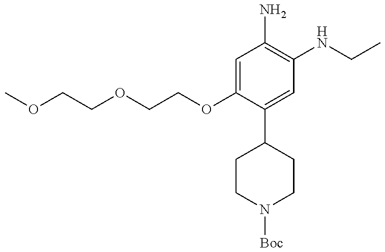 [SMILES: CCNc1cc(C2CCN(C(=O)OC(C)(C)C)CC2)c(OCCOCCOC)cc1N] | 2.77 | 438 | 8 |
| 24j | 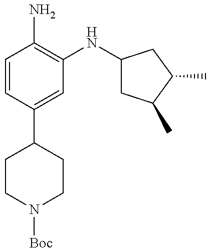 [SMILES: C[C@H]1CC(Nc2cc(C3CCN(C(=O)OC(C)(C)C)CC3)ccc2N)C[C@@H]1C] | 2.57 | 388 | 9 |
| 24k | 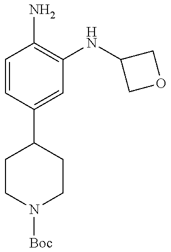 [SMILES: CC(C)(C)OC(=O)N1CCC(c2ccc(N)c(NC3COC3)c2)CC1] | 2.638 | 348 | 8 |
| 24l | 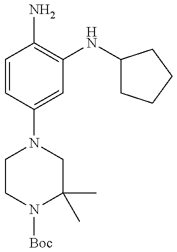 [SMILES: CC(C)(C)OC(=O)N1CCN(c2ccc(N)c(NC3CCCC3)c2)CC1(C)C] | 2.08 | 389 | 9 |

### Experimental Procedure for the Synthesis of 24m

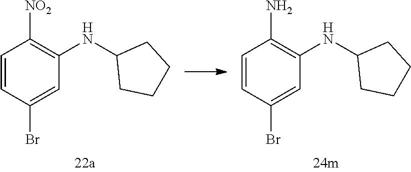 [SMILES: O=[N+]([O-])c1ccc(Br)cc1NC1CCCC1 ; Nc1ccc(Br)cc1NC1CCCC1]

To a stirred solution of 22a (40.0 g, 140 mmol, 1.00 equiv.) in ethanol (280 ml) and water (120 ml), iron powder (39.2 g, 701 mmol, 5.00 equiv.) and ammonium chloride (38.8 g, 701 mmol, 5.00 equiv.) are added and the mixture stirred for 16 h at 80° C. The reaction is cooled to rt and filtered through a Celite bed followed by washing with ethyl acetate. The filtrate is concentrated under reduced pressure. The obtained residue is washed three times with water and dried in vacuum to give 24m (35.0 g, 97.8%).

The following intermediates 24 (table 35) are available in an analogous manner using different starting materials 22 or 23. The crude product 24 is purified by chromatography if

TABLE 35

| # | structure | tret [min] | [M+H]+ | HPLC method |
|---|---|---|---|---|
| 24m | 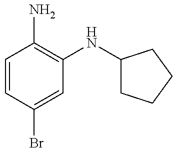 [SMILES: Nc1ccc(Br)cc1NC1CCCC1] | 2.23 | 255/257 | 9 |
| 24n | 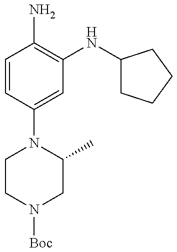 [SMILES: C[C@@H]1CN(C(=O)OC(C)(C)C)CCN1c1ccc(N)c(NC2CCCC2)c1] | 1.88 | 375 | 9 |
| 24o | 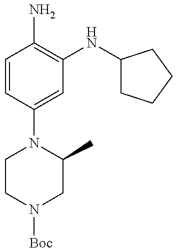 [SMILES: C[C@H]1CN(C(=O)OC(C)(C)C)CCN1c1ccc(N)c(NC2CCCC2)c1] | 3.152 | 375 | 8 |

### Experimental Procedure for the Synthesis of 25a

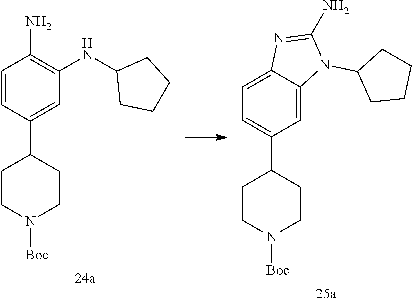 [SMILES: CC(C)(C)OC(=O)N1CCC(c2ccc(N)c(NC3CCCC3)c2)CC1 ; CC(C)(C)OC(=O)N1CCC(c2ccc3nc(N)n(C4CCCC4)c3c2)CC1]

To a stirred solution of 24a (60.0 g, 167 mmol, 1.00 equiv.) in methanol (600 ml) cyanogen bromide (35.4 g, 334 mmol, 2.00 equiv.) is added slowly portion wise at 0° C. and the reaction mixture is stirred well for 2 h at rt. Then the reaction mixture is concentrated, and the obtained residue is dissolved in dichloromethane. The organic phase is washed with saturated sodium bicarbonate solution and water, dried over sodium sulfate, filtered and concentrated under reduced pressure. The crude product is washed with diethyl ether to get pure 25a (42.0 g, 65.4%).

The following intermediates 25 (table 36) are available in an analogous manner using different starting materials 24. The crude product 25 is purified by chromatography if

TABLE 36

| # | structure | tᵣₑₜ [min] | [M + H]⁺ | HPLC method |
|---|---|---|---|---|
| 25a | 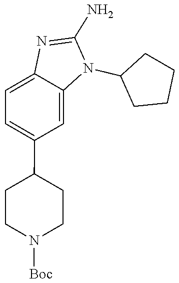 [SMILES: CC(C)(C)OC(=O)N1CCC(c2ccc3nc(N)n(C4CCCC4)c3c2)CC1] | 1.69 | 385 | 11 |
| 25b | 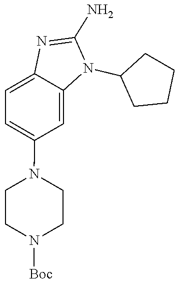 [SMILES: CC(C)(C)OC(=O)N1CCN(c2ccc3nc(N)n(C4CCCC4)c3c2)CC1] | 1.90 | 386 | 9 |
| 25c | 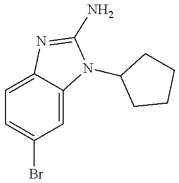 [SMILES: Nc1nc2ccc(Br)cc2n1C1CCCC1] | 1.75 | 280/282 | 9 |
| 25d | 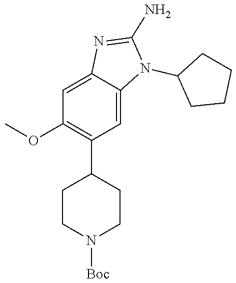 [SMILES: COc1cc2nc(N)n(C3CCCC3)c2cc1C1CCN(C(=O)OC(C)(C)C)CC1] | 1.96 | 415 | 9 |
| 25e | 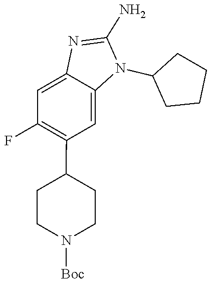 [SMILES: CC(C)(C)OC(=O)N1CCC(c2cc3c(cc2F)nc(N)n3C2CCCC2)CC1] | 2.88 | 403 | 8 |
| 25f | 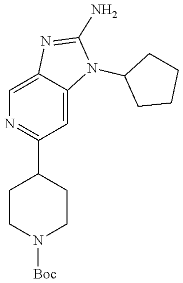 [SMILES: CC(C)(C)OC(=O)N1CCC(c2cc3c(cn2)nc(N)n3C2CCCC2)CC1] | 1.51 | 386 | 11 |
| 25g | 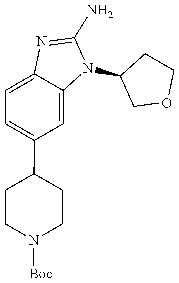 [SMILES: CC(C)(C)OC(=O)N1CCC(c2ccc3nc(N)n([C@H]4CCOC4)c3c2)CC1] | 2.54 | 387 | 8 |
| 25h | 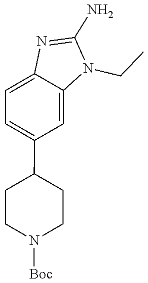 [SMILES: CCn1c(N)nc2ccc(C3CCN(C(=O)OC(C)(C)C)CC3)cc21] | 1.70 | 345 | 9 |
| 25i | 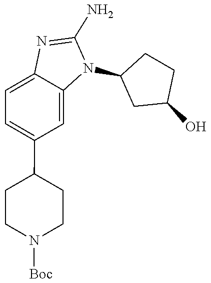 [SMILES: CC(C)(C)OC(=O)N1CCC(c2ccc3nc(N)n([C@H]4CC[C@@H](O)C4)c3c2)CC1] | 1.17 | 401 | 14 |
| 25j | 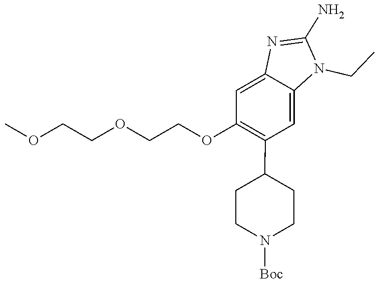 [SMILES: CCn1c(N)nc2cc(OCCOCCOC)c(C3CCN(C(=O)OC(C)(C)C)CC3)cc21] | 2.50 | 463 | 8 |
| 25k | 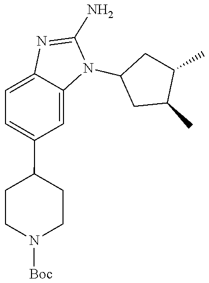 [SMILES: C[C@H]1CC(n2c(N)nc3ccc(C4CCN(C(=O)OC(C)(C)C)CC4)cc32)C[C@@H]1C] | 2.10 | 413 | 9 |
| 25l | 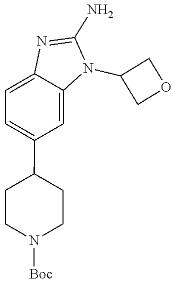 [SMILES: CC(C)(C)OC(=O)N1CCC(c2ccc3nc(N)n(C4COC4)c3c2)CC1] | 1.76 | 373 | 9 |
| 25m | 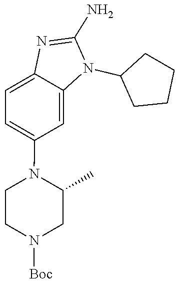 [SMILES: C[C@@H]1CN(C(=O)OC(C)(C)C)CCN1c1ccc2nc(N)n(C3CCCC3)c2c1] | 2.97 | 400/401 | 8 |
| 25n | 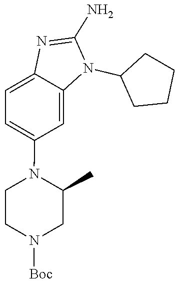 [SMILES: C[C@H]1CN(C(=O)OC(C)(C)C)CCN1c1ccc2nc(N)n(C3CCCC3)c2c1] | 2.85 | 400 | 8 |
| 25o | 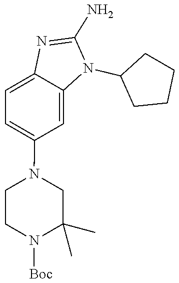 [SMILES: CC(C)(C)OC(=O)N1CCN(c2ccc3nc(N)n(C4CCCC4)c3c2)CC1(C)C] | 2.14 | 414 | 9 |

### Experimental Procedure for the Synthesis of 25p

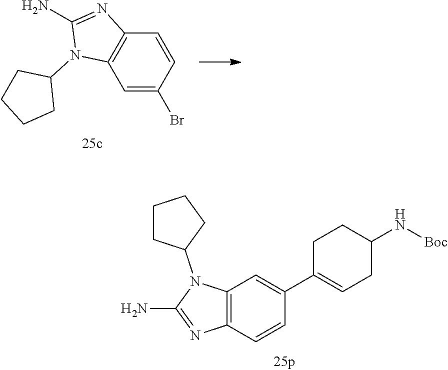 [SMILES: Nc1nc2ccc(Br)cc2n1C1CCCC1 ; CC(C)(C)OC(=O)NC1CC=C(c2ccc3nc(N)n(C4CCCC4)c3c2)CC1]

Amine 25c (250 mg; 0.810 mmol; 1.00 eq.), dioxaborolan (347 mg; 1.05 mmol; 1.30 eq.), cesium carbonate (528 mg; 1.62 mmol; 2.00 eq.) and (2-dicyclohexylphosphino-2′,4′,6′-triisopropyl-1,1′-biphenyl)[2-(2′-amino-1,1′-biphenyl)]palladium(II) methanesulfonate (XPhos 3G) (72.2 mg; 0.081 mmol; 0.10 eq.) are taken up in DME (10.0 mL)/water (2.0 mL). The mixture is degassed with argon and irradiated at 80° C. for 30 minutes in the microwave. The reaction mixture is diluted with DCM and water. The layers are separated. The aqueous layer is extracted with DCM. The combined organic layers are concentrated under reduced pressure. The product is purified by prep. HPLC (acidic conditions) to get the desired product 25p (256 mg, 79.7%).

TABLE 37

| # | Structure | tᵣₑₜ [min] | [M + H]⁺ | HPLC method |
|---|---|---|---|---|
| 25p | 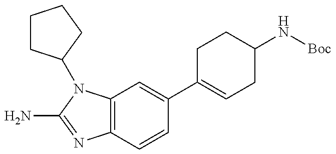 [SMILES: CC(C)(C)OC(=O)NC1CC=C(c2ccc3nc(N)n(C4CCCC4)c3c2)CC1] | 0.54 | 397 | 1 |

### Experimental Procedure for the Synthesis of 25q and 25r

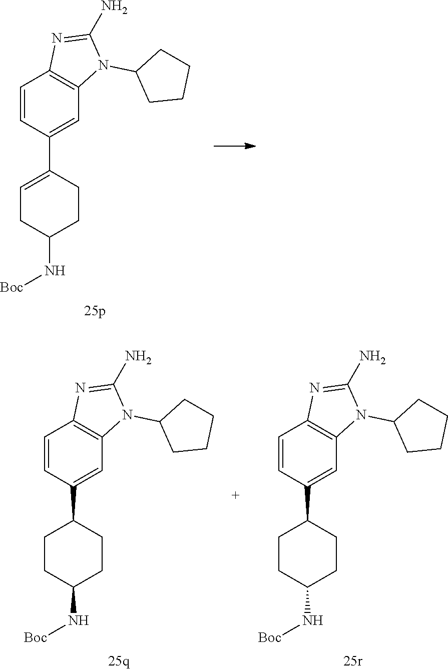 [SMILES: CC(C)(C)OC(=O)NC1CC=C(c2ccc3nc(N)n(C4CCCC4)c3c2)CC1 ; CC(C)(C)OC(=O)N[C@H]1CC[C@@H](c2ccc3nc(N)n(C4CCCC4)c3c2)CC1 ; CC(C)(C)OC(=O)N[C@H]1CC[C@H](c2ccc3nc(N)n(C4CCCC4)c3c2)CC1]

Carbamate 25p (256 mg; 0.646 mmol; 1.00 eq.) is dissolved in MeOH (10.0 mL) and palladium hydroxide (45.3 mg; 0.065 mmol; 0.10 eq.) is added. The reactor is flushed with N₂ and filled with 7bar H₂. The reaction mixture is stirred at rt for 20 h. The catalyst is filtered off through a Celite pad and the solvent is removed under reduced pressure. The product is purified via prep. HPLC to get the desired compounds 25q and 25r as cis/trans mixture (177 mg; 34.4%). The cis/trans-isomers are separated by SFC to get 25r (114.0 mg; 46.9%).

TABLE 38

| # | structure | tᵣₑₜ [min] | [M + H]⁺ | HPLC method |
|---|---|---|---|---|
| 25r | 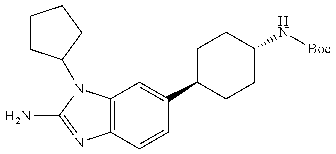 [SMILES: CC(C)(C)OC(=O)N[C@H]1CC[C@H](c2ccc3nc(N)n(C4CCCC4)c3c2)CC1] | 1.37 | 399 | 14 |

### Experimental Procedure for the Synthesis of 26a

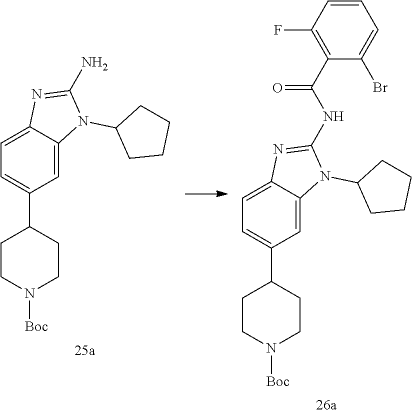 [SMILES: CC(C)(C)OC(=O)N1CCC(c2ccc3nc(N)n(C4CCCC4)c3c2)CC1 ; CC(C)(C)OC(=O)N1CCC(c2ccc3nc(NC(=O)c4c(F)cccc4Br)n(C4CCCC4)c3c2)CC1]

To a stirred solution of 2-bromo-4-fluoro-benzoic acid (27.5 g, 126 mmol, 1.15 equiv.) in 1,4-dioxane (420 ml), CDI (21.2 g, 131 mmol, 1.20 equiv.) is added at rt and the reaction mixture is stirred for 2 h at 95° C. Then the reaction mixture is cooled to rt and added to a solution of 25a (42.0 g, 109 mmol, 1.00 equiv.) and HOBt (22.6 g, 147 mmol, 1.35 equiv.) in 1,4-dioxane (420 ml) at rt. The mixture is heated to 95° C. and stirred 16 h at this temperature. The reaction mixture is cooled to rt and concentrated under reduced pressure. The obtained residue is diluted with water. The precipitated solid is filtered and washed with ethanol to get 26a (52.0 g, 81.3%).

The following intermediates 26 (table 39) are available in an analogous manner using different starting materials 25. The crude product 26 is purified by chromatography if

TABLE 39

| # | structure | tᵣₑₜ [min] | [M + H]⁺ | HPLC method |
|---|---|---|---|---|
| 26a | 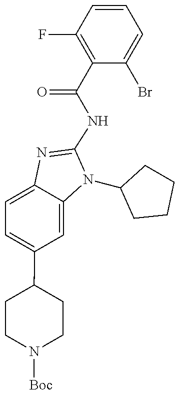 [SMILES: CC(C)(C)OC(=O)N1CCC(c2ccc3nc(NC(=O)c4c(F)cccc4Br)n(C4CCCC4)c3c2)CC1] | 2.64 | 585/587 | 11 |
| 26b | 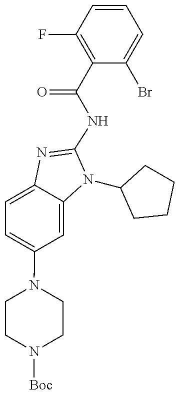 [SMILES: CC(C)(C)OC(=O)N1CCN(c2ccc3nc(NC(=O)c4c(F)cccc4Br)n(C4CCCC4)c3c2)CC1] | 2.67 | 586/588 | 9 |
| 26c | 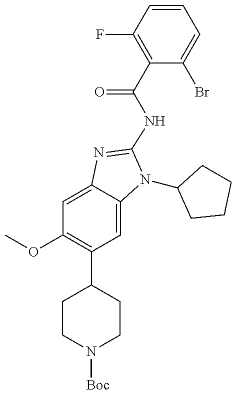 [SMILES: COc1cc2nc(NC(=O)c3c(F)cccc3Br)n(C3CCCC3)c2cc1C1CCN(C(=O)OC(C)(C)C)CC1] | 2.82 | 615/617 | 9 |
| 26d | 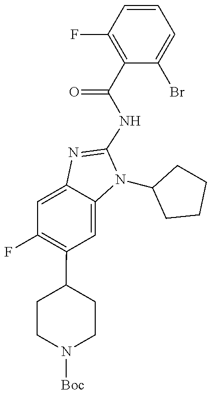 [SMILES: CC(C)(C)OC(=O)N1CCC(c2cc3c(cc2F)nc(NC(=O)c2c(F)cccc2Br)n3C2CCCC2)CC1] | 2.85 | 603/605 | 9 |
| 26e | 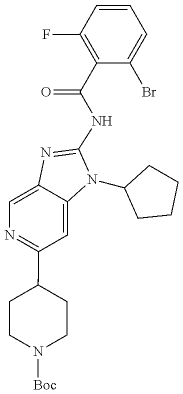 [SMILES: CC(C)(C)OC(=O)N1CCC(c2cc3c(cn2)nc(NC(=O)c2c(F)cccc2Br)n3C2CCCC2)CC1] | 2.57 | 587/589 | 9 |
| 26f | 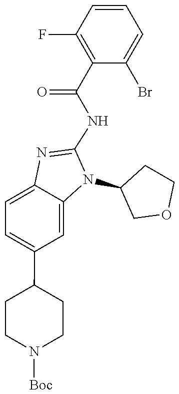 [SMILES: CC(C)(C)OC(=O)N1CCC(c2ccc3nc(NC(=O)c4c(F)cccc4Br)n([C@H]4CCOC4)c3c2)CC1] | 3.26 | 588 | 8 |
| 26g | 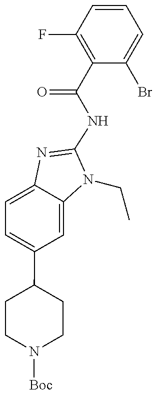 [SMILES: CCn1c(NC(=O)c2c(F)cccc2Br)nc2ccc(C3CCN(C(=O)OC(C)(C)C)CC3)cc21] | na | na | na |
| 26h | 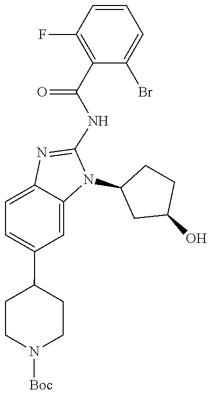 [SMILES: CC(C)(C)OC(=O)N1CCC(c2ccc3nc(NC(=O)c4c(F)cccc4Br)n([C@H]4CC[C@@H](O)C4)c3c2)CC1] | 1.47 | 601 | 14 |
| 26i | 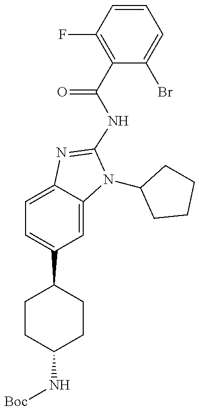 [SMILES: CC(C)(C)OC(=O)N[C@H]1CC[C@H](c2ccc3nc(NC(=O)c4c(F)cccc4Br)n(C4CCCC4)c3c2)CC1] | 0.962 | 599/601 | 4.M |
| 26j | 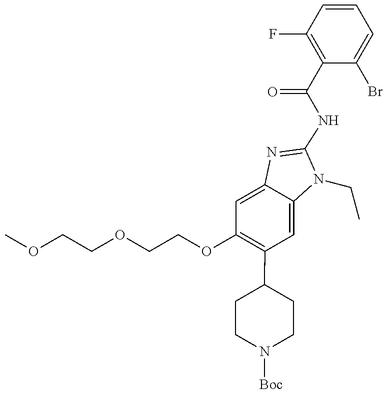 [SMILES: CCn1c(NC(=O)c2c(F)cccc2Br)nc2cc(OCCOCCOC)c(C3CCN(C(=O)OC(C)(C)C)CC3)cc21] | 3.28 | 663/665 | 8 |
| 26k | 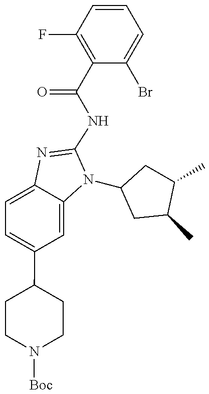 [SMILES: C[C@H]1CC(n2c(NC(=O)c3c(F)cccc3Br)nc3ccc(C4CCN(C(=O)OC(C)(C)C)CC4)cc32)C[C@@H]1C] | 2.96 | 613/615 | 9 |
| 26l | 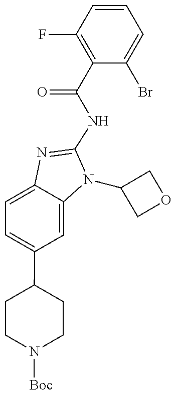 [SMILES: CC(C)(C)OC(=O)N1CCC(c2ccc3nc(NC(=O)c4c(F)cccc4Br)n(C4COC4)c3c2)CC1] | 2.41 | 573/575 | 9 |
| 26m | 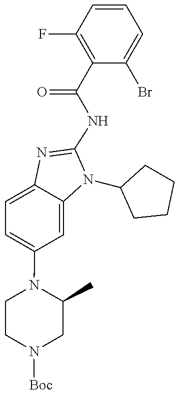 [SMILES: C[C@H]1CN(C(=O)OC(C)(C)C)CCN1c1ccc2nc(NC(=O)c3c(F)cccc3Br)n(C3CCCC3)c2c1] | 2.76 | 600/602 | 9 |
| 26n | 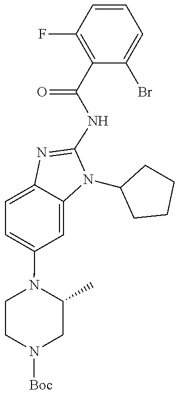 [SMILES: C[C@@H]1CN(C(=O)OC(C)(C)C)CCN1c1ccc2nc(NC(=O)c3c(F)cccc3Br)n(C3CCCC3)c2c1] | 2.73 | 599/601 | 9 |
| 26o | 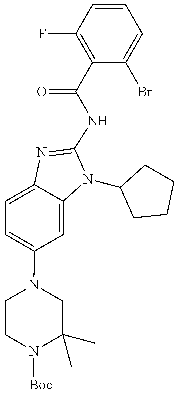 [SMILES: CC(C)(C)OC(=O)N1CCN(c2ccc3nc(NC(=O)c4c(F)cccc4Br)n(C4CCCC4)c3c2)CC1(C)C] | 1.72 | 614/617 | 11 |

### Experimental Procedure for the Synthesis of 29a

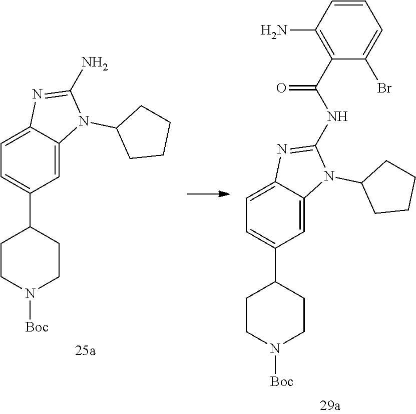 [SMILES: CC(C)(C)OC(=O)N1CCC(c2ccc3nc(N)n(C4CCCC4)c3c2)CC1 ; CC(C)(C)OC(=O)N1CCC(c2ccc3nc(NC(=O)c4c(N)cccc4Br)n(C4CCCC4)c3c2)CC1]

To a stirred solution of 25a (100 g, 260 mmol, 1.00 equiv.) in 1,4-dioxane (1000 ml), 5-bromo-1H-benzo[d][1,3]oxazine-2,4-dione (62.9 g, 260 mmol, 1.00 equiv.) is added at rt and the reaction mixture is stirred well for 14 h at 120° C. Then the reaction mixture is cooled to rt and concentrated under reduced pressure. The obtained residue is purified by column chromatography to get 29a (82.0 g, 54.1%).

The following intermediates 29 (table 40) are available in an analogous manner using different starting materials 25.

TABLE 40

| # | structure | tᵣₑₜ [min] | [M + H]⁺ | HPLC method |
|---|---|---|---|---|
| 29a | 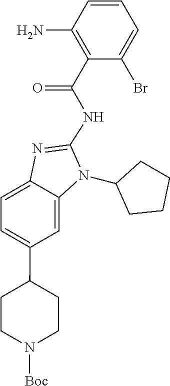 [SMILES: CC(C)(C)OC(=O)N1CCC(c2ccc3nc(NC(=O)c4c(N)cccc4Br)n(C4CCCC4)c3c2)CC1] | 2.66 | 582/584 | 9 |
| 29b | 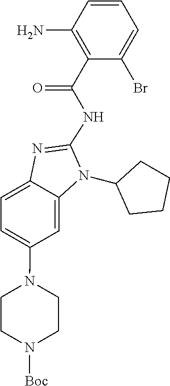 [SMILES: CC(C)(C)OC(=O)N1CCN(c2ccc3nc(NC(=O)c4c(N)cccc4Br)n(C4CCCC4)c3c2)CC1] | 0.86 | 583/585 | 1 |
| 29c | 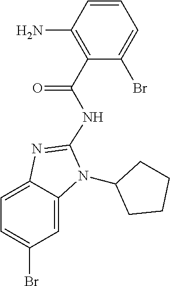 [SMILES: Nc1cccc(Br)c1C(=O)Nc1nc2ccc(Br)cc2n1C1CCCC1] | 0.84 | 479 | 4 |

### Experimental Procedure for the Synthesis of 27a

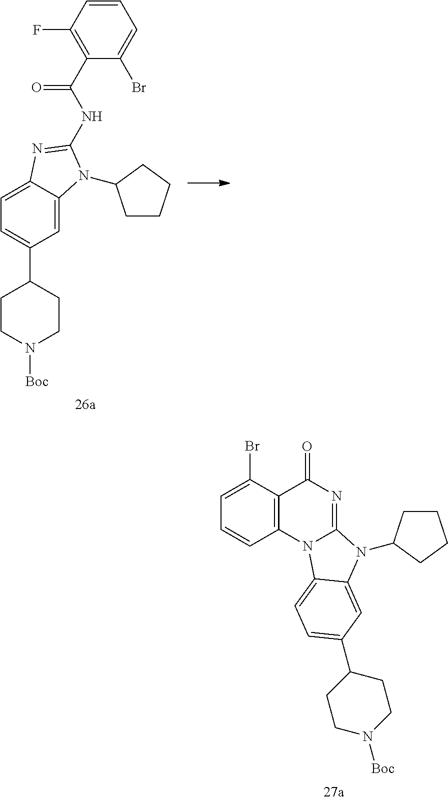 [SMILES: CC(C)(C)OC(=O)N1CCC(c2ccc3nc(NC(=O)c4c(F)cccc4Br)n(C4CCCC4)c3c2)CC1 ; CC(C)(C)OC(=O)N1CCC(c2ccc3c(c2)n(C2CCCC2)c2nc(=O)c4c(Br)cccc4n32)CC1]

To a stirred solution of 26a (52.0 g, 89.0 mmol, 1.00 equiv.) in dimethylformamide (400 ml), potassium phosphate tribasic (28.2 g, 133 mmol, 1.50 equiv.) is added at rt and the reaction mixture is stirred for 16 h at 120° C. Then the reaction mixture is cooled to rt and diluted with ice cold water. The precipitated solid is filtered off and dried under reduced pressure. The crude product is stirred with ethanol, filtered and dried to get 27a (42.0 g, 83.6%).

The following intermediates 27 (table 41) are available in an analogous manner using different starting materials 26. The crude product 27 is purified by chromatography if

TABLE 41

| | | tᵣₑₜ | | HPLC |
|---|---|---|---|---|
| # | structure | [min] | [M + H]⁺ | method |
| 27a | 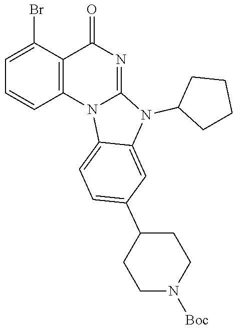 [SMILES: CC(C)(C)OC(=O)N1CCC(c2ccc3c(c2)n(C2CCCC2)c2nc(=O)c4c(Br)cccc4n32)CC1] | 2.38 | 565/567 | 11 |
| 27b | 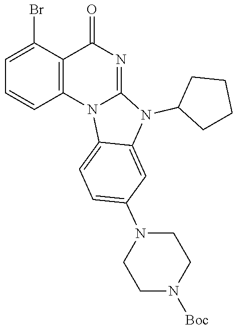 [SMILES: CC(C)(C)OC(=O)N1CCN(c2ccc3c(c2)n(C2CCCC2)c2nc(=O)c4c(Br)cccc4n32)CC1] | 2.44 | 566/568 | 9 |
| 27c | 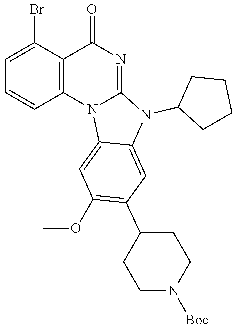 [SMILES: COc1cc2c(cc1C1CCN(C(=O)OC(C)(C)C)CC1)n(C1CCCC1)c1nc(=O)c3c(Br)cccc3n21] | 2.94 | 595/597 | 9 |
| 27d | 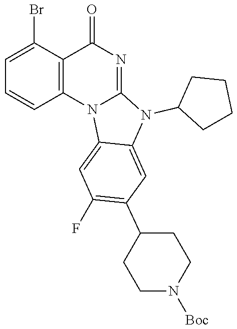 [SMILES: CC(C)(C)OC(=O)N1CCC(c2cc3c(cc2F)n2c4cccc(Br)c4c(=O)nc2n3C2CCCC2)CC1] | 2.44 | 583/585 | 11 |
| 27e | 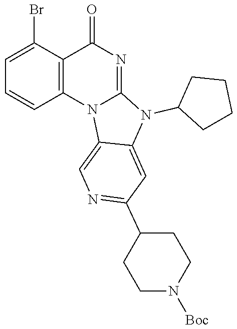 [SMILES: CC(C)(C)OC(=O)N1CCC(c2cc3c(cn2)n2c4cccc(Br)c4c(=O)nc2n3C2CCCC2)CC1] | 2.74 | 566/568 | 9 |
| 27f | 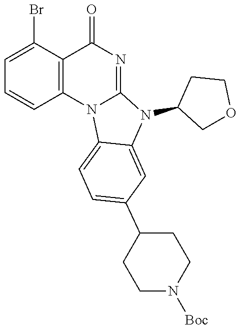 [SMILES: CC(C)(C)OC(=O)N1CCC(c2ccc3c(c2)n([C@H]2CCOC2)c2nc(=O)c4c(Br)cccc4n32)CC1] | 2.13 | 567/569 | 11 |
| 27g | 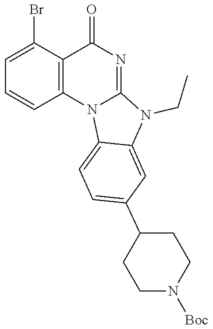 [SMILES: CCn1c2cc(C3CCN(C(=O)OC(C)(C)C)CC3)ccc2n2c3cccc(Br)c3c(=O)nc12] | 2.33 | 525/527 | 9 |
| 27h | 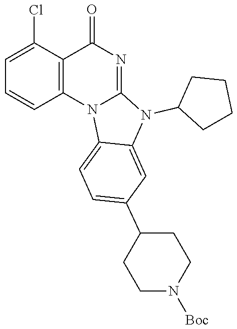 [SMILES: CC(C)(C)OC(=O)N1CCC(c2ccc3c(c2)n(C2CCCC2)c2nc(=O)c4c(Cl)cccc4n32)CC1] | 2.48 | 521 | 9 |
| 27i | 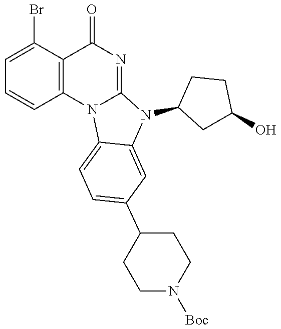 [SMILES: CC(C)(C)OC(=O)N1CCC(c2ccc3c(c2)n([C@H]2CC[C@@H](O)C2)c2nc(=O)c4c(Br)cccc4n32)CC1] | 1.40 | 581 | 14 |
| 27j | 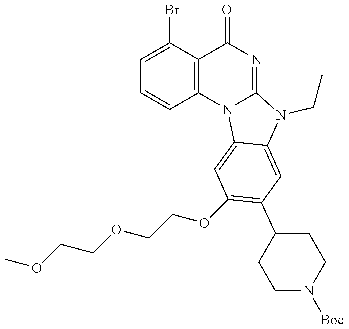 [SMILES: CCn1c2cc(C3CCN(C(=O)OC(C)(C)C)CC3)c(OCCOCCOC)cc2n2c3cccc(Br)c3c(=O)nc12] | 2.93 | 643/645 | 8 |
| 27k | 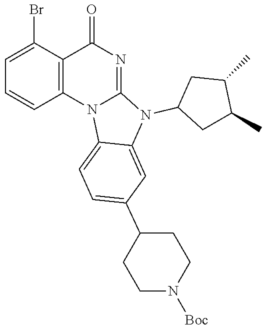 [SMILES: C[C@H]1CC(n2c3cc(C4CCN(C(=O)OC(C)(C)C)CC4)ccc3n3c4cccc(Br)c4c(=O)nc23)C[C@@H]1C] | 2.52 | 593/595 | 11 |
| 27l | 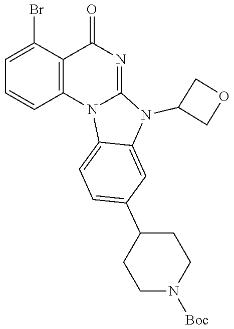 [SMILES: CC(C)(C)OC(=O)N1CCC(c2ccc3c(c2)n(C2COC2)c2nc(=O)c4c(Br)cccc4n32)CC1] | 2.24 | 553/555 | 9 |
| 27m | 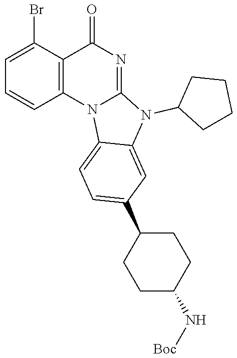 [SMILES: CC(C)(C)OC(=O)N[C@H]1CC[C@H](c2ccc3c(c2)n(C2CCCC2)c2nc(=O)c4c(Br)cccc4n32)CC1] | 0.82 | 579/581 | 4 |
| 27n | 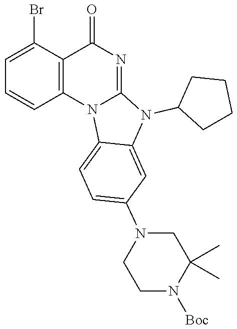 [SMILES: CC(C)(C)OC(=O)N1CCN(c2ccc3c(c2)n(C2CCCC2)c2nc(=O)c4c(Br)cccc4n32)CC1(C)C] | 2.71 | 594/596 | 9 |
| 27o | 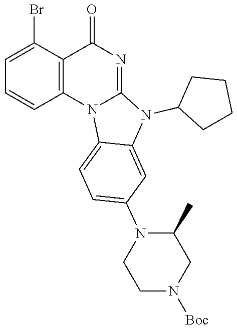 [SMILES: C[C@H]1CN(C(=O)OC(C)(C)C)CCN1c1ccc2c(c1)n(C1CCCC1)c1nc(=O)c3c(Br)cccc3n21] | 2.55 | 580/582 | 9 |
| 27p | 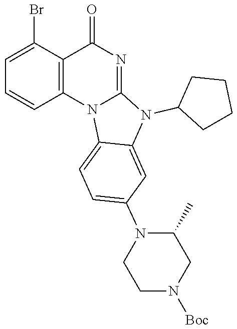 [SMILES: C[C@@H]1CN(C(=O)OC(C)(C)C)CCN1c1ccc2c(c1)n(C1CCCC1)c1nc(=O)c3c(Br)cccc3n21] | 2.55 | 580/582 | 9 |

### Experimental Procedure for the Synthesis of 30a

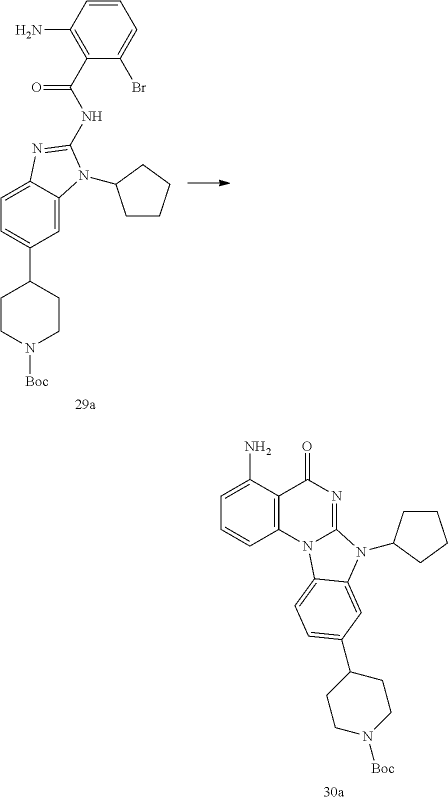 [SMILES: CC(C)(C)OC(=O)N1CCC(c2ccc3nc(NC(=O)c4c(N)cccc4Br)n(C4CCCC4)c3c2)CC1 ; CC(C)(C)OC(=O)N1CCC(c2ccc3c(c2)n(C2CCCC2)c2nc(=O)c4c(N)cccc4n32)CC1]

To a stirred solution of 29a (27.5 g, 47.0 mmol, 1.00 equiv.) in 1-methyl-4-pyrrolidione (270 ml), 1,10-phenonthrolene (1.70 g, 9.44 mmol, 0.20 equiv.), copper iodide (4.49 g, 23.6 mmol, 0.50 equiv.) and cesium carbonate (23.1 g, 70.8 mmol, 1.50 equiv.) are added at rt and the reaction mixture is degassed with nitrogen for 5 min and stirred at 120° C. for 1 h. Then the reaction mixture is cooled to rt and diluted with ice cold water. The mixture is extracted with ethyl acetate and the organic layer is dried with sodium sulfate, filtered off and concentrated under reduce pressure. The product is recrystallized with ethyl acetate to get 30a (6.60 g, 27.9%).

The following intermediates 30 (table 42) are available in an analogous manner using different starting materials 29. The crude product 30 is purified by chromatography if necessary.

TABLE 42

| | | | | | A2 binary |
|---|---|---|---|---|---|
| # | Structure | tᵣₑₜ [min] | [M + H]⁺ | HPLC method | [nM] |
| 30a | 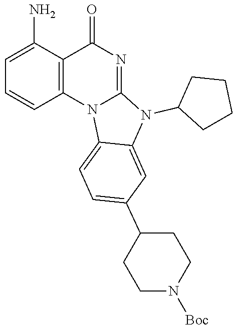 [SMILES: CC(C)(C)OC(=O)N1CCC(c2ccc3c(c2)n(C2CCCC2)c2nc(=O)c4c(N)cccc4n32)CC1] | 2.57 | 502 | 9 | 541 |
| 30b | 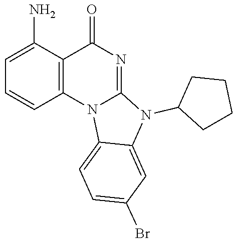 [SMILES: Nc1cccc2c1c(=O)nc1n(C3CCCC3)c3cc(Br)ccc3n21] | 2.33 | 397/399 | 9 | nd |

### Experimental Procedure for the Synthesis of 31a

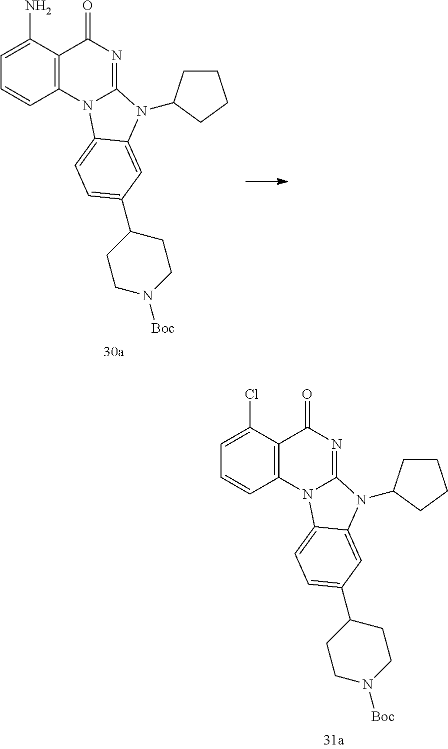 [SMILES: CC(C)(C)OC(=O)N1CCC(c2ccc3c(c2)n(C2CCCC2)c2nc(=O)c4c(N)cccc4n32)CC1 ; CC(C)(C)OC(=O)N1CCC(c2ccc3c(c2)n(C2CCCC2)c2nc(=O)c4c(Cl)cccc4n32)CC1]

To a stirred solution of 30a (5.00 g, 10.0 mmol, 1.00 equiv.) in acetonitrile (100 ml), p-toluenesulfonic acid monohydrate (5.14 g, 30.0 mmol, 3.00 equiv.) and copper(I) chloride (4.93 g, 50.0 mmol, 5.00 equiv.) are added at rt. To this reaction mixture a solution of sodium nitrite (1.38 g, 20.0 mmol, 2.00 equiv.) in water (20 ml) is added dropwise at rt and stirred for 1 h. The reaction mixture is diluted with water and the obtained solid is filtered off and dried. The residue is basified with sodium carbonate solution (pH-8) and extracted 3 times with dichloromethane (with 10% methanol). The organic layer is dried over sodium sulfate, filtered off and concentrated in vacuum. The crude product is purified by column chromatography to get 31a (2.20 g, 42.4%).

TABLE 43

| # | Structure | tᵣₑₜ [min] | [M + H]⁺ | HPLC method |
|---|---|---|---|---|
| 31a | 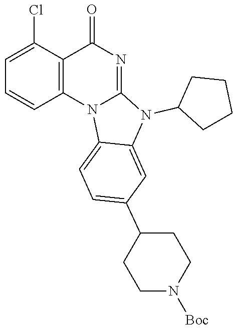 [SMILES: CC(C)(C)OC(=O)N1CCC(c2ccc3c(c2)n(C2CCCC2)c2nc(=O)c4c(Cl)cccc4n32)CC1] | 2.51 | 521 | 9 |

### Experimental Procedure for the Synthesis of 30c

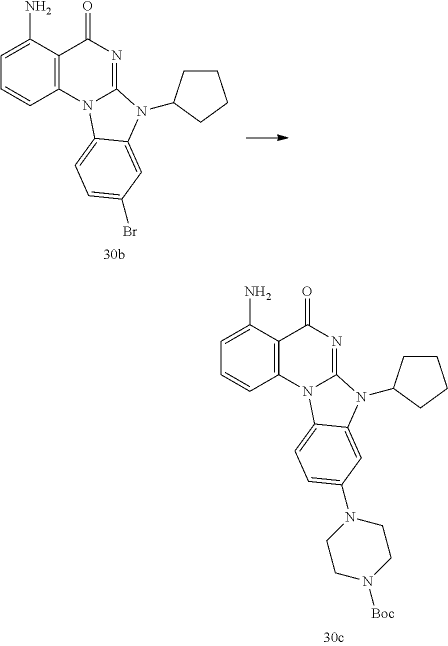 [SMILES: Nc1cccc2c1c(=O)nc1n(C3CCCC3)c3cc(Br)ccc3n21 ; CC(C)(C)OC(=O)N1CCN(c2ccc3c(c2)n(C2CCCC2)c2nc(=O)c4c(N)cccc4n32)CC1]

30b (300 mg, 0.68 mmol, 1.00 equiv.), tert-butyl tetrahydropyrazine-1(2H)-carboxylate (333 mg, 1.70 mmol, 2.50 equiv.) and methanesulfonato(2-dicyclohexylphosphino-2′,6′-di-i-propoxy-1,1′-biphenyl)(2′-amino-1,1′-biphenyl-2-yl)palladium(II) (59.2 mg, 0.07 mmol, 0.10 equiv.) are suspended in THF (6.00 ml). To this reaction mixture lithium bis(trimethylsilyl)amide, 1 M in THF, (4.76 ml, 4.76 mmol, 7.00 equiv.) is added dropwise at rt, flushed 5 min with argon and stirred for 20 min at 85° C. The reaction mixture is cooled to rt and diluted with dichloromethane and saturated ammonium chloride solution. The organic layer is separated and concentrated under reduce pressure. The crude product is purified by column chromatography to get 30c (227 mg, 59.8%).

The following intermediates 30c-30e (table 44) are available in an analogous manner using different starting materials amines.

TABLE 44

| | | tᵣₑₜ | | HPLC |
|---|---|---|---|---|
| # | Structure | [min] | [M + H]⁺ | method |
| 30c | 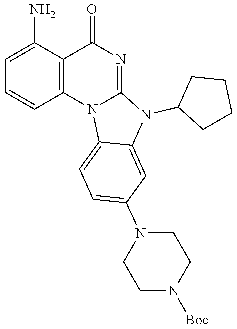 [SMILES: CC(C)(C)OC(=O)N1CCN(c2ccc3c(c2)n(C2CCCC2)c2nc(=O)c4c(N)cccc4n32)CC1] | 0.80 | 503 | 1 |
| 30d | 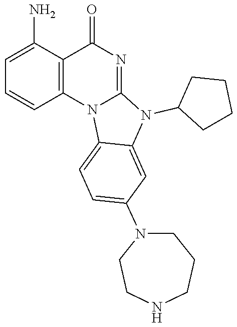 [SMILES: Nc1cccc2c1c(=O)nc1n(C3CCCC3)c3cc(N4CCCNCC4)ccc3n21] | 0.37 | 417 | 1 |
| 30e | 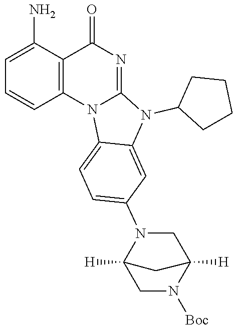 [SMILES: CC(C)(C)OC(=O)N1C[C@@H]2C[C@H]1CN2c1ccc2c(c1)n(C1CCCC1)c1nc(=O)c3c(N)cccc3n21] | 0.87 | 515 | 1 |

### Experimental Procedure for the Synthesis of 30f

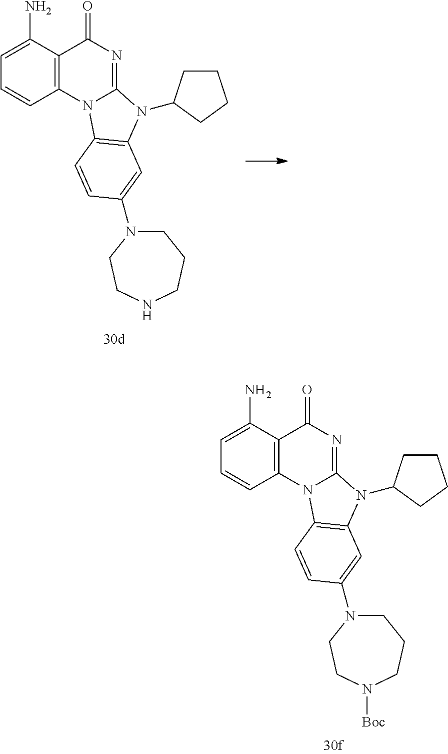 [SMILES: Nc1cccc2c1c(=O)nc1n(C3CCCC3)c3cc(N4CCCNCC4)ccc3n21 ; CC(C)(C)OC(=O)N1CCCN(c2ccc3c(c2)n(C2CCCC2)c2nc(=O)c4c(N)cccc4n32)CC1]

30d (185 mg; 0.444 mmol; 1.00 eq.) (crude) is suspended in DCM (3.00 mL)/DMF (3.00 mL), triethyl amine (0.34 mL; 2.45 mmol; 5.52 eq.) and (Boc)₂O (200 mg; 0.916 mmol; 2.06 eq.) are added. The reaction mixture is stirred at rt for 15 min. The reaction mixture is diluted with DCM (15 mL) and sat. NH₄Cl (10 mL). The layers are separated and the aq. layer is washed with DCM (3×5 mL). The combined organic layers are concentrated. The organic layer is concentrated and purified via NP chromatography to giving the desired product 30f (132 mg, 57.5%).

TABLE 45

| # | structure | tᵣₑₜ [min] | [M + H]⁺ | HPLC method |
|---|---|---|---|---|
| 30f | 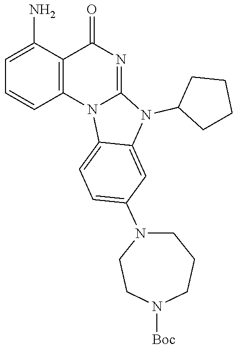 [SMILES: CC(C)(C)OC(=O)N1CCCN(c2ccc3c(c2)n(C2CCCC2)c2nc(=O)c4c(N)cccc4n32)CC1] | 0.81 | 517 | 1 |

### Experimental Procedure for the Synthesis of 31b

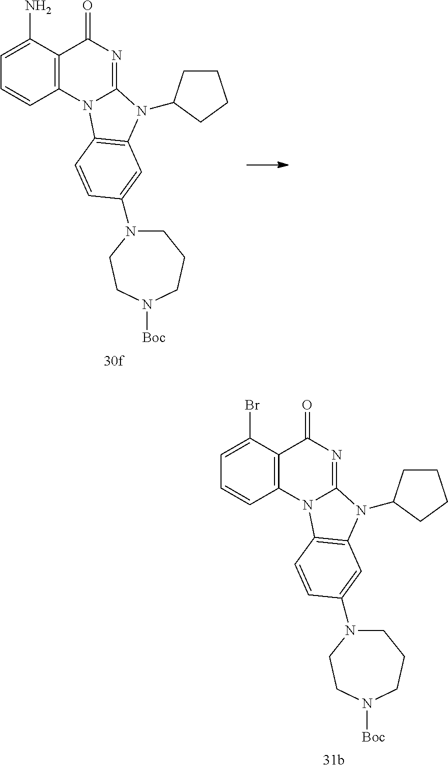 [SMILES: CC(C)(C)OC(=O)N1CCCN(c2ccc3c(c2)n(C2CCCC2)c2nc(=O)c4c(N)cccc4n32)CC1 ; CC(C)(C)OC(=O)N1CCCN(c2ccc3c(c2)n(C2CCCC2)c2nc(=O)c4c(Br)cccc4n32)CC1]

Amine 30f (130 mg; 0.252 mmol; 1.00 eq.) is dissolved in glacial acetic acid (1.50 mL) and acetonitrile (1.00 mLQ. The mixture is cooled to 4° C., then a solution of sodium nitrite (17.4 mg; 0.252 mmol; 1.00 eq.) in water (0.50 mL) is added dropwise. The mixture is stirred at 4° C. for 5 min Copper(I) bromide (40.0 mg; 0.279 mmol; 1.11 eq.) and hydrogen bromide, 47-49% in Water (1.50 mL) are added and the mixture is stirred at rt for 10 min. The mixture is cooled to 0° C. and 4M NaOH is added until pH=5 is reached. The mixture is filtered and the solid is washed with 2M NaOH (2 mL). The solid is dissolved in DCM (20 mL) and water (10 mL). The layers are separated and the aqueous layer is washed with DCM (2×5 mL). The organic layer is dried and concentrated under reduced pressure. The crude product is purified by column chromatography to giving the desired product 31 b (80 mg, 66.2%).

The following intermediates 31 (table 46) are available in an analogous manner using different starting materials 30.

TABLE 46

| | | tᵣₑₜ | | HPLC | A2 binary |
|---|---|---|---|---|---|
| # | structure | [min] | [M + H]⁺ | method | [nM] |
| 31b | 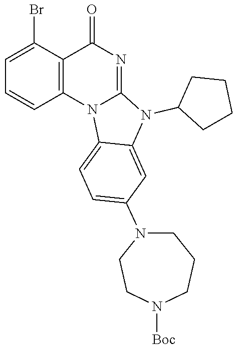 [SMILES: CC(C)(C)OC(=O)N1CCCN(c2ccc3c(c2)n(C2CCCC2)c2nc(=O)c4c(Br)cccc4n32)CC1] | 0.83 | 508/510 | 1 | nd |
| 31c | 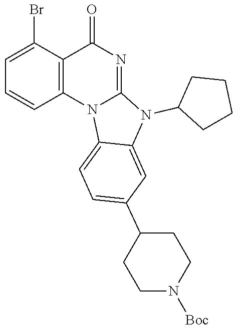 [SMILES: CC(C)(C)OC(=O)N1CCC(c2ccc3c(c2)n(C2CCCC2)c2nc(=O)c4c(Br)cccc4n32)CC1] | 0.42 | 480/482 | 1 | nd |
| 31d | 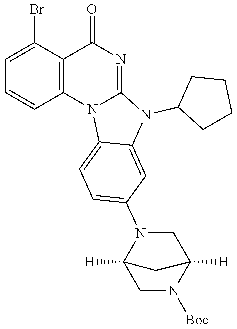 [SMILES: CC(C)(C)OC(=O)N1C[C@@H]2C[C@H]1CN2c1ccc2c(c1)n(C1CCCC1)c1nc(=O)c3c(Br)cccc3n21] | 0.40 | 478/480 | 1 | 104 |

### Experimental Procedure for the Synthesis of 28a

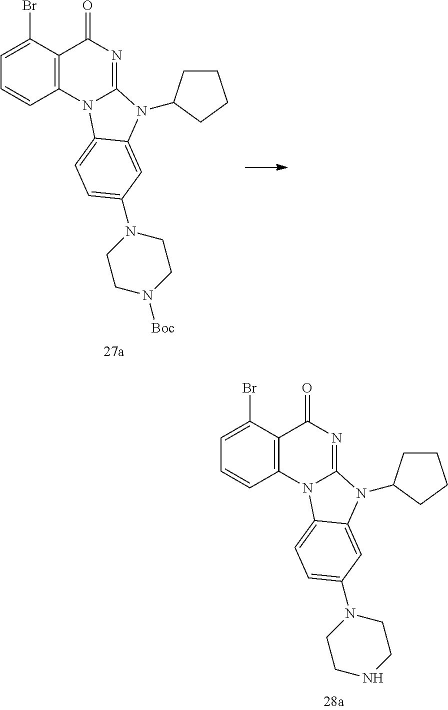 [SMILES: CC(C)(C)OC(=O)N1CCN(c2ccc3c(c2)n(C2CCCC2)c2nc(=O)c4c(Br)cccc4n32)CC1 ; O=c1nc2n(C3CCCC3)c3cc(N4CCNCC4)ccc3n2c2cccc(Br)c12]

To a stirred solution of 27a (42.0 g, 74.3 mmol, 1.00 equiv.) in dichloromethane (420 ml), 4 M HCl in 1,4-dioxane (186 mL, 743 mmol, 10.0 equiv.) is added dropwise at 0° C. and the reaction is stirred at this temperature for 2 h. The solvent is removed under reduce pressure and the obtained solid is dissolved in water. This water solution is basified by saturated sodium bicarbonate solution (pH-8) to get 28a which is filtered, rinsed with water and dried in vacuum (34.0 g, 98.4%).

The following intermediates 28 (table 47) are available in an analogous manner using different starting materials 27, 30 or 31. The crude product 28 is purified by chromatography if necessary.

TABLE 47

| | | | | HPLC | A2 binary |
|---|---|---|---|---|---|
| # | structure | tᵣₑₜ [min] | [M + H]⁺ | method | [nM] |
| 28a | 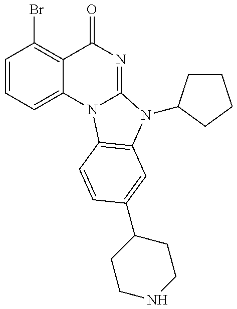 [SMILES: O=c1nc2n(C3CCCC3)c3cc(C4CCNCC4)ccc3n2c2cccc(Br)c12] | 1.44 | 465/467 | 11 | 48 |
| 28b | 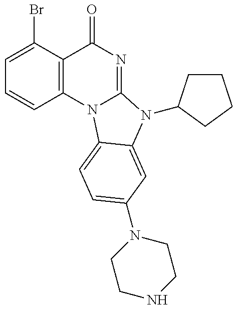 [SMILES: O=c1nc2n(C3CCCC3)c3cc(N4CCNCC4)ccc3n2c2cccc(Br)c12] | 1.56 | 466/468 | 9 | 44 |
| 28c | 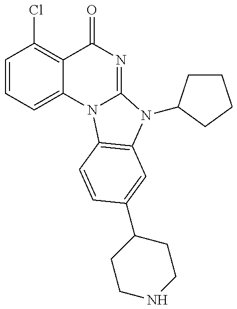 [SMILES: O=c1nc2n(C3CCCC3)c3cc(C4CCNCC4)ccc3n2c2cccc(Cl)c12] | 1.63 | 421 | 9 | nd |
| 28d | 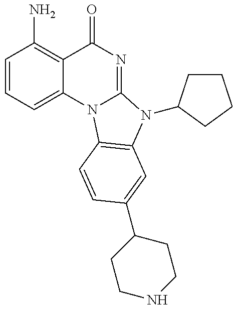 [SMILES: Nc1cccc2c1c(=O)nc1n(C3CCCC3)c3cc(C4CCNCC4)ccc3n21] | 0.38 | 402 | 1 | nd |
| 28e | 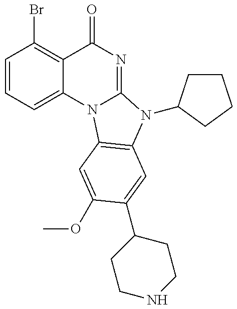 [SMILES: COc1cc2c(cc1C1CCNCC1)n(C1CCCC1)c1nc(=O)c3c(Br)cccc3n21] | 0.43 | 495/497 | 1 | nd |
| 28f | 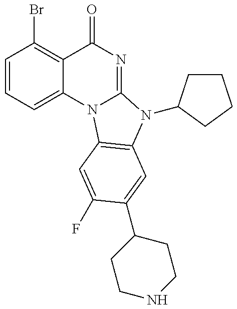 [SMILES: O=c1nc2n(C3CCCC3)c3cc(C4CCNCC4)c(F)cc3n2c2cccc(Br)c12] | 0.52 | 483/485 | 4 | nd |
| 28g | 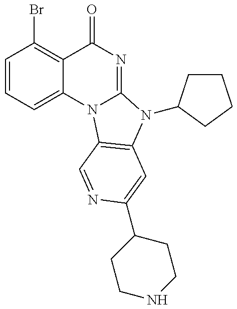 [SMILES: O=c1nc2n(C3CCCC3)c3cc(C4CCNCC4)ncc3n2c2cccc(Br)c12] | 2.09 | 466/468 | 9 | nd |
| 28h | 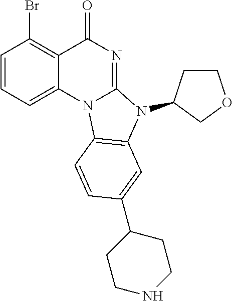 [SMILES: O=c1nc2n([C@H]3CCOC3)c3cc(C4CCNCC4)ccc3n2c2cccc(Br)c12] | 2.25 | 467/469 | 8 | nd |
| 28i | 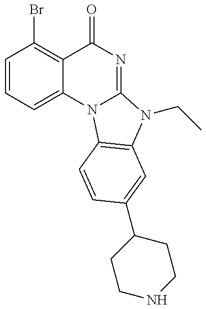 [SMILES: CCn1c2cc(C3CCNCC3)ccc2n2c3cccc(Br)c3c(=O)nc12] | 1.54 | 425/427 | 9 | nd |
| 28j | 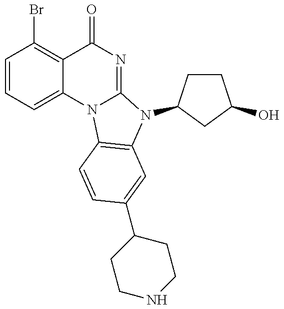 [SMILES: O=c1nc2n([C@H]3CC[C@@H](O)C3)c3cc(C4CCNCC4)ccc3n2c2cccc(Br)c12] | 0.41 | 481/483 | 4 | nd |
| 28k | 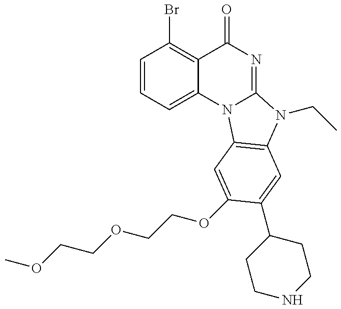 [SMILES: CCn1c2cc(C3CCNCC3)c(OCCOCCOC)cc2n2c3cccc(Br)c3c(=O)nc12] | 1.61 | 545 | 9 | 45 |
| 28l | 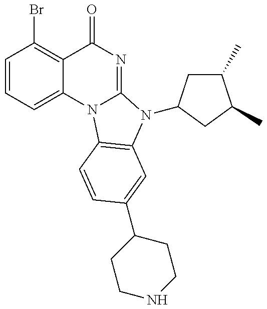 [SMILES: C[C@H]1CC(n2c3cc(C4CCNCC4)ccc3n3c4cccc(Br)c4c(=O)nc23)C[C@@H]1C] | 1.54 | 493/495 | 11 | nd |
| 28m | 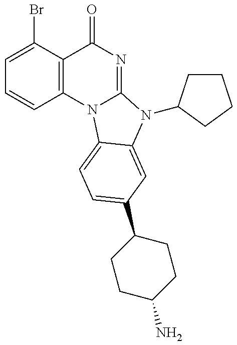 [SMILES: N[C@H]1CC[C@H](c2ccc3c(c2)n(C2CCCC2)c2nc(=O)c4c(Br)cccc4n32)CC1] | 0.54 | 479/481 | 4 | 29 |
| 28n | 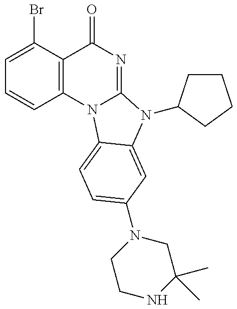 [SMILES: CC1(C)CN(c2ccc3c(c2)n(C2CCCC2)c2nc(=O)c4c(Br)cccc4n32)CCN1] | 1.48 | 494/496 | 11 | nd |
| 28o | 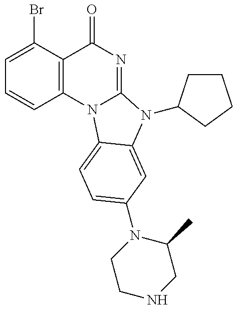 [SMILES: C[C@H]1CNCCN1c1ccc2c(c1)n(C1CCCC1)c1nc(=O)c3c(Br)cccc3n21] | 1.43 | 480/482 | 11 | 40 |
| 28p | 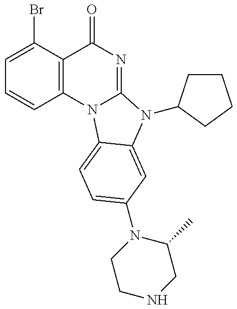 [SMILES: C[C@@H]1CNCCN1c1ccc2c(c1)n(C1CCCC1)c1nc(=O)c3c(Br)cccc3n21] | 1.41 | 480/482 | 11 | 55 |
| 28q | 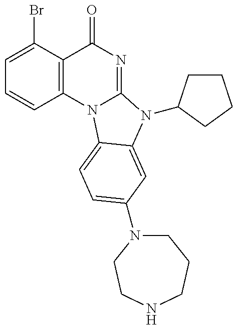 [SMILES: O=c1nc2n(C3CCCC3)c3cc(N4CCCNCC4)ccc3n2c2cccc(Br)c12] | 0.52 | 532 | 1 | nd |
| 28r | 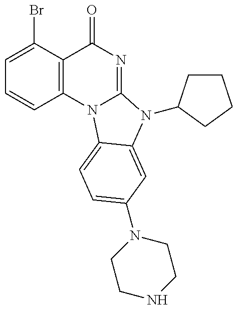 [SMILES: O=c1nc2n(C3CCCC3)c3cc(N4CCNCC4)ccc3n2c2cccc(Br)c12] | 0.43 | 466/468 | 1 | 44 |
| 28s | 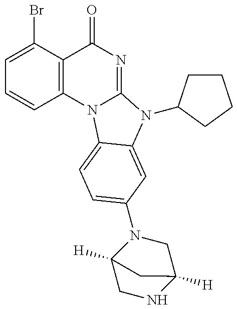 [SMILES: O=c1nc2n(C3CCCC3)c3cc(N4C[C@@H]5C[C@H]4CN5)ccc3n2c2cccc(Br)c12] | 1.43 | 478/480 | 11 | 70 |

### Experimental Procedure for the Synthesis of 28t

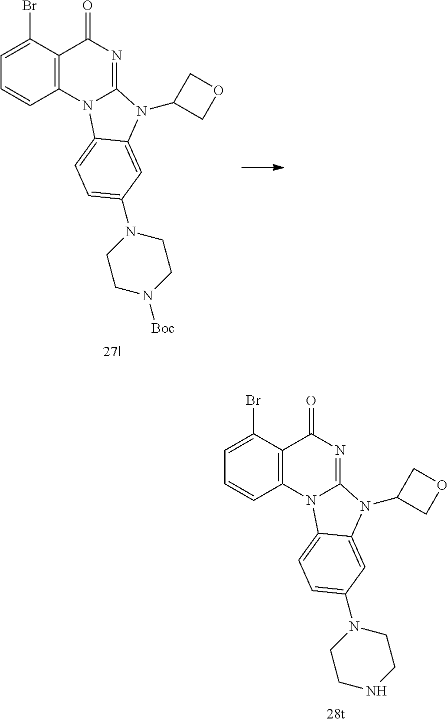 [SMILES: CC(C)(C)OC(=O)N1CCN(c2ccc3c(c2)n(C2COC2)c2nc(=O)c4c(Br)cccc4n32)CC1 ; O=c1nc2n(C3COC3)c3cc(N4CCNCC4)ccc3n2c2cccc(Br)c12]

To a stirred solution of 271 (0.5 g, 0.903 mmol, 1.00 equiv.) in dichloromethane (5 ml), H₃PO₄ (0.13 g, 1.355 mmol, 1.5 equiv.) is added slowly at −20° C. and the reaction is stirred at this temperature for 20 min. The reaction mixture is basified with 2N NaOH solution. The obtained solid is filtered off and dried giving 28t (0.4 g, 97.7%).

TABLE 48

| | | tᵣₑₜ | | HPLC |
|---|---|---|---|---|
| # | structure | [min] | [M + H]⁺ | method |
| 28t | 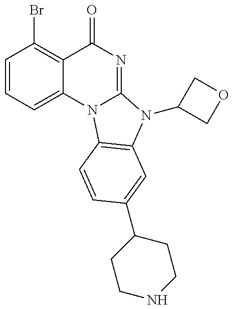 [SMILES: O=c1nc2n(C3COC3)c3cc(C4CCNCC4)ccc3n2c2cccc(Br)c12] | 1.51 | 453/455 | 9 |

### Experimental Procedure for the Synthesis of 28u

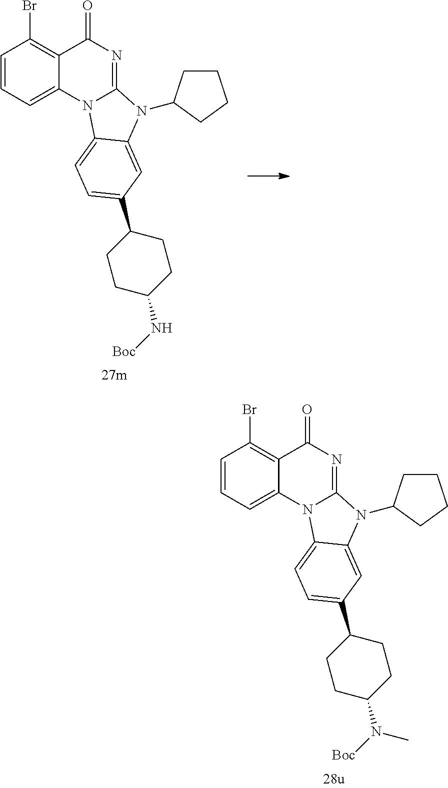 [SMILES: CC(C)(C)OC(=O)N[C@H]1CC[C@H](c2ccc3c(c2)n(C2CCCC2)c2nc(=O)c4c(Br)cccc4n32)CC1 ; CN(C(=O)OC(C)(C)C)[C@H]1CC[C@H](c2ccc3c(c2)n(C2CCCC2)c2nc(=O)c4c(Br)cccc4n32)CC1]

In a 50 mL round-bottom flask amine 27m (170 mg; 0.293 mmol; 1.00 eq.) is dissolved in THF, dry (4.000 mL) and cooled to 0° C. in an ice bath. Iodo methane (0.183 mL; 2.93 mmol; 10.0 eq.) is added followed by the portion wise addition of sodium hydride (128 mg; 2.93 mmol; 10.0 eq.). The ice bath is removed and the mixture is stirred at rt overnight. The mixture is quenched with water and extracted with DCM twice. The combined organic layers are washed with sat. NH₄Cl-solution, dried and concentrated under reduced pressure. The crude product 28u (233.0 mg; 133.8%) is taken to the next step without further purification.

TABLE 49

| | | tᵣₑₜ | | HPLC |
|---|---|---|---|---|
| # | Structure | [min] | [M + H]⁺ | method |
| 28u | 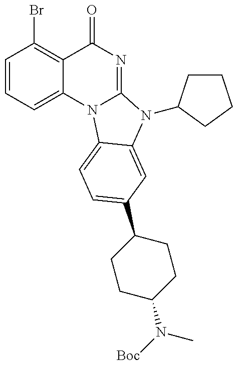 [SMILES: CN(C(=O)OC(C)(C)C)[C@H]1CC[C@H](c2ccc3c(c2)n(C2CCCC2)c2nc(=O)c4c(Br)cccc4n32)CC1] | 0.91 | 593/595 | 4 |

### Synthesis Towards Intermediates 28″

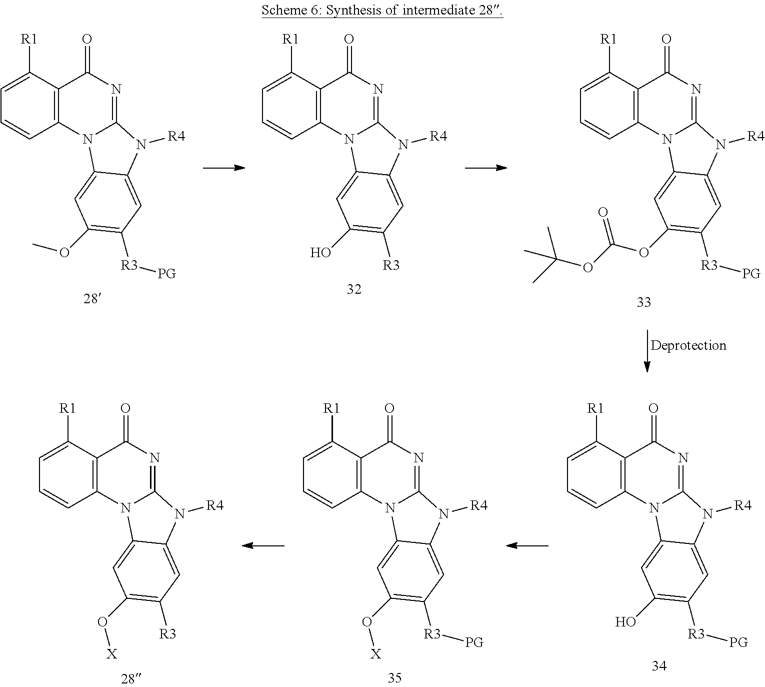

Further modifications on intermediate 28′ can be realized by standard ether cleavage leading to alcohol 32. Orthogonal protecting group strategy is leading to intermediate 33, which after carbonate cleavage is giving intermediate 34. The phenol can be used to install various residues e.g. using Mitsunobu type or alkylation reaction conditions leading to intermediate 35. Deprotection of 35 using acidic conditions is leading to further modified intermediates 28″.

### Experimental Procedure for the Synthesis of 32a

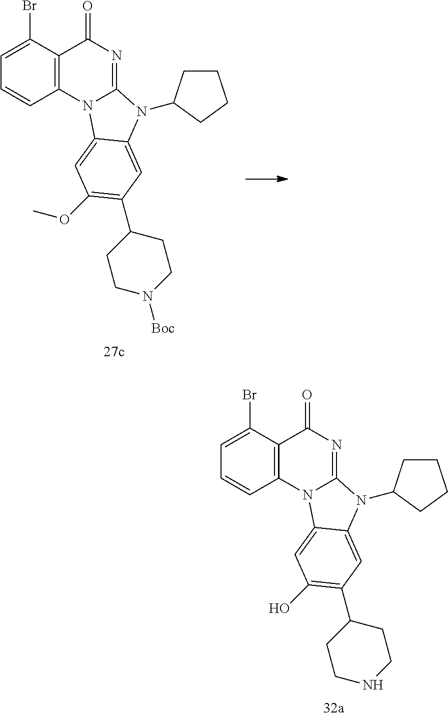 [SMILES: COc1cc2c(cc1C1CCN(C(=O)OC(C)(C)C)CC1)n(C1CCCC1)c1nc(=O)c3c(Br)cccc3n21 ; O=c1nc2n(C3CCCC3)c3cc(C4CCNCC4)c(O)cc3n2c2cccc(Br)c12]

To a stirred solution of 27c (42.0 g, 71.0 mmol, 1.00 equiv.) in dichloromethane (420 ml), boron tribromide (21.2 g, 85.0 mmol, 1.20 equiv.) is added slowly dropwise at 0° C. The reaction mixture is stirred at rt for 16 h. The solvent is removed under reduce pressure and the obtained crude product 32a is used for the next step. (40.0 g, 117.8%).

TABLE 50

| | | tᵣₑₜ | | HPLC |
|---|---|---|---|---|
| # | Structure | [min] | [M + H]⁺ | method |
| 32a | 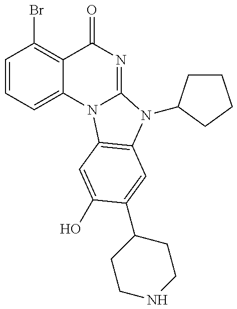 [SMILES: O=c1nc2n(C3CCCC3)c3cc(C4CCNCC4)c(O)cc3n2c2cccc(Br)c12] | 2.03 | 481/483 | 9 |

### Experimental Procedure for the Synthesis of 33a

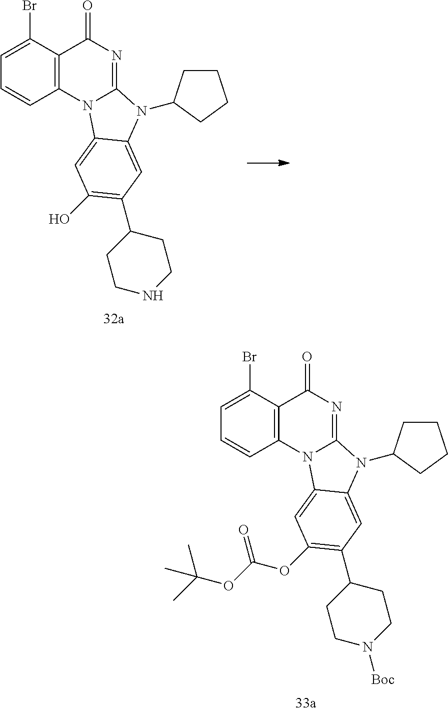 [SMILES: O=c1nc2n(C3CCCC3)c3cc(C4CCNCC4)c(O)cc3n2c2cccc(Br)c12 ; CC(C)(C)OC(=O)Oc1cc2c(cc1C1CCN(C(=O)OC(C)(C)C)CC1)n(C1CCCC1)c1nc(=O)c3c(Br)cccc3n21]

To a stirred solution of 32a (20.0 g, 42.0 mmol, 1.00 equiv.) in dichloromethane (200 ml), triethyl amine (21.0 g, 208 mmol, 5.00 equiv.) is added dropwise at rt. The reaction mixture is stirred at rt for 15 min. Then di-tert-butyl dicarbonate (9.97 g, 46.0 mmol, 1.10 equiv.) is added at rt for 16 h. The reaction mixture is diluted with 10% methanol in dichloromethane and water. The organic layer is separated and the aqueous layer is extracted again with 10% methanol in dichloromethane. The combined organic layer is washed with water and brine and then concentrated under reduce pressure. The obtained residue is purified by column chromatography to get 33a (20.0 g, 70.6%).

TABLE 51

| | | | | | A2 |
|---|---|---|---|---|---|
| | | tᵣₑₜ | [M + | HPLC | binary |
| # | Structure | [min] | H]⁺ | method | [nM] |
| 33a | 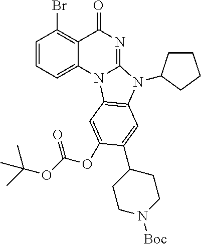 [SMILES: CC(C)(C)OC(=O)Oc1cc2c(cc1C1CCN(C(=O)OC(C)(C)C)CC1)n(C1CCCC1)c1nc(=O)c3c(Br)cccc3n21] | 2.70 | 681/683 | 9 | 157 |

### Experimental Procedure for the Synthesis of 34a

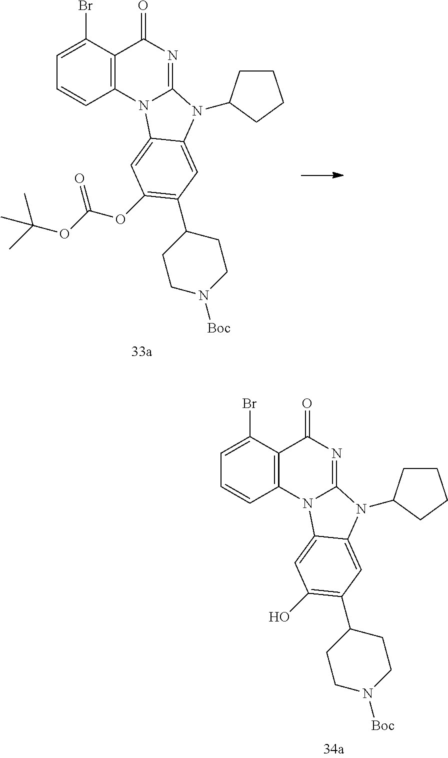 [SMILES: CC(C)(C)OC(=O)Oc1cc2c(cc1C1CCN(C(=O)OC(C)(C)C)CC1)n(C1CCCC1)c1nc(=O)c3c(Br)cccc3n21 ; CC(C)(C)OC(=O)N1CCC(c2cc3c(cc2O)n2c4cccc(Br)c4c(=O)nc2n3C2CCCC2)CC1]

To a stirred solution of 33a (20.0 g, 29.0 mmol, 1.00 equiv.) in methanol (200 ml), sodium hydroxide (4.69 g, 117 mmol, 4.00 equiv.) in water (50 ml) is added at rt. The reaction mixture is stirred at rt for 6 h. The reaction mixture is concentrated under reduce pressure and the obtained residue is acidified with saturated citric acid solution up to pH-6. The precipitated solid is filtered off, washed with ether and dried in vacuum to get 34a (17.0 g, 99.6%).

TABLE 52

| | | tᵣₑₜ | | HPLC |
|---|---|---|---|---|
| # | structure | [min] | [M + H]⁺ | method |
| 34a | 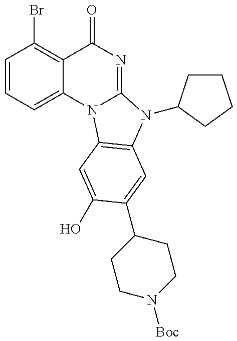 [SMILES: CC(C)(C)OC(=O)N1CCC(c2cc3c(cc2O)n2c4cccc(Br)c4c(=O)nc2n3C2CCCC2)CC1] | 2.37 | 581/583 | 9 |

### Experimental Procedure for the Synthesis of 35a

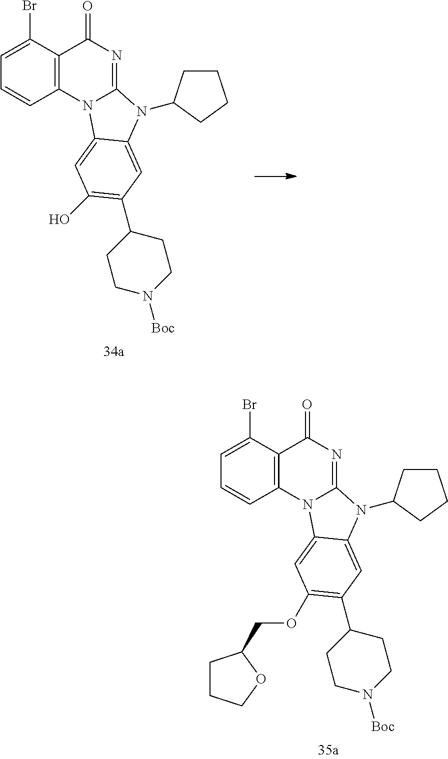 [SMILES: CC(C)(C)OC(=O)N1CCC(c2cc3c(cc2O)n2c4cccc(Br)c4c(=O)nc2n3C2CCCC2)CC1 ; CC(C)(C)OC(=O)N1CCC(c2cc3c(cc2OC[C@@H]2CCCO2)n2c4cccc(Br)c4c(=O)nc2n3C2CCCC2)CC1]

To a stirred solution of alcohol 34a (4.35 g, 6.18 mmol, 1.00 equiv.) and [(2S)-oxolan-2-yl]methyl methane sulfonate (1.67 g, 9.27 mmol, 1.5 equiv.) in DMF (40 ml), dipotassium carbonate (2.56 g, 18.5 mmol, 3.0 equiv.) are added. The reaction mixture is stirred at 85° C. overnight. The reaction mixture is cooled to rt, poured in water (400 mL), stirred at rt for 30 min. The precipitated solid is filtered off, washed with water and dried in vacuum to get 35a (4.7 g, 116%).

The following intermediates 35 (table 53) are available in an analogous manner using different sulfonates.

TABLE 53

| # | Structure | tᵣₑₜ [min] | [M + H]⁺ | HPLC method |
|---|---|---|---|---|
| 35a | 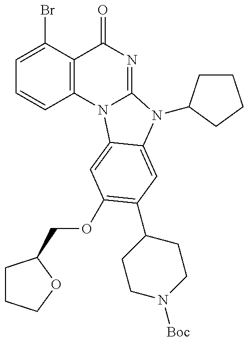 [SMILES: CC(C)(C)OC(=O)N1CCC(c2cc3c(cc2OC[C@@H]2CCCO2)n2c4cccc(Br)c4c(=O)nc2n3C2CCCC2)CC1] | 1.62 | 665 | 14 |
| 35c | 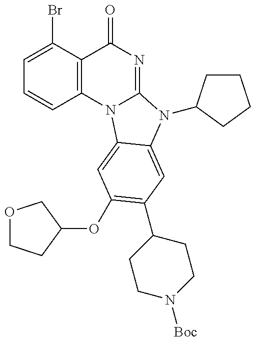 [SMILES: CC(C)(C)OC(=O)N1CCC(c2cc3c(cc2OC2CCOC2)n2c4cccc(Br)c4c(=O)nc2n3C2CCCC2)CC1] | 0.82 | 651/653 | 4 |
| 35e | 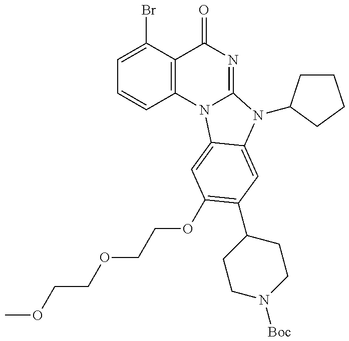 [SMILES: COCCOCCOc1cc2c(cc1C1CCN(C(=O)OC(C)(C)C)CC1)n(C1CCCC1)c1nc(=O)c3c(Br)cccc3n21] | 0.83 | 683/685 | 7 |
| 35f | 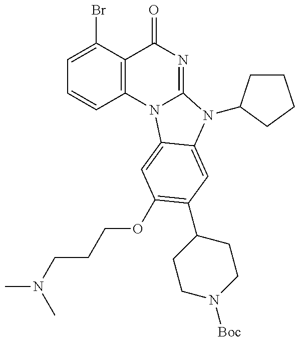 [SMILES: CN(C)CCCOc1cc2c(cc1C1CCN(C(=O)OC(C)(C)C)CC1)n(C1CCCC1)c1nc(=O)c3c(Br)cccc3n21] | 0.83 | 666/668 | 4 |

### Experimental Procedure for the Synthesis of 35g

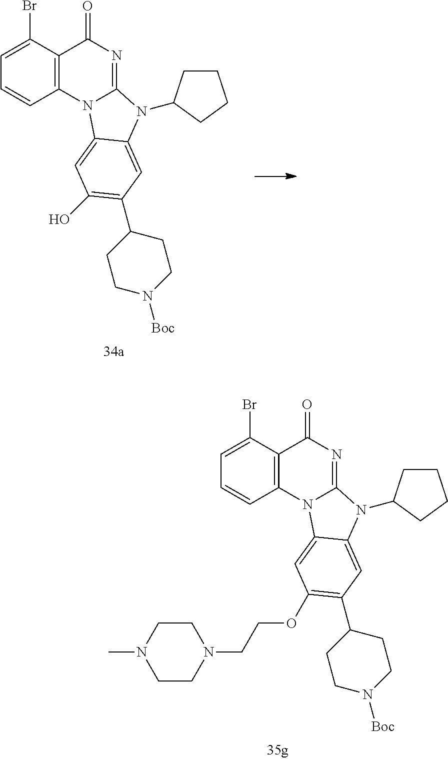 [SMILES: CC(C)(C)OC(=O)N1CCC(c2cc3c(cc2O)n2c4cccc(Br)c4c(=O)nc2n3C2CCCC2)CC1 ; CN1CCN(CCOc2cc3c(cc2C2CCN(C(=O)OC(C)(C)C)CC2)n(C2CCCC2)c2nc(=O)c4c(Br)cccc4n32)CC1]

34a (0.205 g, 0.353 mmol, 1.00 equiv.), triphenylphosphine (0.148 g, 0.564 mmol, 1.60 equiv.) and 1-hydroxyethyl-4-methyl-piperazin (0.076 g, 0.529 mmol, 1.50 equiv.) are dissolved in THF abs. (3 mL). Then diisopropyl azodicarboxylate (0.103 mL, 0.529 mmol, 1.50 equiv.) is added. The reaction is stirred 1h at rt. The mixture is poured into water and stirred for 15 min. The formed solid is filtered off, washed with water and dried. The residue is purified by normal phase chromatography. Product fraction are combined and concentrated under reduce pressure to give 35g (0.12 g, 49.3%).

TABLE 54

| # | structure | tᵣₑₜ [min] | [M + H]⁺ | HPLC method |
|---|---|---|---|---|
| 35g | 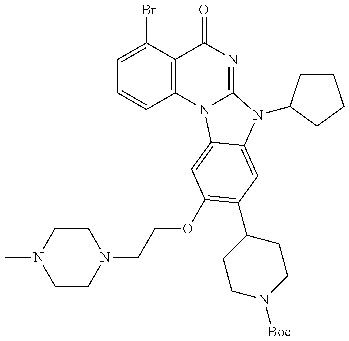 [SMILES: CN1CCN(CCOc2cc3c(cc2C2CCN(C(=O)OC(C)(C)C)CC2)n(C2CCCC2)c2nc(=O)c4c(Br)cccc4n32)CC1] | 0.75 | 707/709 | 7 |

### Experimental Procedure for the Synthesis of 28v

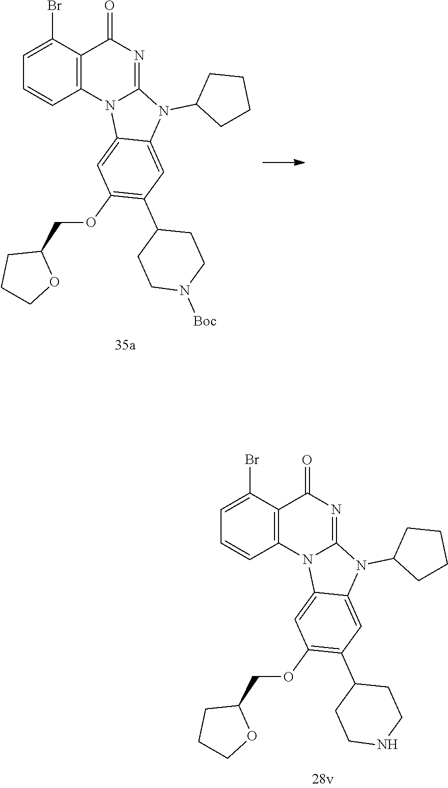 [SMILES: CC(C)(C)OC(=O)N1CCC(c2cc3c(cc2OC[C@@H]2CCCO2)n2c4cccc(Br)c4c(=O)nc2n3C2CCCC2)CC1 ; O=c1nc2n(C3CCCC3)c3cc(C4CCNCC4)c(OC[C@@H]4CCCO4)cc3n2c2cccc(Br)c12]

To a stirred solution of 35a (4.8 g, 7.18 mmol, 1.00 equiv.) in MeOH (25 mL), 4 M HCl in 1,4-dioxane (10 mL, 40.0 mmol, 5.6 equiv.) is added and the reaction is stirred at 50° C. for 1 h. The solvent is removed under reduce pressure and the obtained solid is dissolved in water. This water solution is basified by saturated sodium bicarbonate solution (pH-8) and extracted with DCM (250 ml). The aqueous layer is extracted with DCM (2×250 ml) and the combined organic layer is concentrated under reduce pressure to give 28v (4.1 g, 100%). The following intermediates 28 (table 55) are available in an analogous manner using different starting materials 35. The crude product 28 is purified by chromatography if

TABLE 55

| | | | | HPLC | A2 binary |
|---|---|---|---|---|---|
| # | structure | tᵣₑₜ [min] | [M + H]⁺ | method | [nM] |
| 28v | 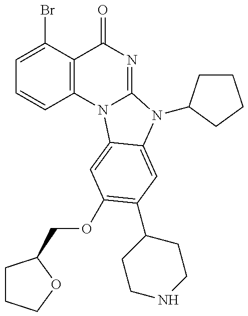 [SMILES: O=c1nc2n(C3CCCC3)c3cc(C4CCNCC4)c(OC[C@@H]4CCCO4)cc3n2c2cccc(Br)c12] | 0.54 | 565/567 | 4 | 45 |
| 28x | 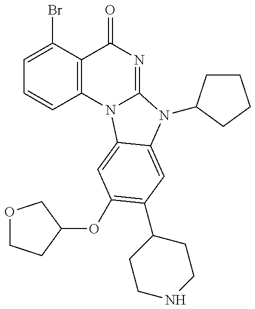 [SMILES: O=c1nc2n(C3CCCC3)c3cc(C4CCNCC4)c(OC4CCOC4)cc3n2c2cccc(Br)c12] | 0.420 | 551/553 | 1 | nd |
| 28y | 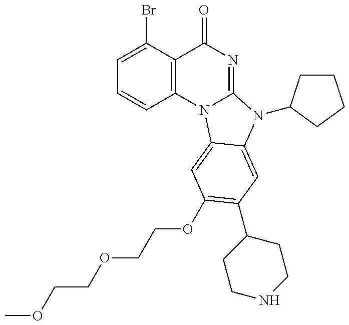 [SMILES: COCCOCCOc1cc2c(cc1C1CCNCC1)n(C1CCCC1)c1nc(=O)c3c(Br)cccc3n21] | 0.43 | 583/585 | 1 | 50 |
| 28z | 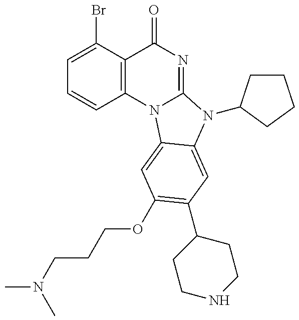 [SMILES: CN(C)CCCOc1cc2c(cc1C1CCNCC1)n(C1CCCC1)c1nc(=O)c3c(Br)cccc3n21] | 0.28 | 566/568 | 1 | nd |
| 28aa | 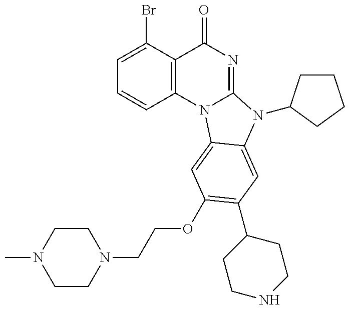 [SMILES: CN1CCN(CCOc2cc3c(cc2C2CCNCC2)n(C2CCCC2)c2nc(=O)c4c(Br)cccc4n32)CC1] | 0.29 | 607/609 | 6 | nd |

### Synthesis Towards Compound 42

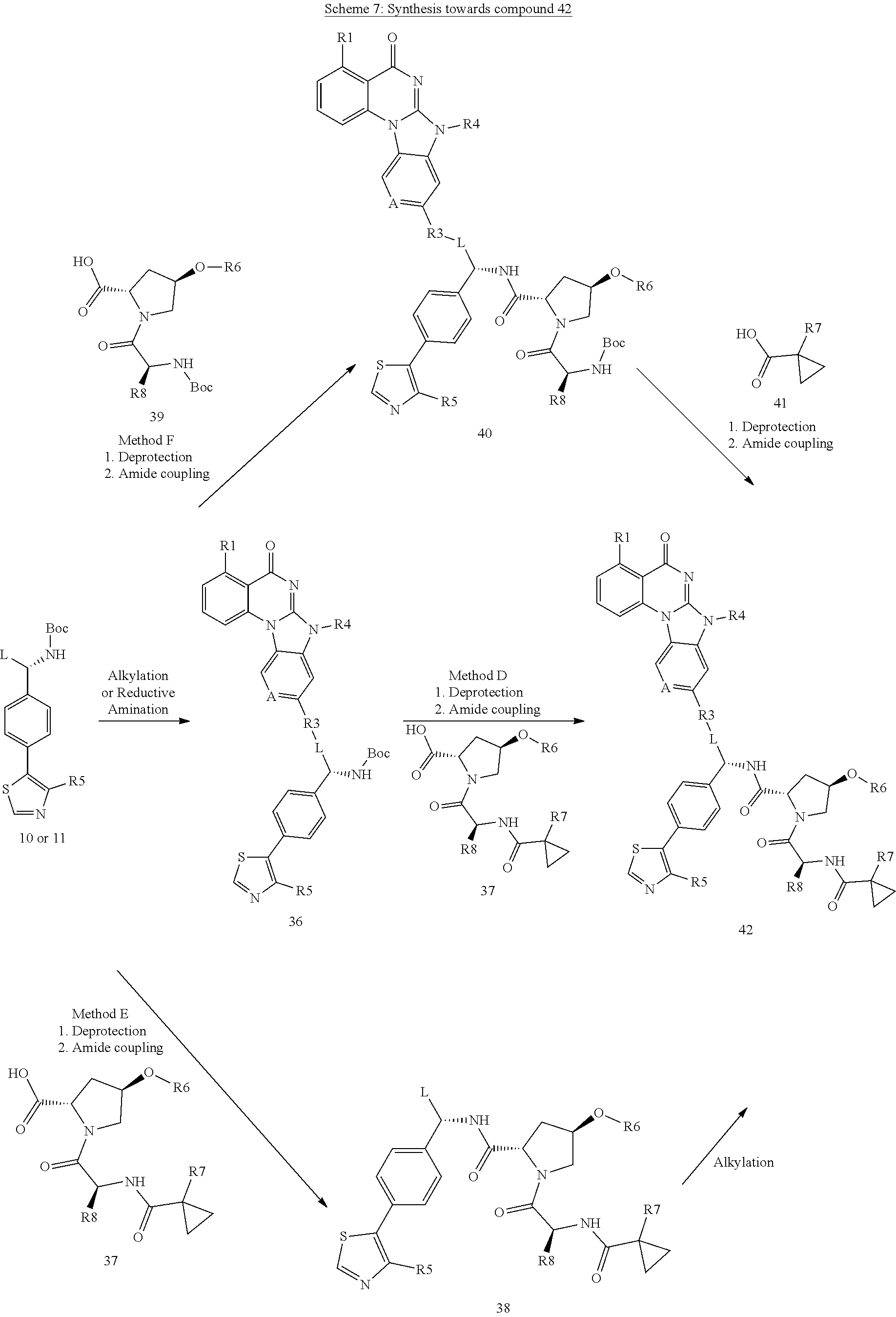

Due to their modular structure, there are various routes towards compounds of formula 42. All methods used start from common intermediates 10 or 11.

Method D: Intermediate 10 or 11 can be attached to compound 28 using e.g. standard alkylation or reductive amination reactions leading to intermediate 36. Intermediate 36 can be transformed via acidic deprotection and subsequent amide coupling with intermediate 37 using coupling reagents such as HATU or T3P to compounds 42.

Method E: Intermediate 10 or 11 is deprotected under acidic conditions and transformed via amide coupling with intermediate 37 using standard coupling reagents such as HATU or T3P into intermediate 38. Intermediate 38 is then alkylated with compound 28under basic alkylation conditions leading to compounds 42.

Method F: Intermediate 36 is deprotected under acidic conditions and coupled with intermediate 39 under standard amide coupling reaction conditions using reagents such as HATU, CDI or T3P to intermediate 40. Subsequent acidic deprotection and another amide coupling are leading to compound 42.

As the skilled person will appreciate, compounds of formula 42 are compounds of formula (III).

### Synthesis Towards Intermediate 37 for Method E

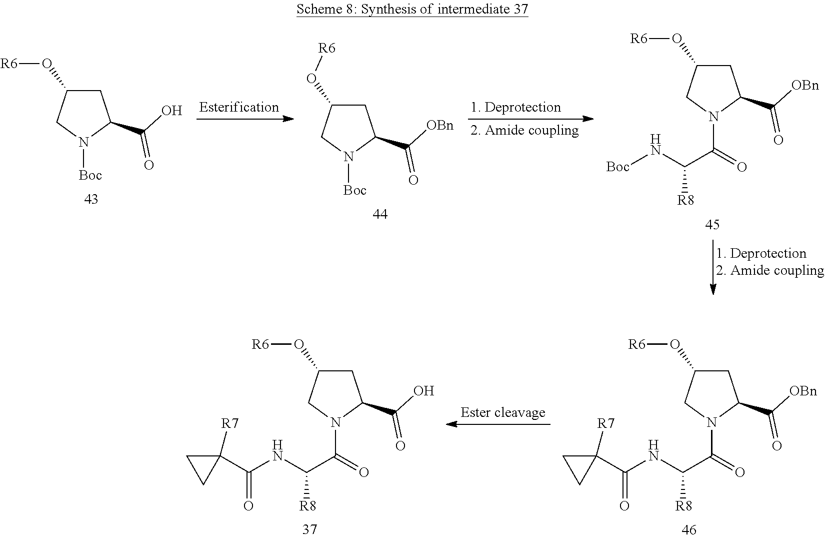

The carboxylic acid in proline 43 is protected as benzylic ester leading to intermediate 44. Subsequent deprotection of the amine under acidic conditions followed by a standard amide coupling leads to intermediate 45. Deprotection of the amine using standard conditions followed by an amide coupling leads to intermediate 46. Cleavage of the benzyl ester using hydrogenolysis leads to intermediate 37.

### Experimental Procedure for the Synthesis of 44a

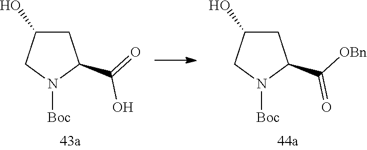 [SMILES: CC(C)(C)OC(=O)N1C[C@H](O)C[C@H]1C(=O)O ; CC(C)(C)OC(=O)N1C[C@H](O)C[C@H]1C(=O)OCc1ccccc1]

To a stirred solution of Boc-Hyp-OH (135 g, 580 mmol, 1.0 equiv.) in THF (700 mL) is added benzyl bromide (76.3 mL, 642 mmol, 1.1 equiv.) under ice cooled condition followed by addition of triethylamine (89.5 mL, 642 mmol, 1.1 equiv.). The reaction mass is stirred at rt for 17 h. After completion of reaction, the resultant mixture is filtered and the filtrate is concentrated under reduced pressure. Then the obtained crude is diluted with water (500 mL) and extracted with ethyl acetate (2×1 L). The combined organic layer is separated, dried over anhydrous magnesium sulfate and concentrated under reduced pressure. The resulting crude is purified by silica gel column chromatography (cyclohexane/EtOAc) to obtain the desired product 44a (150 g, 80.2%).

TABLE 56

| # | Structure | tᵣₑₜ [min] | [M + H]⁺ | HPLC method |
|---|---|---|---|---|
| 44a | 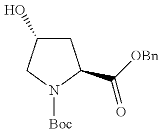 [SMILES: CC(C)(C)OC(=O)N1C[C@H](O)C[C@H]1C(=O)OCc1ccccc1] | 2.09 | 322 | 9 |

### Experimental Procedure for the Synthesis of 45a

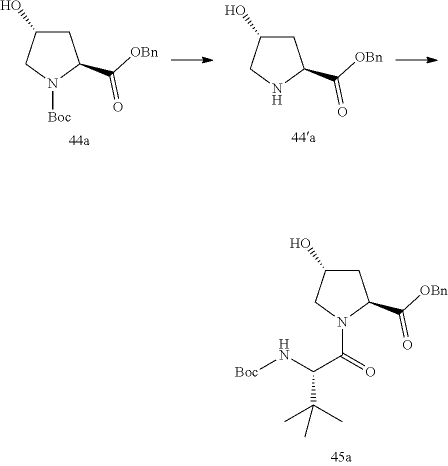 [SMILES: CC(C)(C)OC(=O)N1C[C@H](O)C[C@H]1C(=O)OCc1ccccc1 ; O=C(OCc1ccccc1)[C@@H]1C[C@@H](O)CN1 ; CC(C)(C)OC(=O)N[C@H](C(=O)N1C[C@H](O)C[C@H]1C(=O)OCc1ccccc1)C(C)(C)C]

To a stirred solution of 44a (50.0 g, 155 mmol, 1.0 equiv.) in 1,4-dioxane (100 mL) is added 4 N HCl in 1,4-dioxane (100 mL) under ice cooled conditions. The reaction mixture is then allowed to stir at rt for 2 h. After completion, the reaction mixture is concentrated under reduced pressure and azeotroped with toluene to get the desired product 44′a (34.0 g, 85.1%) as a solid, hydrochloride salt. The product is used crude for the next step.

To a stirred solution of Boc-Tle-OH (500 mg, 2.16 mmol, 1.0 equiv.) in MeCN (12 mL) is added the crude compound 44′a (625 mg, 2.38 mmol, 1.1 equiv.), HATU (1174 mg, 3.03 mmol, 1.4 equiv.) and TEA (1.56 mL, 10.8 mmol, 5.0 equiv.). The reaction mixture is stirred at rt for 1 h. After completion of the reaction, the reaction mixture is concentrated under reduced pressure to get the crude compound which is purified by column chromatography to get pure 45a (852 mg, 91.0%).

The following intermediates 45 (table 57) are available in an analogous manner using different protected aminoacids. The crude product 45 is purified by chromatography if

TABLE 57

| # | structure | tᵣₑₜ [min] | [M + H]⁺ | HPLC method |
|---|---|---|---|---|
| 45a | 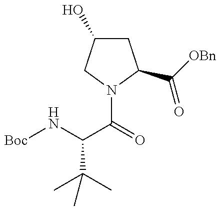 [SMILES: CC(C)(C)OC(=O)N[C@H](C(=O)N1C[C@H](O)C[C@H]1C(=O)OCc1ccccc1)C(C)(C)C] | 0.74 | 435 | 14 |
| 45b | 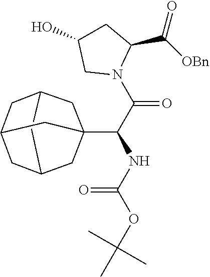 [SMILES: CC(C)(C)OC(=O)N[C@H](C(=O)N1C[C@H](O)C[C@H]1C(=O)OCc1ccccc1)C12CC3CC(CC(C3)C1)C2] | 1.63 | 513 | 14 |
| 45c | 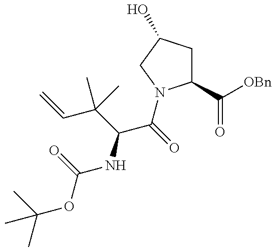 [SMILES: C=CC(C)(C)[C@H](NC(=O)OC(C)(C)C)C(=O)N1C[C@H](O)C[C@H]1C(=O)OCc1ccccc1] | 1.44 | 391 | 14 |
| 45d | 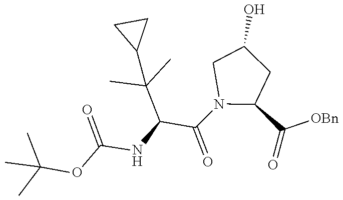 [SMILES: CC(C)(C)OC(=O)N[C@H](C(=O)N1C[C@H](O)C[C@H]1C(=O)OCc1ccccc1)C(C)(C)C1CC1] | 1.38 | 361 | 14 |
| 45e | 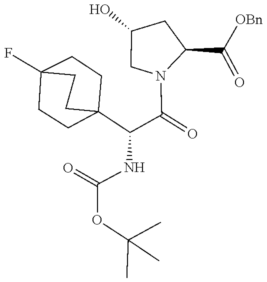 [SMILES: CC(C)(C)OC(=O)N[C@@H](C(=O)N1C[C@H](O)C[C@H]1C(=O)OCc1ccccc1)C12CCC(F)(CC1)CC2] | 1.37 | 405 | 14 |
| 45f | 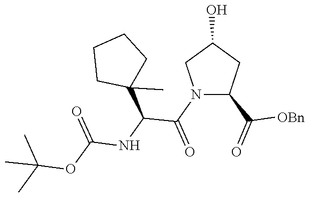 [SMILES: CC(C)(C)OC(=O)N[C@H](C(=O)N1C[C@H](O)C[C@H]1C(=O)OCc1ccccc1)C1(C)CCCC1] | 0.76 | 361 | 1 |

### Experimental Procedure for the Synthesis of 45′a

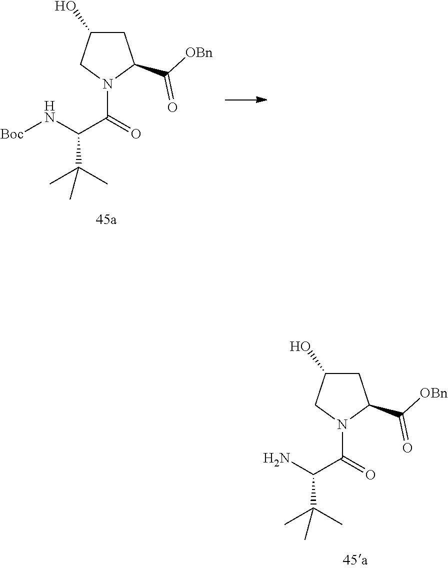 [SMILES: CC(C)(C)OC(=O)N[C@H](C(=O)N1C[C@H](O)C[C@H]1C(=O)OCc1ccccc1)C(C)(C)C ; CC(C)(C)[C@H](N)C(=O)N1C[C@H](O)C[C@H]1C(=O)OCc1ccccc1]

To a stirred solution of 45a (323 mg, 0.743 mmol) in MeOH (3 mL) is added 4 N HCl in 1,4-dioxane (1 mL). The reaction mixture is stirred 20 min at 60° C. After completion, the reaction mixture is concentrated under reduced pressure to get the desired product 45′a (261 mg, 95.0%) as a hydrochloride salt. The crude product is used for the next step.

TABLE 58

| | | | | HPLC |
|---|---|---|---|---|
| # | structure | tᵣₑₜ [min] | [M + H]⁺ | method |
| 45′a | 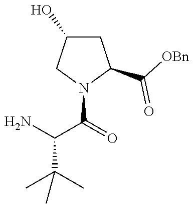 | 0.51 | 349 | 14 |

### Experimental Procedure for the Synthesis of 45′b

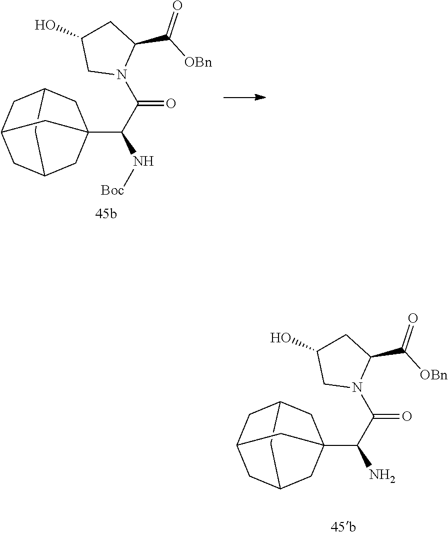 [SMILES: CC(C)(C)OC(=O)N[C@H](C(=O)N1C[C@H](O)C[C@H]1C(=O)OCc1ccccc1)C12CC3CC(CC(C3)C1)C2 ; N[C@H](C(=O)N1C[C@H](O)C[C@H]1C(=O)OCc1ccccc1)C12CC3CC(CC(C3)C1)C2]

To a stirred solution of 45b (1.16 g, 2.26 mmol) in DCE (2.00 mL) is added TFA (1.00 mL). The reaction mixture is stirred 30 min at 60° C. After completion, the reaction mixture is concentrated under reduced pressure to get the desired product 45′b (838 mg, 90%). The crude product is used for the next step.

The following intermediates 45′ (table 59) are available in an analogous manner using different starting materials 45. The crude product 45′ is taken to the next step without further purification.

TABLE 59

| | | tᵣₑₜ | [M + | HPLC |
|---|---|---|---|---|
| # | structure | [min] | H]⁺ | method |
| 45′b | 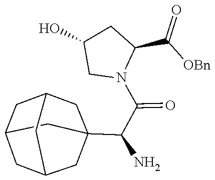 [SMILES: N[C@H](C(=O)N1C[C@H](O)C[C@H]1C(=O)OCc1ccccc1)C12CC3CC(CC(C3)C1)C2] | 0.46 | 413 | 1 |
| 45′c | 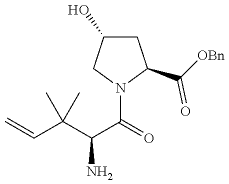 [SMILES: C=CC(C)(C)[C@H](N)C(=O)N1C[C@H](O)C[C@H]1C(=O)OCc1ccccc1] | 0.52 | 347 | 14 |
| 45′c | 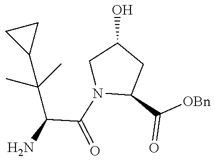 [SMILES: CC(C)(C1CC1)[C@H](N)C(=O)N1C[C@H](O)C[C@H]1C(=O)OCc1ccccc1] | 0.56 | 361 | 4 |
| 45′d | 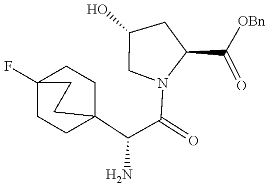 [SMILES: N[C@@H](C(=O)N1C[C@H](O)C[C@H]1C(=O)OCc1ccccc1)C12CCC(F)(CC1)CC2] | 0.52 | 405 | 4 |
| 45′e | 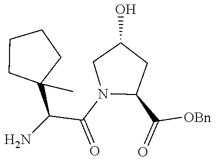 [SMILES: CC1([C@H](N)C(=O)N2C[C@H](O)C[C@H]2C(=O)OCc2ccccc2)CCCC1] | 0.56 | 361 | 4 |

### Experimental Procedure for the Synthesis of 46a

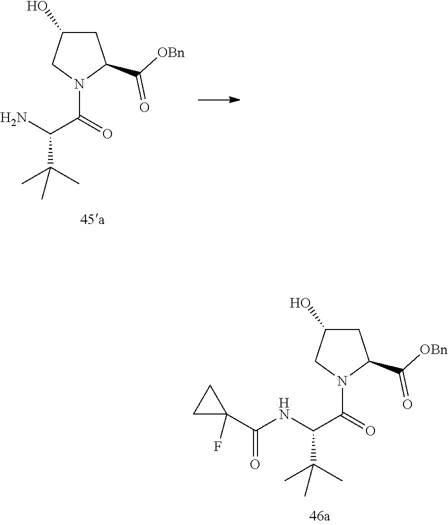 [SMILES: CC(C)(C)[C@H](N)C(=O)N1C[C@H](O)C[C@H]1C(=O)OCc1ccccc1 ; CC(C)(C)[C@H](NC(=O)C1(F)CC1)C(=O)N1C[C@H](O)C[C@H]1C(=O)OCc1ccccc1]

To a stirred solution of 1-fluorocyclopropanecarboxylic acid (18.8 g, 0.181 mol, 1.1 eq) in DCM (641 mL) is added HATU (81.1 g, 0.213 mol, 1.3 eq). The reaction mixture is stirred 20 min at rt. DIPEA (88.6 mL, 0.509 mol, 3.1 eq) and compound 45′a (64.1 g, 0.164 mol, 1 eq) are added, the reaction mixture is stirred at rt for 2 hours, and quenched with 300 mL of citric acid. The extracted organic phase is washed with 300 mL of sat. sodium bicarbonate solution and 100 mL of water. The organic phase is concentrated under reduced pressure to get the desired product 46a (69.1 g, 97.5%).

The following intermediates 46 (table 60) are available in an analogous manner using different compounds 45′ and carboxylic acids. The crude product 46 is purified by chromatography if necessary.

TABLE 60

| | | tᵣₑₜ | | HPLC |
|---|---|---|---|---|
| # | structure | [min] | [M + H]⁺ | method |
| 46a | 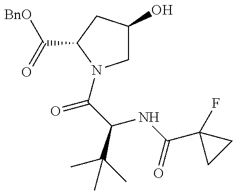 [SMILES: CC(C)(C)[C@H](NC(=O)C1(F)CC1)C(=O)N1C[C@H](O)C[C@H]1C(=O)OCc1ccccc1] | 2.11 | 421 | 9 |
| 46b | 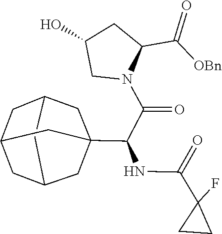 [SMILES: O=C(OCc1ccccc1)[C@@H]1C[C@@H](O)CN1C(=O)[C@@H](NC(=O)C1(F)CC1)C12CC3CC(CC(C3)C1)C2] | 0.81 | 499 | 1 |
| 46c | 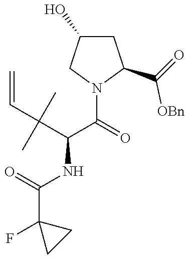 [SMILES: C=CC(C)(C)[C@H](NC(=O)C1(F)CC1)C(=O)N1C[C@H](O)C[C@H]1C(=O)OCc1ccccc1] | 1.21 | 433 | 14 |
| 46d | 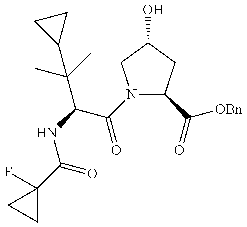 [SMILES: CC(C)(C1CC1)[C@H](NC(=O)C1(F)CC1)C(=O)N1C[C@H](O)C[C@H]1C(=O)OCc1ccccc1] | 1.31 | 447 | 14 |
| 46e | 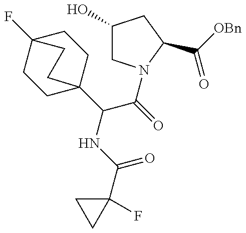 [SMILES: O=C(OCc1ccccc1)[C@@H]1C[C@@H](O)CN1C(=O)C(NC(=O)C1(F)CC1)C12CCC(F)(CC1)CC2] | 1.27 | 491 | 14 |
| 46f | 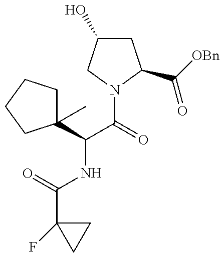 [SMILES: CC1([C@H](NC(=O)C2(F)CC2)C(=O)N2C[C@H](O)C[C@H]2C(=O)OCc2ccccc2)CCCC1] | 0.71 | 447 | 4 |
| 46g | 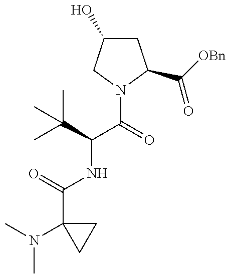 [SMILES: CN(C)C1(C(=O)N[C@H](C(=O)N2C[C@H](O)C[C@H]2C(=O)OCc2ccccc2)C(C)(C)C)CC1] | 1.35 | 446 | 14 |

### Experimental Procedure for the Synthesis of 37a

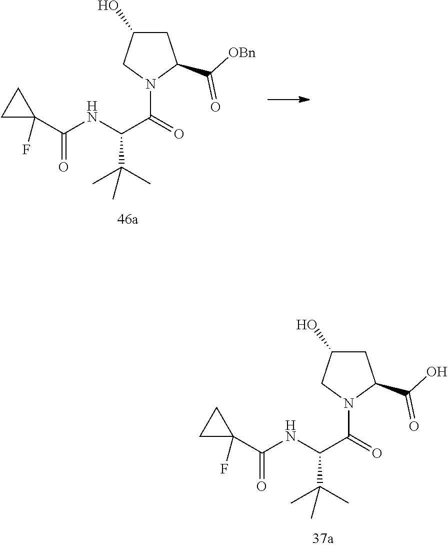 [SMILES: CC(C)(C)[C@H](NC(=O)C1(F)CC1)C(=O)N1C[C@H](O)C[C@H]1C(=O)OCc1ccccc1 ; CC(C)(C)[C@H](NC(=O)C1(F)CC1)C(=O)N1C[C@H](O)C[C@H]1C(=O)O]

Palladium (10% on carbon, 6.81 g) is added to a solution of 46a (13.5 g, 32.1 mmol, 1.0 equiv.) in MeOH (140 mL). The reaction mixture is stirred for 12 h with a hydrogen pressure of 80 PSI at rt. The reaction is filtered through a Celite bed followed by washing twice with ether. The filtrate is concentrated under reduced pressure to give 37a (8.10 g, 76.4%).

The following intermediates 37 (table 61) are available in an analogous manner using different starting materials 46. The crude product 37 is purified by chromatography if necessary.

TABLE 61

| | | tᵣₑₜ | | HPLC |
|---|---|---|---|---|
| # | structure | [min] | [M + H]⁺ | method |
| 37a | 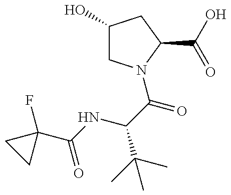 [SMILES: CC(C)(C)[C@H](NC(=O)C1(F)CC1)C(=O)N1C[C@H](O)C[C@H]1C(=O)O] | 1.81 | 331 | 9 |
| 37b | 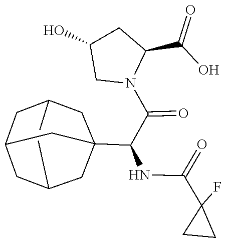 [SMILES: CC1CC2CC(C)CC([C@H](NC(=O)C3(F)CC3)C(=O)N3C[C@H](O)C[C@H]3C(=O)O)(C1)C2] | 0.85 | 409 | 14 |
| 37c | 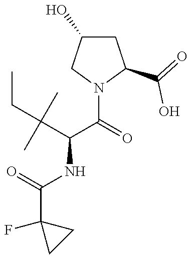 [SMILES: CCC(C)(C)[C@H](NC(=O)C1(F)CC1)C(=O)N1C[C@H](O)C[C@H]1C(=O)O] | 0.60 | 345 | 14 |
| 37d | 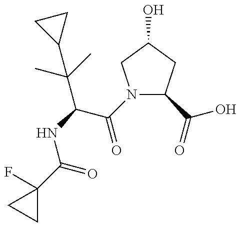 [SMILES: CC(C)(C1CC1)[C@H](NC(=O)C1(F)CC1)C(=O)N1C[C@H](O)C[C@H]1C(=O)O] | 0.63 | 357 | 14 |
| 37e | 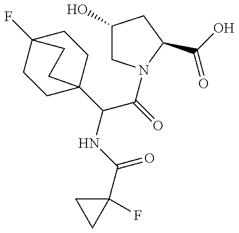 [SMILES: O=C(O)[C@@H]1C[C@@H](O)CN1C(=O)C(NC(=O)C1(F)CC1)C12CCC(F)(CC1)CC2] | 0.65 | 401 | 14 |
| 37f | 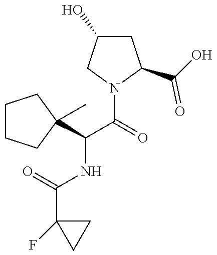 [SMILES: CC1([C@H](NC(=O)C2(F)CC2)C(=O)N2C[C@H](O)C[C@H]2C(=O)O)CCCC1] | 0.40 | 357 | 1 |
| 37g | 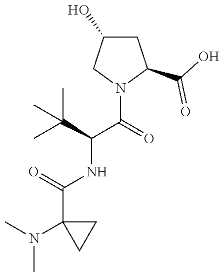 [SMILES: CN(C)C1(C(=O)N[C@H](C(=O)N2C[C@H](O)C[C@H]2C(=O)O)C(C)(C)C)CC1] | 0.18 | 356 | 4 |

Synthesis Towards Intermediates 36 using Method 0

### Experimental Procedure for the Synthesis of 36a

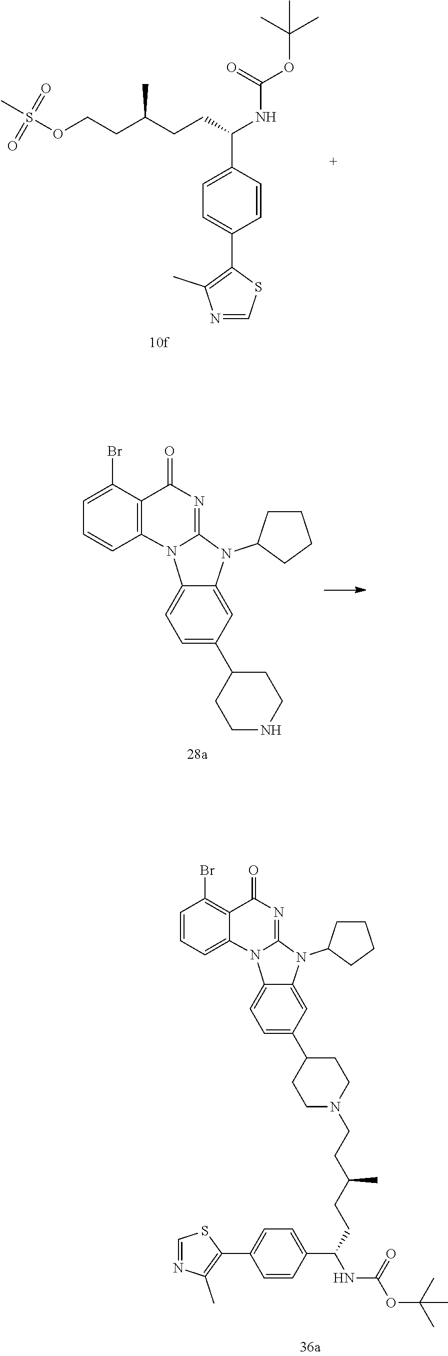 [SMILES: Cc1ncsc1-c1ccc([C@H](CC[C@H](C)CCOS(C)(=O)=O)NC(=O)OC(C)(C)C)cc1 ; O=c1nc2n(C3CCCC3)c3cc(C4CCNCC4)ccc3n2c2cccc(Br)c12 ; Cc1ncsc1-c1ccc([C@H](CC[C@H](C)CCN2CCC(c3ccc4c(c3)n(C3CCCC3)c3nc(=O)c5c(Br)cccc5n43)CC2)NC(=O)OC(C)(C)C)cc1]

In a 50 mL round-bottom flask carbamate 10f (398 mg; 0.825 mmol; 1.00 eq.) and 4-bromo-7-cyclopentyl-9-(4-piperidyl)benzimidazolo[1,2-a]quinazolin-5-one 28a (595 mg; 1.28 mmol; 1.30 eq.) are taken up in NMP (5 mL) and acetonitrile (5 mL). Then potassium carbonate (272 mg; 1.97 mmol; 2.00 eq.) and potassium iodide (327 mg; 1.97 mmol; 2.00 eq.) are added. The mixture is stirred at 80° C. for 2 h. The reaction mixture is diluted with DCM and water. The layers are separated. The aqueous layer is extracted with DCM. The combined organic layers are washed with sat. NH₄Cl-solution, passed through a phase separator cartridge and concentrated. The residue is dissolved in ACN/H₂O, filtered through a syringe filter and purified by column chromatography giving the desired product 36a (616 mg, 74%).

The following intermediates 36 (table 62) are available in an analogous manner using different starting materials 10. The crude product 36 is purified by chromatography if

TABLE 62

| | | | | HPLC |
|---|---|---|---|---|
| # | structure | tᵣₑₜ [min] | [M + H]⁺ | method |
| 36a | 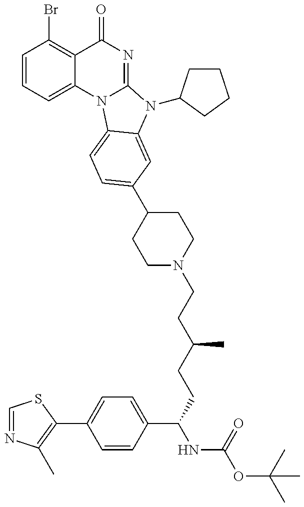 [SMILES: Cc1ncsc1-c1ccc([C@H](CC[C@H](C)CCN2CCC(c3ccc4c(c3)n(C3CCCC3)c3nc(=O)c5c(Br)cccc5n43)CC2)NC(=O)OC(C)(C)C)cc1] | 1.75 | 851 | 14 |
| 36b | 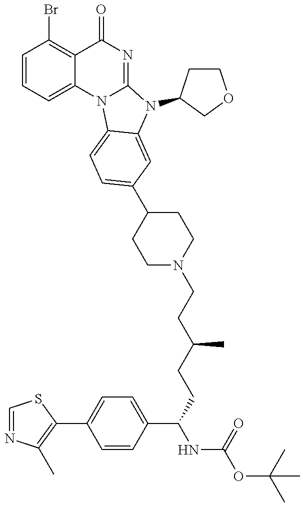 [SMILES: Cc1ncsc1-c1ccc([C@H](CC[C@H](C)CCN2CCC(c3ccc4c(c3)n([C@H]3CCOC3)c3nc(=O)c5c(Br)cccc5n43)CC2)NC(=O)OC(C)(C)C)cc1] | 0.90 | 853/855 | 5 |
| 36c | 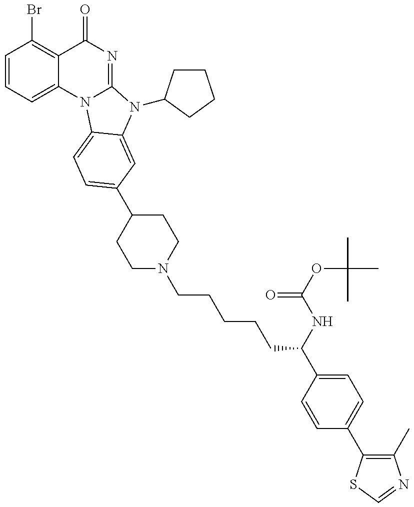 [SMILES: Cc1ncsc1-c1ccc([C@H](CCCCCN2CCC(c3ccc4c(c3)n(C3CCCC3)c3nc(=O)c5c(Br)cccc5n43)CC2)NC(=O)OC(C)(C)C)cc1] | 0.90 | 853/855 | 5 |
| 36d | 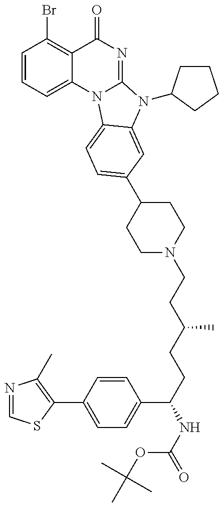 [SMILES: Cc1ncsc1-c1ccc([C@H](CC[C@@H](C)CCN2CCC(c3ccc4c(c3)n(C3CCCC3)c3nc(=O)c5c(Br)cccc5n43)CC2)NC(=O)OC(C)(C)C)cc1] | 1.75 | 851 | 14 |
| 36e | 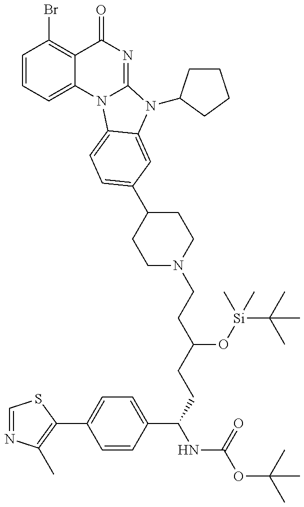 [SMILES: Cc1ncsc1-c1ccc([C@H](CCC(CCN2CCC(c3ccc4c(c3)n(C3CCCC3)c3nc(=O)c5c(Br)cccc5n43)CC2)O[Si](C)(C)C(C)(C)C)NC(=O)OC(C)(C)C)cc1] | 2.01 | 484 | 14 |
| 36f | 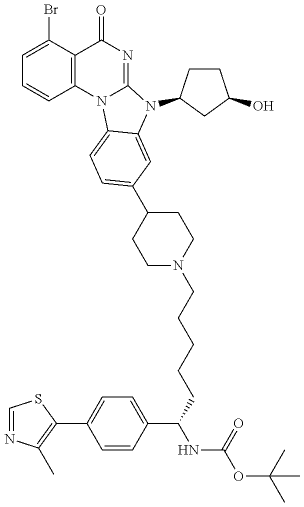 [SMILES: Cc1ncsc1-c1ccc([C@H](CCCCCN2CCC(c3ccc4c(c3)n([C@H]3CC[C@@H](O)C3)c3nc(=O)c5c(Br)cccc5n43)CC2)NC(=O)OC(C)(C)C)cc1] | 0.87 | 853/855 | 5 |
| 36g | 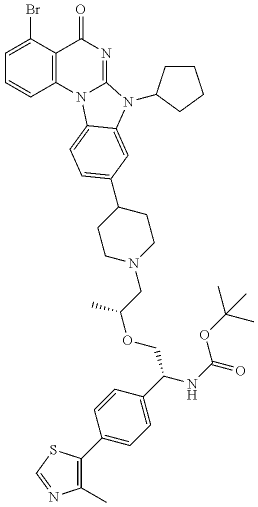 [SMILES: Cc1ncsc1-c1ccc([C@H](CO[C@H](C)CN2CCC(c3ccc4c(c3)n(C3CCCC3)c3nc(=O)c5c(Br)cccc5n43)CC2)NC(=O)OC(C)(C)C)cc1] | 1.71 | 420 | 14 |
| 36h | 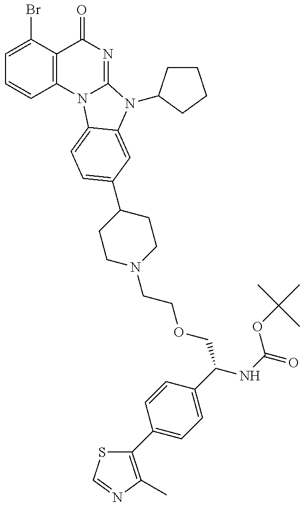 [SMILES: Cc1ncsc1-c1ccc([C@H](COCCN2CCC(c3ccc4c(c3)n(C3CCCC3)c3nc(=O)c5c(Br)cccc5n43)CC2)NC(=O)OC(C)(C)C)cc1] | 1.60 | 825 | 14 |
| 36i | 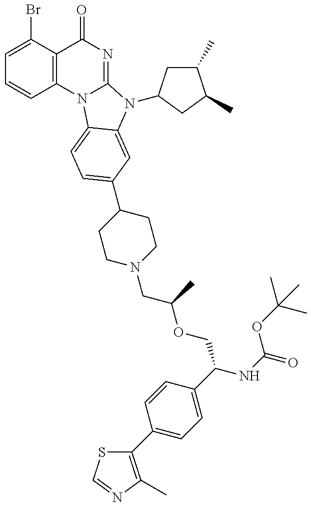 [SMILES: Cc1ncsc1-c1ccc([C@H](CO[C@H](C)CN2CCC(c3ccc4c(c3)n(C3C[C@H](C)[C@@H](C)C3)c3nc(=O)c5c(Br)cccc5n43)CC2)NC(=O)OC(C)(C)C)cc1] | 1.02 | 867/869 | 7 |
| 36j | 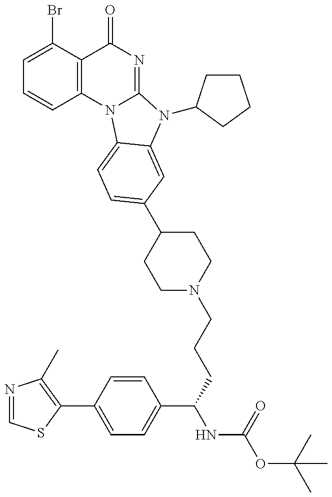 [SMILES: Cc1ncsc1-c1ccc([C@H](CCCN2CCC(c3ccc4c(c3)n(C3CCCC3)c3nc(=O)c5c(Br)cccc5n43)CC2)NC(=O)OC(C)(C)C)cc1] | 0.90 | 809/811 | 7 |
| 36k | 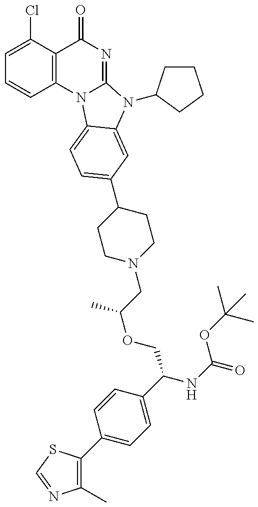 [SMILES: Cc1ncsc1-c1ccc([C@H](CO[C@H](C)CN2CCC(c3ccc4c(c3)n(C3CCCC3)c3nc(=O)c5c(Cl)cccc5n43)CC2)NC(=O)OC(C)(C)C)cc1] | 0.62 | 795/797 | 6 |
| 36l | 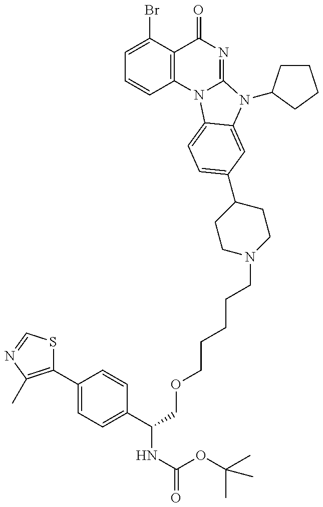 [SMILES: Cc1ncsc1-c1ccc([C@H](COCCCCCN2CCC(c3ccc4c(c3)n(C3CCCC3)c3nc(=O)c5c(Br)cccc5n43)CC2)NC(=O)OC(C)(C)C)cc1] | 0.96 | 867/869 | 12 |
| 36m | 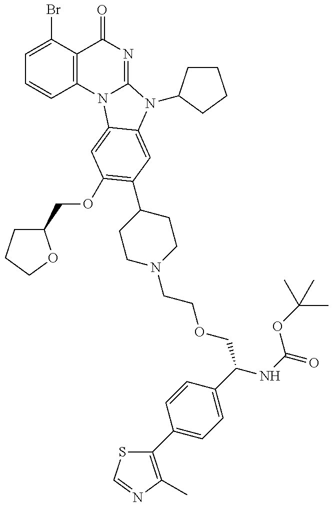 [SMILES: Cc1ncsc1-c1ccc([C@H](COCCN2CCC(c3cc4c(cc3OC[C@@H]3CCCO3)n3c5cccc(Br)c5c(=O)nc3n4C3CCCC3)CC2)NC(=O)OC(C)(C)C)cc1] | 0.92 | 925/927 | 7 |
| 36n | 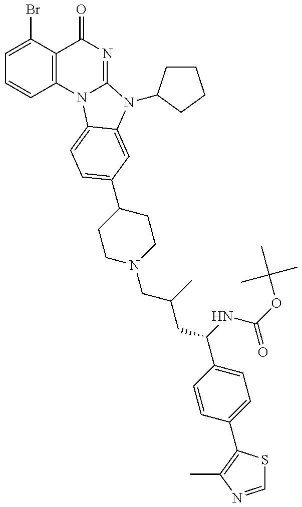 [SMILES: Cc1ncsc1-c1ccc([C@H](CC(C)CN2CCC(c3ccc4c(c3)n(C3CCCC3)c3nc(=O)c5c(Br)cccc5n43)CC2)NC(=O)OC(C)(C)C)cc1] | 1.79 | 823 | 14 |
| 36o | 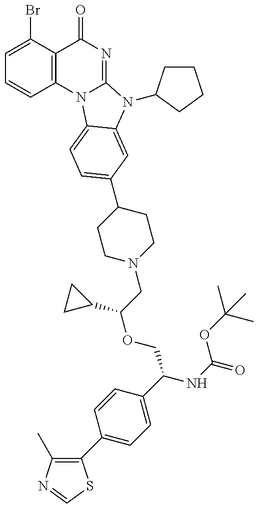 [SMILES: Cc1ncsc1-c1ccc([C@H](CO[C@@H](CN2CCC(c3ccc4c(c3)n(C3CCCC3)c3nc(=O)c5c(Br)cccc5n43)CC2)C2CC2)NC(=O)OC(C)(C)C)cc1] | 0.98 | 865/867 | 7 |
| 36p | 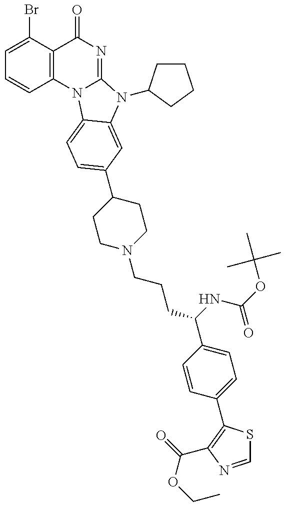 [SMILES: CCOC(=O)c1ncsc1-c1ccc([C@H](CCCN2CCC(c3ccc4c(c3)n(C3CCCC3)c3nc(=O)c5c(Br)cccc5n43)CC2)NC(=O)OC(C)(C)C)cc1] | 1.61 | 867 | 14 |
| 36q | 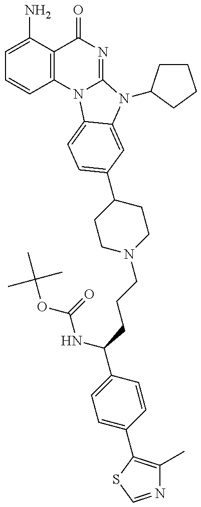 [SMILES: Cc1ncsc1-c1ccc([C@H](CCCN2CCC(c3ccc4c(c3)n(C3CCCC3)c3nc(=O)c5c(N)cccc5n43)CC2)NC(=O)OC(C)(C)C)cc1] | 1.67 | 746 | 14 |
| 36r | 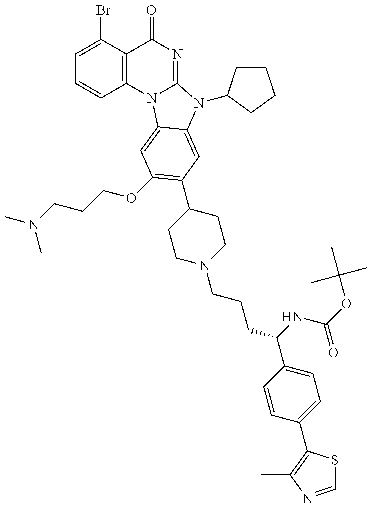 [SMILES: Cc1ncsc1-c1ccc([C@H](CCCN2CCC(c3cc4c(cc3OCCCN(C)C)n3c5cccc(Br)c5c(=O)nc3n4C3CCCC3)CC2)NC(=O)OC(C)(C)C)cc1] | 0.90 | 912 | 7 |
| 36s | 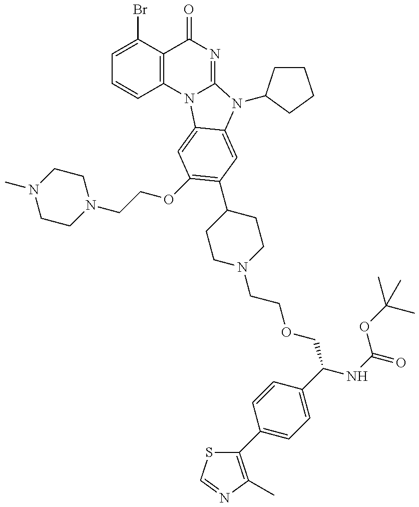 [SMILES: Cc1ncsc1-c1ccc([C@H](COCCN2CCC(c3cc4c(cc3OCCN3CCN(C)CC3)n3c5cccc(Br)c5c(=O)nc3n4C3CCCC3)CC2)NC(=O)OC(C)(C)C)cc1] | 0.81 | 484/485 [M + 2H]⁺⁺ | 7 |
| 36t | 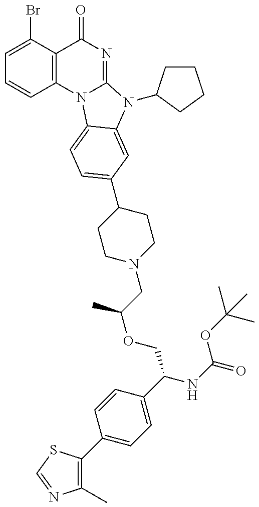 [SMILES: Cc1ncsc1-c1ccc([C@H](CO[C@@H](C)CN2CCC(c3ccc4c(c3)n(C3CCCC3)c3nc(=O)c5c(Br)cccc5n43)CC2)NC(=O)OC(C)(C)C)cc1] | 1.71 | 420 | 14 |
| 36u | 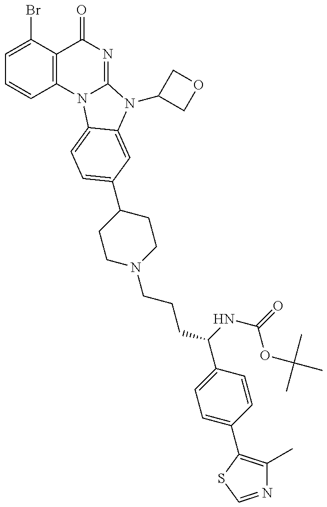 [SMILES: Cc1ncsc1-c1ccc([C@H](CCCN2CCC(c3ccc4c(c3)n(C3COC3)c3nc(=O)c5c(Br)cccc5n43)CC2)NC(=O)OC(C)(C)C)cc1] | 1.51 | 797 | 14 |
| 36v | 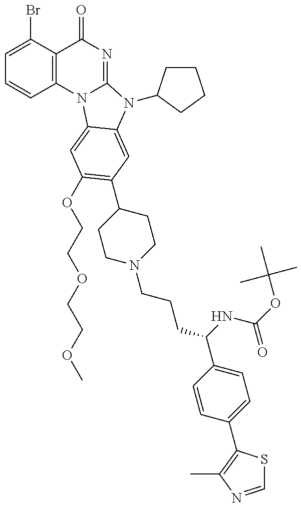 [SMILES: COCCOCCOc1cc2c(cc1C1CCN(CCC[C@H](NC(=O)OC(C)(C)C)c3ccc(-c4scnc4C)cc3)CC1)n(C1CCCC1)c1nc(=O)c3c(Br)cccc3n21] | 0.688 | 927/929 | 6 |
| 36w | 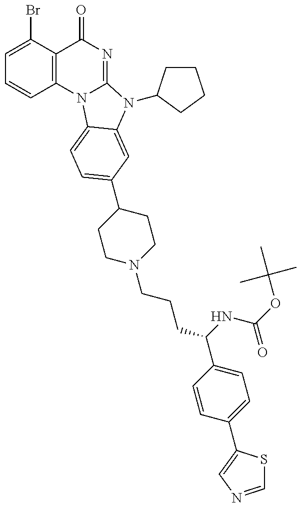 [SMILES: CC(C)(C)OC(=O)N[C@@H](CCCN1CCC(c2ccc3c(c2)n(C2CCCC2)c2nc(=O)c4c(Br)cccc4n32)CC1)c1ccc(-c2cncs2)cc1] | 0.59 | 795/797 | 6 |
| 36x | 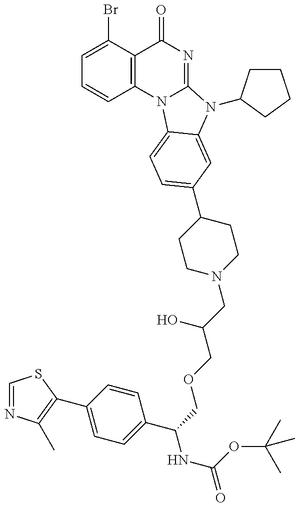 [SMILES: Cc1ncsc1-c1ccc([C@H](COCC(O)CN2CCC(c3ccc4c(c3)n(C3CCCC3)c3nc(=O)c5c(Br)cccc5n43)CC2)NC(=O)OC(C)(C)C)cc1] | 1.58 | 855 | 14 |
| 36y | 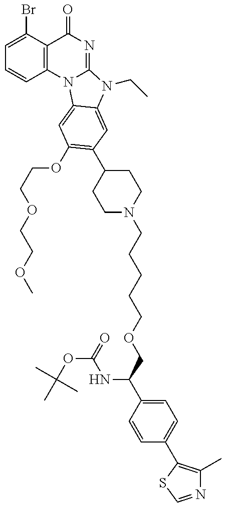 [SMILES: CCn1c2cc(C3CCN(CCCCCOC[C@H](NC(=O)OC(C)(C)C)c4ccc(-c5scnc5C)cc4)CC3)c(OCCOCCOC)cc2n2c3cccc(Br)c3c(=O)nc12] | 0.64 | 945/947 | 1 |
| 36z | 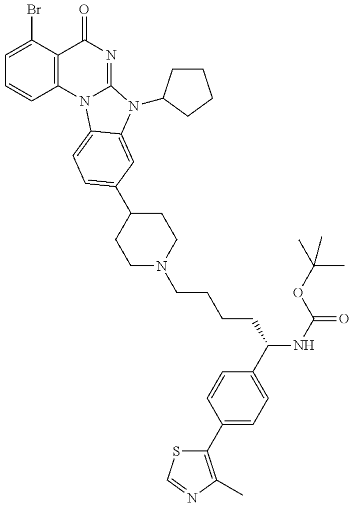 [SMILES: Cc1ncsc1-c1ccc([C@H](CCCCN2CCC(c3ccc4c(c3)n(C3CCCC3)c3nc(=O)c5c(Br)cccc5n43)CC2)NC(=O)OC(C)(C)C)cc1] | 0.92 | 823/825 | 7 |
| 36aa | 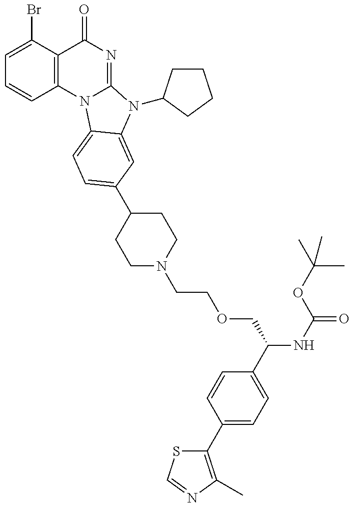 [SMILES: Cc1ncsc1-c1ccc([C@H](COCCN2CCC(c3ccc4c(c3)n(C3CCCC3)c3nc(=O)c5c(Br)cccc5n43)CC2)NC(=O)OC(C)(C)C)cc1] | 0.59 | 825/827 | 1 |
| 36ab | 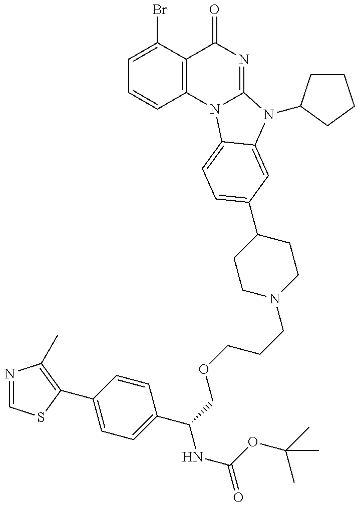 [SMILES: Cc1ncsc1-c1ccc([C@H](COCCCN2CCC(c3ccc4c(c3)n(C3CCCC3)c3nc(=O)c5c(Br)cccc5n43)CC2)NC(=O)OC(C)(C)C)cc1] | 0.88 | 839/841 | 4 |

### Experimental Procedure for the Synthesis of 36ac

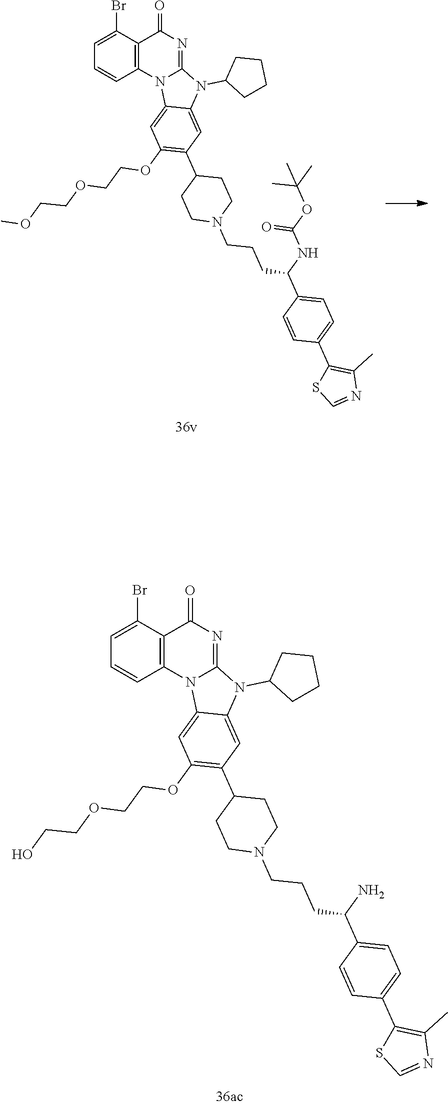

36v (0.227 g, 0.245 mmol, 1.00 equiv.) is dissolved in DCM abs. (6.7 mL) and cooled to 0° C. Then trimethylsilyliodide (1M in DCM) (2.201 mL, 2.202 mmol, 9.00 equiv.) is added and the reaction is stirred 2 days at rt. The mixture is diluted with water/methanol, filtrated and purified by reverse phase chromatography. Product fractions are combined and lyophilizated to give 36ac (0.128 g, 64.3%).

TABLE 63

| | | | | HPLC |
|---|---|---|---|---|
| # | structure | tᵣₑₜ [min] | [M + H]⁺ | method |
| 36ac | 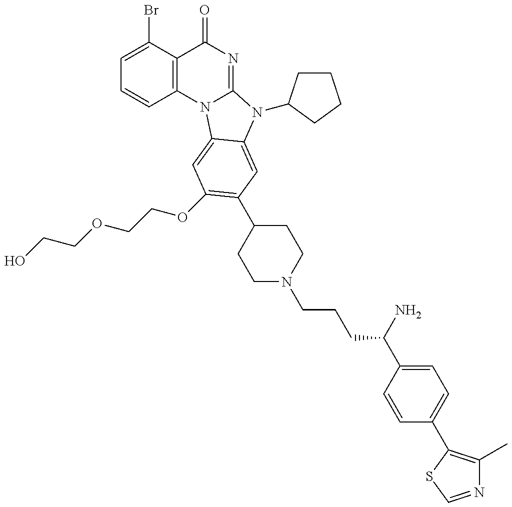 [SMILES: Cc1ncsc1-c1ccc([C@@H](N)CCCN2CCC(c3cc4c(cc3OCCOCCO)n3c5cccc(Br)c5c(=O)nc3n4C3CCCC3)CC2)cc1] | 0.601 | 813/815 | 7 |

### Experimental Procedure for the Synthesis of 36ad

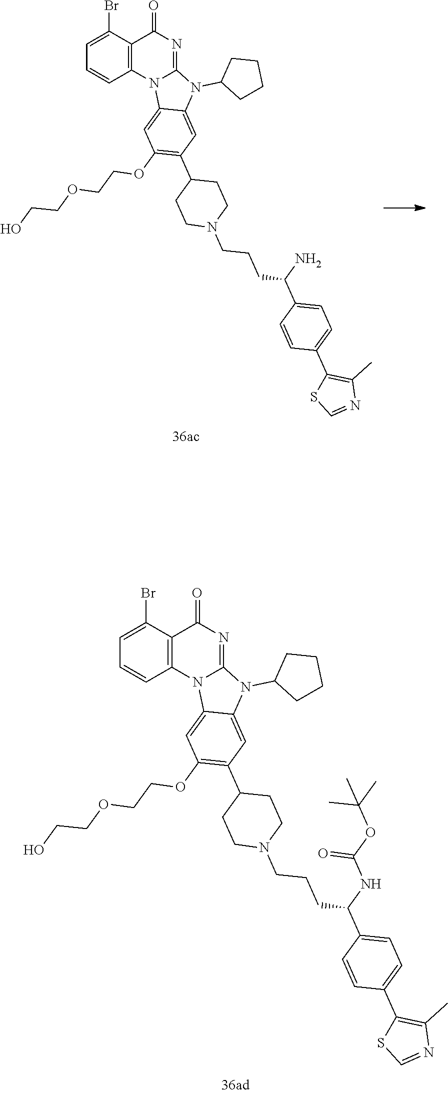 [SMILES: Cc1ncsc1-c1ccc([C@@H](N)CCCN2CCC(c3cc4c(cc3OCCOCCO)n3c5cccc(Br)c5c(=O)nc3n4C3CCCC3)CC2)cc1 ; Cc1ncsc1-c1ccc([C@H](CCCN2CCC(c3cc4c(cc3OCCOCCO)n3c5cccc(Br)c5c(=O)nc3n4C3CCCC3)CC2)NC(=O)OC(C)(C)C)cc1]

36ac (0.128 g, 0.157 mmol, 1.00 equiv.) is dissolved in DCM abs. (2 mL). Then di-tert-butyl dicarbonate (0.052 g, 0.236 mmol, 1.50 equiv.) and triethylamine (0.087 mL, 0.629 mmol, 4.00 equiv.) are added and the reaction is stirred 3h at rt. The mixture is diluted with water and saturated sodium bicarbonate solution. The organic phase is separated, dried and concentrated under reduced pressure to give crude 36ad (0.143 g, 99.5%).

TABLE 64

| | | | | HPLC |
|---|---|---|---|---|
| # | structure | tᵣₑₜ [min] | [M + H]⁺ | method |
| 36ad | 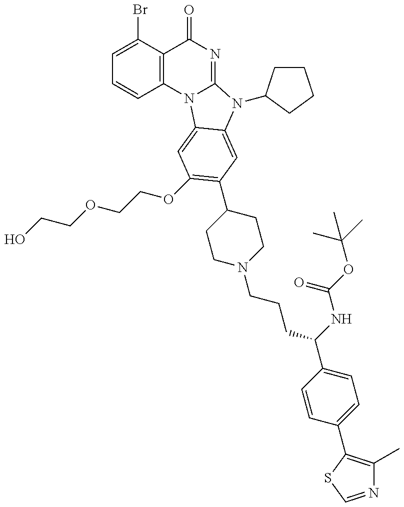 [SMILES: Cc1ncsc1-c1ccc([C@H](CCCN2CCC(c3cc4c(cc3OCCOCCO)n3c5cccc(Br)c5c(=O)nc3n4C3CCCC3)CC2)NC(=O)OC(C)(C)C)cc1] | 0.771 | 913/915 | 7 |

### Experimental Procedure for the Synthesis of 36ae

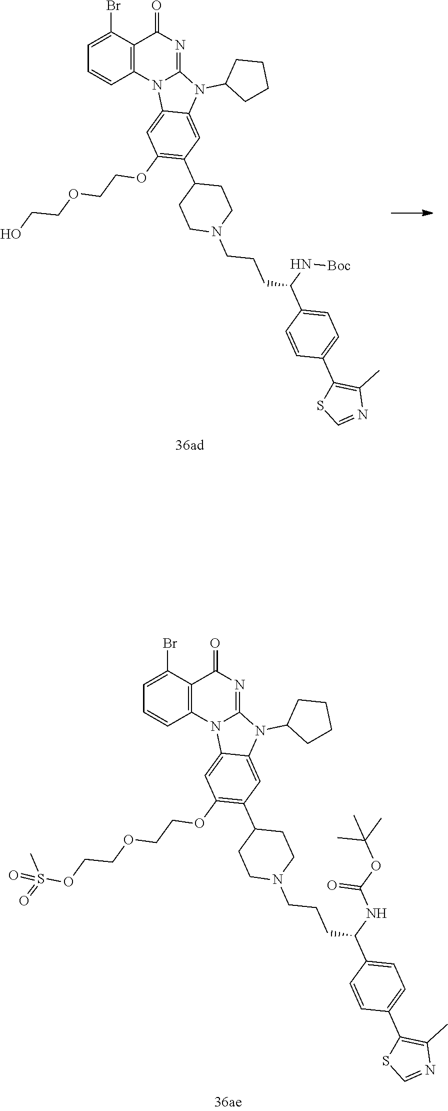 [SMILES: Cc1ncsc1-c1ccc([C@H](CCCN2CCC(c3cc4c(cc3OCCOCCO)n3c5cccc(Br)c5c(=O)nc3n4C3CCCC3)CC2)NC(=O)OC(C)(C)C)cc1 ; Cc1ncsc1-c1ccc([C@H](CCCN2CCC(c3cc4c(cc3OCCOCCOS(C)(=O)=O)n3c5cccc(Br)c5c(=O)nc3n4C3CCCC3)CC2)NC(=O)OC(C)(C)C)cc1]

36ad (0.116 g, 0.127 mmol, 1.00 equiv.) is dissolved together with triethylamine (0.044 mL, 0.317 mmol, 2.50 equiv.) in DCM abs. (1 mL). Then methanesulfonyl chloride (0.020 mL, 0.254 mmol, 2.00 equiv.) is added dropwise at rt. The mixture is stirred for 1 h. The reaction mixture is diluted with water and saturated sodium bicarbonate solution and stirred for 5 min. The organic phase is separated, dried and concentrated under reduced pressure to give crude 36ae (0.131 g, 100%).

TABLE 65

| | | | | HPLC |
|---|---|---|---|---|
| # | structure | tᵣₑₜ [min] | [M + H]⁺ | method |
| 36ae | 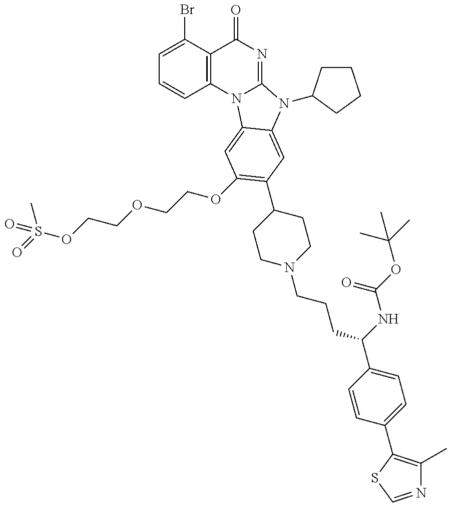 [SMILES: Cc1ncsc1-c1ccc([C@H](CCCN2CCC(c3cc4c(cc3OCCOCCOS(C)(=O)=O)n3c5cccc(Br)c5c(=O)nc3n4C3CCCC3)CC2)NC(=O)OC(C)(C)C)cc1] | 0.650 | 992/994 | 6 |

### Experimental Procedure for the Synthesis of 36af

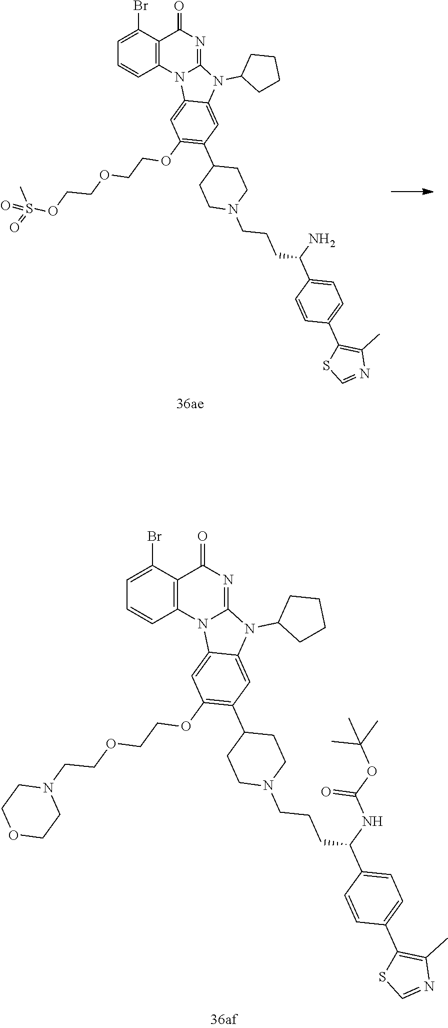 [SMILES: Cc1ncsc1-c1ccc([C@@H](N)CCCN2CCC(c3cc4c(cc3OCCOCCOS(C)(=O)=O)n3c5cccc(Br)c5c(=O)nc3n4C3CCCC3)CC2)cc1 ; Cc1ncsc1-c1ccc([C@H](CCCN2CCC(c3cc4c(cc3OCCOCCN3CCOCC3)n3c5cccc(Br)c5c(=O)nc3n4C3CCCC3)CC2)NC(=O)OC(C)(C)C)cc1]

36ae (0.043 g, 0.043 mmol, 1.00 equiv.) is dissolved together with morpholine (0.005 mL, 0.052 mmol, 1.20 equiv.) in NMP abs. (1 mL). Then DIPEA (0.037 mL, 0.217 mmol, 5.00 equiv.) is added and the mixture is stirred over night at 75° C. The reaction mixture is diluted with water and acetonitrile, filtrated and purified by reverse phase chromatography to give 36af (0.018 g, 42.2%).

TABLE 66

| | | | | HPLC |
|---|---|---|---|---|
| # | structure | tᵣₑₜ [min] | [M + H]⁺ | method |
| 36af | 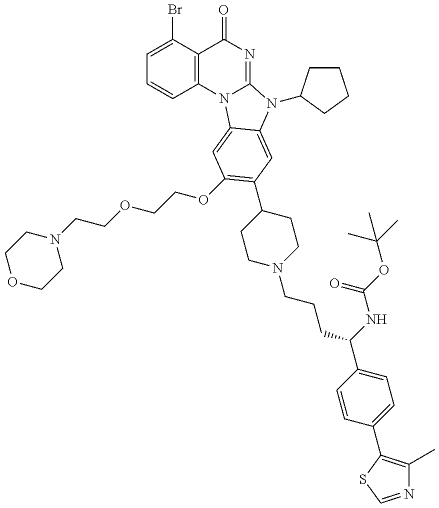 [SMILES: Cc1ncsc1-c1ccc([C@H](CCCN2CCC(c3cc4c(cc3OCCOCCN3CCOCC3)n3c5cccc(Br)c5c(=O)nc3n4C3CCCC3)CC2)NC(=O)OC(C)(C)C)cc1] | 0.853 | 993/995 | 13 |

### Experimental Procedure for the Synthesis of 36aq

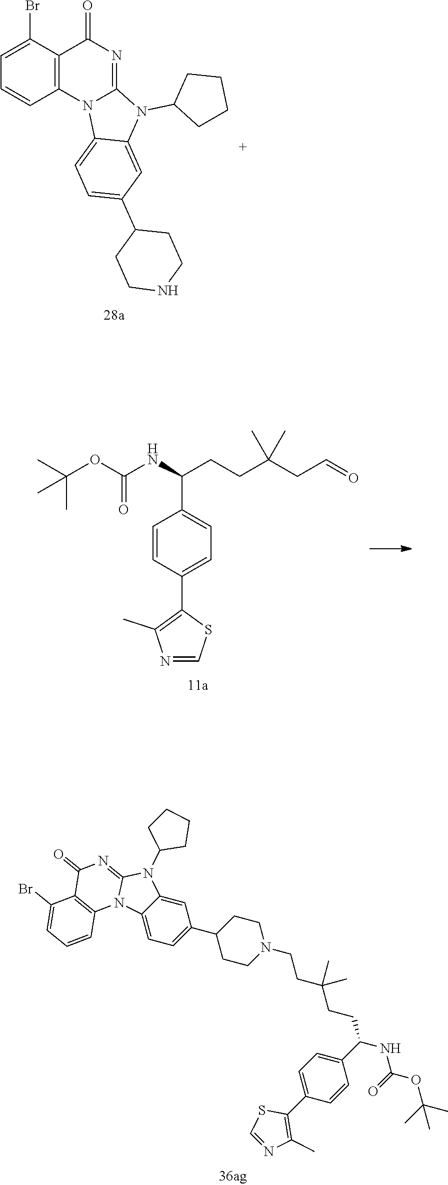 [SMILES: O=c1nc2n(C3CCCC3)c3cc(C4CCNCC4)ccc3n2c2cccc(Br)c12 ; Cc1ncsc1-c1ccc([C@H](CCC(C)(C)CC=O)NC(=O)OC(C)(C)C)cc1 ; Cc1ncsc1-c1ccc([C@H](CCC(C)(C)CCN2CCC(c3ccc4c(c3)n(C3CCCC3)c3nc(=O)c5c(Br)cccc5n43)CC2)NC(=O)OC(C)(C)C)cc1]

Intermediate 28a (28.5 mg; 0.061 mmol; 150 mol %) is suspended dichloroethane (600 μL) and carbamate 11a (17.0 mg; 0.041 mmol; 100 mol %) is added. The mixture is heated to 50° C., sodium triacetoxy borohydride (17.3 mg; 0.082 mmol; 200 mol %) is added at this temperature and the mixture is stirred at 50° C. for 30 min. The reaction is cooled down to rt and quenched with water. The solvent is removed under reduce pressure. The residue is dissolved in water/methanol and basified before purification by reverse phase chromatography giving the desired product 36ah (33 mg, 93% yield).

TABLE 67

| | | tᵣₑₜ | [M + | HPLC |
|---|---|---|---|---|
| # | structure | [min] | H]⁺ | method |
| 36ag | 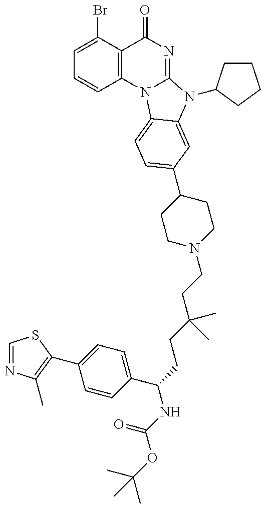 [SMILES: Cc1ncsc1-c1ccc([C@H](CCC(C)(C)CCN2CCC(c3ccc4c(c3)n(C3CCCC3)c3nc(=O)c5c(Br)cccc5n43)CC2)NC(=O)OC(C)(C)C)cc1] | 1.03 | 865/ 867 | 5 |

### Experimental Procedure for the Synthesis of 36ah

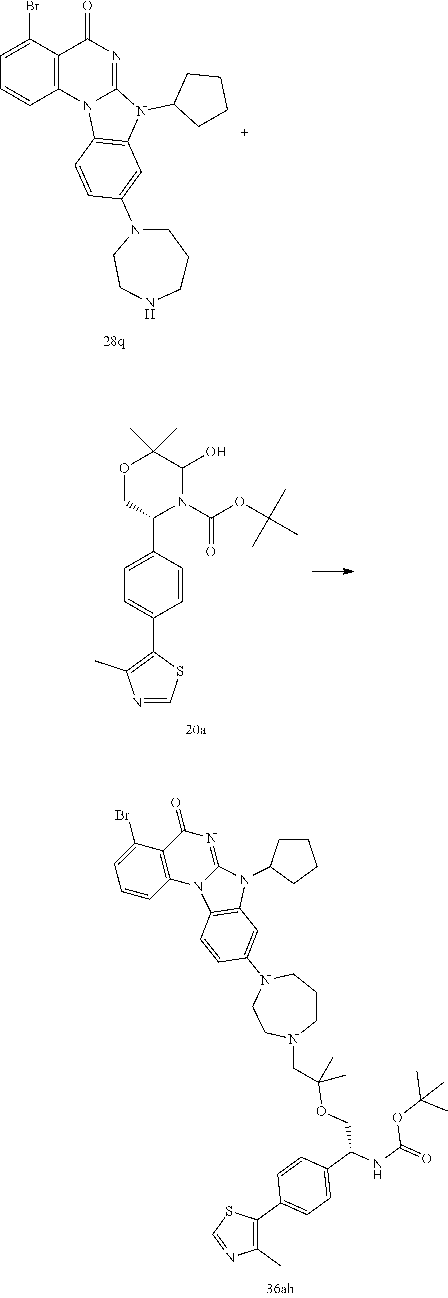

A suspension of intermediate 28q (30.0 mg; 0.062 mmol; 1.00 eq.) in dichloroethane (1.00 mL) and sodium triacetoxy borohydride (70.0 mg; 0.314 mmol; 5.02 eq.) is heated to 60° C. Then a solution of carbamate 20a (29.0 mg; 0.072 mmol; 1.15 eq.) in dichloroethane (1.00 mL) is added. The reaction mixture is stirred at 60° C. for 2.5 hours. The reaction mixture is diluted with DCM (3 mL) and water (7 mL). The layers are separated and the aqueous layer is washed with DCM (2×4 mL). The combined organic layers are dried and concentrated under reduced pressure. The residue is purified by column chromatography giving the desired product 36ah (24 mg, 44% yield).

The following intermediates 36 (table 68) are available in an analogous manner using different starting materials 20. The crude product 36 is purified by chromatography if necessary.

TABLE 68

| | | | | HPLC |
|---|---|---|---|---|
| # | structure | tᵣₑₜ [min] | [M + H]⁺ | method |
| 36ah | 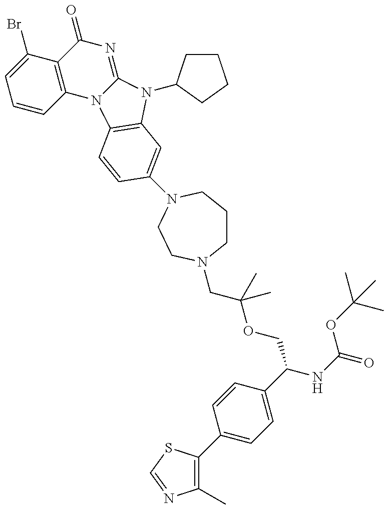 [SMILES: Cc1ncsc1-c1ccc([C@H](COC(C)(C)CN2CCCN(c3ccc4c(c3)n(C3CCCC3)c3nc(=O)c5c(Br)cccc5n43)CC2)NC(=O)OC(C)(C)C)cc1] | 1.74 | 868 | 14 |
| 36ai | 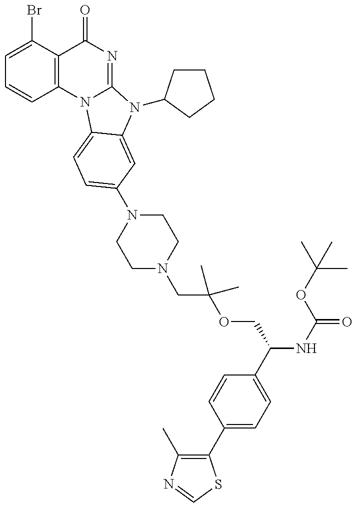 [SMILES: Cc1ncsc1-c1ccc([C@H](COC(C)(C)CN2CCN(c3ccc4c(c3)n(C3CCCC3)c3nc(=O)c5c(Br)cccc5n43)CC2)NC(=O)OC(C)(C)C)cc1] | 1.73 | 854 | 14 |
| 36aj | 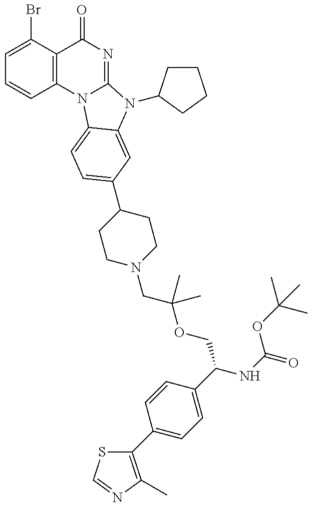 [SMILES: Cc1ncsc1-c1ccc([C@H](COC(C)(C)CN2CCC(c3ccc4c(c3)n(C3CCCC3)c3nc(=O)c5c(Br)cccc5n43)CC2)NC(=O)OC(C)(C)C)cc1] | 1.79 | 853 | 14 |

### Method D:

### Experimental Procedure for the Synthesis of Compound 42a

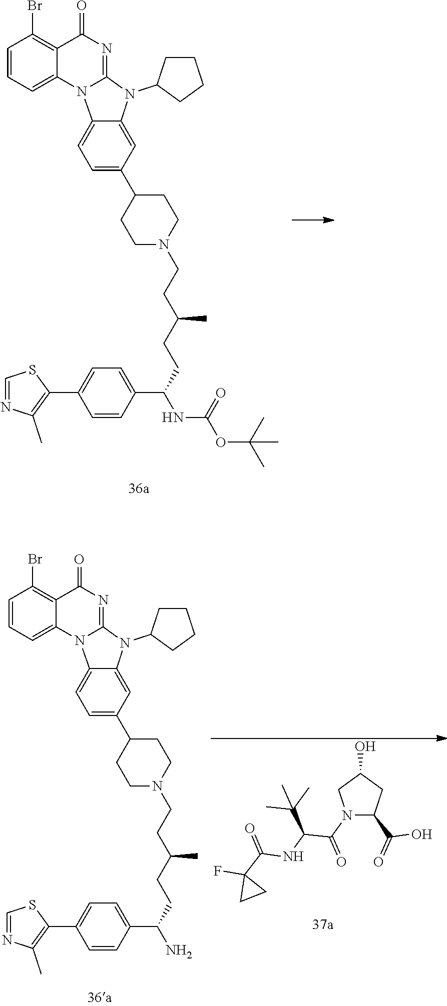

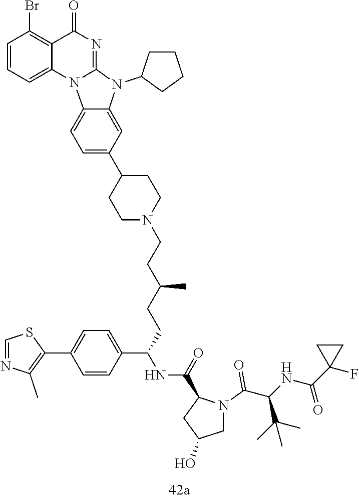 [SMILES: Cc1ncsc1-c1ccc([C@H](CC[C@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1]

In a 100 mL round-bottom flask Carbamate 36a (615 mg; 0.722 mmol; 1.00 eq.) is dissolved in methanol (5 mL) and 4N HCl in dioxane (5 mL; 20.0 mmol; 27.7 eq.) is added. The mixture is stirred at rt for 30 minutes. The solvents are removed under reduced pressure and intermediate 36′a is taken to the next step without further purification (100% yield assumed). In a 100 mL round-bottom flask carboxylic acid 37a (300 mg; 0.865 mmol; 1.20 eq.), amine 36′a (542 mg; 0.721 mmol; 1.00 eq.) and HATU (420 mg; 1.08 mmol; 1.50 eq.) are taken up in DMF (5 mL) and N,N-diisopropylethylamine (0.992 mL; 5.77 mmol; 8.00 eq.) is added. The mixture is stirred at rt for 20 minutes. The reaction mixture is diluted with H₂O and extracted with DCM twice. The combined organic layers are washed with water and sat. NH₄Cl-solution, passed through a phase separator cartridge and concentrated under reduced pressure. The residue is diluted with ACN/H₂O, filtered through a syringe filter and purified by prep. HPLC. The product 42a is isolated via column chromatography (71% yield, 540 mg).

The following intermediates 42 (table 69) are available in an analogous manner using different starting materials 36 and 37. The crude product 42 is purified by chromatography if necessary. Analytical methods show usually [M+2H]⁺⁺ or [M+2H]⁺⁺/2.

TABLE 69

| | | | | | DC50 | DC50 |
|---|---|---|---|---|---|---|
| | | tᵣₑₜ | [M + 2H]⁺⁺ | HPLC | A2 | A4 |
| # | structure | [min] | [M + H]⁺ | method | [nM] | [nM] |
| 42a | 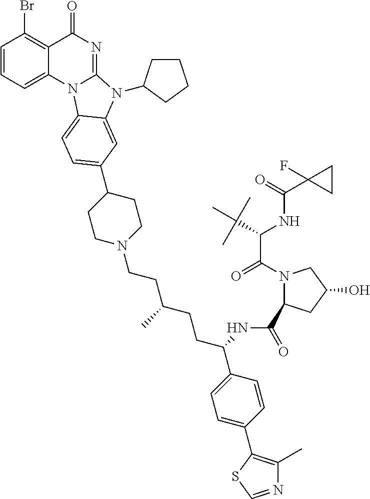 [SMILES: Cc1ncsc1-c1ccc([C@H](CC[C@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.58 | 1063 | 14 | 0.9 | 25 |
| 42b | 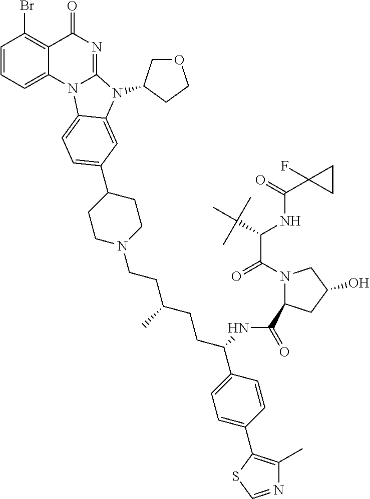 [SMILES: Cc1ncsc1-c1ccc([C@H](CC[C@H](C)CCN2CCC(c3ccc4c(c3)n([C@H]3CCOC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.49 | 533 | 14 | 2 | >833 |
| 42c | 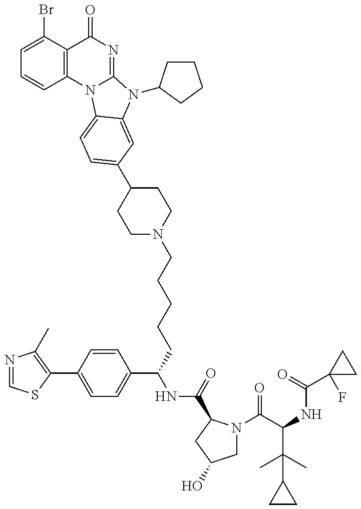 [SMILES: Cc1ncsc1-c1ccc([C@H](CCCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C2CC2)cc1] | 1.60 | 538 | 14 | 0.7 | 11 |
| 42d | 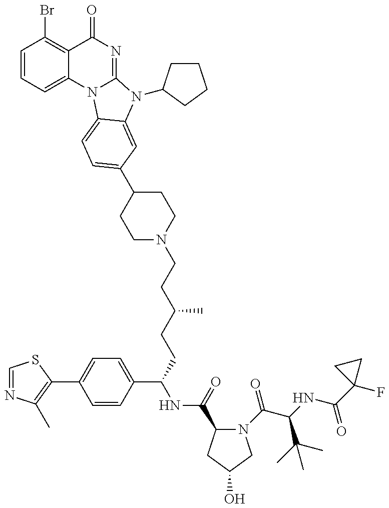 [SMILES: Cc1ncsc1-c1ccc([C@H](CC[C@@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.59 | 1063 | 14 | 0.7 | 7 |
| 42e | 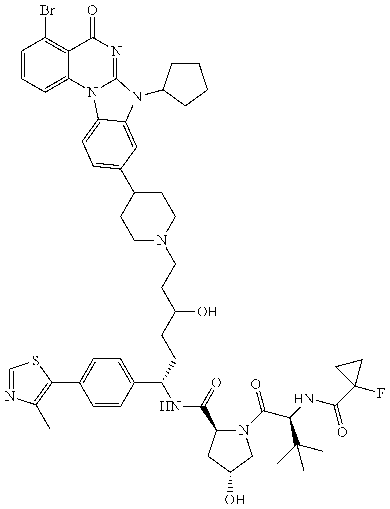 [SMILES: Cc1ncsc1-c1ccc([C@H](CCC(O)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 0.77 | 533 | 14 | 4.1 | 80 |
| 42f | 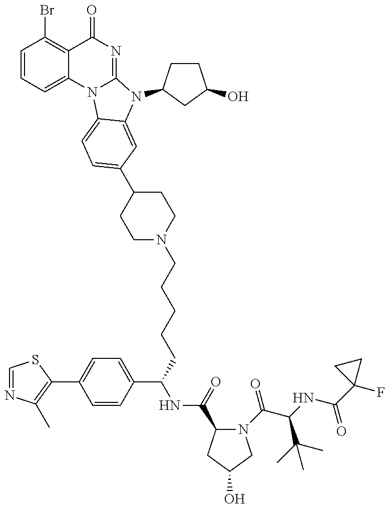 [SMILES: Cc1ncsc1-c1ccc([C@H](CCCCCN2CCC(c3ccc4c(c3)n([C@H]3CC[C@@H](O)C3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.42 | 533 | 14 | 2 | 46 |
| 42g | 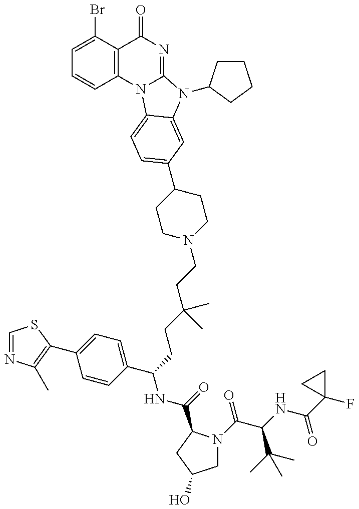 [SMILES: Cc1ncsc1-c1ccc([C@H](CCC(C)(C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.62 | 539 | 14 | 3 | 40 |
| 42h | 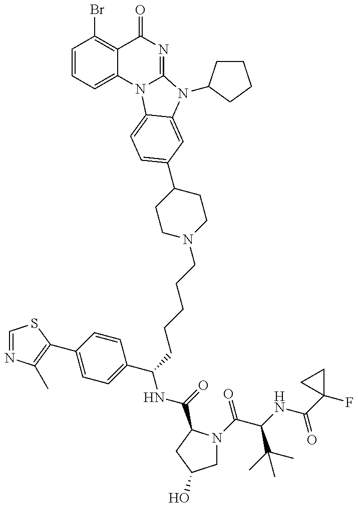 [SMILES: Cc1ncsc1-c1ccc([C@H](CCCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.56 | 525 | 14 | 0.3 | 4 |
| 42i | 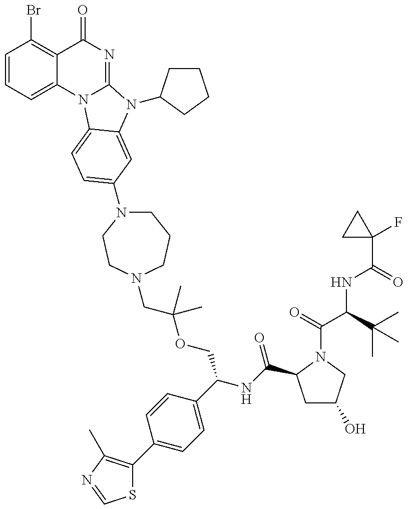 [SMILES: Cc1ncsc1-c1ccc([C@H](COC(C)(C)CN2CCCN(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.58 | 540 | 14 | 9 | 24 |
| 42j | 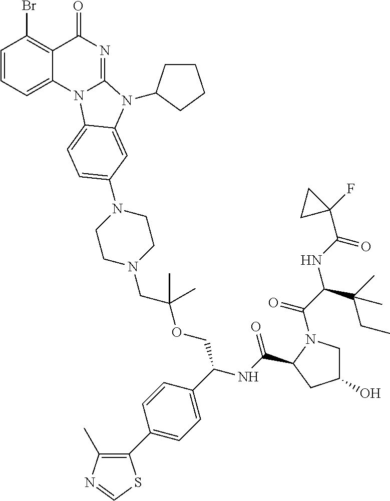 [SMILES: CCC(C)(C)[C@H](NC(=O)C1(F)CC1)C(=O)N1C[C@H](O)C[C@H]1C(=O)N[C@@H](COC(C)(C)CN1CCN(c2ccc3c(c2)n(C2CCCC2)c2nc(=O)c4c(Br)cccc4n32)CC1)c1ccc(-c2scnc2C)cc1] | 1.65 | 540 | 14 | 4 | >833 |
| 42k | 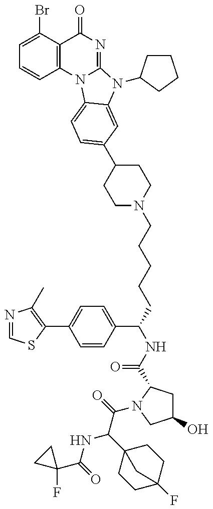 [SMILES: Cc1ncsc1-c1ccc([C@H](CCCCCN2CCC(c3ccc4c(c3)n(C3CCCC3)c3nc(=O)c5c(Br)cccc5n43)CC2)NC(=O)[C@@H]2C[C@@H](O)CN2C(=O)C(NC(=O)C2(F)CC2)C23CCC(F)(CC2)C3)cc1] | 1.53 | 560 | 14 | 4 | 41 |
| 42l | 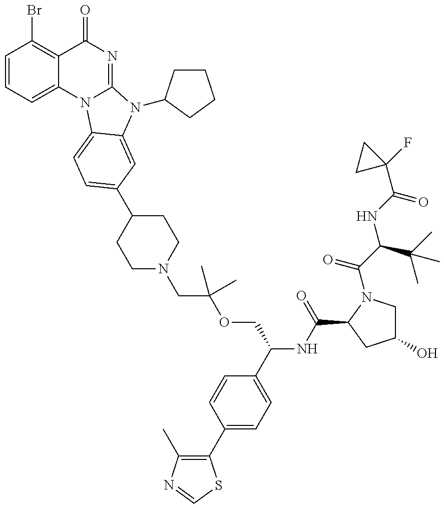 [SMILES: Cc1ncsc1-c1ccc([C@H](COC(C)(C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.62 | 533 | 14 | 3 | >833 |
| 42m | 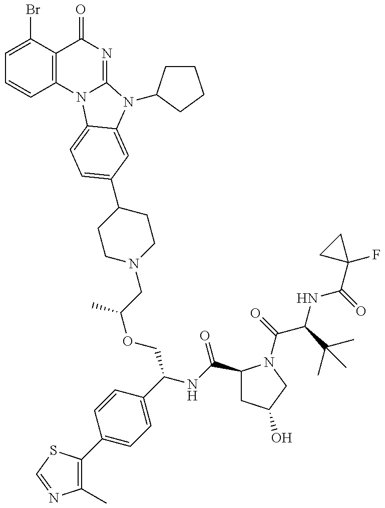 [SMILES: Cc1ncsc1-c1ccc([C@H](CO[C@H](C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.54 | 526 | 14 | 4 | >833 |
| 42n | 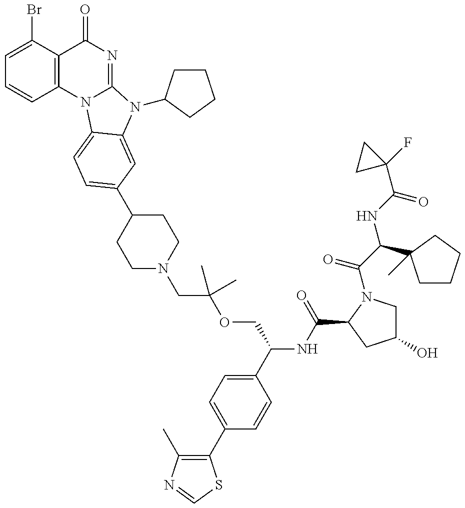 [SMILES: Cc1ncsc1-c1ccc([C@H](COC(C)(C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C2(C)CCCC2)cc1] | 1.67 | 546 | 14 | 3 | 800 |
| 42o | 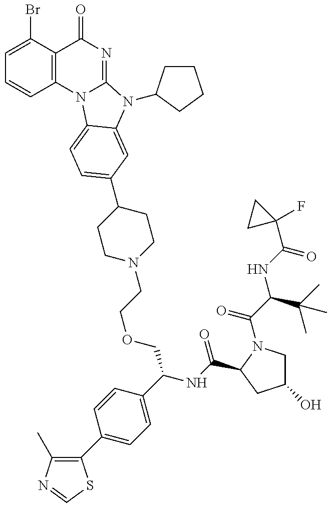 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.46 | 519 | 14 | 5 | 90 |
| 42p | 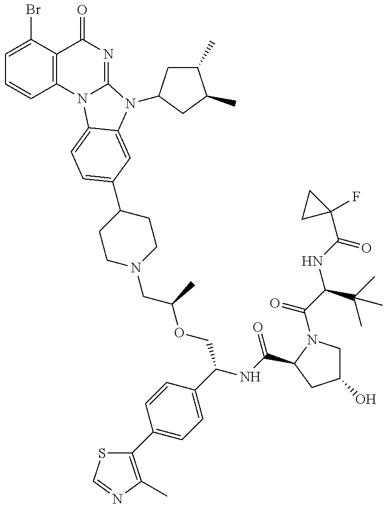 [SMILES: Cc1ncsc1-c1ccc([C@H](CO[C@H](C)CN2CCC(c3ccc4c(c3)n(C3C[C@H](C)[C@@H](C)C3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.61 | 540 | 14 | 13 | >833 |
| 42q | 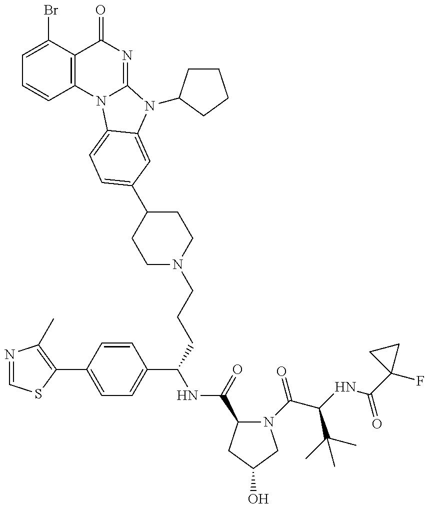 [SMILES: Cc1ncsc1-c1ccc([C@H](CCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.54 | 511 | 14 | 0.4 | 1 |
| 42r | 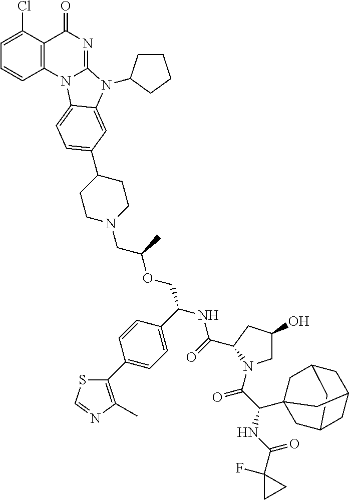 [SMILES: Cc1ncsc1-c1ccc([C@H](CO[C@H](C)CN2CCC(c3ccc4c(c3)n(C3CCCC3)c3nc(=O)c5c(Cl)cccc5n43)CC2)NC(=O)[C@@H]2C[C@@H](O)CN2C(=O)[C@@H](NC(=O)C2(F)CC2)C23CC4CC(CC(C4)C2)C3)cc1] | 1.66 | 543 | 14 | 2 | 27 |
| 42s | 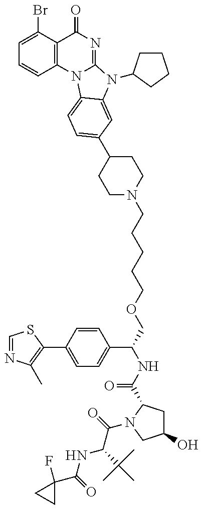 [SMILES: Cc1ncsc1-c1ccc([C@H](COCCCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.55 | 540 | 14 | 0.7 | 8 |
| 42t | 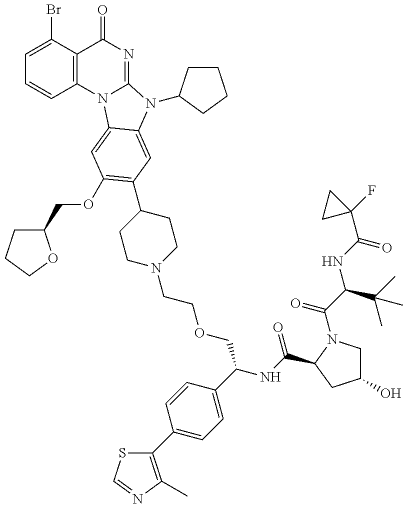 [SMILES: Cc1ncsc1-c1ccc([C@H](COCCN2CCC(c3cc4c(cc3OC[C@@H]3CCCO3)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] | 1.57 | 569 | 14 | 3 | 67 |
| 42u | 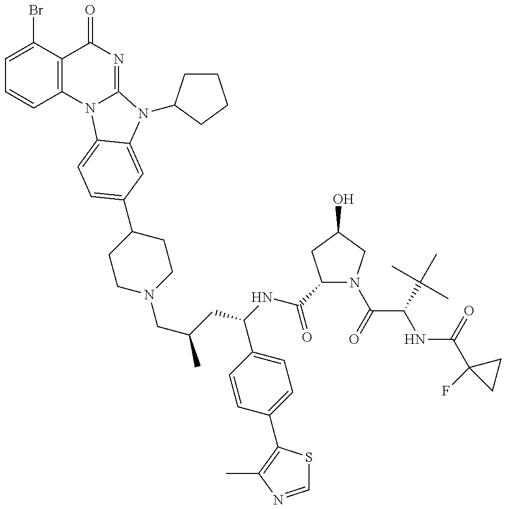 [SMILES: Cc1ncsc1-c1ccc([C@H](C[C@@H](C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.63 | 518 | 14 | 0.3 | 0.7 |
| 42v | 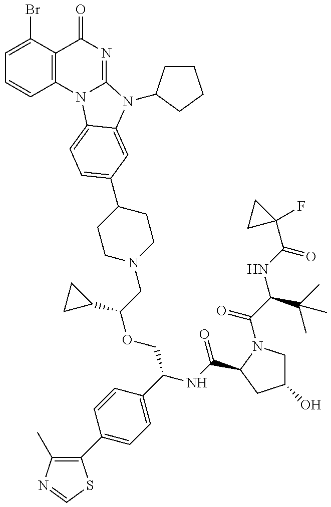 [SMILES: Cc1ncsc1-c1ccc([C@H](CO[C@@H](CN2CCC(c3ccc4c(c3)n(C3CCCC3)c3nc(=O)c5c(Br)cccc5n43)CC2)C2CC2)NC(=O)[C@@H]2C[C@@H](O)CN2C(=O)[C@@H](NC(=O)C2(F)CC2)C(C)(C)C)cc1] | 1.59 | 539 | 14 | 11 | >833 |
| 42w | 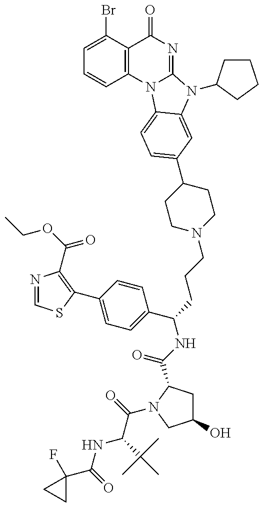 [SMILES: CCOC(=O)c1ncsc1-c1ccc([C@H](CCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.46 | 540 | 14 | 11 | 36 |
| 42x | 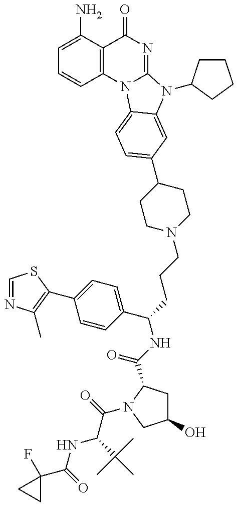 [SMILES: Cc1ncsc1-c1ccc([C@H](CCCN2CCC(c3ccc4c(c3)n(C3CCCC3)c3nc(=O)c5c(N)cccc5n43)CC2)NC(=O)[C@@H]2C[C@@H](O)CN2C(=O)[C@@H](NC(=O)C2(F)CC2)C(C)(C)C)cc1] | 1.43 | 480 | 14 | 0.3 | 2 |
| 42y | 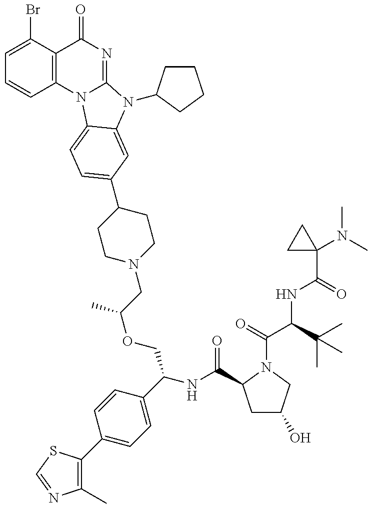 [SMILES: Cc1ncsc1-c1ccc([C@H](CO[C@H](C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(N(C)C)CC2)C(C)(C)C)cc1] | 1.55 | 538 | 14 | 6 | 101 |
| 42z | 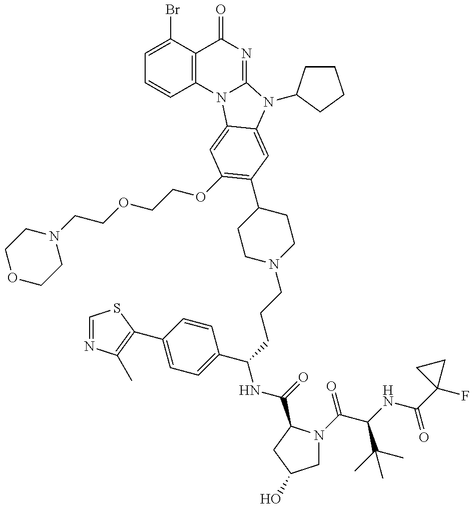 [SMILES: Cc1ncsc1-c1ccc([C@H](CCCN2CCC(c3cc4c(cc3OCCOCCN3CCOCC3)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] | 1.42 | 598 | 14 | 20 | 41 |
| 42aa | 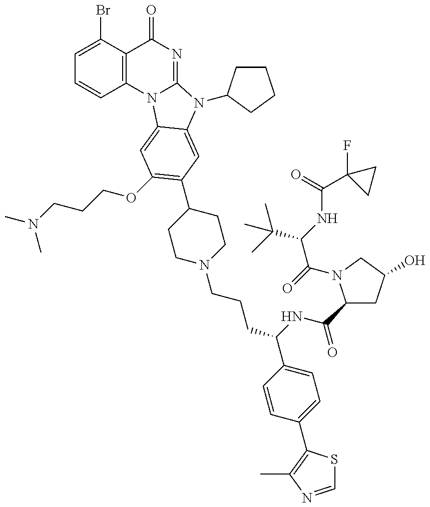 [SMILES: Cc1ncsc1-c1ccc([C@H](CCCN2CCC(c3cc4c(cc3OCCCN(C)C)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] | 1.51 | 562 | 14 | 10 | 20 |
| 42ab | 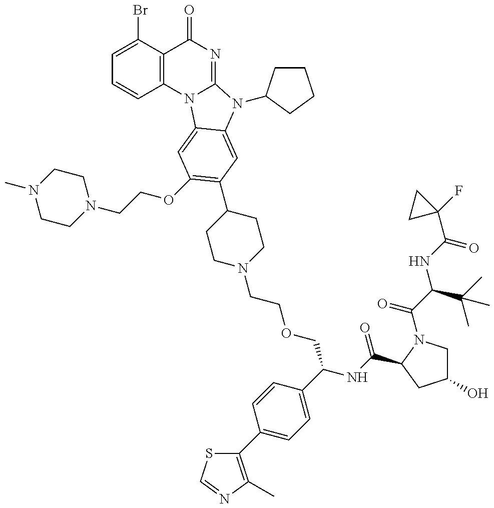 [SMILES: Cc1ncsc1-c1ccc([C@H](COCCN2CCC(c3cc4c(cc3OCCN3CCN(C)CC3)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] | 1.46 | 590 | 14 | 21 | >200 |
| 42ac | 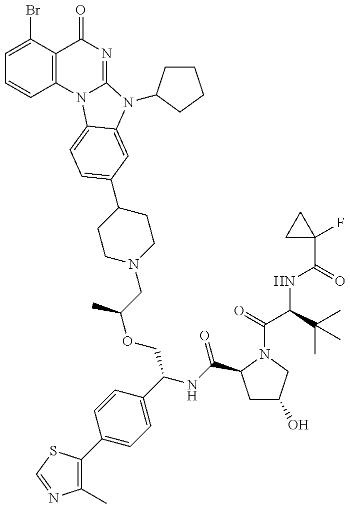 [SMILES: Cc1ncsc1-c1ccc([C@H](CO[C@@H](C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.53 | 526 | 14 | 20 | >200 |
| 42ad | 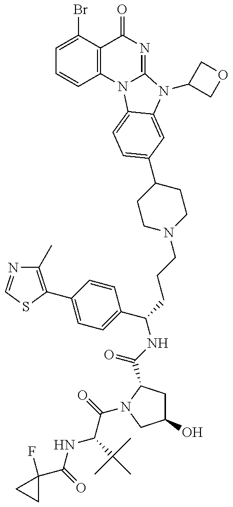 [SMILES: Cc1ncsc1-c1ccc([C@H](CCCN2CCC(c3ccc4c(c3)n(C3COC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.38 | 505 | 14 | 25 | >200 |
| 42ae | 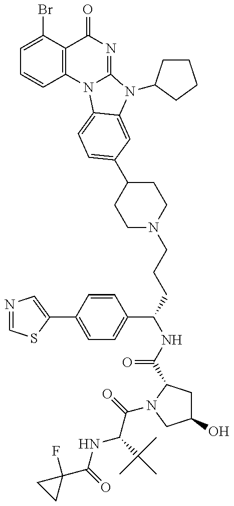 [SMILES: CC(C)(C)[C@H](NC(=O)C1(F)CC1)C(=O)N1C[C@H](O)C[C@H]1C(=O)N[C@@H](CCCN1CCC(c2ccc3c(c2)n(C2CCCC2)c2nc(=O)c4c(Br)cccc4n32)CC1)c1ccc(-c2cncs2)cc1] | 1.50 | 504 | 14 | 1 | 5 |
| 42af | 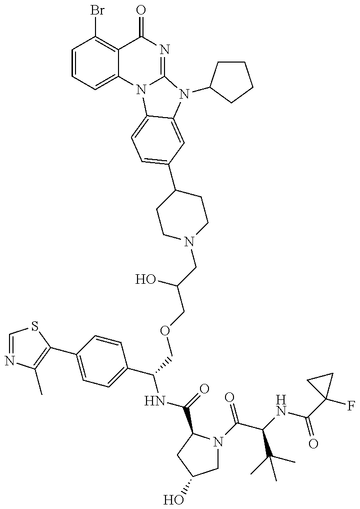 [SMILES: Cc1ncsc1-c1ccc([C@H](COCC(O)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.46 | 534 | 14 | 6 | 25 |
| 42ag | 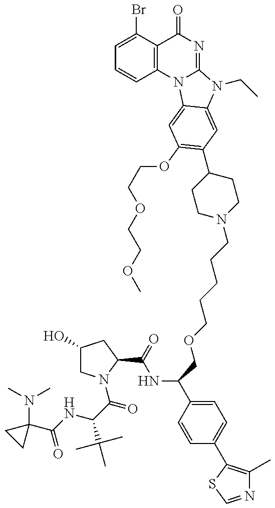 [SMILES: CCn1c2cc(C3CCN(CCCCCOC[C@H](NC(=O)[C@@H]4C[C@@H](O)CN4C(=O)[C@@H](NC(=O)C4(N(C)C)CC4)C(C)(C)C)c4ccc(-c5scnc5C)cc4)CC3)c(OCCOCCOC)cc2n2c3cccc(Br)c3c(=O)nc12] | 1.55 | 592 | 14 | 9 | >200 |
| 42ah | 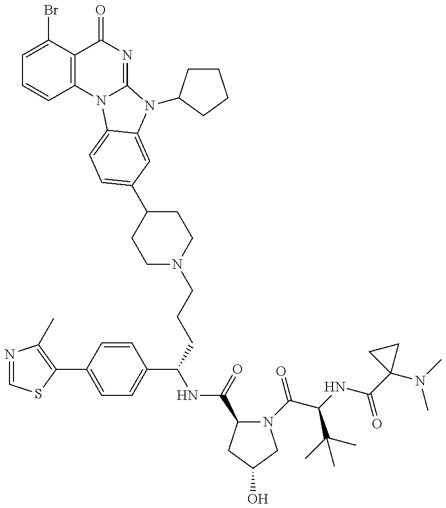 [SMILES: Cc1ncsc1-c1ccc([C@H](CCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(N(C)C)CC2)C(C)(C)C)cc1] | 1.57 | 524 | 14 | 2 | 17 |
| 42ai | 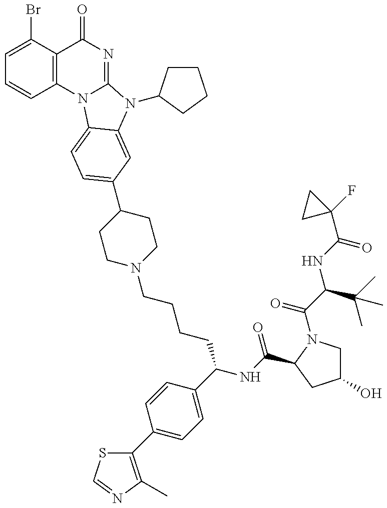 [SMILES: Cc1ncsc1-c1ccc([C@H](CCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.56 | 518 | 14 | 2 | 9 |
| 42aj | 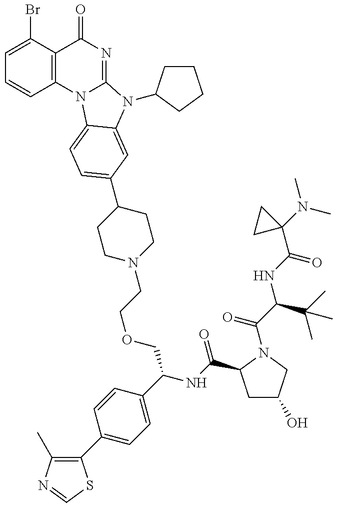 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(N(C)C)CC2)C(C)(C)C)cc1] | 1.53 | 531 | 14 | 4 | 62 |
| 42ak | 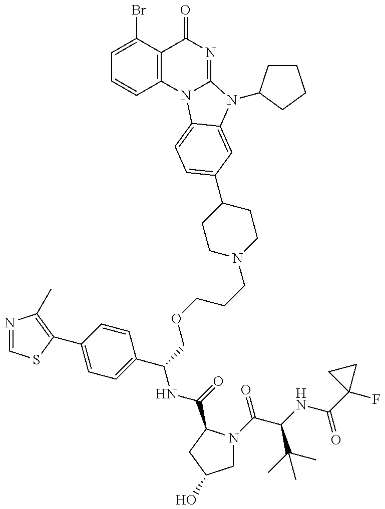 [SMILES: Cc1ncsc1-c1ccc([C@H](COCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.51 | 526 | 14 | 2 | 11 |
| 42al | 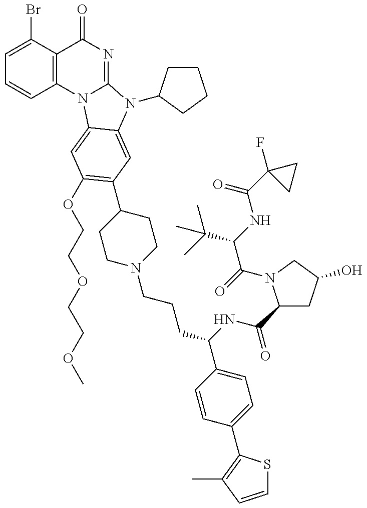 [SMILES: COCCOCCOc1cc2c(cc1C1CCN(CCC[C@H](NC(=O)[C@@H]3C[C@@H](O)CN3C(=O)[C@@H](NC(=O)C3(F)CC3)C(C)(C)C)c3ccc(-c4sccc4C)cc3)CC1)n(C1CCCC1)c1nc(=O)c3c(Br)cccc3n21] | 1.51 | 570 | 14 | 4 | 24 |

### Experimental Procedure for the Synthesis of 42am

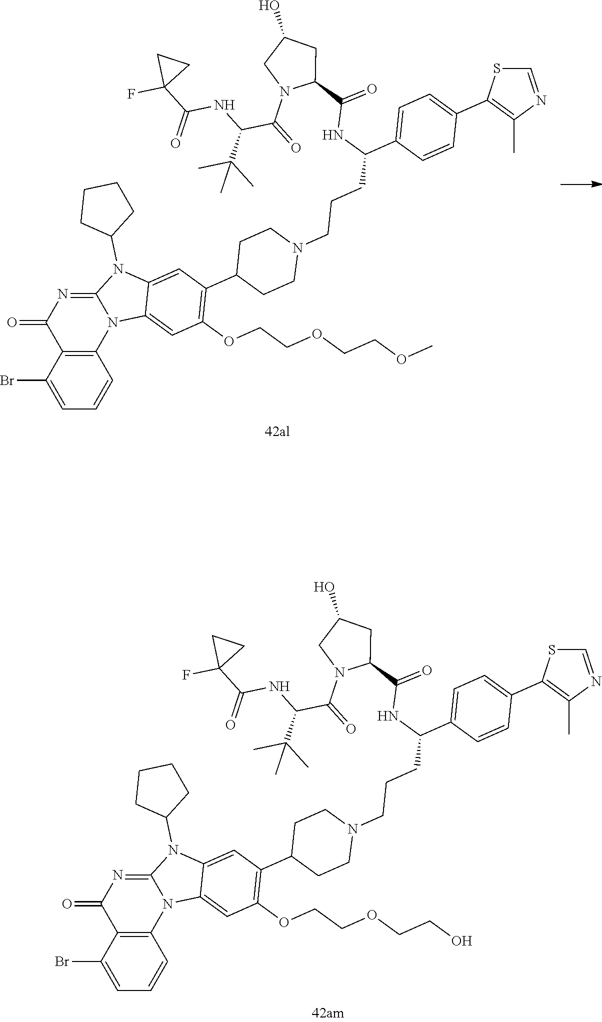 [SMILES: COCCOCCOc1cc2c(cc1C1CCN(CCC[C@H](NC(=O)[C@@H]3C[C@@H](O)CN3C(=O)[C@@H](NC(=O)C3(F)CC3)C(C)(C)C)c3ccc(-c4scnc4C)cc3)CC1)n(C1CCCC1)c1nc(=O)c3c(Br)cccc3n21 ; Cc1ncsc1-c1ccc([C@H](CCCN2CCC(c3cc4c(cc3OCCOCCO)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1]

Compound 42al is (30.0 mg; 26.3 μmol; 100 mol %) is dissolved in dichloro methane, extra dry (1.00 mL). Iodo trimethyl silane (0.05 mL; 52.0 μmol; 198 mol %) is added and the reaction mixture stirred for 24 h at rt. Additional iodo trimethyl silane (0.10 mL; 104 μmol; 395 mol %) is added and the reaction mixture stirred for 24 h at rt. The solvent is removed under reduced pressure and the residue purified by column chromatography giving the desired product 42am (20 mg, 68%).

TABLE 70

| | | | | | DC50 | DC50 |
|---|---|---|---|---|---|---|
| | | tᵣₑₜ | [M + 2H] ⁺⁺ | HPLC | A2 | A4 |
| # | structure | [min] | [M/2 + 2H]⁺⁺ | method | [nM] | [nM] |
| 42am | 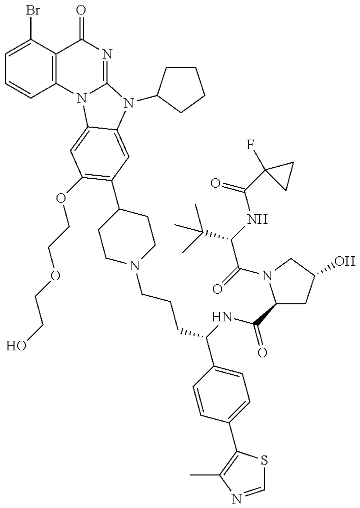 [SMILES: Cc1ncsc1-c1ccc([C@H](CCCN2CCC(c3cc4c(cc3OCCOCCO)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] | 1.39 | 563 | 14 | 0.9 | 25 |

### Method E:

### Experimental Procedure for the Synthesis of 10′a

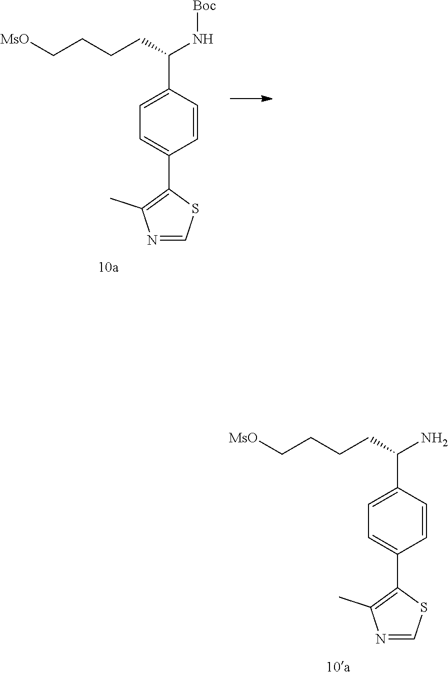 [SMILES: Cc1ncsc1-c1ccc([C@H](CCCCOS(C)(=O)=O)NC(=O)OC(C)(C)C)cc1 ; Cc1ncsc1-c1ccc([C@@H](N)CCCCOS(C)(=O)=O)cc1]

Alcohol 10a (1.61 g, 344 mmol, 1 equiv.) is dissolved in MeOH (10 mL) and 4 N HCl in 1,4-dioxane (2 mL) is added. The reaction mixture is stirred at 60° C. for 1.5 h. After completion, the reaction mixture is concentrated under reduced pressure to get the desired product 10′a (1.20 g, 95%) as hydrochloride salt. The product is used crude for the next step.

The following intermediates 10′ (table 71) are available in an analogous manner using different starting materials 10.

TABLE 71

| # | structure | tᵣₑₜ [min] | [M + H]⁺ | HPLC method |
|---|---|---|---|---|
| 10′a | 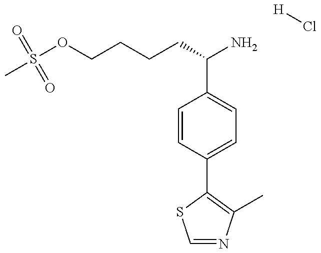 | 0.29 | 355 | 1 |
| 10′b | 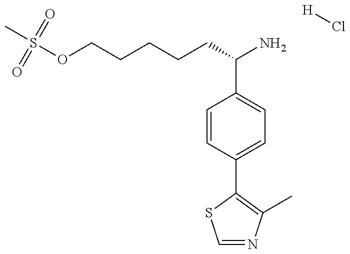 | 0.50 | 369 | 4 |
| 10′c | 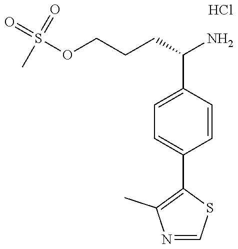 [SMILES: Cc1ncsc1-c1ccc([C@@H](N)CCCOS(C)(=O)=O)cc1 ; Cl] | 0.25 | 341 | 1 |
| 10′d | 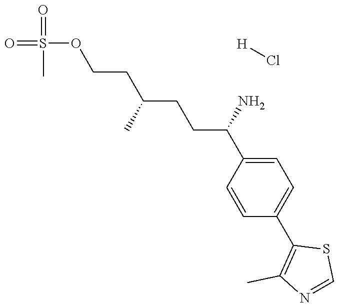 [SMILES: Cc1ncsc1-c1ccc([C@@H](N)CC[C@H](C)CCOS(C)(=O)=O)cc1 ; Cl] | 0.52 | 383 | 4 |
| 10′e | 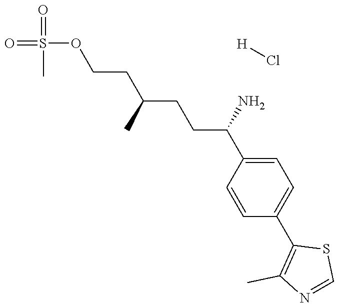 [SMILES: Cc1ncsc1-c1ccc([C@@H](N)CC[C@@H](C)CCOS(C)(=O)=O)cc1 ; Cl] | 0.53 | 383 | 4 |
| 10′f | 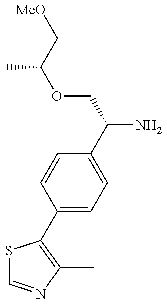 [SMILES: COC[C@@H](C)OC[C@H](N)c1ccc(-c2scnc2C)cc1] | 0.41 | 371 | 4 |

### Experimental Procedure for the Synthesis of 38a

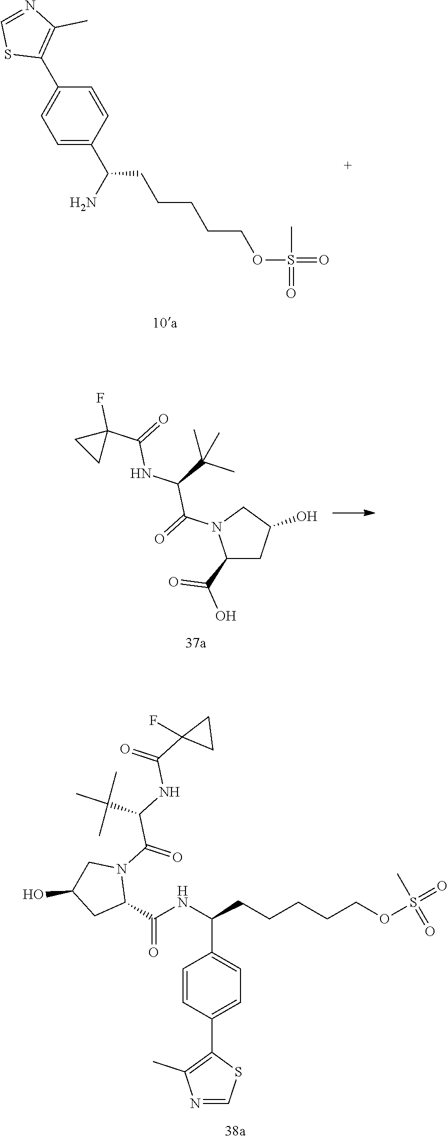 [SMILES: Cc1ncsc1-c1ccc([C@@H](N)CCCCCOS(C)(=O)=O)cc1 ; CC(C)(C)[C@H](NC(=O)C1(F)CC1)C(=O)N1C[C@H](O)C[C@H]1C(=O)O ; Cc1ncsc1-c1ccc([C@H](CCCCCOS(C)(=O)=O)NC(=O)[C@@H]2C[C@@H](O)CN2C(=O)[C@@H](NC(=O)C2(F)CC2)C(C)(C)C)cc1]

Carboxylic acid 37a (1.48 g, 4.08 mmol, 1.3 equiv.) is dissolved in DMF (10 mL) and HATU (2.14 g, 5.64 mmol, 1.8 equiv.) and DIPEA (3.1 mL, 18.8 mmol, 6 equiv.) are added. The reaction mixture is stirred at rt for 5 min. To this solution is added amine 10′a (1.16 g, 3.13 mmol, 1 equiv.), dissolved in DMF (1 mL). The reaction mixture is stirred another 30 min at rt. After completion of the reaction, the reaction mixture is concentrated under reduce pressure to get the crude compound which is purified by column chromatography to get pure 38a (1.55 g, 72.8%).

The following intermediates 38 (table 72) are available in an analogous manner using different starting materials 10′ and 37. The crude product 38 is purified by chromatography if necessary.

TABLE 72

| | | tᵣₑₜ | | HPLC |
|---|---|---|---|---|
| # | structure | [min] | [M + H]⁺ | method |
| 38a | 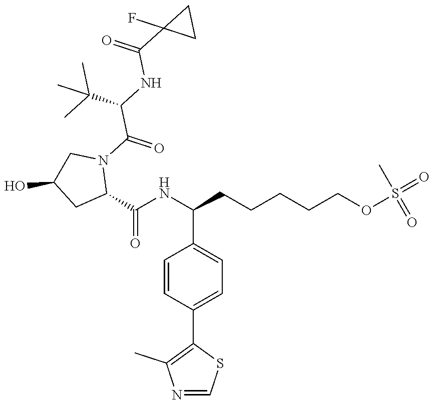 [SMILES: Cc1ncsc1-c1ccc([C@H](CCCCCOS(C)(=O)=O)NC(=O)[C@@H]2C[C@@H](O)CN2C(=O)[C@@H](NC(=O)C2(F)CC2)C(C)(C)C)cc1] | 0.62 | 681 | 4 |
| 38b | 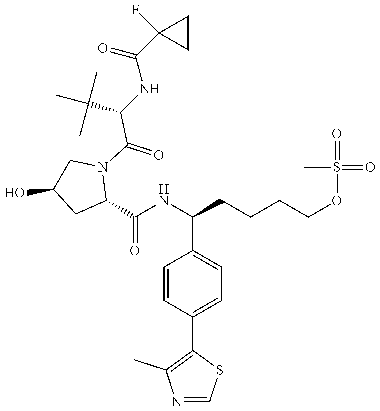 [SMILES: Cc1ncsc1-c1ccc([C@H](CCCCOS(C)(=O)=O)NC(=O)[C@@H]2C[C@@H](O)CN2C(=O)[C@@H](NC(=O)C2(F)CC2)C(C)(C)C)cc1] | 0.55 | 667 | 4 |
| 38c | 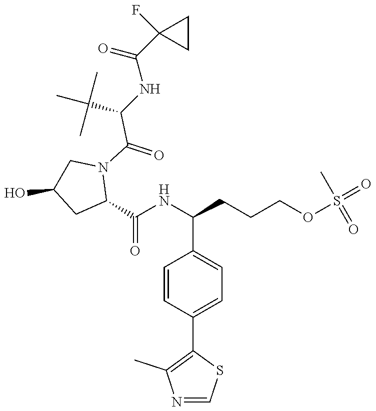 [SMILES: Cc1ncsc1-c1ccc([C@H](CCCOS(C)(=O)=O)NC(=O)[C@@H]2C[C@@H](O)CN2C(=O)[C@@H](NC(=O)C2(F)CC2)C(C)(C)C)cc1] | 0.53 | 653 | 4 |
| 38d | 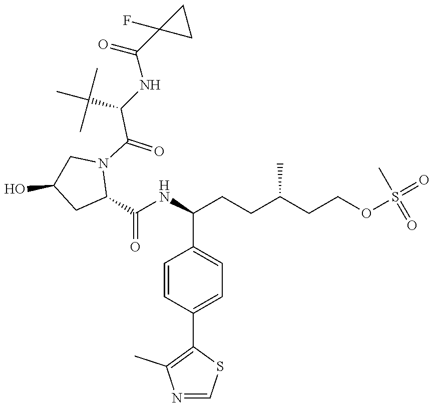 [SMILES: Cc1ncsc1-c1ccc([C@H](CC[C@H](C)CCOS(C)(=O)=O)NC(=O)[C@@H]2C[C@@H](O)CN2C(=O)[C@@H](NC(=O)C2(F)CC2)C(C)(C)C)cc1] | 0.64 | 695 | 4 |
| 38e | 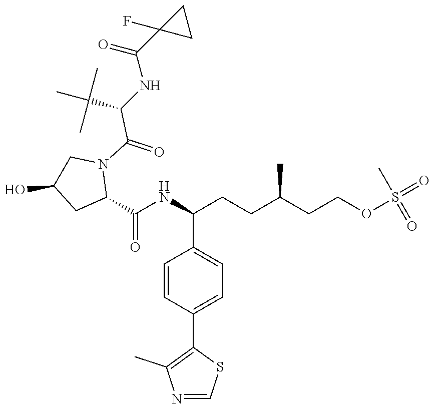 [SMILES: Cc1ncsc1-c1ccc([C@H](CC[C@@H](C)CCOS(C)(=O)=O)NC(=O)[C@@H]2C[C@@H](O)CN2C(=O)[C@@H](NC(=O)C2(F)CC2)C(C)(C)C)cc1] | 0.66 | 695 | 4 |
| 38f | 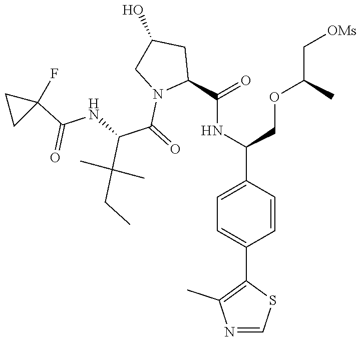 [SMILES: CCC(C)(C)[C@H](NC(=O)C1(F)CC1)C(=O)N1C[C@H](O)C[C@H]1C(=O)N[C@@H](CO[C@H](C)COS(C)(=O)=O)c1ccc(-c2scnc2C)cc1] | 1.24 | 697 | 14 |
| 38g | 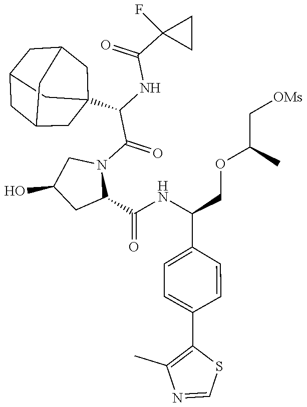 | 0.68 | 761/764 | 4 |
| 38h | 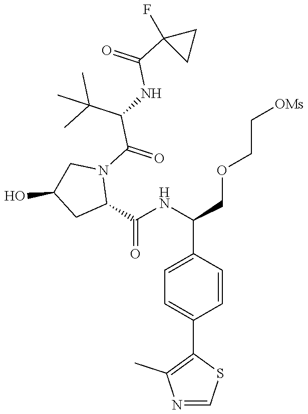 [SMILES: Cc1ncsc1-c1ccc([C@H](COCCOS(C)(=O)=O)NC(=O)[C@@H]2C[C@@H](O)CN2C(=O)[C@@H](NC(=O)C2(F)CC2)C(C)(C)C)cc1] | 1.71 | 669 | 11 |

### Synthesis Towards Compounds 42 Via Method E

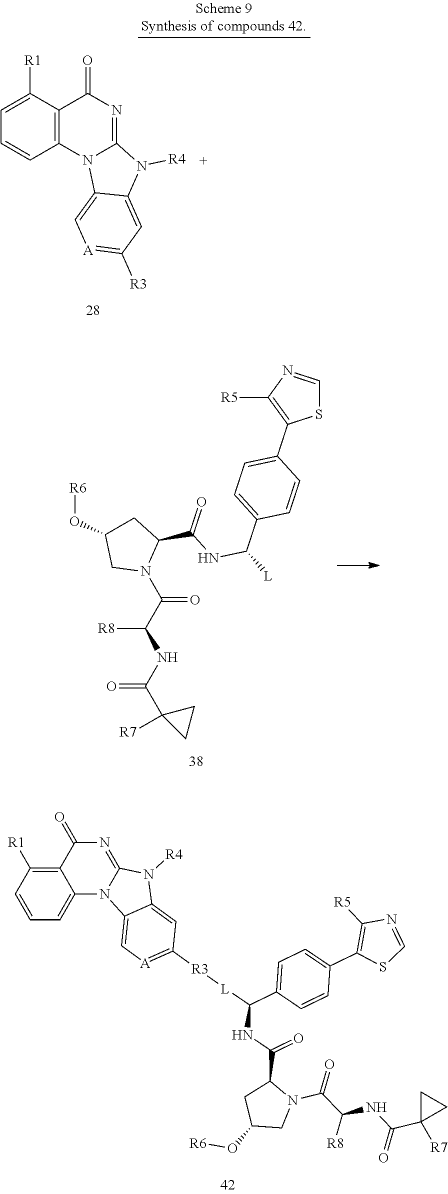

Synthesis of final molecules 42 can be realized using standard alkylation reaction conditions using e.g. an amine 28 and intermediate 38 bearing a leaving group such as e.g. a mesylate.

### Experimental Procedure for the Synthesis of Compound 42an

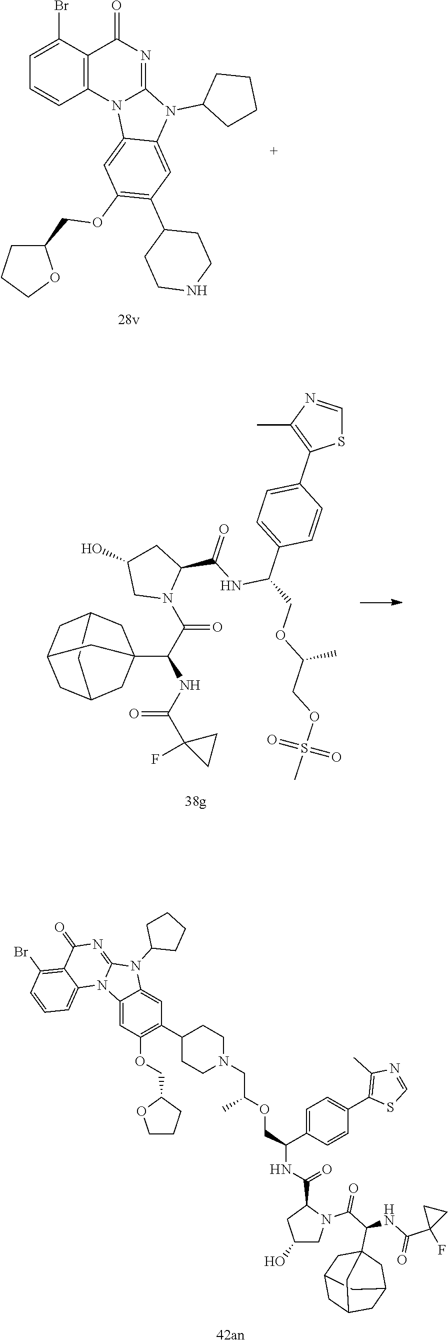 [SMILES: O=c1nc2n(C3CCCC3)c3cc(C4CCNCC4)c(OC[C@@H]4CCCO4)cc3n2c2cccc(Br)c12 ; Cc1ncsc1-c1ccc([C@H](CO[C@H](C)COS(C)(=O)=O)NC(=O)[C@@H]2C[C@@H](O)CN2C(=O)[C@@H](NC(=O)C2(F)CC2)C23CC4CC(CC(C4)C2)C3)cc1 ; Cc1ncsc1-c1ccc([C@H](CO[C@H](C)CN2CCC(c3cc4c(cc3OC[C@@H]3CCCO3)n3c5cccc(Br)c5c(=O)nc3n4C3CCCC3)CC2)NC(=O)[C@@H]2C[C@@H](O)CN2C(=O)[C@@H](NC(=O)C2(F)CC2)C23CC4CC(CC(C4)C2)C3)cc1]

Intermediate 28v (25.0 mg; 0.044 mmol; 1.00 eq.), mesylate 38g (33.6 mg; 0.044 mmol; 1.00 eq.), potassium iodide (14.7 mg; 0.088 mmol; 2.00 eq.) and DIPEA (23 μL; 0.133 mmol; 3.00 eq.) are suspended in NMP (1.50 mL). The mixture is flushed with argon for 3 min. Then the reaction mixture is heated to 95° C. and stirred at this temperature for 22 h. The reaction mixture is diluted with DCM (3 mL) and half sat. NH4Cl solution (3 mL). The organic layer is separated and the aqueous layer washed with DCM (2×3 mL). The organic layers are combined and concentrated and purified by column chromatography giving the desired product 42an (17 mg, 31% yield).

The following intermediates 42 (table 73) are available in an analogous manner using different starting materials 28 and 38. The crude product 42 is purified by chromatography if necessary.

TABLE 73

| | | | | | DC50 | DC50 |
|---|---|---|---|---|---|---|
| | | tᵣₑₜ | | HPLC | A2 | A4 |
| # | structure | [min] | [M + H]⁺ | method | [nM] | [nM] |
| 42an | 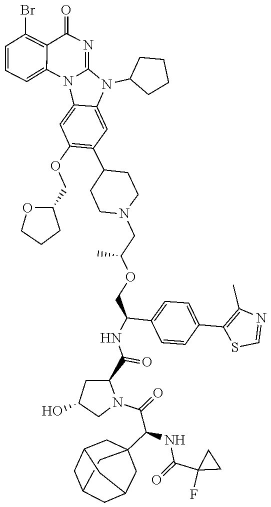 [SMILES: Cc1ncsc1-c1ccc([C@H](CO[C@H](C)CN2CCC(c3cc4c(cc3OC[C@@H]3CCCO3)n3c5cccc(Br)c5c(=O)nc3n4C3CCCC3)CC2)NC(=O)[C@@H]2C[C@@H](O)CN2C(=O)[C@@H](NC(=O)C2(F)CC2)C23CC4CC(CC(C4)C2)C3)cc1] | 1.72 | 615 | 14 | 0.6 | 7 |
| 42ao | 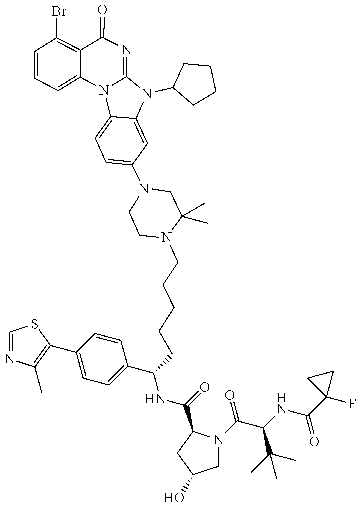 [SMILES: Cc1ncsc1-c1ccc([C@H](CCCCCN2CCN(c3ccc4c(c3)n(C3CCCC3)c3nc(=O)c5c(Br)cccc5n43)CC2(C)C)NC(=O)[C@@H]2C[C@@H](O)CN2C(=O)[C@@H](NC(=O)C2(F)CC2)C(C)(C)C)cc1] | 1.62 | 1078 | 14 | 1 | 17 |
| 42ap | 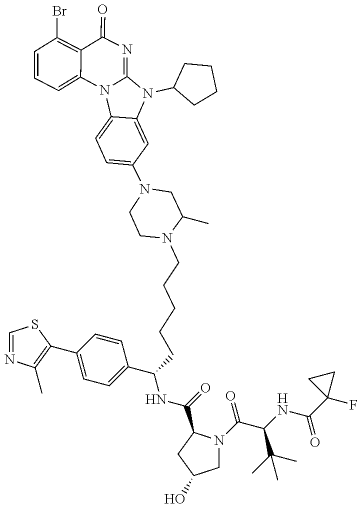 [SMILES: Cc1ncsc1-c1ccc([C@H](CCCCCN2CCN(c3ccc4c(c3)n(C3CCCC3)c3nc(=O)c5c(Br)cccc5n43)CC2C)NC(=O)[C@@H]2C[C@@H](O)CN2C(=O)[C@@H](NC(=O)C2(F)CC2)C(C)(C)C)cc1] | 1.50 | 526 | 14 | 1 | 7 |
| 42aq | 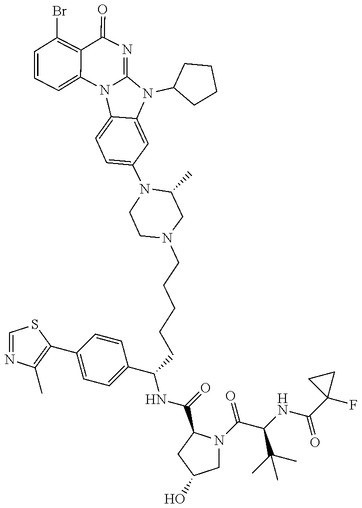 [SMILES: Cc1ncsc1-c1ccc([C@H](CCCCCN2CCN(c3ccc4c(c3)n(C3CCCC3)c3nc(=O)c5c(Br)cccc5n43)[C@H](C)C2)NC(=O)[C@@H]2C[C@@H](O)CN2C(=O)[C@@H](NC(=O)C2(F)CC2)C(C)(C)C)cc1] | 1.58 | 532 | 14 | 1 | 9 |
| 42ar | 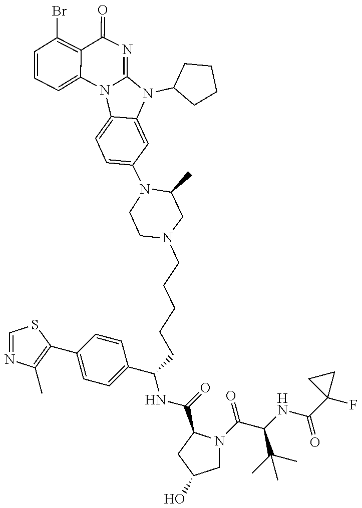 [SMILES: Cc1ncsc1-c1ccc([C@H](CCCCCN2CCN(c3ccc4c(c3)n(C3CCCC3)c3nc(=O)c5c(Br)cccc5n43)[C@@H](C)C2)NC(=O)[C@@H]2C[C@@H](O)CN2C(=O)[C@@H](NC(=O)C2(F)CC2)C(C)(C)C)cc1] | 1.58 | 532 | 14 | 0.5 | 2 |
| 42as | 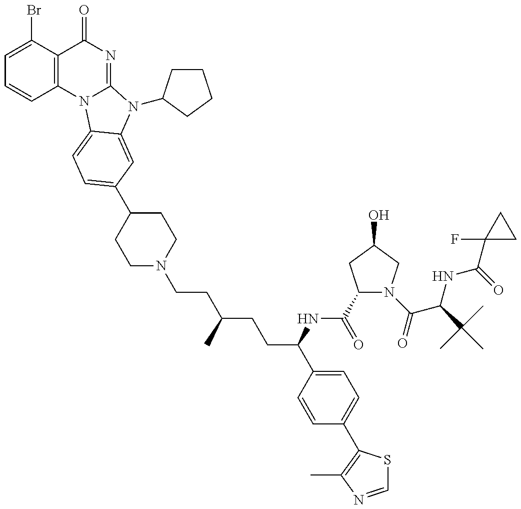 [SMILES: Cc1ncsc1-c1ccc([C@@H](CC[C@@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.56 | 532 | 14 | 82 | >833 |
| 42at | 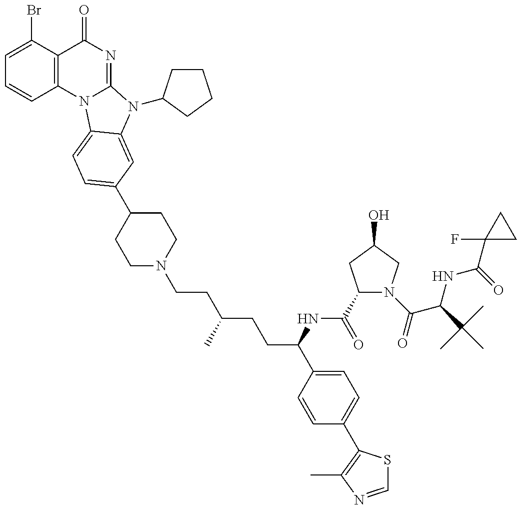 [SMILES: Cc1ncsc1-c1ccc([C@@H](CC[C@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] | 1.56 | 532 | 14 | 57 | >833 |
| 42au | 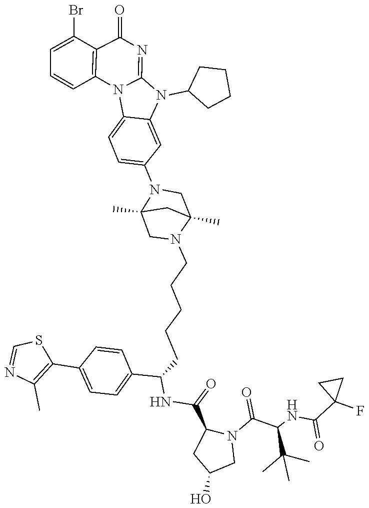 [SMILES: Cc1ncsc1-c1ccc([C@H](CCCCCN2C[C@]3(C)C[C@@]2(C)CN3c2ccc3c(c2)n(C2CCCC2)c2nc(=O)c4c(Br)cccc4n32)NC(=O)[C@@H]2C[C@@H](O)CN2C(=O)[C@@H](NC(=O)C2(F)CC2)C(C)(C)C)cc1] | 1.56 | 532 | 14 | 7 | >833 |
| 42av | 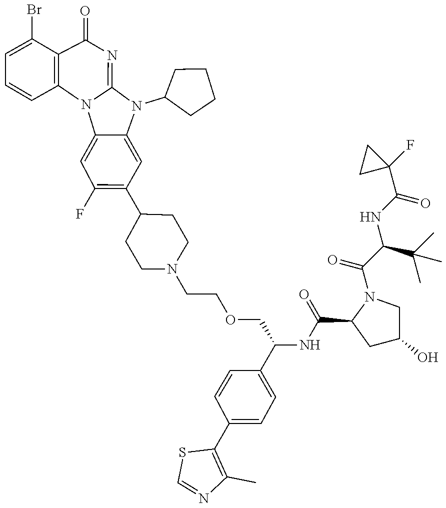 [SMILES: Cc1ncsc1-c1ccc([C@H](COCCN2CCC(c3cc4c(cc3F)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] | 1.50 | 528 | 14 | 13 | 162 |
| 42aw | 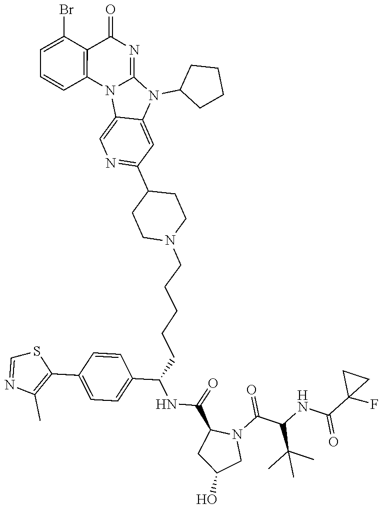 | 1.44 | 526 | 14 | 6 | >833 |
| 42ax | 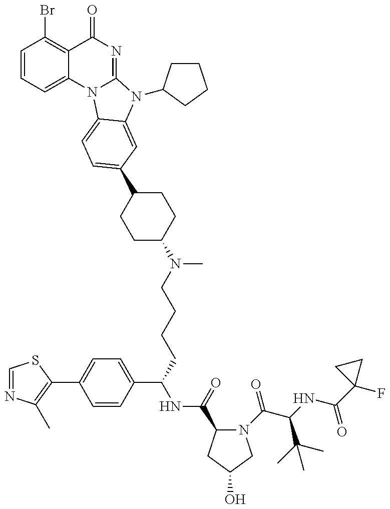 | 1.48 | 533 | 14 | 7 | 29 |
| 42ay | 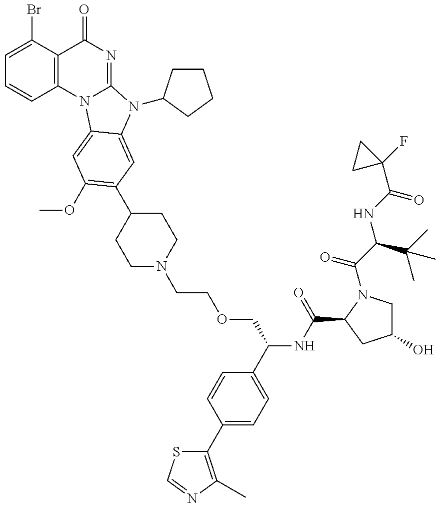 [SMILES: COc1cc2c(cc1C1CCN(CCOC[C@H](NC(=O)[C@@H]3C[C@@H](O)CN3C(=O)[C@@H](NC(=O)C3(F)CC3)C(C)(C)C)c3ccc(-c4scnc4C)cc3)CC1)n(C1CCCC1)c1nc(=O)c3c(Br)cccc3n21] | 1.48 | 534 | 14 | 15 | 109 |
| 42az | 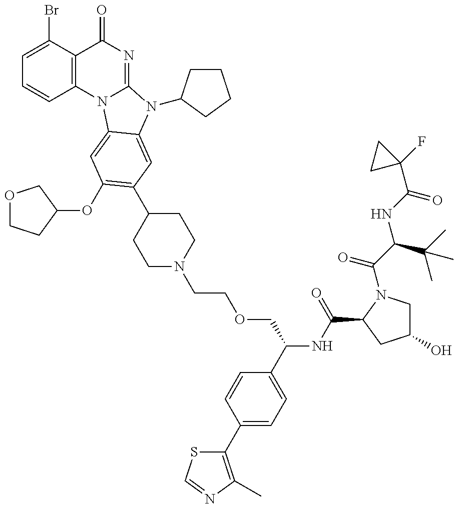 [SMILES: Cc1ncsc1-c1ccc([C@H](COCCN2CCC(c3cc4c(cc3OC3CCOC3)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] | 1.44 | 562 | 14 | 36 | 800 |
| 42ba | 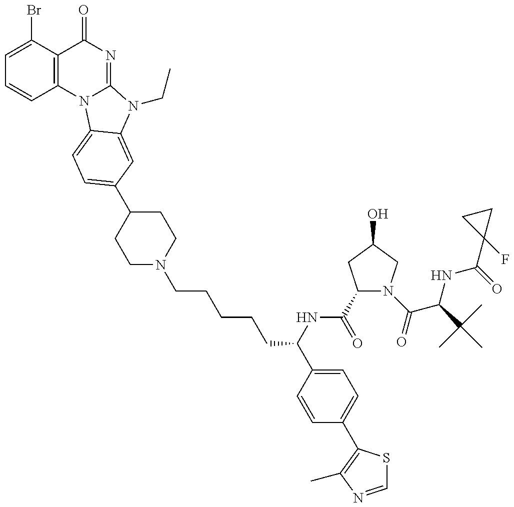 [SMILES: CCn1c2cc(C3CCN(CCCCC[C@H](NC(=O)[C@@H]4C[C@@H](O)CN4C(=O)[C@@H](NC(=O)C4(F)CC4)C(C)(C)C)c4ccc(-c5scnc5C)cc4)CC3)ccc2n2c3cccc(Br)c3c(=O)nc12] | 1.46 | 505 | 14 | 4 | 22 |

### Synthesis Towards Compounds 42 Using Method F

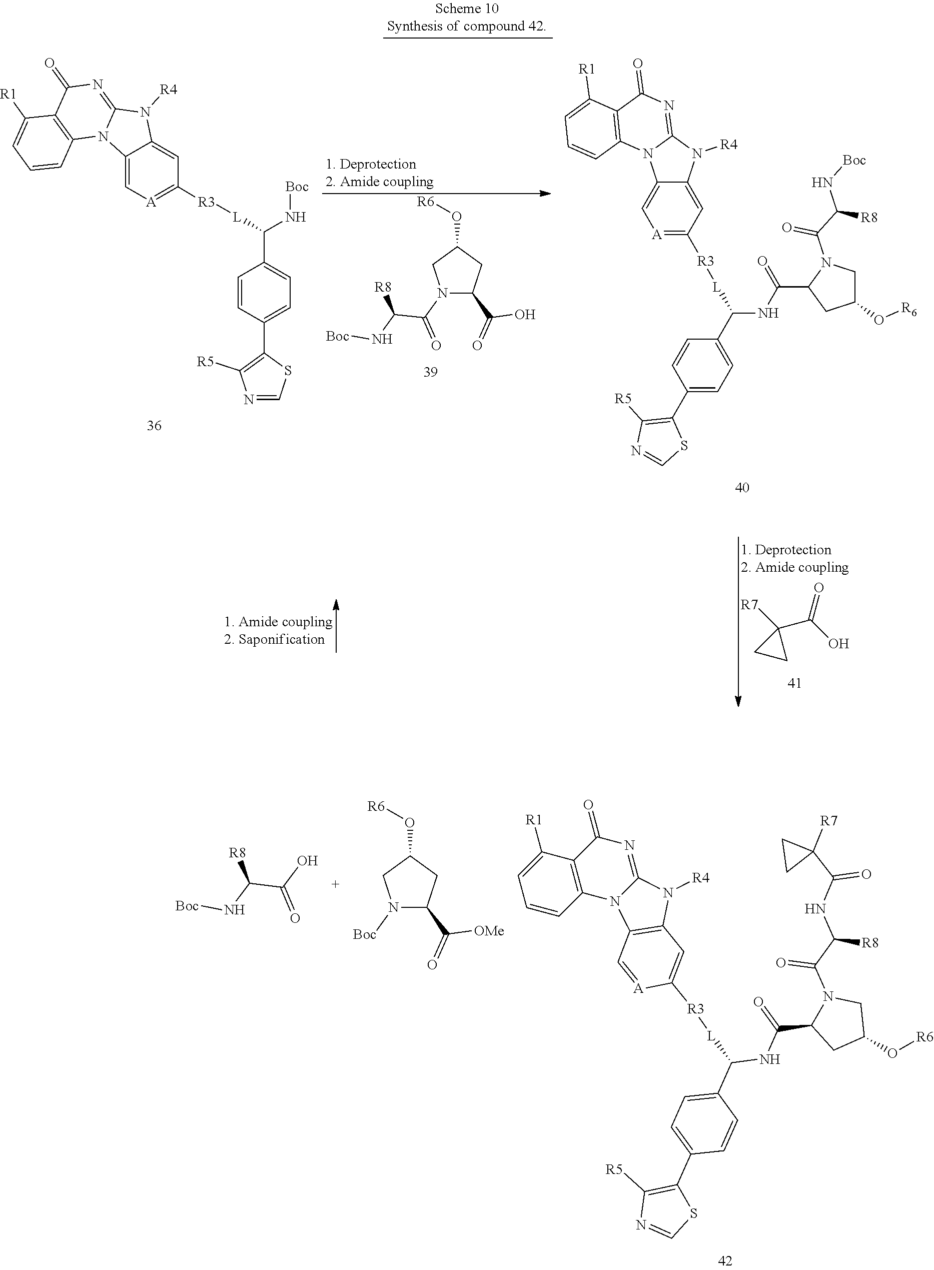

Intermediate 39 is synthesized via standard amide coupling followed by saponification of the ester under basic conditions. After acidic deprotection compound 36 is used in an amide coupling with intermediate 39 leading to intermediate 40. Subsequent deprotection under acidic conditions followed by standard amide coupling using e.g. HATU or T3P as coupling reagent compound 42 is obtained.

### Experimental Procedure for the Synthesis of 39a

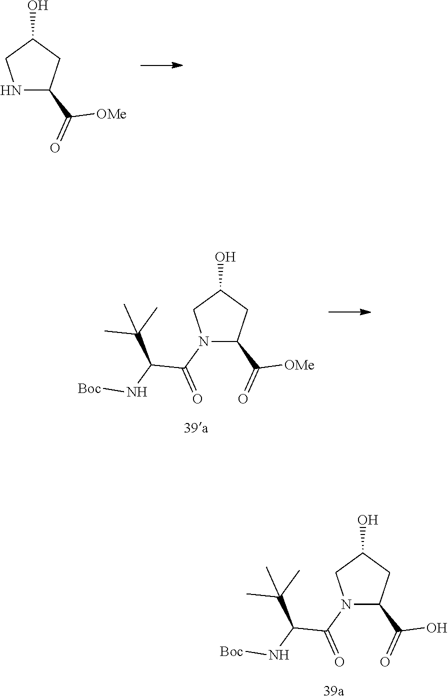 [SMILES: COC(=O)[C@@H]1C[C@@H](O)CN1 ; COC(=O)[C@@H]1C[C@@H](O)CN1C(=O)[C@@H](NC(=O)OC(C)(C)C)C(C)(C)C ; CC(C)(C)OC(=O)N[C@H](C(=O)N1C[C@H](O)C[C@H]1C(=O)O)C(C)(C)C]

To a stirred solution of (S)-2-tert-Butoxycarbonylamino-3,3-dimethyl-butyric acid (10.0 g; 43.2 mmol; 1.0 equiv.) in DMF (70.0 mL), DIPEA (37.7 mL; 216 mmol; 5.0 equiv.) is added dropwise at 0° C. and after 5 min HATU (19.7 g; 51.9 mmol; 1.2 equiv.) and (2S,4R)-4-Hydroxy-pyrrolidine-2-carboxylic acid methyl ester hydrochloride (7.07 g; 38.9 mmol; 0.90 equiv.) is added and stirred at rt for 16 h. The reaction mixture is quenched with ice water and extracted with EtOAc. The crude compound is purified by column chromatography to get the desired product 39′a (12.0 g, 77.4%).

To a stirred solution of methyl ester 39′a (12.0 g; 33.5 mmol; 1.0 equiv.) in water:THF (1:2) (130 mL), LiOH H₂O (2.81 g; 66.9 mmol; 2.0 equiv.) is added and the reaction mixture stirred for 16 h at rt. The reaction mixture is concentrated under reduced pressure and then acidified with 1M HCl solution. The formed precipitate is filtered off to give 39a (11.0 g, 95.4%).

TABLE 74

| # | structure | tᵣₑₜ [min] | [M + H]⁺ | HPLC method |
|---|---|---|---|---|
| 39a | 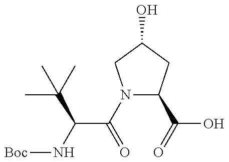 [SMILES: CC(C)(C)OC(=O)N[C@H](C(=O)N1C[C@H](O)C[C@H]1C(=O)O)C(C)(C)C] | 1.48 | 345 | 11 |

### Experimental Procedure for the Synthesis of 40a

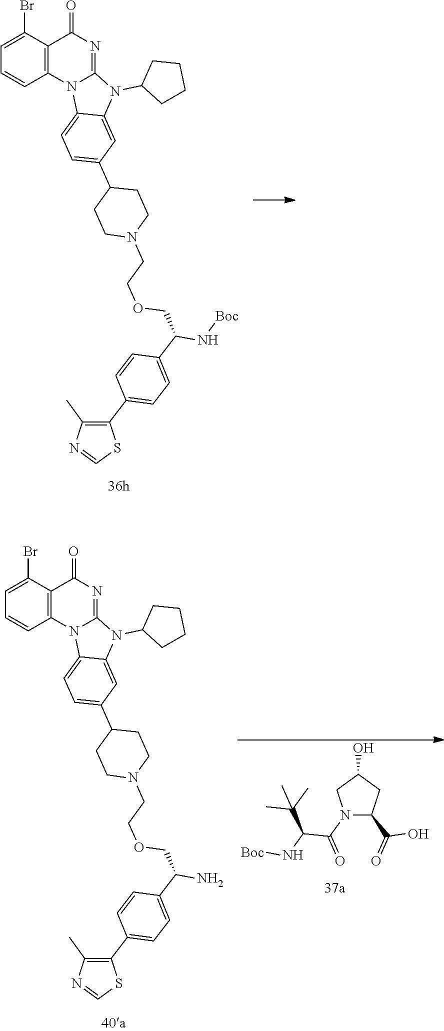 [SMILES: Cc1ncsc1-c1ccc([C@H](COCCN2CCC(c3ccc4c(c3)n(C3CCCC3)c3nc(=O)c5c(Br)cccc5n43)CC2)NC(=O)OC(C)(C)C)cc1 ; Cc1ncsc1-c1ccc([C@@H](N)COCCN2CCC(c3ccc4c(c3)n(C3CCCC3)c3nc(=O)c5c(Br)cccc5n43)CC2)cc1 ; CC(C)(C)OC(=O)N[C@H](C(=O)N1C[C@H](O)C[C@H]1C(=O)O)C(C)(C)C]

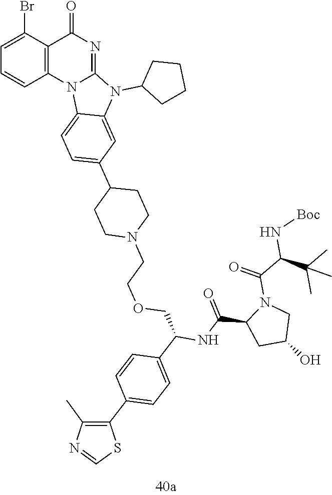 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)OC(C)(C)C)C(C)(C)C)cc1]

Carbamate 36h (1.10 g; 1.33 mmol; 1.0 equiv.) is dissolved in methanol (20 mL) and 4N HCl in dioxane (8.0 mL; 32.0 mmol; 24.1 equiv.) is added. The reaction mixture is stirred at 45° C. for 1 h. The solvent is removed under reduced pressure giving the desired amine 40′a (estimated yield: 1.00 g, 98.5%) as a hydrochloride salt.

Carboxylic acid 40′a (0.87 g; 2.48 mmol; 1.50 equiv.) and HATU (0.94 g; 2.48 mmol; 1.50 equiv.) are dissolved in MeCN (6.0 mL) and TEA (0.72 mL; 4.98 mmol; 3.0 equiv.) is added. The mixture is stirred at rt for 5 min, then added to a stirred solution of amine 40′a (1.20 g; 1.65 mmol; 1.0 equiv.) in DMF (2.0 mL) and the reaction mixture is stirred at rt for 10 min. The mixture is filtered and purified by column chromatography giving the desired product 40a (1.31 g, 75%).

The following intermediates 40 (table 75) are available in an analogous manner using different starting materials 36. The crude product 40 is purified by chromatography if

TABLE 75

| # | structure | tᵣₑₜ [min] | [M + 2H]⁺⁺ | HPLC method |
|---|---|---|---|---|
| 40a | 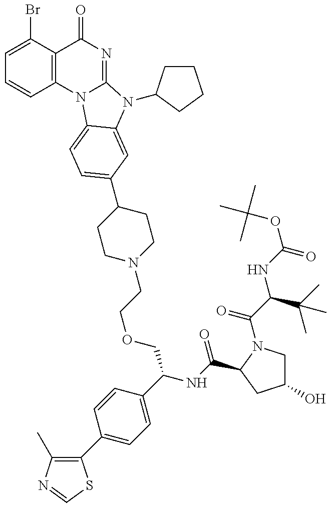 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)OC(C)(C)C)C(C)(C)C)cc1] | 0.61 | 1052 | 7 |
| 40b | 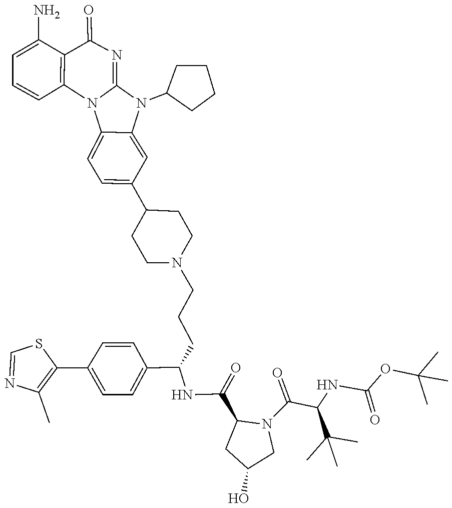 [SMILES: Cc1ncsc1-c1ccc([C@H](CCCN2CCC(c3ccc4c(c3)n(C3CCCC3)c3nc(=O)c5c(N)cccc5n43)CC2)NC(=O)[C@@H]2C[C@@H](O)CN2C(=O)[C@@H](NC(=O)OC(C)(C)C)C(C)(C)C)cc1] | 0.78 | 973 | 7 |

### Experimental Procedure for the Synthesis of Compound 42bb

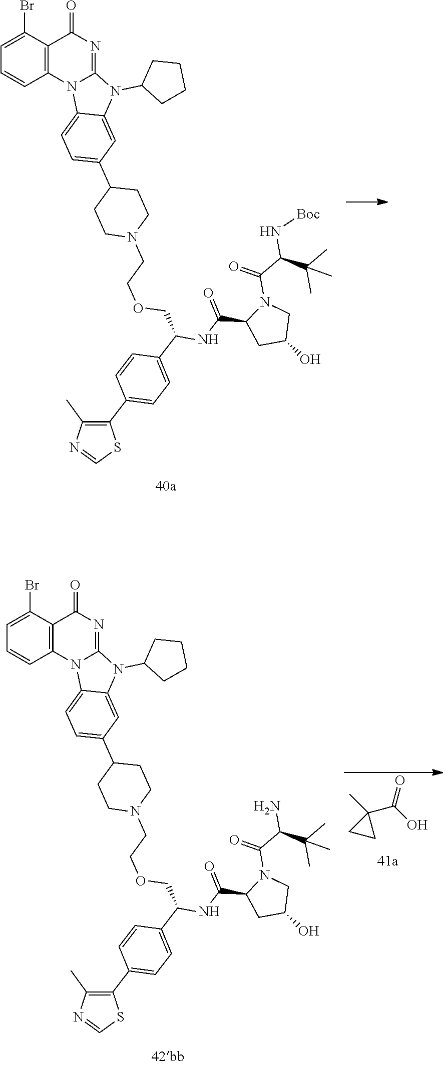

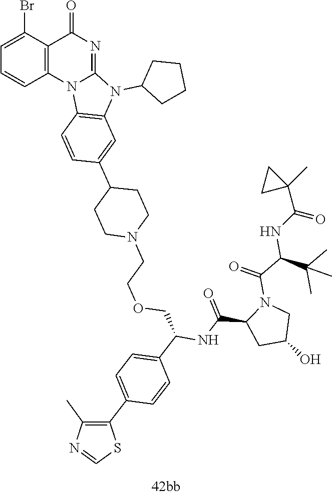 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C)CC2)C(C)(C)C)cc1]

Carbamate 40a (1.40 g; 1.33 mmol; 1.00 eq.) is dissolved in MeOH (7.00 mL) and 4M HCl in Dioxane (5.00 mL; 20.0 mmol; 15.0 eq.) is added. The reaction mixture is stirred at 45° C. for 30 min. The reaction mixture is concentrated under reduced pressure. The crude product is dissolved in DCM (50 mL) and washed with 2M NaOH (40 mL). The aqueous layer is washed with DCM (2×15 mL). The combined organic layers are washed with water (30 mL). The organic layer is concentrated, dissolved in ACN/water and freeze dried to give the desired product 42′bb (1.26 g, 99% yield). The crude product is taken into the next step without further purification.

Carboxylic acid 41a (2.73 mg; 0.027 mmol; 1.30 eq.) and HATU (11.9 mg; 0.032 mmol; 1.50 eq.) are dissolved in DMF (0.300 mL) and TEA (10.0 μL; 0.069 mmol; 3.29 eq.) is added. The mixture is stirred at rt for 15 min. Then carboxamide 42′bb (20.0 mg; 0.021 mmol; 1.00 eq.) is added and the mixture is stirred at rt for 40 min. The reaction mixture is diluted with ACN/water, filtered through a syringe filter and purified by column chromatography giving the desired product 42bb (15 mg, 69% yield).

The following intermediates 42 (table 76) are available in an analogous manner using different starting materials 40 and 41. The crude product 42 is purified by chromatography if necessary.

TABLE 76

| | | | | | DC50 | DC50 |
|---|---|---|---|---|---|---|
| | | tᵣₑₜ | | HPLC | A2 | A4 |
| # | structure | [min] | [M + 2H]⁺⁺ | method | [nM] | [nM] |
| 42bb | 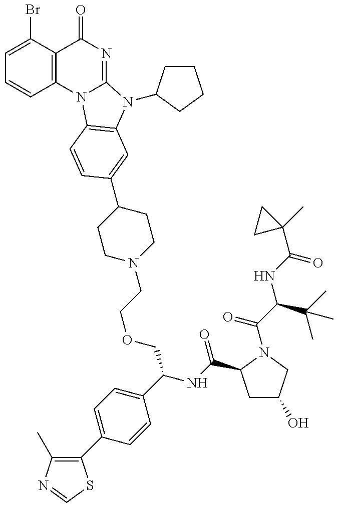 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C)CC2)C(C)(C)C)cc1] | 1.46 | 517 | 14 | 17 | 142 |
| 42bc | 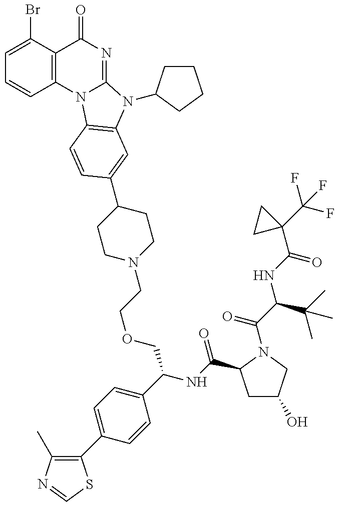 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C(F)(F)F)CC2)C(C)(C)C)cc1] | 1.53 | 544 | 14 | 19 | 133 |
| 42bd | 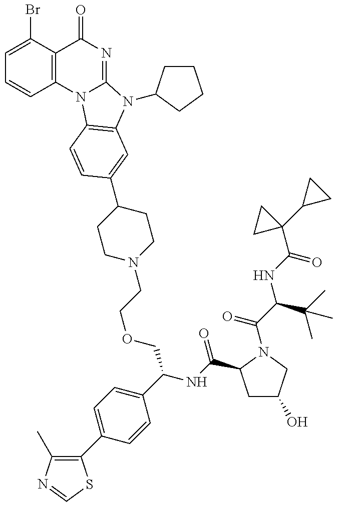 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C3CC3)CC2)C(C)(C)C)cc1] | 1.52 | 530 | 14 | 11 | 64 |
| 42be | 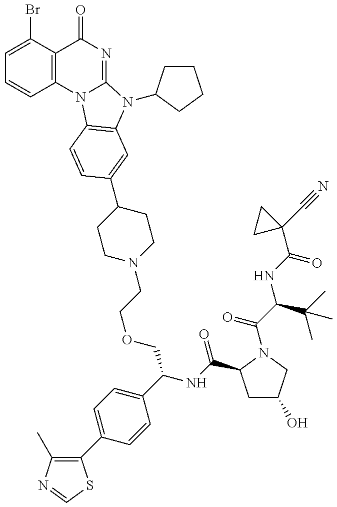 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C#N)CC2)C(C)(C)C)cc1] | 1.44 | 522 | 14 | 12 | 67 |
| 42bf | 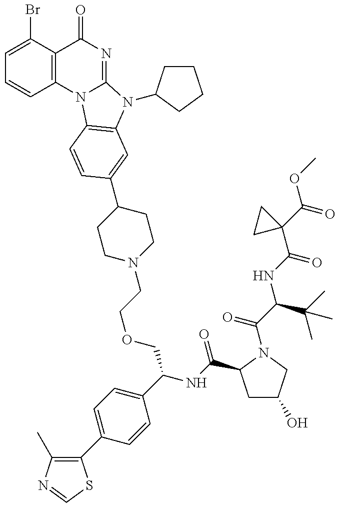 [SMILES: COC(=O)C1(C(=O)N[C@H](C(=O)N2C[C@H](O)C[C@H]2C(=O)N[C@@H](COCCN2CCC(c3ccc4c(c3)n(C3CCCC3)c3nc(=O)c5c(Br)cccc5n43)CC2)c2ccc(-c3scnc3C)cc2)C(C)(C)C)CC1] | 1.47 | 539 | 14 | 8 | 87 |
| 42bg | 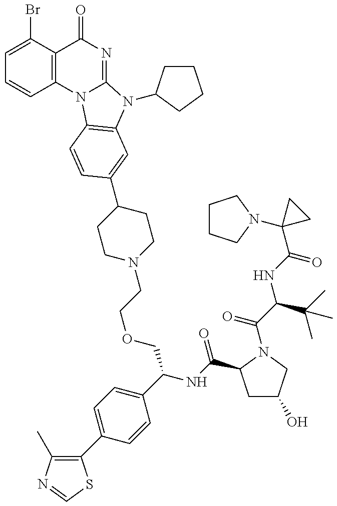 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(N3CCCC3)CC2)C(C)(C)C)cc1] | 1.57 | 544 | 14 | 15 | 53 |
| 42bh | 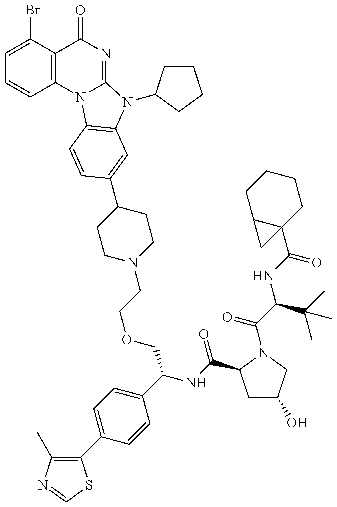 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C23CCCCC2C3)C(C)(C)C)cc1] | 1.55 | 1073 [M + H]⁺ | 14 | 6 | 153 |
| 42bi | 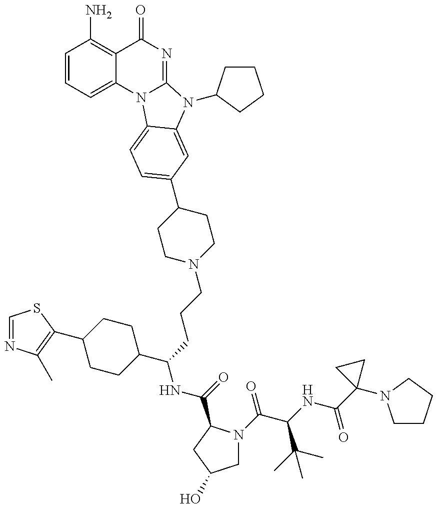 [SMILES: Cc1ncsc1C1CCC([C@H](CCCN2CCC(c3ccc4c(c3)n(C3CCCC3)c3nc(=O)c5c(N)cccc5n43)CC2)NC(=O)[C@@H]2C[C@@H](O)CN2C(=O)[C@@H](NC(=O)C2(N3CCCC3)CC2)C(C)(C)C)CC1] | 1.59 | 505 | 14 | 3 | 6 |

### Synthesis Towards Compounds 42′″

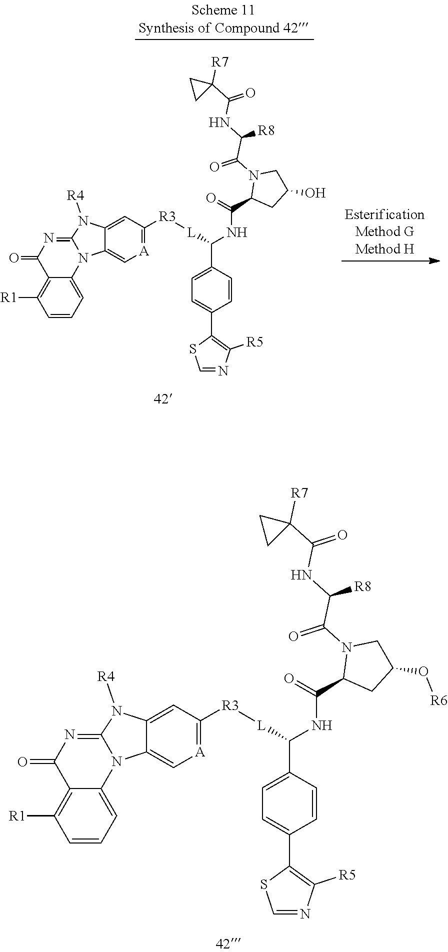

### Experimental Procedure for the Synthesis of Compound 42bj Via Method G

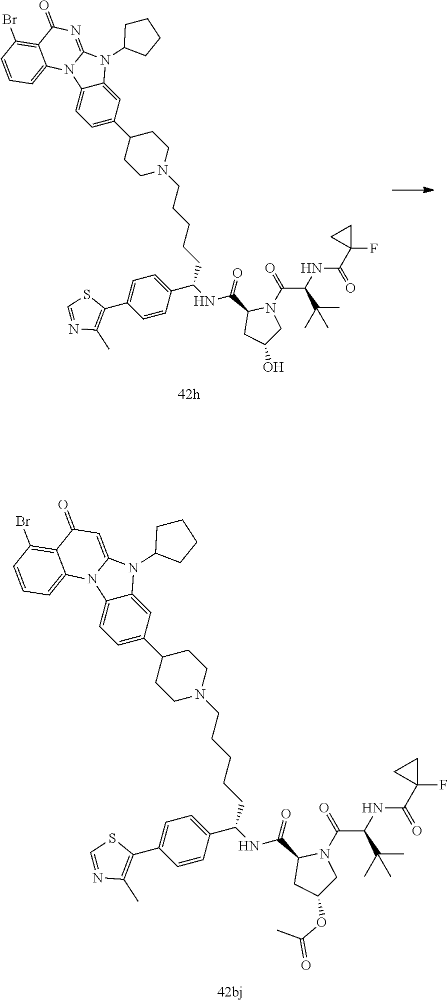 [SMILES: Cc1ncsc1-c1ccc([C@H](CCCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; CC(=O)O[C@@H]1C[C@@H](C(=O)N[C@@H](CCCCCN2CCC(c3ccc4c(c3)n(C3CCCC3)c3cc(=O)c5c(Br)cccc5n43)CC2)c2ccc(-c3scnc3C)cc2)N(C(=O)[C@@H](NC(=O)C2(F)CC2)C(C)(C)C)C1]

Secondary alcohol 42h (50 mg; 0.048 mmol; 1.0 equiv.) is dissolved in pyridine (1.00 mL) and acetic anhydride (0.500 mL; 5.22 mmol; 110 equiv.) is added and the mixture is stirred at 50° C. for 30 min. The reaction is cooled to rt, diluted with MeCN/water and purified by RP-chromatography to give 42bj (42 mg, 69%).

TABLE 77

| | | | | | DC50 | DC50 |
|---|---|---|---|---|---|---|
| | | tᵣₑₜ | | HPLC | A2 | A4 |
| # | structure | [min] | [M + 2H]⁺⁺ | method | [nM] | [nM] |
| 42bj | 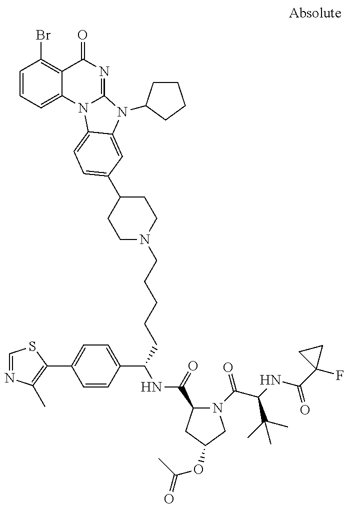 [SMILES: CC(=O)O[C@@H]1C[C@@H](C(=O)N[C@@H](CCCCCN2CCC(c3ccc4c(c3)n(C3CCCC3)c3nc(=O)c5c(Br)cccc5n43)CC2)c2ccc(-c3scnc3C)cc2)N(C(=O)[C@@H](NC(=O)C2(F)CC2)C(C)(C)C)C1] | 1.62 | 546 | 14 | 8 | 83 |

### Experimental Procedure for the Synthesis of Compound 42bk Via Method H

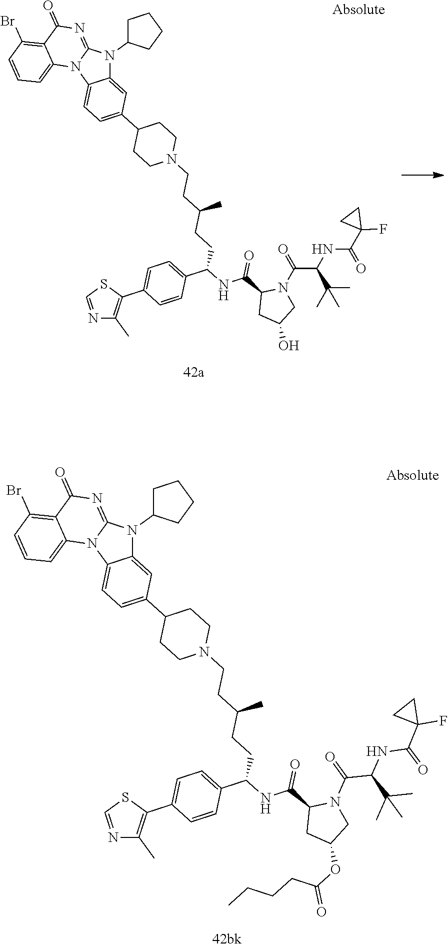 [SMILES: Cc1ncsc1-c1ccc([C@H](CC[C@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; CCCCC(=O)O[C@@H]1C[C@@H](C(=O)N[C@@H](CC[C@H](C)CCN2CCC(c3ccc4c(c3)n(C3CCCC3)c3nc(=O)c5c(Br)cccc5n43)CC2)c2ccc(-c3scnc3C)cc2)N(C(=O)[C@@H](NC(=O)C2(F)CC2)C(C)(C)C)C1]

Secondary alcohol 42a (40 mg; 0.038 mmol; 1.0 equiv.) is dissolved in dry DCM (0.400 mL) and cooled to 0° C. Then valeryl chloride (0.027 mL; 0.226 mmol; 6.0 equiv.) is added slowly and the mixture is stirred at rt for 16 h. The reaction is quenched with MeOH and stirred for 20 minutes. The solvents are removed under reduced pressure. The crude is dissolved in MeCN/water, basified with DIPEA and purified by RP-chromatography to give 42bk (27 mg, 62%).

TABLE 78

| | | | | | DC50 | |
|---|---|---|---|---|---|---|
| | | tᵣₑₜ | | HPLC | A2 | DC50 A4 |
| # | structure | [min] | [M + 2H]⁺⁺ | method | [nM] | [nM] |
| 42bk | 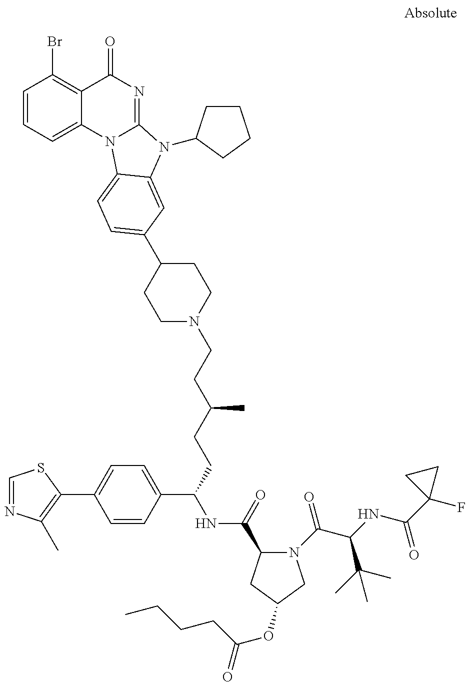 [SMILES: CCCCC(=O)O[C@@H]1C[C@@H](C(=O)N[C@@H](CC[C@H](C)CCN2CCC(c3ccc4c(c3)n(C3CCCC3)c3nc(=O)c5c(Br)cccc5n43)CC2)c2ccc(-c3scnc3C)cc2)N(C(=O)[C@@H](NC(=O)C2(F)CC2)C(C)(C)C)C1] | 1.84 | 574 | 14 | 3 | 60 |

### Example of Pharmaceutical Formulation

### Ampoule Solution

| | |
|---|---|
| active substance according to formula (I) | 50 mg |
| sodium chloride | 50 mg |
| water for inj. | 5 mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. A compound of formula (I): 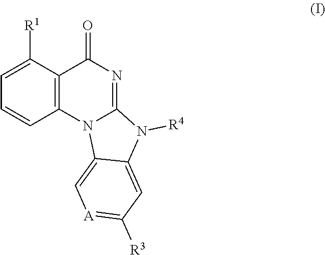
wherein:
A is —C(R²)— or —N—;
R¹ is halogen or —NH₂;
R² is selected from the group consisting of: hydrogen, halogen and —OX;
R³ is selected from the group consisting of: halogen, C₅₋₇-carbocyclyl and 4-12 membered heterocyclyl, wherein said C₅₋₇-carbocyclyl or 4-12 membered heterocyclyl is optionally substituted with at least one substituent selected from the group consisting of: C₁₋₃-alkyl, —NR^{a}R^{b}, —N(R^{a})COOR^{b} and —COOR^{a};
R⁴ is selected from the group consisting of: C₁₋₄-alkyl, C₃₋₆-carbocyclyl and 4-6 membered heterocyclyl, wherein said C₃₋₆-carbocyclyl or 4-6 membered heterocyclyl is optionally substituted with at least one substituent selected from the group consisting of: C₁₋₃-alkyl and —OH;
X is selected from the group consisting of: hydrogen, C₁₋₄-alkyl, —C(O)OC₁₋₄-alkyl, —(CH₂)ₙ—[O(CH₂)₂]ₘ—Y and 4-7 membered heterocyclyl, wherein said C₁₋₄-alkyl is optionally substituted with at least one substituent selected from the group consisting of: —COOR^{a} and —NR^{a}R^{b};
Y is selected from the group consisting of: —OR^{a}, —NR^{a}R^{b} and 4-7 membered heterocyclyl optionally substituted with at least one substituent selected from the group consisting of:
C₁₋₃-alkyl, —COOR^{a} and —NR^{a}R^{b};
R^{a} and R^{b} are independently at each occurrence hydrogen or C₁₋₄-alkyl;
n is an integer selected from the group consisting of: 1, 2, 3 and 4;
m is an integer selected from the group consisting of: 0, 1, 2, 3, 4 and 5;
or a salt thereof.

2. The compound according to claim 1, wherein:
n is 1, 2 or 3; and/or
m is 0 or 1 or a salt thereof.

3. The compound according to claim 1, wherein R² is selected from the group consisting of: hydrogen, fluorine, —OCH₃, 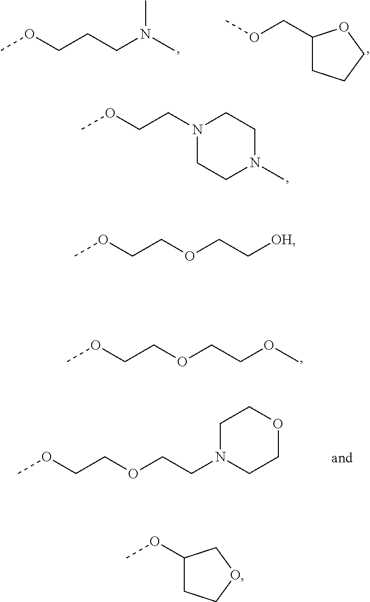
or a salt thereof.

4. The compound according to claim 1, wherein R⁴ is selected from the group consisting of: ethyl, 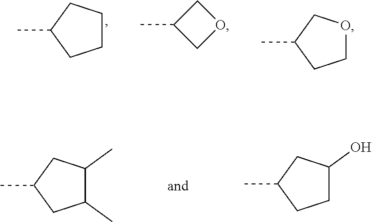
or a salt thereof.

5. The compound according to claim 1, being selected from the group consisting of: 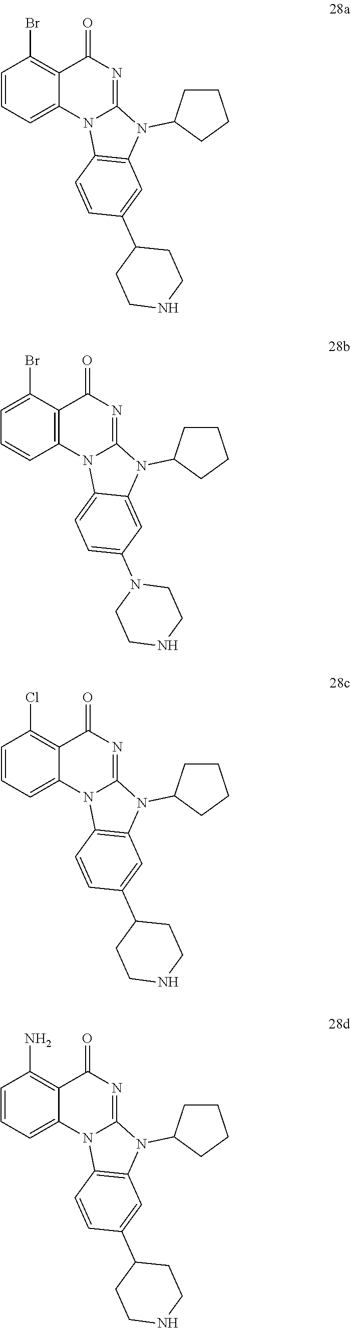 [SMILES: O=c1nc2n(C3CCCC3)c3cc(C4CCNCC4)ccc3n2c2cccc(Br)c12 ; O=c1nc2n(C3CCCC3)c3cc(N4CCNCC4)ccc3n2c2cccc(Br)c12 ; O=c1nc2n(C3CCCC3)c3cc(C4CCNCC4)ccc3n2c2cccc(Cl)c12 ; Nc1cccc2c1c(=O)nc1n(C3CCCC3)c3cc(C4CCNCC4)ccc3n21] 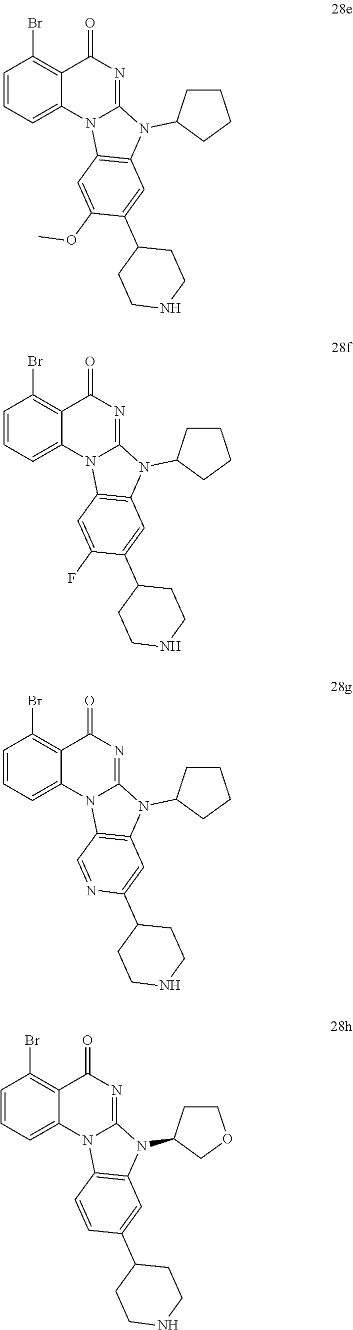 [SMILES: COc1cc2c(cc1C1CCNCC1)n(C1CCCC1)c1nc(=O)c3c(Br)cccc3n21 ; O=c1nc2n(C3CCCC3)c3cc(C4CCNCC4)c(F)cc3n2c2cccc(Br)c12 ; O=c1nc2n(C3CCCC3)c3cc(C4CCNCC4)ncc3n2c2cccc(Br)c12 ; O=c1nc2n([C@H]3CCOC3)c3cc(C4CCNCC4)ccc3n2c2cccc(Br)c12] 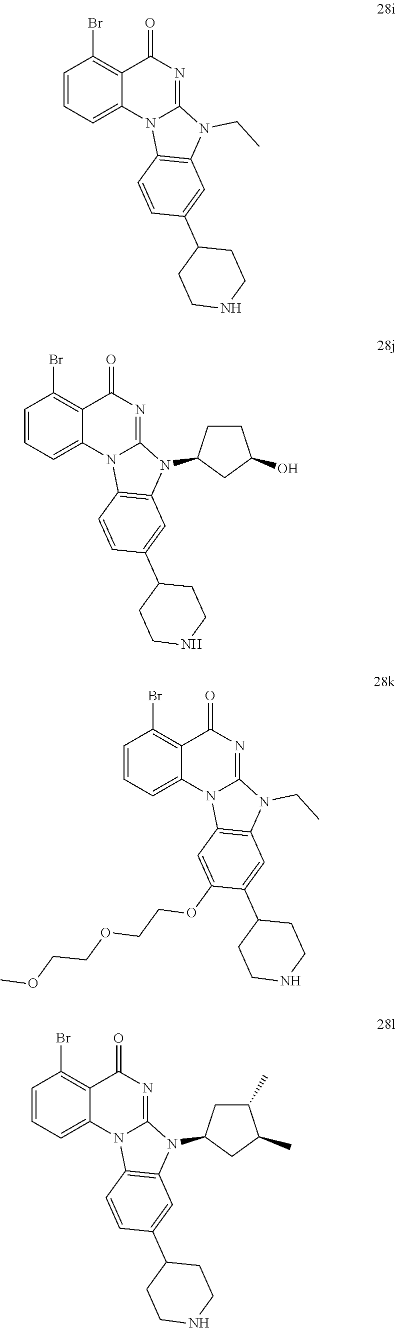 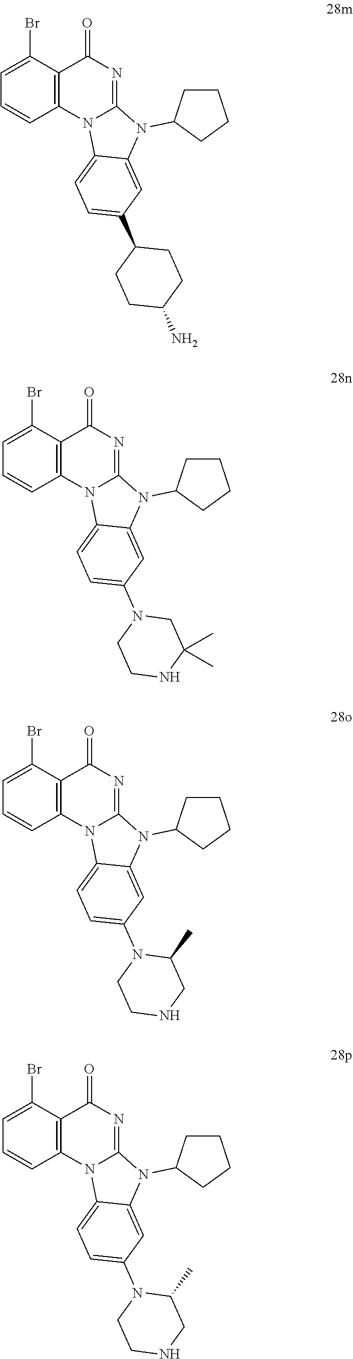 [SMILES: N[C@H]1CC[C@H](c2ccc3c(c2)n(C2CCCC2)c2nc(=O)c4c(Br)cccc4n32)CC1 ; CC1(C)CN(c2ccc3c(c2)n(C2CCCC2)c2nc(=O)c4c(Br)cccc4n32)CCN1 ; C[C@H]1CNCCN1c1ccc2c(c1)n(C1CCCC1)c1nc(=O)c3c(Br)cccc3n21 ; C[C@@H]1CNCCN1c1ccc2c(c1)n(C1CCCC1)c1nc(=O)c3c(Br)cccc3n21] 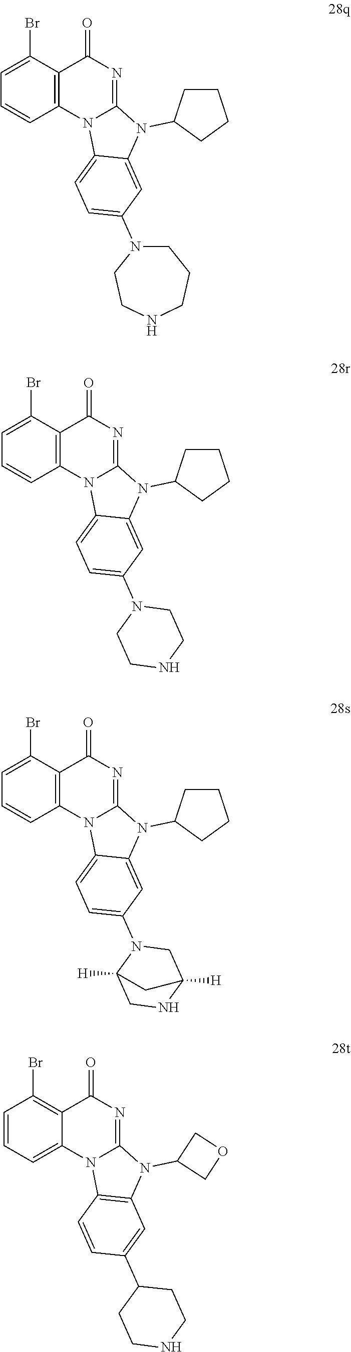 [SMILES: O=c1nc2n(C3CCCC3)c3cc(N4CCCNCC4)ccc3n2c2cccc(Br)c12 ; O=c1nc2n(C3CCCC3)c3cc(N4CCNCC4)ccc3n2c2cccc(Br)c12 ; O=c1nc2n(C3CCCC3)c3cc(N4C[C@@H]5C[C@H]4CN5)ccc3n2c2cccc(Br)c12 ; O=c1nc2n(C3COC3)c3cc(C4CCNCC4)ccc3n2c2cccc(Br)c12] 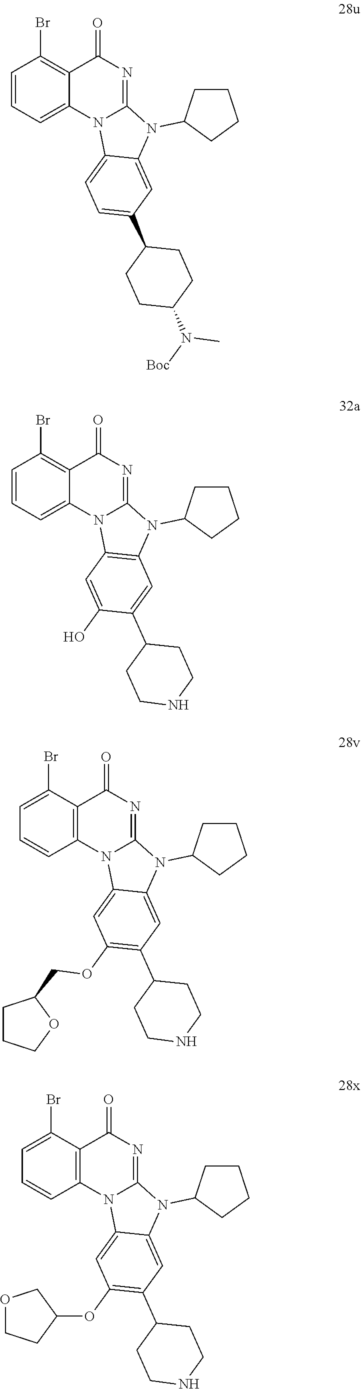 [SMILES: CN(C(=O)OC(C)(C)C)[C@H]1CC[C@H](c2ccc3c(c2)n(C2CCCC2)c2nc(=O)c4c(Br)cccc4n32)CC1 ; O=c1nc2n(C3CCCC3)c3cc(C4CCNCC4)c(O)cc3n2c2cccc(Br)c12 ; O=c1nc2n(C3CCCC3)c3cc(C4CCNCC4)c(OC[C@@H]4CCCO4)cc3n2c2cccc(Br)c12 ; O=c1nc2n(C3CCCC3)c3cc(C4CCNCC4)c(OC4CCOC4)cc3n2c2cccc(Br)c12] 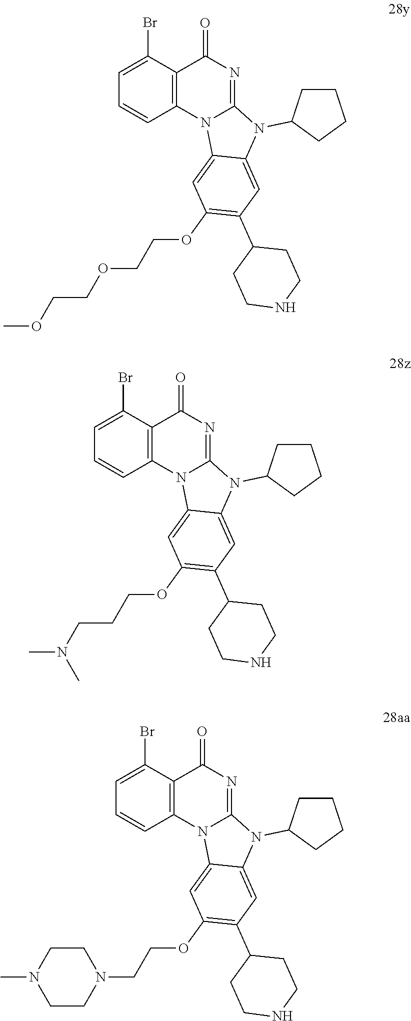 [SMILES: COCCOCCOc1cc2c(cc1C1CCNCC1)n(C1CCCC1)c1nc(=O)c3c(Br)cccc3n21 ; CN(C)CCCOc1cc2c(cc1C1CCNCC1)n(C1CCCC1)c1nc(=O)c3c(Br)cccc3n21 ; CN1CCN(CCOc2cc3c(cc2C2CCNCC2)n(C2CCCC2)c2nc(=O)c4c(Br)cccc4n32)CC1]
or a salt thereof.

6. A compound of formula (II): 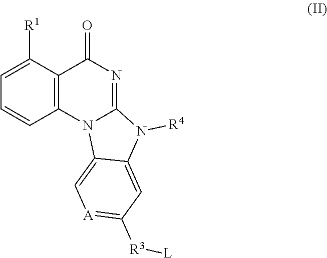
wherein:
A is —C(R²)— or —N—;
R¹ is halogen or —NH₂;
R⁴ is selected from the group consisting of: C₁₋₄-alkyl, C₃₋₆-carbocyclyl and 4-6 membered heterocyclyl, wherein said C₃₋₆-carbocyclyl or 4-6 membered heterocyclyl is optionally substituted with at least one substituent selected from the group consisting of: C₁₋₃-alkyl and —OH;
R³ is C₅₋₇-carbocyclyl or 4-12 membered heterocyclyl, wherein said C₅₋₇-carbocyclyl or 4-12 membered heterocyclyl is optionally substituted with at least one substituent selected from the group consisting of: C₁₋₃-alkyl and —NR^{a}R^{b}; wherein R^{a} and R^{b} are independently at each occurrence hydrogen or C₁₋₄-alkyl;
L is C₁₋₁₅-alkyl optionally substituted by one or more substituents each independently selected from the group consisting of: C₃₋₅-carbocyclyl and —OH, wherein any one or more carbon atom of said C₁₋₁₅-alkyl is optionally replaced by oxygen or nitrogen, or a salt thereof.

7. The compound according to claim 6, wherein R³ is selected from the group consisting of: 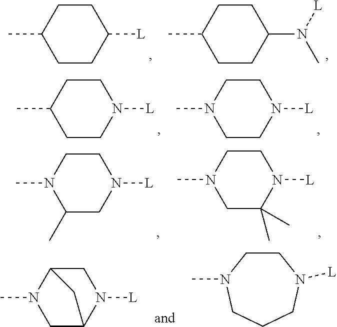
or a salt thereof.

8. The compound according to claim 6, wherein:
L is linear C₁₋₈-alkyl optionally substituted by one or more substituents each independently selected from the group consisting of: C₁₋₃-alkyl, C₃₋₅-carbocyclyl and —OH, wherein any one or more carbon atom of said linear C₁-s-alkyl is optionally replaced by oxygen or nitrogen,
or a salt thereof.

9. A conjugate comprising:
a compound of formula (I) as defined in claim 1,
a linker, and
an E3 ubiquitin ligase binding moiety,
wherein said linker connects said compound of formula (I) to said E3 ubiquitin ligase binding moiety,
or a salt thereof.

10. A compound of formula (III): 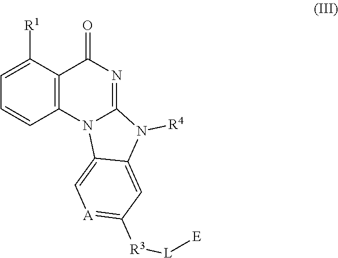
wherein:
A is —C(R²)— or —N—;
R¹ is halogen or —NH₂;
R⁴ is selected from the group consisting of: C₁₋₄-alkyl, C₃₋₆-carbocyclyl and 4-6 membered heterocyclyl, wherein said C₃₋₆-carbocyclyl or 4-6 membered heterocyclyl is optionally substituted with at least one substituent selected from the group consisting of: C₁₋₃-alkyl and —OH;
R³ is C₅₋₇-carbocyclyl or 4-12 membered heterocyclyl, wherein said C₅₋₇-carbocyclyl or 4-12 membered heterocyclyl is optionally substituted with at least one substituent selected from the group consisting of: C₁₋₃-alkyl and —NR^{a}R^{b}; wherein R^{a} and R^{b} are independently at each occurrence hydrogen or C₁₋₄-alkyl;
L is C₁₋₁₅-alkyl optionally substituted by one or more substituents each independently selected from the group consisting of: C₃_5-carbocyclyl and —OH, wherein any one or more carbon atom of said C₁₋₁₅-alkyl is optionally replaced by oxygen or nitrogen,
E is a group of formula (IIIa): 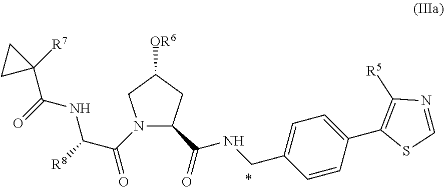
wherein:
R⁵ is selected from the group consisting of: hydrogen, C₁₋₃-alkyl and —COOR^{a};
R⁶ is hydrogen or —C(O)C₁₋₅alkyl;
R⁷ is selected from the group consisting of: halogen, —NR^{a}R^{b}, —CN, C₁₋₃-alkyl, C₁₋₃-haloalkyl, —C(O)OC₁₋₃-alkyl, C₃₋₇-carbocyclyl and 4-7 membered heterocyclyl;
or R⁷ is a C₃₋₅-alkyl forming a carbocyclyl together with the cyclopropyl to which R⁷ is bonded;
R⁸ is:
a branched C₃₋₆-alkyl optionally substituted by C₃₋₄-carbocyclyl;
or C₄₋₁₁carbocyclyl optionally substituted with at least one substituent selected from the group consisting of: C₁₋₃-alkyl and halogen;
R^{a} and R^{b} are independently at each occurrence hydrogen or C₁₋₄-alkyl;
* represents the atom to which L is bonded;
or a salt thereof.

11. The compound according to claim 10, wherein:
R⁵ is selected from the group consisting of: hydrogen, methyl and —C(O)OCH₂CH₃;
R⁶ is selected from the group consisting of: hydrogen, —C(O)CH₃ and —C(O)(CH₂)₃CH₃;
R⁷ is selected from the group consisting of: fluorine, —N(CH₃)₂, —CN, methyl, —CF₃, 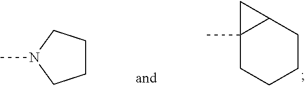
and
R⁸ is selected from the group consisting of: 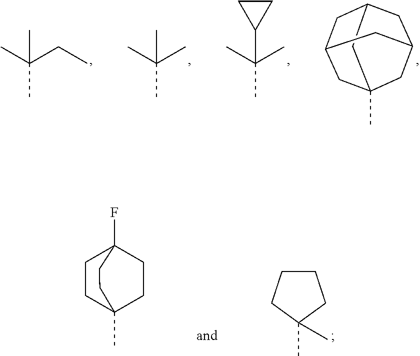
or a salt thereof.

12. The compound according to claim 10, wherein L is selected from the group consisting of: 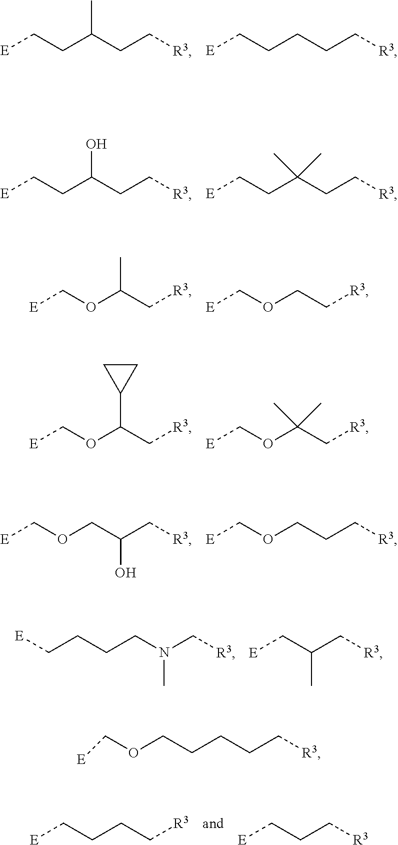
wherein [symbol] R³ denotes the bond between L and R³ and E [symbol] denotes the bond between E and L,
or a salt thereof.

13. The conjugate according to claim 9 being selected from the group consisting of: 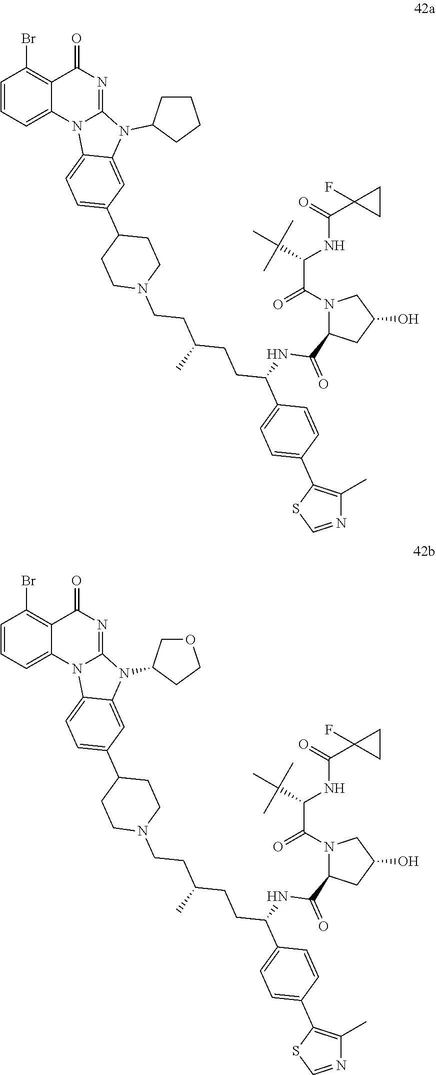 [SMILES: Cc1ncsc1-c1ccc([C@H](CC[C@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CC[C@H](C)CCN2CCC(c3ccc4c(c3)n([C@H]3CCOC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 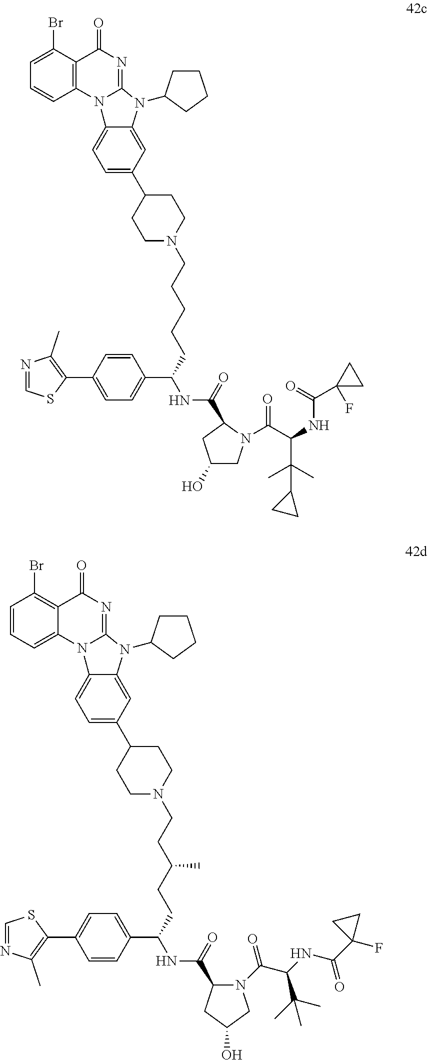 [SMILES: Cc1ncsc1-c1ccc([C@H](CCCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C2CC2)cc1 ; Cc1ncsc1-c1ccc([C@H](CC[C@@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 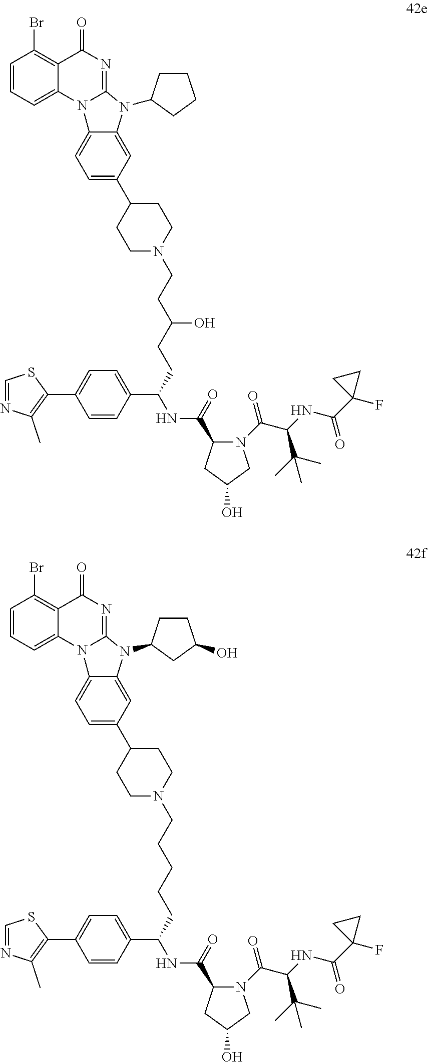 [SMILES: Cc1ncsc1-c1ccc([C@H](CCC(O)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CCCCCN2CCC(c3ccc4c(c3)n([C@H]3CC[C@@H](O)C3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 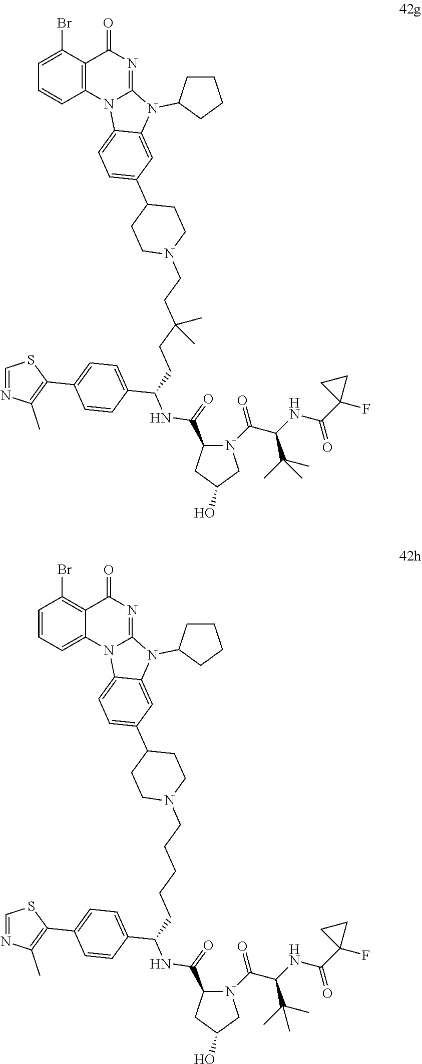 [SMILES: Cc1ncsc1-c1ccc([C@H](CCC(C)(C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CCCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 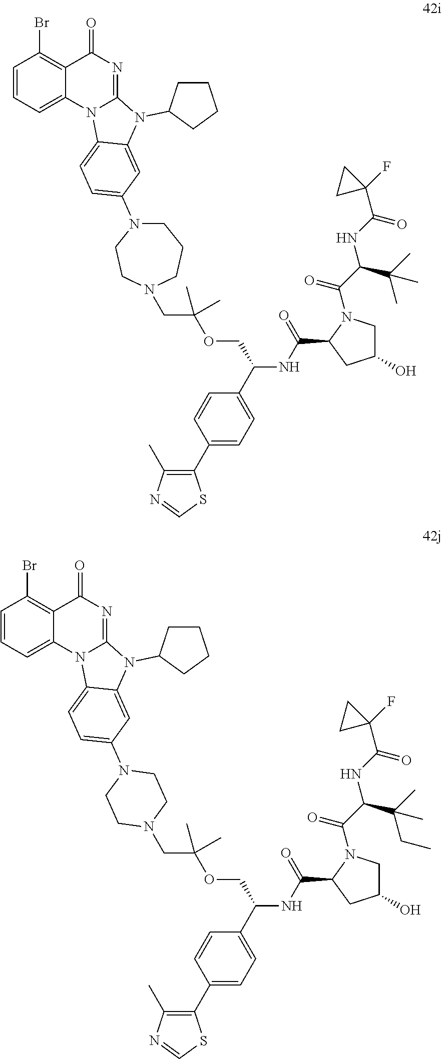 [SMILES: Cc1ncsc1-c1ccc([C@H](COC(C)(C)CN2CCCN(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; CCC(C)(C)[C@H](NC(=O)C1(F)CC1)C(=O)N1C[C@H](O)C[C@H]1C(=O)N[C@@H](COC(C)(C)CN1CCN(c2ccc3c(c2)n(C2CCCC2)c2nc(=O)c4c(Br)cccc4n32)CC1)c1ccc(-c2scnc2C)cc1] 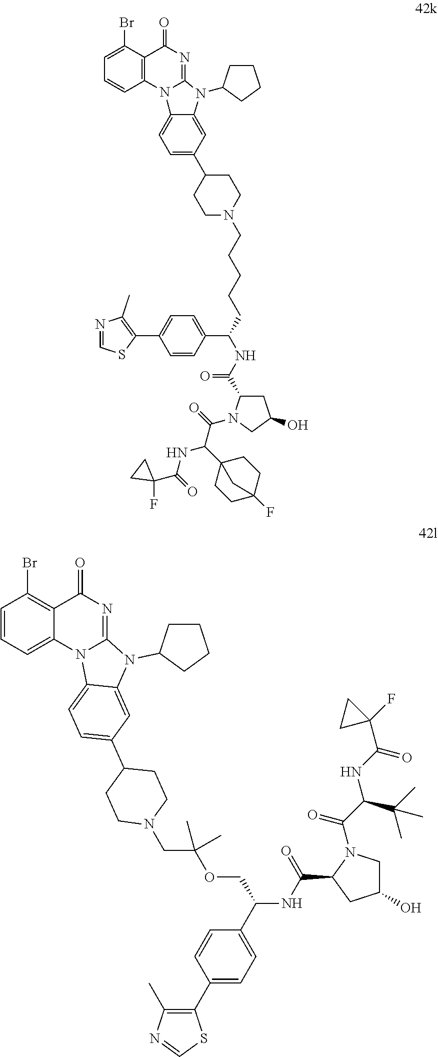 [SMILES: Cc1ncsc1-c1ccc([C@H](CCCCCN2CCC(c3ccc4c(c3)n(C3CCCC3)c3nc(=O)c5c(Br)cccc5n43)CC2)NC(=O)[C@@H]2C[C@@H](O)CN2C(=O)C(NC(=O)C2(F)CC2)C23CCC(F)(CC2)C3)cc1 ; Cc1ncsc1-c1ccc([C@H](COC(C)(C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 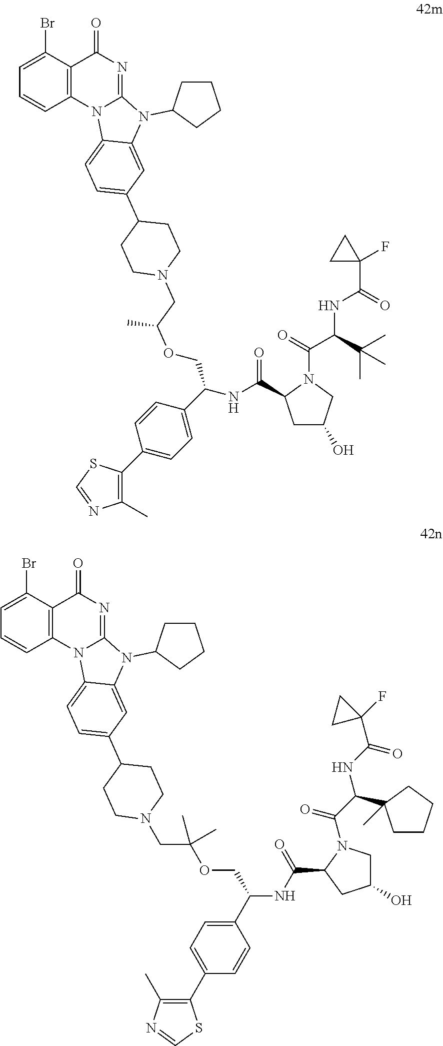 [SMILES: Cc1ncsc1-c1ccc([C@H](CO[C@H](C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](COC(C)(C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C2(C)CCCC2)cc1] 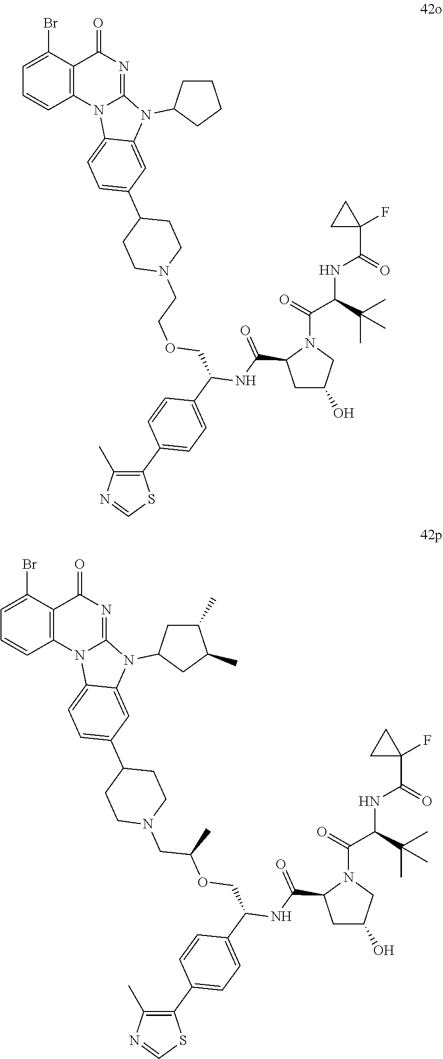 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CO[C@H](C)CN2CCC(c3ccc4c(c3)n(C3C[C@H](C)[C@@H](C)C3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 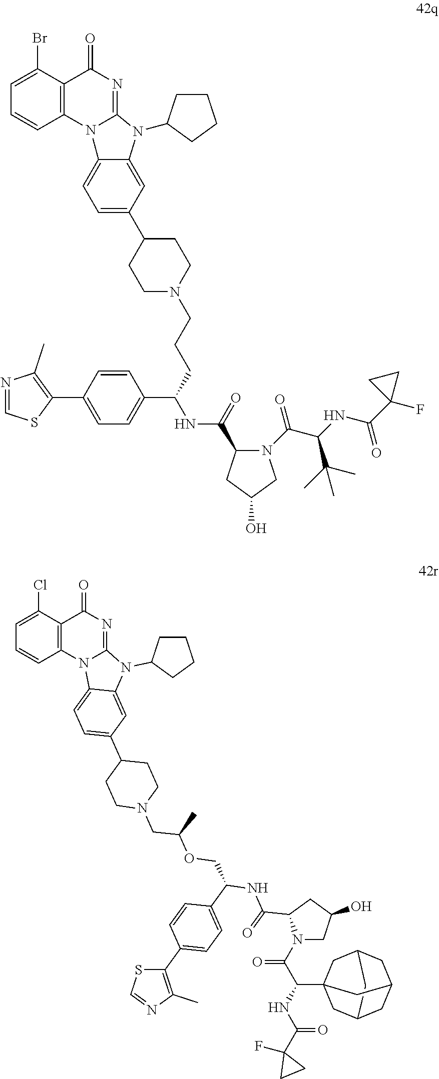 [SMILES: Cc1ncsc1-c1ccc([C@H](CCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CO[C@H](C)CN2CCC(c3ccc4c(c3)n(C3CCCC3)c3nc(=O)c5c(Cl)cccc5n43)CC2)NC(=O)[C@@H]2C[C@@H](O)CN2C(=O)[C@@H](NC(=O)C2(F)CC2)C23CC4CC(CC(C4)C2)C3)cc1] 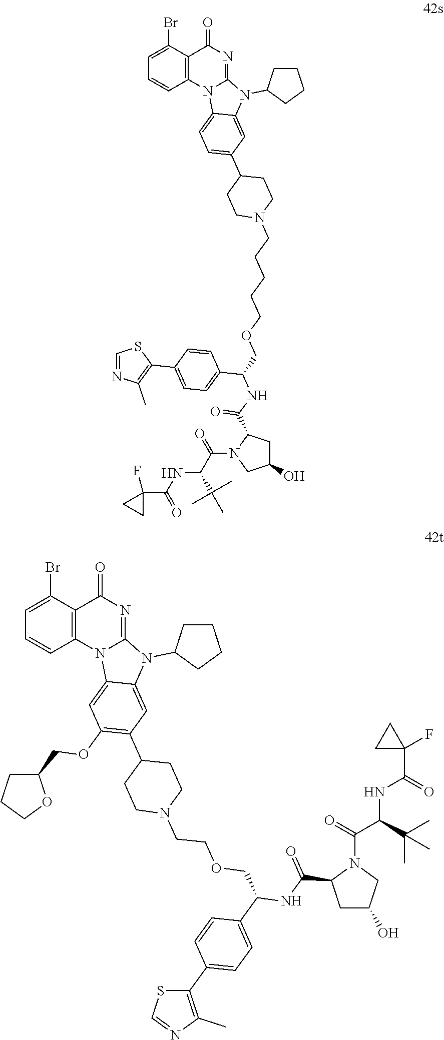 [SMILES: Cc1ncsc1-c1ccc([C@H](COCCCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](COCCN2CCC(c3cc4c(cc3OC[C@@H]3CCCO3)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] 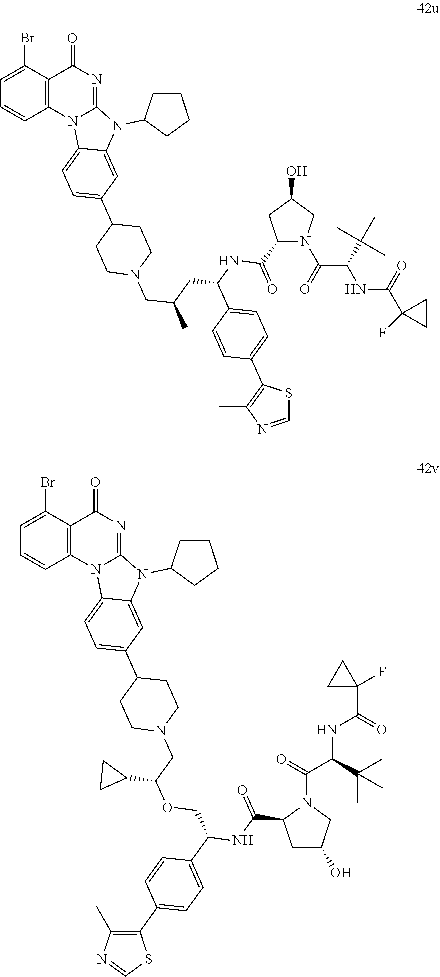 [SMILES: Cc1ncsc1-c1ccc([C@H](C[C@@H](C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CO[C@@H](CN2CCC(c3ccc4c(c3)n(C3CCCC3)c3nc(=O)c5c(Br)cccc5n43)CC2)C2CC2)NC(=O)[C@@H]2C[C@@H](O)CN2C(=O)[C@@H](NC(=O)C2(F)CC2)C(C)(C)C)cc1] 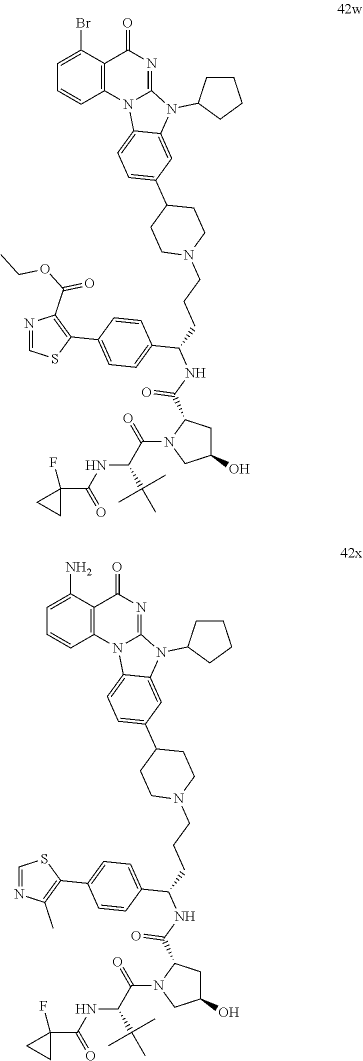 [SMILES: CCOC(=O)c1ncsc1-c1ccc([C@H](CCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CCCN2CCC(c3ccc4c(c3)n(C3CCCC3)c3nc(=O)c5c(N)cccc5n43)CC2)NC(=O)[C@@H]2C[C@@H](O)CN2C(=O)[C@@H](NC(=O)C2(F)CC2)C(C)(C)C)cc1] 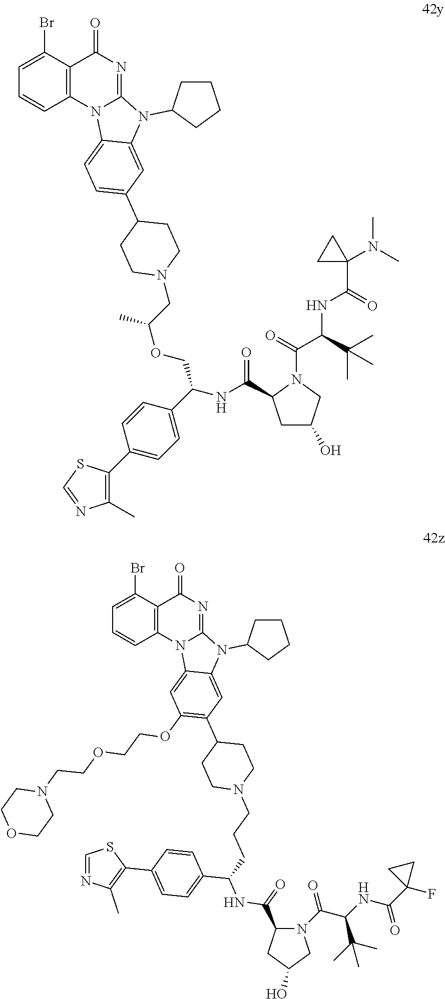 [SMILES: Cc1ncsc1-c1ccc([C@H](CO[C@H](C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(N(C)C)CC2)C(C)(C)C)cc1 ; Cc1ncsc1-c1ccc([C@H](CCCN2CCC(c3cc4c(cc3OCCOCCN3CCOCC3)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] 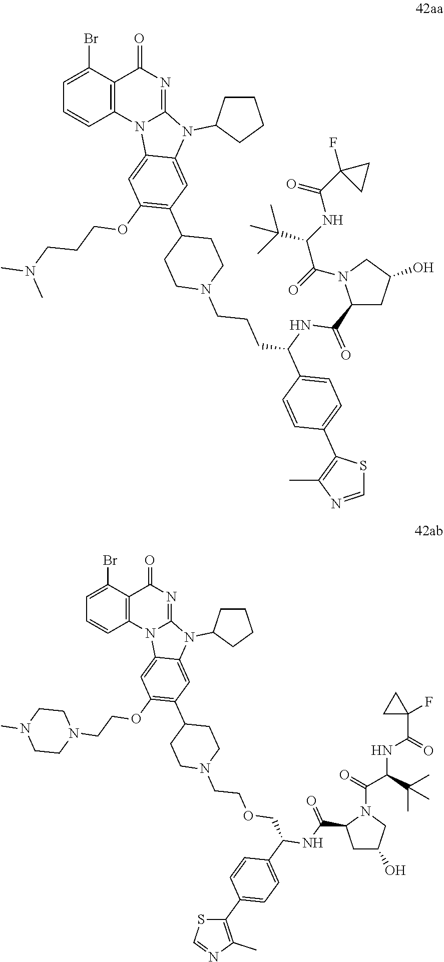 [SMILES: Cc1ncsc1-c1ccc([C@H](CCCN2CCC(c3cc4c(cc3OCCCN(C)C)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](COCCN2CCC(c3cc4c(cc3OCCN3CCN(C)CC3)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] 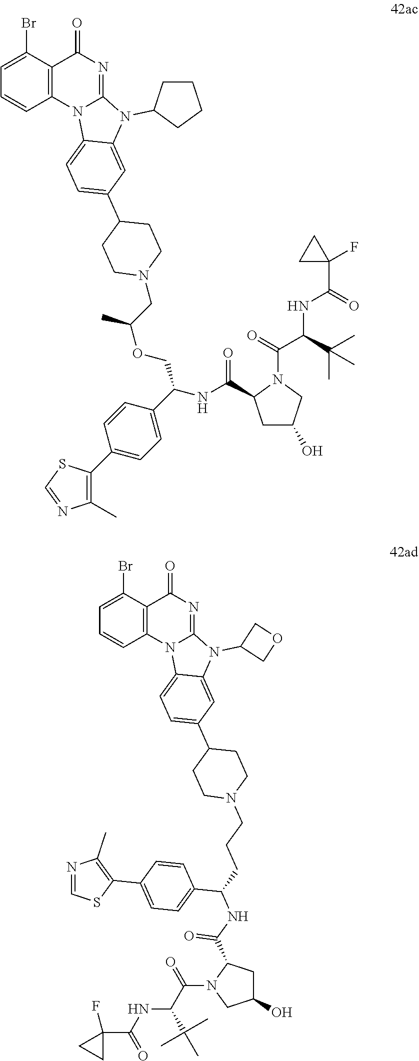 [SMILES: Cc1ncsc1-c1ccc([C@H](CO[C@@H](C)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](CCCN2CCC(c3ccc4c(c3)n(C3COC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 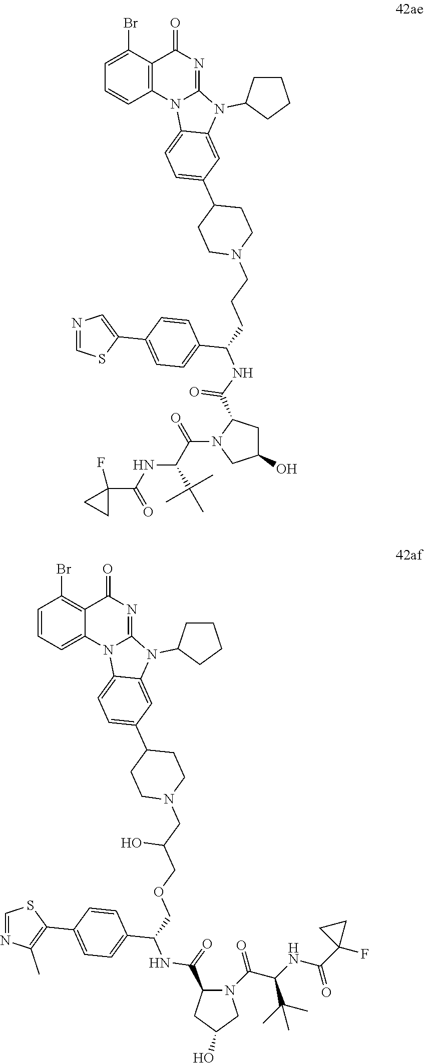 [SMILES: CC(C)(C)[C@H](NC(=O)C1(F)CC1)C(=O)N1C[C@H](O)C[C@H]1C(=O)N[C@@H](CCCN1CCC(c2ccc3c(c2)n(C2CCCC2)c2nc(=O)c4c(Br)cccc4n32)CC1)c1ccc(-c2cncs2)cc1 ; Cc1ncsc1-c1ccc([C@H](COCC(O)CN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 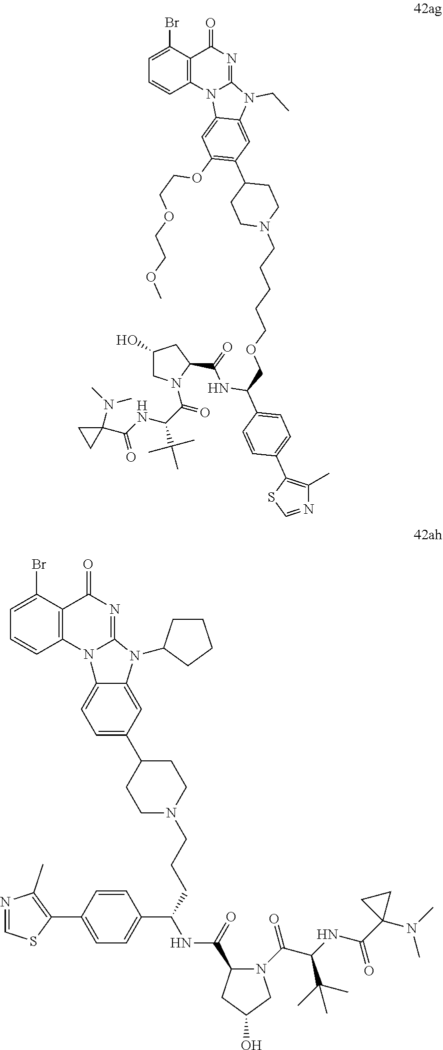 [SMILES: CCn1c2cc(C3CCN(CCCCCOC[C@H](NC(=O)[C@@H]4C[C@@H](O)CN4C(=O)[C@@H](NC(=O)C4(N(C)C)CC4)C(C)(C)C)c4ccc(-c5scnc5C)cc4)CC3)c(OCCOCCOC)cc2n2c3cccc(Br)c3c(=O)nc12 ; Cc1ncsc1-c1ccc([C@H](CCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(N(C)C)CC2)C(C)(C)C)cc1] 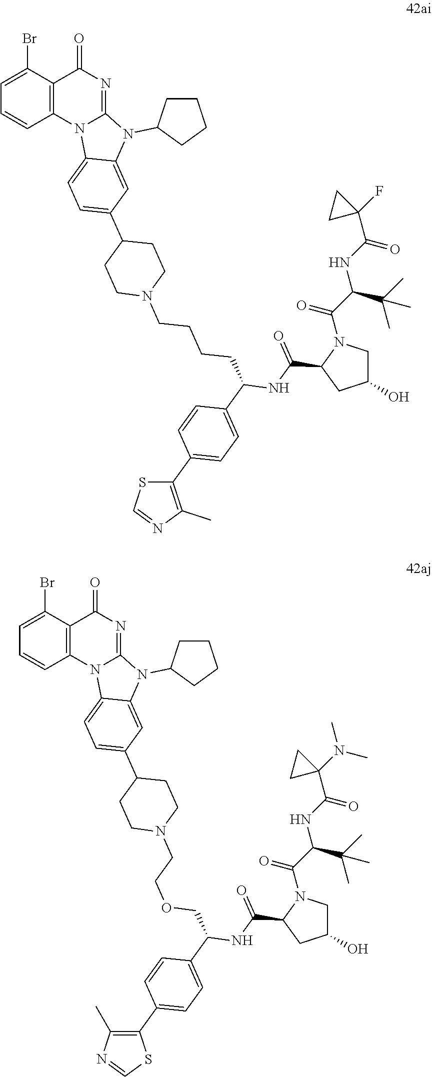 [SMILES: Cc1ncsc1-c1ccc([C@H](CCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(N(C)C)CC2)C(C)(C)C)cc1] 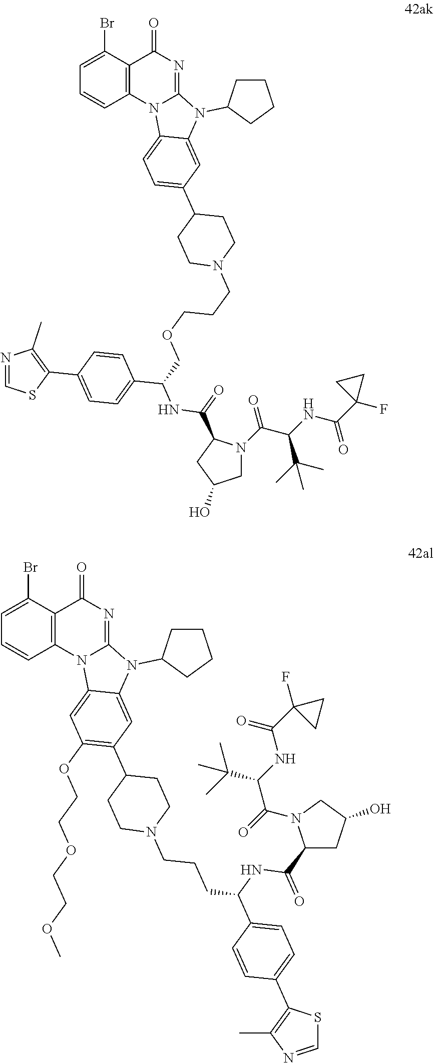 [SMILES: Cc1ncsc1-c1ccc([C@H](COCCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; COCCOCCOc1cc2c(cc1C1CCN(CCC[C@H](NC(=O)[C@@H]3C[C@@H](O)CN3C(=O)[C@@H](NC(=O)C3(F)CC3)C(C)(C)C)c3ccc(-c4scnc4C)cc3)CC1)n(C1CCCC1)c1nc(=O)c3c(Br)cccc3n21] 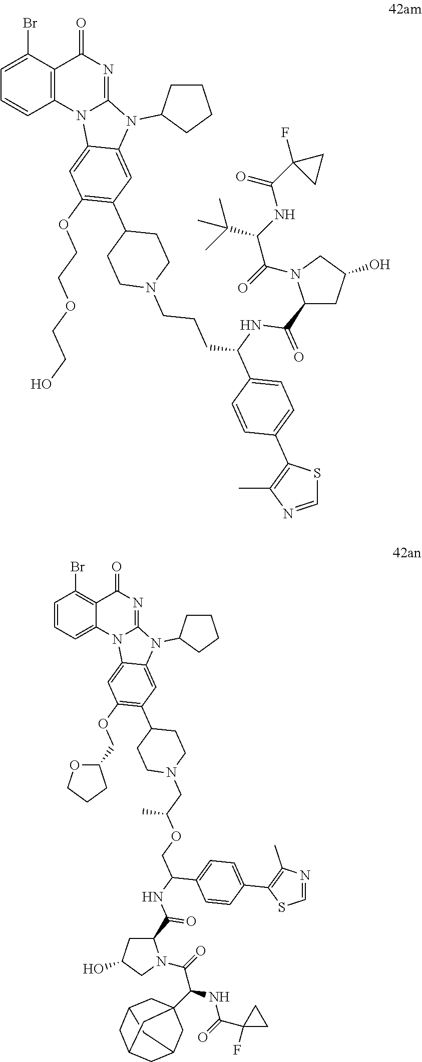 [SMILES: Cc1ncsc1-c1ccc([C@H](CCCN2CCC(c3cc4c(cc3OCCOCCO)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1 ; Cc1ncsc1-c1ccc(C(CO[C@H](C)CN2CCC(c3cc4c(cc3OC[C@@H]3CCCO3)n3c5cccc(Br)c5c(=O)nc3n4C3CCCC3)CC2)NC(=O)[C@@H]2C[C@@H](O)CN2C(=O)[C@@H](NC(=O)C2(F)CC2)C23CC4CC(CC(C4)C2)C3)cc1] 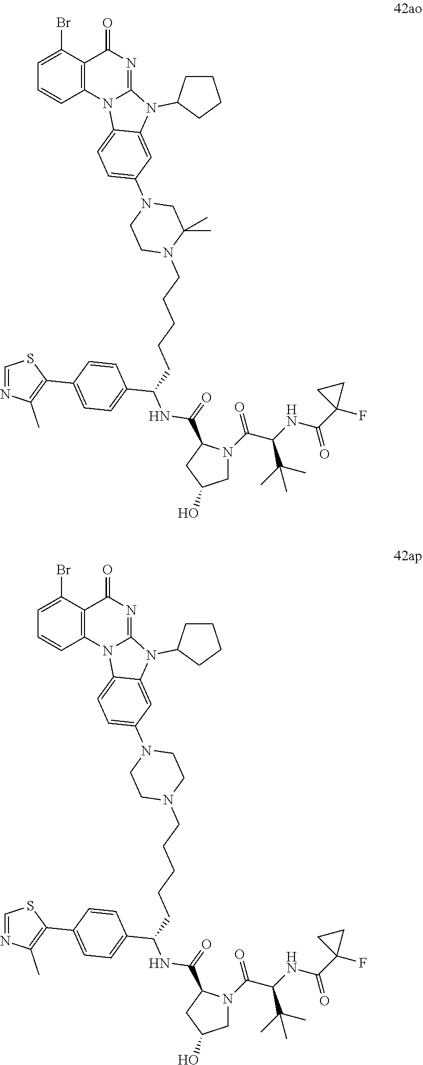 [SMILES: Cc1ncsc1-c1ccc([C@H](CCCCCN2CCN(c3ccc4c(c3)n(C3CCCC3)c3nc(=O)c5c(Br)cccc5n43)CC2(C)C)NC(=O)[C@@H]2C[C@@H](O)CN2C(=O)[C@@H](NC(=O)C2(F)CC2)C(C)(C)C)cc1 ; Cc1ncsc1-c1ccc([C@H](CCCCCN2CCN(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 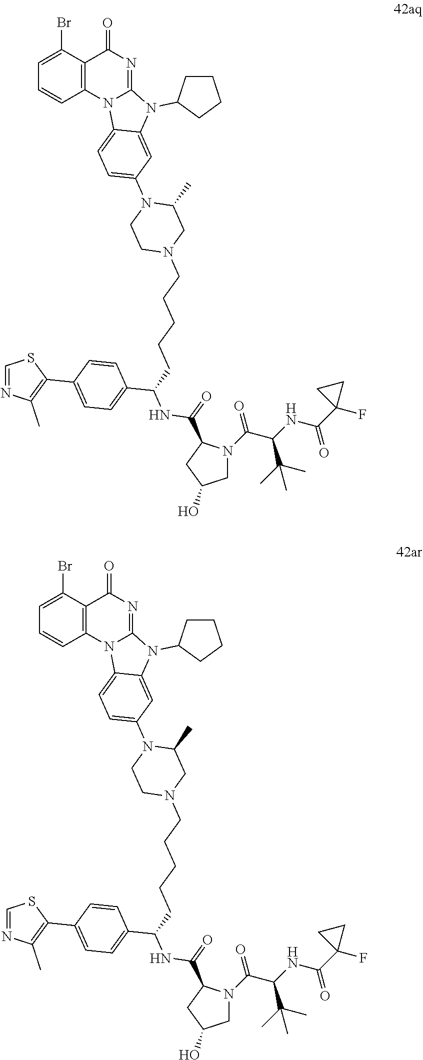 [SMILES: Cc1ncsc1-c1ccc([C@H](CCCCCN2CCN(c3ccc4c(c3)n(C3CCCC3)c3nc(=O)c5c(Br)cccc5n43)[C@H](C)C2)NC(=O)[C@@H]2C[C@@H](O)CN2C(=O)[C@@H](NC(=O)C2(F)CC2)C(C)(C)C)cc1 ; Cc1ncsc1-c1ccc([C@H](CCCCCN2CCN(c3ccc4c(c3)n(C3CCCC3)c3nc(=O)c5c(Br)cccc5n43)[C@@H](C)C2)NC(=O)[C@@H]2C[C@@H](O)CN2C(=O)[C@@H](NC(=O)C2(F)CC2)C(C)(C)C)cc1] 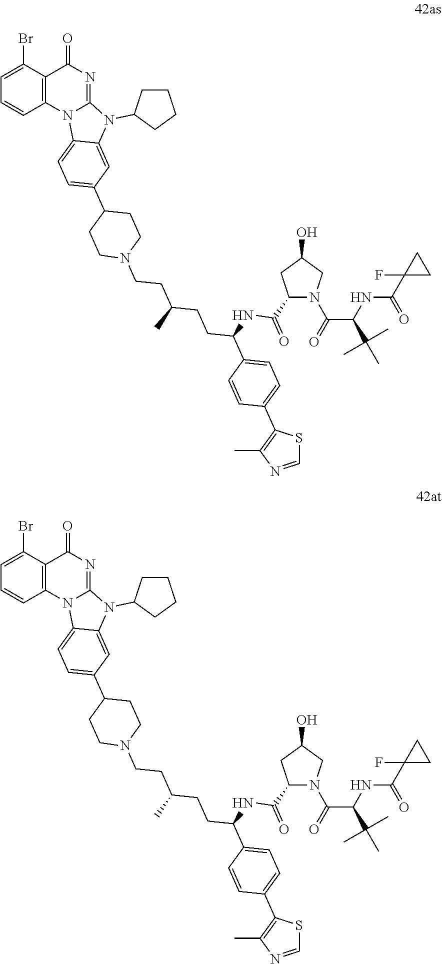 [SMILES: Cc1ncsc1-c1ccc([C@@H](CC[C@@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1 ; Cc1ncsc1-c1ccc([C@@H](CC[C@H](C)CCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(F)CC2)C(C)(C)C)cc1] 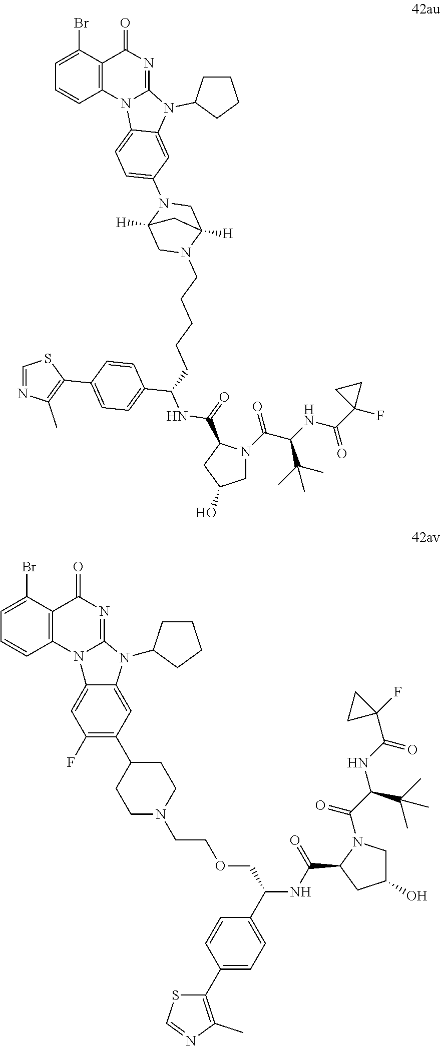 [SMILES: Cc1ncsc1-c1ccc([C@H](CCCCCN2C[C@@H]3C[C@H]2CN3c2ccc3c(c2)n(C2CCCC2)c2nc(=O)c4c(Br)cccc4n32)NC(=O)[C@@H]2C[C@@H](O)CN2C(=O)[C@@H](NC(=O)C2(F)CC2)C(C)(C)C)cc1 ; Cc1ncsc1-c1ccc([C@H](COCCN2CCC(c3cc4c(cc3F)n3c5cccc(Br)c5c(=O)nc3n4C3CCCC3)CC2)NC(=O)[C@@H]2C[C@@H](O)CN2C(=O)[C@@H](NC(=O)C2(F)CC2)C(C)(C)C)cc1] 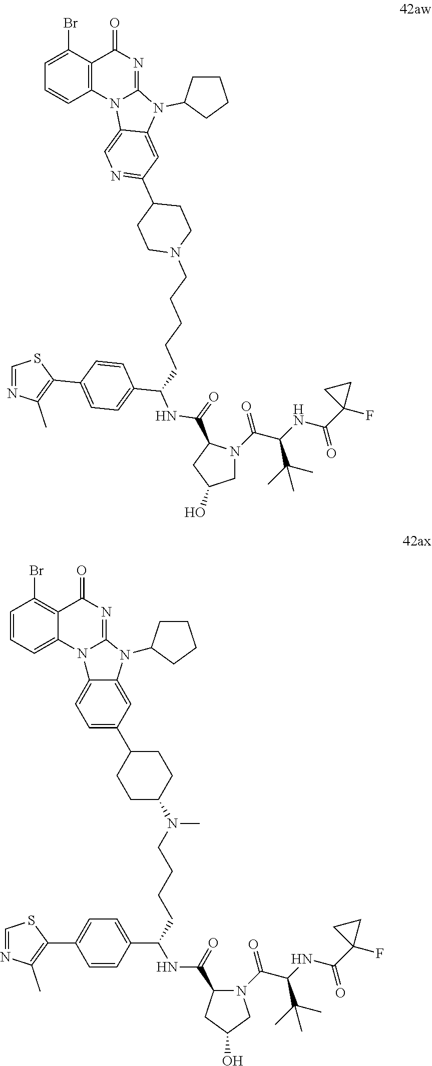 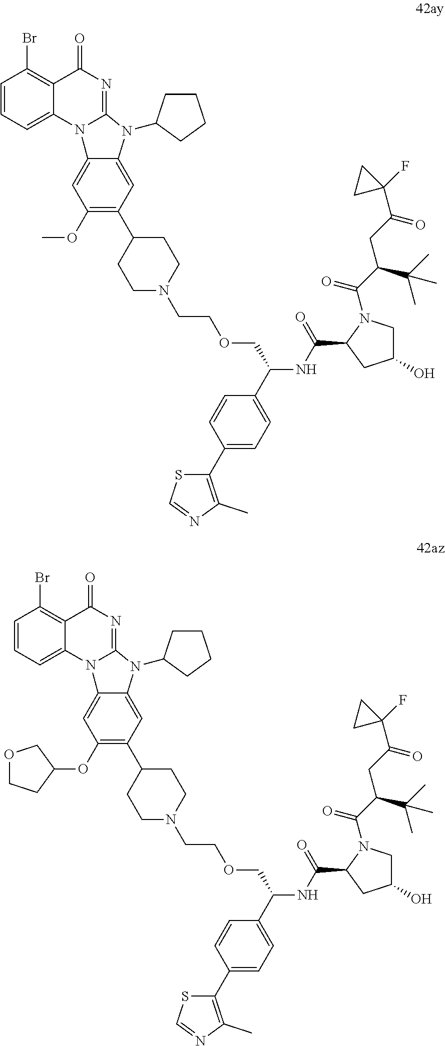 [SMILES: COc1cc2c(cc1C1CCN(CCOC[C@H](NC(=O)[C@@H]3C[C@@H](O)CN3C(=O)[C@@H](CC(=O)C3(F)CC3)C(C)(C)C)c3ccc(-c4scnc4C)cc3)CC1)n(C1CCCC1)c1nc(=O)c3c(Br)cccc3n21 ; Cc1ncsc1-c1ccc([C@H](COCCN2CCC(c3cc4c(cc3OC3CCOC3)n3c5cccc(Br)c5c(=O)nc3n4C3CCCC3)CC2)NC(=O)[C@@H]2C[C@@H](O)CN2C(=O)[C@@H](CC(=O)C2(F)CC2)C(C)(C)C)cc1] 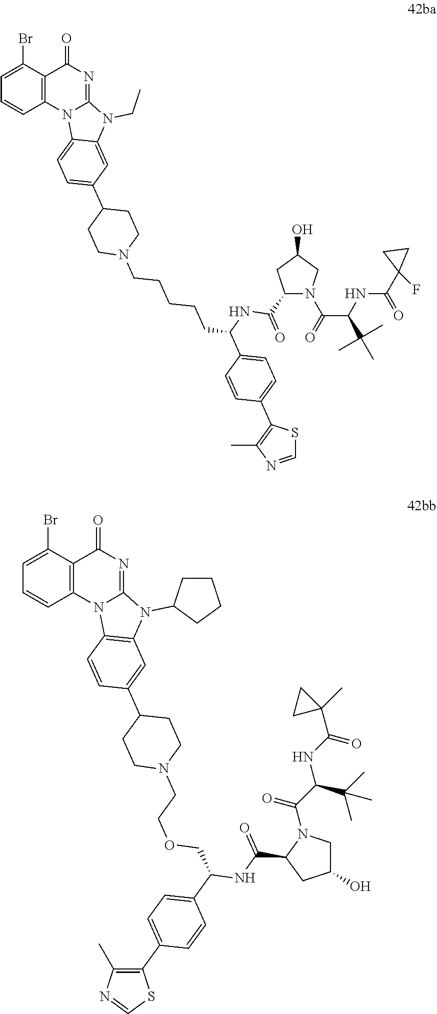 [SMILES: CCn1c2cc(C3CCN(CCCCC[C@H](NC(=O)[C@@H]4C[C@@H](O)CN4C(=O)[C@@H](NC(=O)C4(F)CC4)C(C)(C)C)c4ccc(-c5scnc5C)cc4)CC3)ccc2n2c3cccc(Br)c3c(=O)nc12 ; Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C)CC2)C(C)(C)C)cc1] 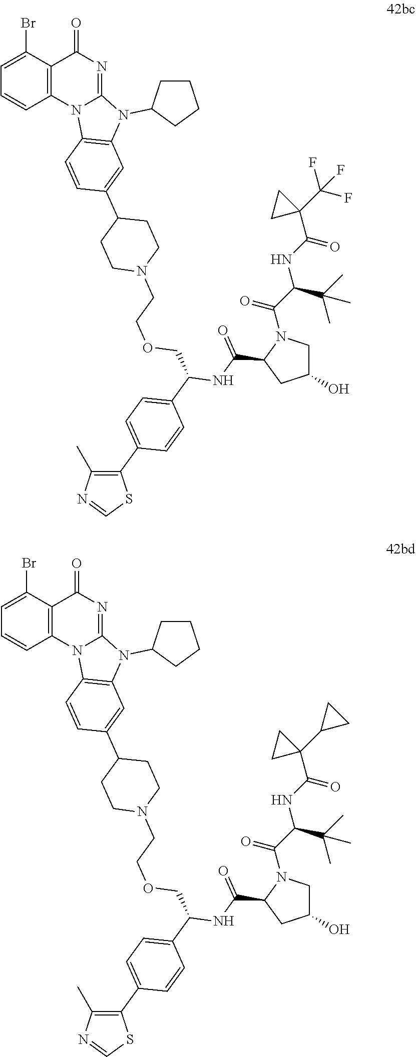 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C(F)(F)F)CC2)C(C)(C)C)cc1 ; Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C3CC3)CC2)C(C)(C)C)cc1] 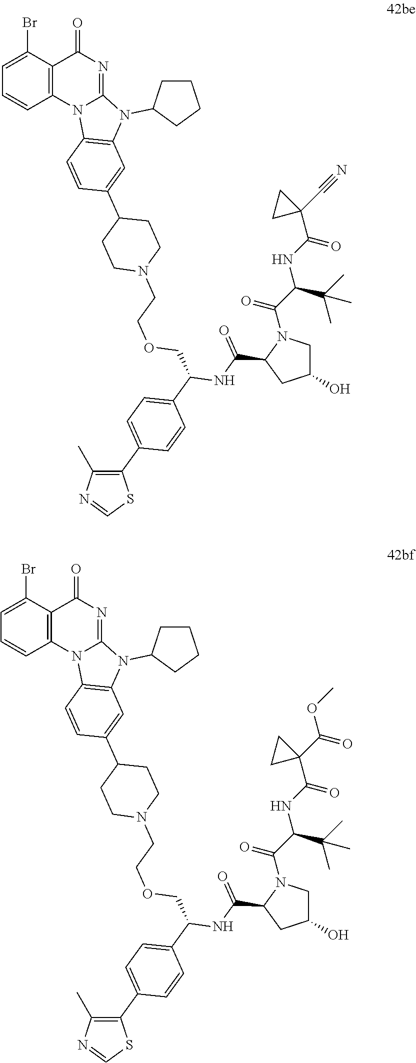 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C#N)CC2)C(C)(C)C)cc1 ; COC(=O)C1(C(=O)N[C@H](C(=O)N2C[C@H](O)C[C@H]2C(=O)N[C@@H](COCCN2CCC(c3ccc4c(c3)n(C3CCCC3)c3nc(=O)c5c(Br)cccc5n43)CC2)c2ccc(-c3scnc3C)cc2)C(C)(C)C)CC1] 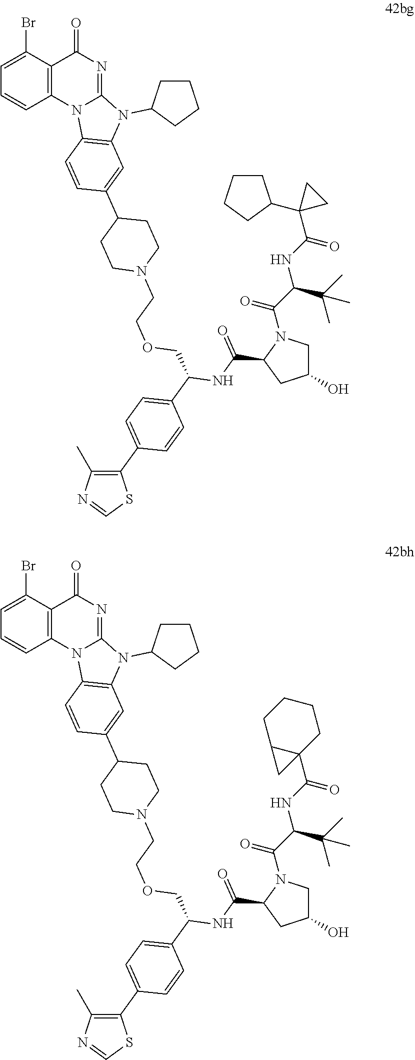 [SMILES: Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C2(C3CCCC3)CC2)C(C)(C)C)cc1 ; Cc1ncsc1-c1ccc([C@H](COCCN2CCC(c3ccc4c(c3)n(C3CCCC3)c3nc(=O)c5c(Br)cccc5n43)CC2)NC(=O)[C@@H]2C[C@@H](O)CN2C(=O)[C@@H](NC(=O)C23CCCCC2C3)C(C)(C)C)cc1] 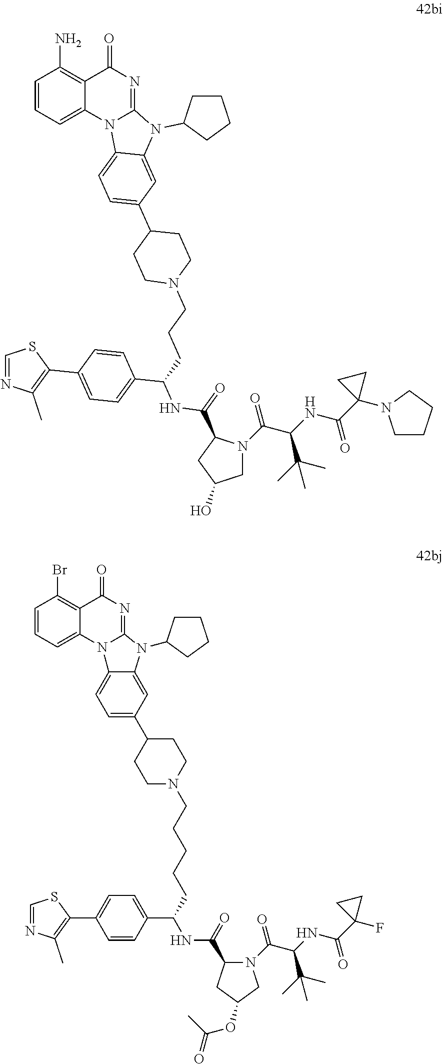 [SMILES: Cc1ncsc1-c1ccc([C@H](CCCN2CCC(c3ccc4c(c3)n(C3CCCC3)c3nc(=O)c5c(N)cccc5n43)CC2)NC(=O)[C@@H]2C[C@@H](O)CN2C(=O)[C@@H](NC(=O)C2(N3CCCC3)CC2)C(C)(C)C)cc1 ; CC(=O)O[C@@H]1C[C@@H](C(=O)N[C@@H](CCCCCN2CCC(c3ccc4c(c3)n(C3CCCC3)c3nc(=O)c5c(Br)cccc5n43)CC2)c2ccc(-c3scnc3C)cc2)N(C(=O)[C@@H](NC(=O)C2(F)CC2)C(C)(C)C)C1] 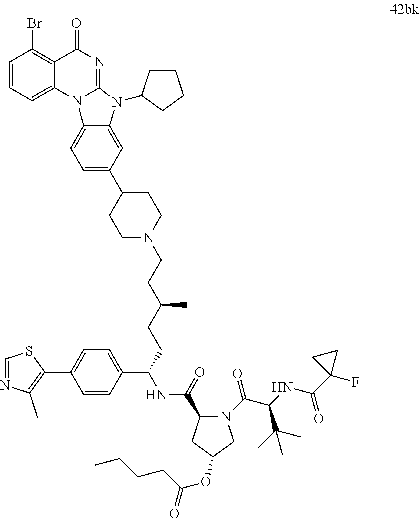 [SMILES: CCCCC(=O)O[C@@H]1C[C@@H](C(=O)N[C@@H](CC[C@H](C)CCN2CCC(c3ccc4c(c3)n(C3CCCC3)c3nc(=O)c5c(Br)cccc5n43)CC2)c2ccc(-c3scnc3C)cc2)N(C(=O)[C@@H](NC(=O)C2(F)CC2)C(C)(C)C)C1]
or a salt thereof.

14. (canceled)

15. (canceled)

16. (canceled)

17. A method for the treatment and/or prevention of cancer comprising administering to a human being in need thereof a therapeutically effective amount of the conjugate as defined in claim 9, or a pharmaceutically acceptable salt thereof.

18. The method according to claim 17, wherein the conjugate, compound or pharmaceutically acceptable salt is administered in combination with a therapeutically effective amount of at least one other pharmacologically active substance.

19. A pharmaceutical composition comprising the conjugate as defined in claim 9, or a pharmaceutically acceptable salt thereof, and one or more pharmaceutically acceptable excipient(s).
